SEQUENCE LISTING (119 sequences)

SEQ ID NO: 1 - DNA, 1410 bp, Rubus suavissimus
atggcttgcacttccaccggaagcacaaagcctcatgttgtttgtactaatgttgcttctcaaagccacattaaggcaatgcttaaattagccaaaatccttcaccatagaggctttcacgtaacctttgttaacacagaatccatccacaagcgttttcttgaatctcaaggacccaattccctcaacggcttagctgattttcgcttcgaaacccttccagattcagacgaaaatgcccccagaaaccatctgttggctccatttcgtgacctattgatgaaactcaacgatactagtcctccggtgacttgcattgtttcaaatggtttcatgtccacattcacaatcactgctgcagaagaactaagtgtccctattgcattgttctacagtttttctgcttgcagctttatgggattaaagcaattccgcactttacgagaaaaaggccttacaccactcaaagatgagagctgtttgacaaatggatttttggataaagttatagattgggttccaggaatgaagggtatctgtttaaagcatctcccaaccttctttagaactacaaatcctgatgataaattgttcaacatcagcatggaaacaacagaagcagtagacaaagcttcagcagttgttcttctcacctttgatgctttggaaaaagatgttttggcagctctctccgcttctatttctccacctgtttatacaattggtcccctccaattacttctcaaccaaataccagaagaccctctggaggctatgggatacagtattttgaaagaagaatcagaatgtctccaatggctgaactccaaacctccaaattcagttgtttatgtcaactttggcagcatagcggtcttgacagcagaacagcttctggaatttggatggggacttgcaaatactaagcttcccttcttctgggttattaggcctgatttggttgttggcaagtcggcggttttgcctccagagttcgaagctgaaaccaaagaccggggtctaatcgcaagttggtgcccccaagaacaagtcctaaaccacccatcagttggagggtttctgacacatagtggttggaattcaaccattgaaagcgtgacagctggagtgccgatgctgtgttggccattttttgcagaccagccaacaaacagttactacacttgcaataaatggggaattggcatggagatcaacaatgatgtcaagagaaatgatgtagaaaagcttgtaaaggagttaatggagggagagaagggtaagaaaatgaaaagcaaggtcttggagtggaagaaacttgcagaagaagcaactgttccacatggttcttcatcgattaatttagataatctagtgaatcaagttctacacagtgtaagacaaagctag

SEQ ID NO: 2 - protein, 469 aa, Rubus suavissimus
MACTSTGSTKPHVVCTNVASQSHIKAMLKLAKILHHRGFHVTFVNTESIHKRFLESQGPNSLNGLADFRFETLPDSDENAPRNHLLAPFRDLLMKLNDTSPPVTCIVSNGFMSTFTITAAEELSVPIALFYSFSACSFMGLKQFRTLREKGLTPLKDESCLTNGFLDKVIDWVPGMKGICLKHLPTFFRTTNPDDKLFNISMETTEAVDKASAVVLLTFDALEKDVLAALSASISPPVYTIGPLQLLLNQIPEDPLEAMGYSILKEESECLQWLNSKPPNSVVYVNFGSIAVLTAEQLLEFGWGLANTKLPFFWVIRPDLVVGKSAVLPPEFEAETKDRGLIASWCPQEQVLNHPSVGGFLTHSGWNSTIESVTAGVPMLCWPFFADQPTNSYYTCNKWGIGMEINNDVKRNDVEKLVKELMEGEKGKKMKSKVLEWKKLAEEATVPHGSSSINLDNLVNQVLHSVRQS

SEQ ID NO: 3 - DNA, 1383 bp, Stevia rebaudiana
atggcagagcaacaaaagatcaaaaagtcacctcacgtcttacttattccatttcctctgcaaggacatatcaacccattcatacaatttgggaaaagattgattagtaagggtgtaaagacaacactggtaaccactatccacactttgaattctactctgaaccactcaaatactactactacaagtatagaaattcaagctatatcagacggatgcgatgagggtggctttatgtctgccggtgaatcttacttggaaacattcaagcaagtgggatccaagtctctggccgatctaatcaaaaagttacagagtgaaggcaccacaattgacgccataatctacgattctatgacagagtgggttttagacgttgctatcgaatttggtattgatggaggttcctttttcacacaagcatgtgttgtgaattctctatactaccatgtgcataaagggttaatctctttaccattgggtgaaactgtttcagttccaggttttccagtgttacaacgttgggaaaccccattgatcttacaaaatcatgaacaaatacaatcaccttggtcccagatgttgtttggtcaattcgctaacatcgatcaagcaagatgggtctttactaattcattctataagttagaggaagaggtaattgaatggactaggaagatctggaatttgaaagtcattggtccaacattgccatcaatgtatttggacaaaagacttgatgatgataaagataatggtttcaatttgtacaaggctaatcatcacgaatgtatgaattggctggatgacaaaccaaaggaatcagttgtatatgttgctttcggctctcttgttaaacatggtccagaacaagttgaggagattacaagagcacttatagactctgacgtaaactttttgtgggtcattaagcacaaagaggaggggaaactgccagaaaacctttctgaagtgataaagaccggaaaaggtctaatcgttgcttggtgtaaacaattggatgttttagctcatgaatctgtaggctgttttgtaacacattgcggattcaactctacactagaagccatttccttaggcgtacctgtcgttgcaatgcctcagttctccgatcagacaaccaacgctaaacttttggacgaaatactaggggtgggtgtcagagttaaagcagacgagaatggtatcgtcagaagagggaacctagcttcatgtatcaaaatgatcatggaagaggaaagaggagttatcataaggaaaaacgcagttaagtggaaggatcttgcaaaggttgccgtccatgaaggcggctcttcagataatgatattgttgaatttgtgtccgaactaatcaaagcctaa

SEQ ID NO: 4 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 5 - DNA, 1586 bp, Stevia rebaudiana
atggatgcaatggctacaactgagaagaaaccacacgtcatcttcataccatttccagcacaaagccacattaaagccatgctcaaactagcacaacttctccaccacaaaggactccagataaccttcgtcaacaccgacttcatccacaaccagtttcttgaatcatcgggcccacattgtctagacggtgcaccgggtttccggttcgaaaccattccggatggtgtttctcacagtccggaagcgagcatcccaatcagagaatcactcttgagatccattgaaaccaacttcttggatcgtttcattgatcttgtaaccaaacttccggatcctccgacttgtattatctcagatgggttcttgtcggttttcacaattgacgctgcaaaaaagcttggaattccggtcatgatgtattggacacttgctgcctgtgggttcatgggtttttaccatattcattctctcattgagaaaggatttgcaccacttaaagatgcaagttacttgacaaatgggtatttggacaccgtcattgattgggttccgggaatggaaggcatccgtctcaaggatttcccgctggactggagcactgacctcaatgacaaagttttgatgttcactacggaagctcctcaaaggtcacacaaggtttcacatcatattttccacacgttcgatgagttggagcctagtattataaaaactttgtcattgaggtataatcacatttacaccatcggcccactgcaattacttcttgatcaaatacccgaagagaaaaagcaaactggaattacgagtctccatggatacagtttagtaaaagaagaaccagagtgtttccagtggcttcagtctaaagaaccaaattccgtcgtttatgtaaattttggaagtactacagtaatgtctttagaagacatgacggaatttggttggggacttgctaatagcaaccattatttcctttggatcatccgatcaaacttggtgataggggaaaatgcagttttgccccctgaacttgaggaacatataaagaaaagaggctttattgctagctggtgttcacaagaaaaggtcttgaagcacccttcggttggagggttcttgactcattgtgggtggggatcgaccatcgagagcttgtctgctggggtgccaatgatatgctggccttattcgtgggaccagctgaccaactgtaggtatatatgcaaagaatgggaggttgggctcgagatgggaaccaaagtgaaacgagatgaagtcaagaggcttgtacaagagttgatgggagaaggaggtcacaaaatgaggaacaaggctaaagattggaaagaaaaggctcgcattgcaatagctcctaacggttcatcttctttgaacatagacaaaatggtcaaggaaatcaccgtgctagcaagaaactagttacaaagttgtttcacattgtgctttctatttaagatgtaactttgttctaatttaatattgtctagatgtattgaaccataagtttagttggtctcaggaattgatttttaatgaaataatggtcattaggggtgagt

SEQ ID NO: 6 - DNA, 1446 bp, Artificial Sequence
  Codon-optimized UGT85C2
atggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 7 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 8 - DNA, 1377 bp, Artificial Sequence
  Codon-optimized UGT76G1
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 9 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 10 - DNA, 1422 bp, Artificial Sequence
  Codon-optimized UGT91D2e
atggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaa

SEQ ID NO: 11 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 12 - DNA, 1422 bp, Artificial Sequence
  Codon-optimized UGT91D2e-b
atggctacttctgattccatcgttgacgatagaaagcaattgcatgttgctacttttccatggttggctttcggtcatattttgccatacttgcaattgtccaagttgattgctgaaaagggtcacaaggtttcattcttgtctaccaccagaaacatccaaagattgtcctctcatatctccccattgatcaacgttgttcaattgactttgccaagagtccaagaattgccagaagatgctgaagctactactgatgttcatccagaagatatcccttacttgaaaaaggcttccgatggtttacaaccagaagttactagattcttggaacaacattccccagattggatcatctacgattatactcattactggttgccatccattgctgcttcattgggtatttctagagcccatttctctgttactactccatgggctattgcttatatgggtccatctgctgatgctatgattaacggttctgatggtagaactaccgttgaagatttgactactccaccaaagtggtttccatttccaacaaaagtctgttggagaaaacacgatttggctagattggttccatacaaagctccaggtatttctgatggttacagaatgggtatggttttgaaaggttccgattgcttgttgtctaagtgctatcatgaattcggtactcaatggttgcctttgttggaaacattgcatcaagttccagttgttccagtaggtttgttgccaccagaaattccaggtgacgaaaaagacgaaacttgggtttccatcaaaaagtggttggatggtaagcaaaagggttctgttgtttatgttgctttgggttccgaagctttggtttctcaaaccgaagttgttgaattggctttgggtttggaattgtctggtttgccatttgtttgggcttacagaaaacctaaaggtccagctaagtctgattctgttgaattgccagatggtttcgttgaaagaactagagatagaggtttggtttggacttcttgggctccacaattgagaattttgtctcatgaatccgtctgtggtttcttgactcattgtggttctggttctatcgttgaaggtttgatgtttggtcacccattgattatgttgccaatctttggtgaccaaccattgaacgctagattattggaagataagcaagtcggtatcgaaatcccaagaaatgaagaagatggttgcttgaccaaagaatctgttgctagatctttgagatccgttgtcgttgaaaaagaaggtgaaatctacaaggctaacgctagagaattgtccaagatctacaacgataccaaggtcgaaaaagaatacgtttcccaattcgttgactacttggaaaagaatgctagagctgttgccattgatcatgaatcttga

SEQ ID NO: 13 - protein, 473 aa, Artificial Sequence
  UGT91D2e-b
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 14 - DNA, 1389 bp, Oryza sativa
atggactccggctactcctcctcctacgccgccgccgccgggatgcacgtcgtgatctgcccgtggctcgccttcggccacctgctcccgtgcctcgacctcgcccagcgcctcgcgtcgcggggccaccgcgtgtcgttcgtctccacgccgcggaacatatcccgcctcccgccggtgcgccccgcgctcgcgccgctcgtcgccttcgtggcgctgccgctcccgcgcgtcgaggggctccccgacggcgccgagtccaccaacgacgtcccccacgacaggccggacatggtcgagctccaccggagggccttcgacgggctcgccgcgcccttctcggagttcttgggcaccgcgtgcgccgactgggtcatcgtcgacgtcttccaccactgggccgcagccgccgctctcgagcacaaggtgccatgtgcaatgatgttgttgggctctgcacatatgatcgcttccatagcagacagacggctcgagcgcgcggagacagagtcgcctgcggctgccgggcagggacgcccagcggcggcgccaacgttcgaggtggcgaggatgaagttgatacgaaccaaaggctcatcgggaatgtccctcgccgagcgcttctccttgacgctctcgaggagcagcctcgtcgtcgggcggagctgcgtggagttcgagccggagaccgtcccgctcctgtcgacgctccgcggtaagcctattaccttccttggccttatgccgccgttgcatgaaggccgccgcgaggacggcgaggatgccaccgtccgctggctcgacgcgcagccggccaagtccgtcgtgtacgtcgcgctaggcagcgaggtgccactgggagtggagaaggtccacgagctcgcgctcgggctggagctcgccgggacgcgcttcctctgggctcttaggaagcccactggcgtctccgacgccgacctcctccccgccggcttcgaggagcgcacgcgcggccgcggcgtcgtggcgacgagatgggttcctcagatgagcatactggcgcacgccgccgtgggcgcgttcctgacccactgcggctggaactcgaccatcgaggggctcatgttcggccacccgcttatcatgctgccgatcttcggcgaccagggaccgaacgcgcggctaatcgaggcgaagaacgccggattgcaggtggcaagaaacgacggcgatggatcgttcgaccgagaaggcgtcgcggcggcgattcgtgcagtcgcggtggaggaagaaagcagcaaagtgtttcaagccaaagccaagaagctgcaggagatcgtcgcggacatggcctgccatgagaggtacatcgacggattcattcagcaattgagatcttacaaggattga

SEQ ID NO: 15 - DNA, 1389 bp, Artificial Sequence
  Codon-optimized EUGT11
atggatagtggctactcctcatcttatgctgctgccgctggtatgcacgttgtgatctgcccttggttggcctttggtcacctgttaccatgtctggatttagcccaaagactggcctcaagaggccatagagtatcatttgtgtctactcctagaaatatctctcgtttaccaccagtcagacctgctctagctcctctagttgcattcgttgctcttccacttccaagagtagaaggattgccagacggcgctgaatctactaatgacgtaccacatgatagacctgacatggtcgaattgcatagaagagcctttgatggattggcagctccattttctgagttcctgggcacagcatgtgcagactgggttatagtcgatgtatttcatcactgggctgctgcagccgcattggaacataaggtgccttgtgctatgatgttgttagggtcagcacacatgatcgcatccatagctgatagaagattggaaagagctgaaacagaatccccagccgcagcaggacaaggtaggccagctgccgccccaacctttgaagtggctagaatgaaattgattcgtactaaaggtagttcagggatgagtcttgctgaaaggttttctctgacattatctagatcatcattagttgtaggtagatcctgcgtcgagttcgaacctgaaacagtacctttactatctactttgagaggcaaacctattactttccttggtctaatgcctccattacatgaaggaaggagagaagatggtgaagatgctactgttaggtggttagatgcccaacctgctaagtctgttgtttacgttgcattgggttctgaggtaccactaggggtggaaaaggtgcatgaattagcattaggacttgagctggccggaacaagattcctttgggctttgagaaaaccaaccggtgtttctgacgccgacttgctaccagctgggttcgaagagagaacaagaggccgtggtgtcgttgctactagatgggtcccacaaatgagtattctagctcatgcagctgtaggggcctttctaacccattgcggttggaactcaacaatagaaggactgatgtttggtcatccacttattatgttaccaatctttggcgatcagggacctaacgcaagattgattgaggcaaagaacgcaggtctgcaggttgcacgtaatgatggtgatggttcctttgatagagaaggcgttgcagctgccatcagagcagtcgccgttgaggaagagtcatctaaagttttccaagctaaggccaaaaaattacaagagattgtggctgacatggcttgtcacgaaagatacatcgatggtttcatccaacaattgagaagttataaagactaa

SEQ ID NO: 16 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 17 - protein, 465 aa, Artificial Sequence
  UGT91D2e-b-EUGT11 chimera 3
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIARNDGDGSFDREGVAAAIRAVAVEEESSKVFQAKAKKLQEIVADMACHERYIDGFIQQLRSYKD

SEQ ID NO: 18 - protein, 470 aa, Artificial Sequence
  UGT91D2e-b-EUGT11 chimera 7
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVPRNEEDGCLTKESVARSLRSVVVEKEGEIYKANARELSKIYNDTKVEKEYVSQFVDYLEKNARAVAIDHES

SEQ ID NO: 19 - DNA, 1086 bp, Artificial Sequence
  Codon-optimized GGPPS
atggctttggtaaacccaaccgctcttttctatggtacctctatcagaacaagacctacaaacttactaaatccaactcaaaagctaagaccagtttcatcatcttccttaccttctttctcatcagttagtgcgattcttactgaaaaacatcaatctaatccttctgagaacaacaatttgcaaactcatctagaaactcctttcaactttgatagttatatgttggaaaaagtcaacatggttaacgaggcgcttgatgcatctgtcccactaaaagacccaatcaaaatccatgaatccatgagatactctttattggcaggcggtaagagaatcagaccaatgatgtgtattgcagcctgcgaaatagtcggaggtaatatccttaacgccatgccagccgcatgtgccgtggaaatgattcatactatgtctttggtgcatgacgatcttccatgtatggataatgatgacttcagaagaggtaaacctatttcacacaaggtctacggggaggaaatggcagtattgaccggcgatgctttactaagtttatctttcgaacatatagctactgctacaaagggtgtatcaaaggatagaatcgtcagagctataggggagttggcccgttcagttggctccgaaggtttagtggctggacaagttgtagatatcttgtcagagggtgctgatgttggattagatcacctagaatacattcacatccacaaaacagcaatgttgcttgagtcctcagtagttattggcgctatcatgggaggaggatctgatcagcagatcgaaaagttgagaaaattcgctagatctattggtctactattccaagttgtggatgacattttggatgttacaaaatctaccgaagagttggggaaaacagctggtaaggatttgttgacagataagacaacttacccaaagttgttaggtatagaaaagtccagagaatttgccgaaaaacttaacaaggaagcacaagagcaattaagtggctttgatagacgtaaggcagctcctttgatcgcgttagccaactacaatgcgtaccgtcaaaattga

SEQ ID NO: 20 - protein, 361 aa, Stevia rebaudiana
MALVNPTALFYGTSIRTRPTNLLNPTQKLRPVSSSSLPSFSSVSAILTEKHQSNPSENNNLQTHLETPFNFDSYMLEKVNMVNEALDASVPLKDPIKIHESMRYSLLAGGKRIRPMMCIAACEIVGGNILNAMPAACAVEMIHTMSLVHDDLPCMDNDDFRRGKPISHKVYGEEMAVLTGDALLSLSFEHIATATKGVSKDRIVRAIGELARSVGSEGLVAGQVVDILSEGADVGLDHLEYIHIHKTAMLLESSVVIGAIMGGGSDQQIEKLRKFARSIGLLFQVVDDILDVTKSTEELGKTAGKDLLTDKTTYPKLLGIEKSREFAEKLNKEAQEQLSGFDRRKAAPLIALANYNAYRQN

SEQ ID NO: 21 - DNA, 1029 bp, Artificial Sequence
  Codon-optimized GGPPS
atggctgagcaacaaatatctaacttgctgtctatgtttgatgcttcacatgctagtcagaaattagaaattactgtccaaatgatggacacataccattacagagaaacgcctccagattcctcatcttctgaaggcggttcattgtctagatacgacgagagaagagtctctttgcctctcagtcataatgctgcctctccagatattgtatcacaactatgtttttccactgcaatgtcttcagagttgaatcacagatggaaatctcaaagattaaaggtggccgattctccttacaactatatcctaacattaccatcaaaaggaattagaggtgcctttatcgattccctgaacgtatggttggaggttccagaggatgaaacatcagtcatcaaggaagttattggtatgctccacaactcttcattaatcattgatgacttccaagataattctccacttagaagaggaaagccatctacccatacagtcttcggccctgcccaggctatcaatactgctacttacgttatagttaaagcaatcgaaaagatacaagacatagtgggacacgatgcattggcagatgttacgggtactattacaactattttccaaggtcaggccatggacttgtggtggacagcaaatgcaatcgttccatcaatacaggaatacttacttatggtaaacgataaaaccggtgctctctttagactgagtttggagttgttagctctgaattccgaagccagtatttctgactctgctttagaaagtttatctagtgctgtttccttgctaggtcaatacttccaaatcagagacgactatatgaacttgatcgataacaagtatacagatcagaaaggcttctgcgaagatcttgatgaaggcaagtactcactaacacttattcatgccctccaaactgattcatccgatctactgaccaacatcctttcaatgagaagagtgcaaggaaagttaacggcacaaaagagatgttggttctggaaatga

SEQ ID NO: 22 - protein, 342 aa, Gibberella fujikuroi
MAEQQISNLLSMFDASHASQKLEITVQMMDTYHYRETPPDSSSSEGGSLSRYDERRVSLPLSHNAASPDIVSQLCFSTAMSSELNHRWKSQRLKVADSPYNYILTLPSKGIRGAFIDSLNVWLEVPEDETSVIKEVIGMLHNSSLIIDDFQDNSPLRRGKPSTHTVFGPAQAINTATYVIVKAIEKIQDIVGHDALADVTGTITTIFQGQAMDLWWTANAIVPSIQEYLLMVNDKTGALFRLSLELLALNSEASISDSALESLSSAVSLLGQYFQIRDDYMNLIDNKYTDQKGFCEDLDEGKYSLTLIHALQTDSSDLLTNILSMRRVQGKLTAQKRCWFWK

SEQ ID NO: 23 - DNA, 903 bp, Artificial Sequence
  Codon-optimized GGPPS
atggaaaagactaaggagaaagcagaacgtatcttgctggagccatacagatacttattacaactaccaggaaagcaagtccgttctaaactatcacaagcgttcaatcactggttaaaagttcctgaagataagttacaaatcattattgaagtcacagaaatgctacacaatgcttctttactgatcgatgatatagaggattcttccaaactgagaagaggttttcctgtcgctcattccatatacggggtaccaagtgtaatcaactcagctaattacgtctacttcttgggattggaaaaagtattgacattagatcatccagacgctgtaaagctattcaccagacaacttcttgaattgcatcaaggtcaaggtttggatatctattggagagacacttatacttgcccaacagaagaggagtacaaagcaatggttctacaaaagactggcggtttgttcggacttgccgttggtctgatgcaacttttctctgattacaaggaggacttaaagcctctgttggataccttgggcttgtttttccagattagagatgactacgctaacttacattcaaaggaatattcagaaaacaaatcattctgtgaagatttgactgaagggaagtttagttttccaacaatccacgccatttggtcaagaccagaatctactcaagtgcaaaacattctgcgtcagagaacagagaatattgacatcaaaaagtattgtgttcagtacttggaagatgttggttcttttgcttacacaagacatacacttagagaattagaggcaaaagcatacaagcaaatagaagcctgtggaggcaatccttctctagtggcattggttaaacatttgtccaaaatgttcaccgaggaaaacaagtaa

SEQ ID NO: 24 - protein, 300 aa, Mus musculus
MEKTKEKAERILLEPYRYLLQLPGKQVRSKLSQAFNHWLKVPEDKLQIIIEVTEMLHNASLLIDDIEDSSKLRRGFPVAHSIYGVPSVINSANYVYFLGLEKVLTLDHPDAVKLFTRQLLELHQGQGLDIYWRDTYTCPTEEEYKAMVLQKTGGLFGLAVGLMQLFSDYKEDLKPLLDTLGLFFQIRDDYANLHSKEYSENKSFCEDLTEGKFSFPTIHAIWSRPESTQVQNILRQRTENIDIKKYCVQYLEDVGSFAYTRHTLRELEAKAYKQIEACGGNPSLVALVKHLSKMFTEENK

SEQ ID NO: 25 - DNA, 1020 bp, Artificial Sequence
  Codon-optimized GGPPS
atggcaagattctattttcttaacgcactattgatggttatctcattacaatcaactacagccttcactccagctaaacttgcttatccaacaacaacaacagctctaaatgtcgcctccgccgaaacttctttcagtctagatgaatacttggcctctaagataggacctatagagtctgccttggaagcatcagtcaaatccagaattccacagaccgataagatctgcgaatctatggcctactctttgatggcaggaggcaagagaattagaccagtgttgtgtatcgctgcatgtgagatgttcggtggatcccaagatgtcgctatgcctactgctgtggcattagaaatgatacacacaatgtctttgattcatgatgatttgccatccatggataacgatgacttgagaagaggtaaaccaacaaaccatgtcgttttcggcgaagatgtagctattcttgcaggtgactctttattgtcaacttccttcgagcacgtcgctagagaaacaaaaggagtgtcagcagaaaagatcgtggatgttatcgctagattaggcaaatctgttggtgccgagggccttgctggcggtcaagttatggacttagaatgtgaagctaaaccaggtaccacattagacgacttgaaatggattcatatccataaaaccgctacattgttacaagttgctgtagcttctggtgcagttctaggtggtgcaactcctgaagaggttgctgcatgcgagttgtttgctatgaatataggtcttgcctttcaagttgccgacgatatccttgatgtaaccgcttcatcagaagatttgggtaaaactgcaggcaaagatgaagctactgataagacaacttacccaaagttattaggattagaagagagtaaggcatacgcaagacaactaatcgatgaagccaaggaaagtttggctccttttggagatagagctgcccctttattggccattgcagatttcattattgatagaaagaattga

SEQ ID NO: 26 - protein, 339 aa, Thalassiosira pseudonana
MARFYFLNALLMVISLQSTTAFTPAKLAYPTTTTALNVASAETSFSLDEYLASKIGPIESALEASVKSRIPQTDKICESMAYSLMAGGKRIRPVLCIAACEMFGGSQDVAMPTAVALEMIHTMSLIHDDLPSMDNDDLRRGKPTNHVVFGEDVAILAGDSLLSTSFEHVARETKGVSAEKIVDVIARLGKSVGAEGLAGGQVMDLECEAKPGTTLDDLKWIHIHKTATLLQVAVASGAVLGGATPEEVAACELFAMNIGLAFQVADDILDVTASSEDLGKTAGKDEATDKTTYPKLLGLEESKAYARQLIDEAKESLAPFGDRAAPLLAIADFIIDRKN

SEQ ID NO: 27 - DNA, 1068 bp, Artificial Sequence
  Codon-optimized GGPPS
atgcacttagcaccacgtagagtccctagaggtagaagatcaccacctgacagagttcctgaaagacaaggtgccttgggtagaagacgtggagctggctctactggctgtgcccgtgctgctgctggtgttcaccgtagaagaggaggaggcgaggctgatccatcagctgctgtgcatagaggctggcaagccggtggtggcaccggtttgcctgatgaggtggtgtctaccgcagccgccttagaaatgtttcatgcttttgctttaatccatgatgatatcatggatgatagtgcaactagaagaggctccccaactgttcacagagccctagctgatcgtttaggcgctgctctggacccagatcaggccggtcaactaggagtttctactgctatcttggttggagatctggctttgacatggtccgatgaattgttatacgctccattgactccacatagactggcagcagtactaccattggtaacagctatgagagctgaaaccgttcatggccaatatcttgatataactagtgctagaagacctgggaccgatacttctcttgcattgagaatagccagatataagacagcagcttacacaatggaacgtccactgcacattggtgcagccctggctggggcaagaccagaactattagcagggctttcagcatacgccttgccagctggagaagccttccaattggcagatgacctgctaggcgtcttcggtgatccaagacgtacagggaaacctgacctagatgatcttagaggtggaaagcatactgtcttagtcgccttggcaagagaacatgccactccagaacagagacacacattggatacattattgggtacaccaggtcttgatagacaaggcgcttcaagactaagatgcgtattggtagcaactggtgcaagagccgaagccgaaagacttattacagagagaagagatcaagcattaactgcattgaacgcattaacactgccacctcctttagctgaggcattagcaagattgacattagggtctacagctcatcctgcctaa

SEQ ID NO: 28 - protein, 355 aa, Streptomyces clavuligerus
MHLAPRRVPRGRRSPPDRVPERQGALGRRRGAGSTGCARAAAGVHRRRGGGEADPSAAVHRGWQAGGGTGLPDEVVSTAAALEMFHAFALIHDDIMDDSATRRGSPTVHRALADRLGAALDPDQAGQLGVSTAILVGDLALTWSDELLYAPLTPHRLAAVLPLVTAMRAETVHGQYLDITSARRPGTDTSLALRIARYKTAAYTMERPLHIGAALAGARPELLAGLSAYALPAGEAFQLADDLLGVFGDPRRTGKPDLDDLRGGKHTVLVALAREHATPEQRHTLDTLLGTPGLDRQGASRLRCVLVATGARAEAERLITERRDQALTALNALTLPPPLAEALARLTLGSTAHPA

SEQ ID NO: 29 - DNA, 993 bp, Artificial Sequence
  Codon-optimized GGPPS
atgtcatatttcgataactacttcaatgagatagttaattccgtgaacgacatcattaagtcttacatctctggcgacgtaccaaaactatacgaagcctcctaccatttgtttacatcaggaggaaagagactaagaccattgatccttacaatttcttctgatcttttcggtggacagagagaaagagcatactatgctggcgcagcaatcgaagttttgcacacattcactttggttcacgatgatatcatggatcaagataacattcgtagaggtcttcctactgtacatgtcaagtatggcctacctttggccattttagctggtgacttattgcatgcaaaagcctttcaattgttgactcaggcattgagaggtctaccatctgaaactatcatcaaggcgtttgatatctttacaagatctatcattatcatatcagaaggtcaagctgtcgatatggaattcgaagatagaattgatatcaaggaacaagagtatttggatatgatatctcgtaaaaccgctgccttattctcagcttcttcttccattggggcgttgatagctggagctaatgataacgatgtgagattaatgtccgatttcggtacaaatcttgggatcgcatttcaaattgtagatgatatacttggtttaacagctgatgaaaaagagctaggaaaacctgttttcagtgatatcagagaaggtaaaaagaccatattagtcattaagactttagaattgtgtaaggaagacgagaaaaagattgtgttaaaagcgctaggcaacaagtcagcatcaaaggaagagttgatgagttctgctgacataatcaaaaagtactcattggattacgcctacaacttagctgagaaatactacaaaaacgccatcgattctctaaatcaagtttcaagtaaaagtgatattccagggaaggcattgaaatatcttgctgaattcaccatcagaagacgtaagtaa

SEQ ID NO: 30 - protein, 330 aa, Sulfolobus acidocaldarius
MSYFDNYFNEIVNSVNDIIKSYISGDVPKLYEASYHLFTSGGKRLRPLILTISSDLFGGQRERAYYAGAAIEVLHTFTLVHDDIMDQDNIRRGLPTVHVKYGLPLAILAGDLLHAKAFQLLTQALRGLPSETIIKAFDIFTRSIIIISEGQAVDMEFEDRIDIKEQEYLDMISRKTAALFSASSSIGALIAGANDNDVRLMSDFGTNLGIAFQIVDDILGLTADEKELGKPVFSDIREGKKTILVIKTLELCKEDEKKIVLKALGNKSASKEELMSSADIIKKYSLDYAYNLAEKYYKNAIDSLNQVSSKSDIPGKALKYLAEFTIRRRK

SEQ ID NO: 31 - DNA, 894 bp, Artificial Sequence
  Codon-optimized GGPPS
atggtcgcacaaactttcaacctggatacctacttatcccaaagacaacaacaagttgaagaggccctaagtgctgctcttgtgccagcttatcctgagagaatatacgaagctatgagatactccctcctggcaggtggcaaaagattaagacctatcttatgtttagctgcttgcgaattggcaggtggttctgttgaacaagccatgccaactgcgtgtgcacttgaaatgatccatacaatgtcactaattcatgatgacctgccagccatggataacgatgatttcagaagaggaaagccaactaatcacaaggtgttcggggaagatatagccatcttagcgggtgatgcgcttttagcttacgcttttgaacatattgcttctcaaacaagaggagtaccacctcaattggtgctacaagttattgctagaatcggacacgccgttgctgcaacaggcctcgttggaggccaagtcgtagaccttgaatctgaaggtaaagctatttccttagaaacattggagtatattcactcacataagactggagccttgctggaagcatcagttgtctcaggcggtattctcgcaggggcagatgaagagcttttggccagattgtctcattacgctagagatataggcttggcttttcaaatcgtcgatgatatcctggatgttactgctacatctgaacagttggggaaaaccgctggtaaagaccaggcagccgcaaaggcaacttatccaagtctattgggtttagaagcctctagacagaaagcggaagagttgattcaatctgctaaggaagccttaagaccttacggttcacaagcagagccactcctagcgctggcagacttcatcacacgtcgtcagcattaa

SEQ ID NO: 32 - protein, 297 aa, Synechococcus sp.
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

SEQ ID NO: 33 - DNA, 1680 bp, Artificial Sequence
  Codon-optimized CDPS
atgaaaaccgggtttatctcaccagcaacagtatttcatcacagaatctcaccagcgaccactttcagacatcacttatcacctgctactacaaactctacaggcattgtcgccttaagagacatcaacttcagatgtaaagcagtttctaaagagtactctgatctgttgcagaaagatgaggcttctttcacaaaatgggacgatgacaaggtgaaagatcatcttgataccaacaaaaacttatacccaaatgatgagattaaggaatttgttgaatcagtaaaggctatgttcggtagtatgaatgacggggagataaacgtctctgcatacgatactgcatgggttgctttggttcaagatgtcgatggatcaggtagtcctcagttcccttcttctttagaatggattgccaacaatcaattgtcagatggatcatggggagatcatttgctgttctcagctcacgatagaatcatcaacacattagcatgcgttattgcacttacaagttggaatgttcatccttctaagtgtgaaaaaggtttgaattttctgagagaaaacatttgcaaattagaagatgaaaacgcagaacatatgccaattggttttgaagtaacattcccatcactaattgatatcgcgaaaaagttgaacattgaagtacctgaggatactccagcacttaaagagatctacgcacgtagagatatcaagttaactaagatcccaatggaagttcttcacaaggtacctactactttgttacattctttggaaggaatgcctgatttggagtgggaaaaactgttaaagctacaatgtaaagatggtagtttcttgttttccccatctagtaccgcattcgccctaatgcaaacaaaagatgagaaatgcttacagtatctaacaaatatcgtcactaagttcaacggtggcgtgcctaatgtgtacccagtcgatttgtttgaacatatttgggttgttgatagactgcagagattggggattgccagatacttcaaatcagagataaaagattgtgtagagtatatcaataagtactggaccaaaaatggaatttgttgggctagaaatactcacgttcaagatatcgatgatacagccatgggattcagagtgttgagagcgcacggttatgacgtcactccagatgtttttagacaatttgaaaaagatggtaaattcgtttgctttgcagggcaatcaacacaagccgtgacaggaatgtttaacgtttacagagcctctcaaatgttgttcccaggggagagaattttggaagatgccaaaaagttctcttacaattacttaaaggaaaagcaaagtaccaacgaattgctggataaatggataatcgctaaagatctacctggtgaagttggttatgctctggatatcccatggtatgcttccttaccaagattggaaactcgttattaccttgaacaatacggcggtgaagatgatgtctggataggcaagacattatacagaatgggttacgtgtccaataacacatatctagaaatggcaaagctggattacaataactatgttgcagtccttcaattagaatggtacacaatacaacaatggtacgtcgatattggtatagagaagttcgaatctgacaacatcaagtcagtcctg

SEQ ID NO: 34 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 35 - DNA, 1584 bp, Artificial Sequence
  Codon-optimized CDPS
atgcctgatgcacacgatgctccacctccacaaataagacagagaacactagtagatgaggctacccaactgctaactgagtccgcagaagatgcatggggtgaagtcagtgtgtcagaatacgaaacagcaaggctagttgcccatgctacatggttaggtggacacgccacaagagtggccttccttctggagagacaacacgaagacgggtcatggggtccaccaggtggatataggttagtccctacattatctgctgttcacgcattattgacatgtcttgcctctcctgctcaggatcatggcgttccacatgatagacttttaagagctgttgacgcaggcttgactgccttgagaagattggggacatctgactccccacctgatactatagcagttgagctggttatcccatctttgctagagggcattcaacacttactggaccctgctcatcctcatagtagaccagccttctctcaacatagaggctctcttgtttgtcctggtggactagatgggagaactctaggagctttgagatcacacgccgcagcaggtacaccagtaccaggaaaagtctggcacgcttccgagactttgggcttgagtaccgaagctgcttctcacttgcaaccagcccaaggtataatcggtggctctgctgctgccacagcaacatggctaaccagggttgcaccatctcaacagtcagattctgccagaagataccttgaggaattacaacacagatactctggcccagttccttccattacccctatcacatacttcgaaagagcatggttattgaacaattttgcagcagccggtgttccttgtgaggctccagctgctttgttggattccttagaagcagcacttacaccacaaggtgctcctgctggagcaggattgcctccagatgctgatgatacagccgctgtgttgcttgcattggcaacacatgggagaggtagaagaccagaagtactgatggattacaggactgacgggtatttccaatgctttattggggaaaggactccatcaatttcaacaaacgctcacgtattggaaacattagggcatcatgtggcccaacatccacaagatagagccagatacggatcagccatggataccgcatcagcttggctgctggcagctcaaaagcaagatggctcttggttagataaatggcatgcctcaccatactacgctactgtttgttgcacacaagccctagccgctcatgcaagtcctgcaactgcaccagctagacagagagctgtcagatgggttttagccacacaaagatccgatggcggttggggtctatggcattcaactgttgaagagactgcttatgccttacagatcttggccccaccttctggtggtggcaatatcccagtccaacaagcacttactagaggcagagcaagattgtgtggagccttgccactgactcctttatggcatgataaggatttgtatactccagtaagagtagtcagagctgccagagctgctgctctgtacactaccagagatctattgttaccaccattgtaa

SEQ ID NO: 36 - protein, 527 aa, Streptomyces clavuligerus
MPDAHDAPPPQIRQRTLVDEATQLLTESAEDAWGEVSVSEYETARLVAHATWLGGHATRVAFLLERQHEDGSWGPPGGYRLVPTLSAVHALLTCLASPAQDHGVPHDRLLRAVDAGLTALRRLGTSDSPPDTIAVELVIPSLLEGIQHLLDPAHPHSRPAFSQHRGSLVCPGGLDGRTLGALRSHAAAGTPVPGKVWHASETLGLSTEAASHLQPAQGIIGGSAAATATWLTRVAPSQQSDSARRYLEELQHRYSGPVPSITPITYFERAWLLNNFAAAGVPCEAPAALLDSLEAALTPQGAPAGAGLPPDADDTAAVLLALATHGRGRRPEVLMDYRTDGYFQCFIGERTPSISTNAHVLETLGHHVAQHPQDRARYGSAMDTASAWLLAAQKQDGSWLDKWHASPYYATVCCTQALAAHASPATAPARQRAVRWVLATQRSDGGWGLWHSTVEETAYALQILAPPSGGGNIPVQQALTRGRARLCGALPLTPLWHDKDLYTPVRVVRAARAAALYTTRDLLLPPL

SEQ ID NO: 37 - DNA, 1551 bp, Artificial Sequence
  Codon-optimized CDPS
atgaacgccctatccgaacacattttgtctgaattgagaagattattgtctgaaatgagtgatggcggatctgttggtccatctgtgtatgatacggcccaggccctaagattccacggtaacgtaacaggtagacaagatgcatatgcttggttgatcgcccagcaacaagcagatggaggttggggctctgccgactttccactctttagacatgctccaacatgggctgcacttctcgcattacaaagagctgatccacttcctggcgcagcagacgcagttcagaccgcaacaagattcttgcaaagacaaccagatccatacgctcatgccgttcctgaggatgcccctattggtgctgaactgatcttgcctcagttttgtggagaggctgcttggttgttgggaggtgtggccttccctagacacccagccctattaccattaagacaggcttgtttagtcaaactgggtgcagtcgccatgttgccttcaggacacccattgctccactcctgggaggcatggggtacttctccaacaacagcctgtccagacgatgatggttctataggtatctcaccagcagctacagccgcctggagagcccaggctgtgaccagaggctcaactcctcaagtgggcagagctgacgcatacttacaaatggcttcaagagcaacgagatcaggcatagaaggagtcttccctaatgtttggcctataaacgtattcgaaccatgctggtcactgtacactctccatcttgccggtctgttcgcccatccagcactggctgaggctgtaagagttatcgttgctcaacttgaagcaagattgggagtgcatggcctcggaccagctttacattttgctgccgacgctgatgatactgcagttgccttatgcgttctgcatttggctggcagagatcctgcagttgacgcattgagacattttgaaattggtgagctctttgttacattcccaggagagagaaatgctagtgtctctacgaacattcacgctcttcatgctttgagattgttaggtaaaccagctgccggagcaagtgcatacgtcgaagcaaatagaaatccacatggtttgtgggacaacgaaaaatggcacgtttcatggctttatccaactgcacacgccgttgcagctctagctcaaggcaagcctcaatggagagatgaaagagcactagccgctctactacaagctcaaagagatgatggtggttggggagctggtagaggatccactttcgaggaaaccgcctacgctcttttcgctttacacgttatggacggatctgaggaagccacaggcagaagaagaatcgctcaagtcgtcgcaagagccttagaatggatgctagctagacatgccgcacatggattaccacaaacaccactctggattggtaaggaattgtactgtcctactagagtcgtaagagtagctgagctagctggcctgtggttagcattaagatggggtagaagagtattagctgaaggtgctggtgctgcaccttaa

SEQ ID NO: 38 - protein, 516 aa, Bradyrhizobium japonicum
MNALSEHILSELRRLLSEMSDGGSVGPSVYDTAQALRFHGNVTGRQDAYAWLIAQQQADGGWGSADFPLFRHAPTWAALLALQRADPLPGAADAVQTATRFLQRQPDPYAHAVPEDAPIGAELILPQFCGEAAWLLGGVAFPRHPALLPLRQACLVKLGAVAMLPSGHPLLHSWEAWGTSPTTACPDDDGSIGISPAATAAWRAQAVTRGSTPQVGRADAYLQMASRATRSGIEGVFPNVWPINVFEPCWSLYTLHLAGLFAHPALAEAVRVIVAQLEARLGVHGLGPALHFAADADDTAVALCVLHLAGRDPAVDALRHFEIGELFVTFPGERNASVSTNIHALHALRLLGKPAAGASAYVEANRNPHGLWDNEKWHVSWLYPTAHAVAALAQGKPQWRDERALAALLQAQRDDGGWGAGRGSTFEETAYALFALHVMDGSEEATGRRRIAQVVARALEWMLARHAAHGLPQTPLWIGKELYCPTRVVRVAELAGLWLALRWGRRVLAEGAGAAP

SEQ ID NO: 39 - DNA, 2490 bp, Artificial Sequence
  Codon-optimized CDPS
atggttttgtcttcttcttgtactacagtaccacacttatcttcattagctgtcgtgcaacttggtccttggagcagtaggattaaaaagaaaaccgatactgttgcagtaccagccgctgcaggaaggtggagaagggccttggctagagcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaaggacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 40 - protein, 827 aa, Zea mays
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 41 - DNA, 2570 bp, Artificial Sequence
  Codon-optimized CDPS
cttcttcactaaatacttagacagagaaaacagagctttttaaagccatgtctcttcagtatcatgttctaaactccattccaagtacaacctttctcagttctactaaaacaacaatatcttcttctttccttaccatctcaggatctcctctcaatgtcgctagagacaaatccagaagcggttccatacattgttcaaagcttcgaactcaagaatacattaattctcaagaggttcaacatgatttgcctctaatacatgagtggcaacagcttcaaggagaagatgctcctcagattagtgttggaagtaatagtaatgcattcaaagaagcagtgaagagtgtgaaaacgatcttgagaaacctaacggacggggaaattacgatatcggcttacgatacagcttgggttgcattgatcgatgccggagataaaactccggcgtttccctccgccgtgaaatggatcgccgagaaccaactttccgatggttcttggggagatgcgtatctcttctcttatcatgatcgtctcatcaatacccttgcatgcgtcgttgctctaagatcatggaatctctttcctcatcaatgcaacaaaggaatcacgtttttccgggaaaatattgggaagctagaagacgaaaatgatgagcatatgccaatcggattcgaagtagcattcccatcgttgcttgagatagctcgaggaataaacattgatgtaccgtacgattctccggtcttaaaagatatatacgccaagaaagagctaaagcttacaaggataccaaaagagataatgcacaagataccaacaacattgttgcatagtttggaggggatgcgtgatttagattgggaaaagctcttgaaacttcaatctcaagacggatctttcctcttctctccttcctctaccgcttttgcattcatgcagacccgagacagtaactgcctcgagtatttgcgaaatgccgtcaaacgtttcaatggaggagttcccaatgtctttcccgtggatcttttcgagcacatatggatagtggatcggttacaacgtttagggatatcgagatactttgaagaagagattaaagagtgtcttgactatgtccacagatattggaccgacaatggcatatgttgggctagatgttcccatgtccaagacatcgatgatacagccatggcatttaggctcttaagacaacatggataccaagtgtccgcagatgtattcaagaactttgagaaagagggagagtttttctgctttgtggggcaatcaaaccaagcagtaaccggtatgttcaacctataccgggcatcacaattggcgtttccaagggaagagatattgaaaaacgccaaagagttttcttataattatctgctagaaaaacgggagagagaggagttgattgataagtggattataatgaaagacttacctggcgagattgggtttgcgttagagattccatggtacgcaagcttgcctcgagtagagacgagattctatattgatcaatatggtggagaaaacgacgtttggattggcaagactctttataggatgccatacgtgaacaataatggatatctggaattagcaaaacaagattacaacaattgccaagctcagcatcagctcgaatgggacatattccaaaagtggtatgaagaaaataggttaagtgagtggggtgtgcgcagaagtgagcttctcgagtgttactacttagcggctgcaactatatttgaatcagaaaggtcacatgagagaatggtttgggctaagtcaagtgtattggttaaagccatttcttcttcttttggggaatcctctgactccagaagaagcttctccgatcagtttcatgaatacattgccaatgctcgacgaagtgatcatcactttaatgacaggaacatgagattggaccgaccaggatcggttcaggccagtcggcttgccggagtgttaatcgggactttgaatcaaatgtcttttgaccttttcatgtctcatggccgtgacgttaacaatctcctctatctatcgtggggagattggatggaaaaatggaaactatatggagatgaaggagaaggagagctcatggtgaagatgataattctaatgaagaacaatgacctaactaacttcttcacccacactcacttcgttcgtctcgcggaaatcatcaatcgaatctgtcttcctcgccaatacttaaaggcaaggagaaacgatgagaaggagaagacaataaagagtatggagaaggagatggggaaaatggttgagttagcattgtcggagagtgacacatttcgtgacgtcagcatcacgtttcttgatgtagcaaaagcattttactactttgctttatgtggcgatcatctccaaactcacatctccaaagtcttgtttcaaaaagtctagtaacctcatcatcatcatcgatccattaacaatcagtggatcgatgtatccatagatgcgtgaataatatttcatgtagagaaggagaacaaattagatcatgtagggttatca

SEQ ID NO: 42 - protein, 802 aa, Arabidopsis thaliana
MSLQYHVLNSIPSTTFLSSTKTTISSSFLTISGSPLNVARDKSRSGSIHCSKLRTQEYINSQEVQHDLPLIHEWQQLQGEDAPQISVGSNSNAFKEAVKSVKTILRNLTDGEITISAYDTAWVALIDAGDKTPAFPSAVKWIAENQLSDGSWGDAYLFSYHDRLINTLACVVALRSWNLFPHQCNKGITFFRENIGKLEDENDEHMPIGFEVAFPSLLEIARGINIDVPYDSPVLKDIYAKKELKLTRIPKEIMHKIPTTLLHSLEGMRDLDWEKLLKLQSQDGSFLFSPSSTAFAFMQTRDSNCLEYLRNAVKRFNGGVPNVFPVDLFEHIWIVDRLQRLGISRYFEEEIKECLDYVHRYWTDNGICWARCSHVQDIDDTAMAFRLLRQHGYQVSADVFKNFEKEGEFFCFVGQSNQAVTGMFNLYRASQLAFPREEILKNAKEFSYNYLLEKREREELIDKWIIMKDLPGEIGFALEIPWYASLPRVETRFYIDQYGGENDVWIGKTLYRMPYVNNNGYLELAKQDYNNCQAQHQLEWDIFQKWYEENRLSEWGVRRSELLECYYLAAATIFESERSHERMVWAKSSVLVKAISSSFGESSDSRRSFSDQFHEYIANARRSDHHFNDRNMRLDRPGSVQASRLAGVLIGTLNQMSFDLFMSHGRDVNNLLYLSWGDWMEKWKLYGDEGEGELMVKMIILMKNNDLTNFFTHTHFVRLAEIINRICLPRQYLKARRNDEKEKTIKSMEKEMGKMVELALSESDTFRDVSITFLDVAKAFYYFALCGDHLQTHISKVLFQKV

SEQ ID NO: 43 - DNA, 2355 bp, Artificial Sequence
  Codon-optimized KS
atgaatttgagtttgtgtatagcatctccactattgaccaaatctaatagaccagctgctttatcagcaattcatacagctagtacatcccatggtggccaaaccaaccctacgaatctgataatcgatacgaccaaggagagaatacaaaaacaattcaaaaatgttgaaatttcagtttcttcttatgatactgcgtgggttgccatggttccatcacctaattctccaaagtctccatgtttcccagaatgtttgaattggctgattaacaaccagttgaatgatggatcttggggtttagtcaatcacacgcacaatcacaaccatccacttttgaaagattctttatcctcaactttggcttgcatcgtggccctaaagagatggaacgtaggtgaggatcagattaacaaggggcttagtttcattgaatctaacttggcttccgcgactgaaaaatctcaaccatctccaataggattcgatatcatctttccaggtctgttagagtacgccaaaaatctagatatcaacttactgtctaagcaaactgatttctcactaatgttacacaagagagaattagaacaaaagagatgtcattcaaacgaaatggatggttacctagcttatatctctgaaggtcttggtaatctttacgattggaatatggtgaaaaagtaccagatgaaaaatggctcagttttcaattccccttctgcaactgcggcagcattcattaaccatcaaaatccaggatgcctgaactatttgaattcactactagacaaattcggcaacgcagttccaactgtataccctcacgatttgtttatcagattgagtatggtggatacaattgaaagacttggtatatcccaccactttagagtcgagatcaaaaatgttttggatgagacataccgttgttgggtggagagagatgaacaaatctttatggatgttgtgacgtgcgcgttggcctttagattgttgcgtattaacggttacgaagttagtccagatccacttgccgaaattacaaacgaattagctttaaaggatgaatacgccgctcttgaaacatatcatgcgtcacatatcctttaccaagaggacttatcatctggaaaacaaattcttaaatctgctgatttcctgaaggaaatcatatccactgatagtaatagactgtccaaactgatccataaagaggttgaaaatgcacttaagttccctattaacaccggcttagaacgtattaacacaagacgtaacatccagctttacaacgtagacaatactagaatcttgaaaaccacttaccattcttccaacatatcaaacactgattacctaagattagctgttgaagatttctacacatgtcagtctatctatagagaagagctgaaaggattagagagatgggtcgttgagaataagctagatcaattgaaatttgccagacaaaagacagcttattgttacttctcagttgccgccactttatcaagtccagaattgtcagatgcacgtatttcttgggctaaaaacggaattttgacaactgttgttgatgatttctttgatattggcgggacaatcgacgaattgacaaacctgattcaatgcgttgaaaagtggaatgtcgatgtcgataaagactgttgctcagaacatgttagaatactgttcttggctctgaaagatgctatctgttggatcggggatgaggctttcaaatggcaagctagagatgtgacgtctcacgtcattcaaacctggctagaactgatgaactctatgttgagagaagcaatttggactagagatgcatacgttcctacattaaacgagtatatggaaaacgcttatgtctcctttgctttgggtcctatcgttaagcctgccatatactttgtaggaccaaagctatccgaggaaatcgtcgaatcatcagaataccataacttgttcaagttaatgtccacacaaggcagattacttaatgatattcattctttcaaaagagagtttaaggaaggaaagttaaatgctgttgctctgcatctttctaatggcgaaagtggtaaagtcgaagaggaagtagttgaggaaatgatgatgatgatcaaaaacaagagaaaggagttgatgaaactaatcttcgaagagaacggttcaattgttcctagagcatgtaaggatgcattttggaacatgtgtcatgtgctaaactttttctacgcaaacgacgatggttttactgggaacacaatactagatacagtaaaagacatcatatacaaccctttggtcttagtaaacgaaaacgaggagcaaagataa

SEQ ID NO: 44 - protein, 784 aa, Stevia rebaudiana
MNLSLCIASPLLTKSNRPAALSAIHTASTSHGGQTNPTNLIIDTTKERIQKQFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATEKSQPSPIGFDIIFPGLLEYAKNLDINLLSKQTDFSLMLHKRELEQKRCHSNEMDGYLAYISEGLGNLYDWNMVKKYQMKNGSVFNSPSATAAAFINHQNPGCLNYLNSLLDKFGNAVPTVYPHDLFIRLSMVDTIERLGISHHFRVEIKNVLDETYRCWVERDEQIFMDVVTCALAFRLLRINGYEVSPDPLAEITNELALKDEYAALETYHASHILYQEDLSSGKQILKSADFLKEIISTDSNRLSKLIHKEVENALKFPINTGLERINTRRNIQLYNVDNTRILKTTYHSSNISNTDYLRLAVEDFYTCQSIYREELKGLERWVVENKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 45 - DNA, 2355 bp, Artificial Sequence
  Codon-optimized KS
atgaatctgtccctttgtatagctagtccactgttgacaaaatcttctagaccaactgctctttctgcaattcatactgccagtactagtcatggaggtcaaacaaacccaacaaatttgataatcgatactactaaggagagaatccaaaagctattcaaaaatgttgaaatctcagtatcatcttatgacaccgcatgggttgcaatggtgccatcacctaattccccaaaaagtccatgttttccagagtgcttgaattggttaatcaataatcagttaaacgatggttcttggggtttagtcaaccacactcataaccacaatcatccattattgaaggactctttatcatcaacattagcctgtattgttgcattgaaaagatggaatgtaggtgaagatcaaatcaacaagggtttatcattcatagaatccaatctagcttctgctaccgacaaatcacaaccatctccaatcgggttcgacataatcttccctggtttgctggagtatgccaaaaaccttgatatcaacttactgtctaaacaaacagatttctctttgatgctacacaaaagagagttagagcagaaaagatgccattctaacgaaattgacgggtacttagcatatatctcagaaggtttgggtaatttgtatgactggaacatggtcaaaaagtatcagatgaaaaatggatccgtattcaattctccttctgcaactgccgcagcattcattaatcatcaaaaccctgggtgtcttaactacttgaactcactattagataagtttggaaatgcagttccaacagtctatcctttggacttgtacatcagattatctatggttgacactatagagagattaggtatttctcatcatttcagagttgagatcaaaaatgttttggacgagacatacagatgttgggtcgaaagagatgagcaaatctttatggatgtcgtgacctgcgctctggcttttagattgctaaggatacacggatacaaagtatctcctgatcaactggctgagattacaaacgaactggctttcaaagacgaatacgccgcattagaaacataccatgcatcccaaatactttaccaggaagacctaagttcaggaaaacaaatcttgaagtctgcagatttcctgaaaggcattctgtctacagatagtaataggttgtctaaattgatacacaaggaagtagaaaacgcactaaagtttcctattaacactggtttagagagaatcaatactaggagaaacattcagctgtacaacgtagataatacaaggattcttaagaccacctaccatagttcaaacatttccaacacctattacttaagattagctgtcgaagacttttacacttgtcaatcaatctacagagaggagttaaagggcctagaaagatgggtagttcaaaacaagttggatcaactgaagtttgctagacagaagacagcatactgttatttctctgttgctgctaccctttcatccccagaattgtctgatgccagaataagttgggccaaaaatggtattcttacaactgtagtcgatgatttctttgatattggaggtactattgatgaactgacaaatcttattcaatgtgttgaaaagtggaacgtggatgtagataaggattgctgcagtgaacatgtgagaatacttttcctggctctaaaagatgcaatatgttggattggcgacgaggccttcaagtggcaagctagagatgttacatctcatgtcatccaaacttggcttgaactgatgaactcaatgctaagagaagcaatctggacaagagatgcatacgttccaacattgaacgaatacatggaaaacgcttacgtctcatttgccttgggtcctattgttaagccagccatatactttgttgggccaaagttatccgaagagattgttgagtcttccgaatatcataacctattcaagttaatgtcaacacaaggcagacttctgaacgatatccactccttcaaaagagaattcaaggaaggtaagctaaacgctgttgctttgcacttgtctaatggtgaatctggcaaagtggaagaggaagtcgttgaggaaatgatgatgatgatcaaaaacaagagaaaggaattgatgaaattgattttcgaggaaaatggttcaatcgtacctagagcttgtaaagatgctttttggaatatgtgccatgttcttaacttcttttacgctaatgatgatggcttcactggaaatacaatattggatacagttaaagatatcatctacaacccacttgttttggtcaatgagaacgaggaacaaagataa

SEQ ID NO: 46 - protein, 784 aa, Stevia rebaudiana
MNLSLCIASPLLTKSSRPTALSAIHTASTSHGGQTNPTNLIIDTTKERIQKLFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATDKSQPSPIGFDIIFPGLLEYAKNLDINLLSKQTDFSLMLHKRELEQKRCHSNEIDGYLAYISEGLGNLYDWNMVKKYQMKNGSVFNSPSATAAAFINHQNPGCLNYLNSLLDKFGNAVPTVYPLDLYIRLSMVDTIERLGISHHFRVEIKNVLDETYRCWVERDEQIFMDVVTCALAFRLLRIHGYKVSPDQLAEITNELAFKDEYAALETYHASQILYQEDLSSGKQILKSADFLKGILSTDSNRLSKLIHKEVENALKFPINTGLERINTRRNIQLYNVDNTRILKTTYHSSNISNTYYLRLAVEDFYTCQSIYREELKGLERWVVQNKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 47 - DNA, 1773 bp, Artificial Sequence
  Codon-optimized KS
atggctatgccagtgaagctaacacctgcgtcattatccttaaaagctgtgtgctgcagattctcatccggtggccatgctttgagattcgggagtagtctgccatgttggagaaggacccctacccaaagatctacttcttcctctactactagaccagctgccgaagtgtcatcaggtaagagtaaacaacatgatcaggaagctagtgaagcgactatcagacaacaattacaacttgtggatgtcctggagaatatgggaatatccagacattttgctgcagagataaagtgcatactagacagaacttacagatcttggttacaaagacacgaggaaatcatgctggacactatgacatgtgctatggcttttagaatcctaagattgaacggatacaacgtttcatcagatgaactataccacgttgtagaggcatctggtctgcataattctttgggtgggtatcttaacgataccagaacactacttgaattacacaaggcttcaacagttagtatctctgaggatgaatctatcttagattcaattggctctagatccagaacattgcttagagaacaattggagtctggtggcgcactgagaaagccttctttattcaaagaggttgaacatgcactggatggacctttttacaccacacttgatagacttcatcataggtggaatattgaaaacttcaacattattgagcaacacatgttggagactccatacttatctaaccagcatacatcaagggatatcctagcattgtcaattagagatttttcctcctcacaattcacttatcaacaagagctacagcatctggagagttgggttaaggaatgtagattagatcaactacagttcgcaagacagaaattagcgtacttttacctatcagccgcaggcaccatgttttctcctgagctttctgatgcgagaacattatgggccaaaaacggggtgttgacaactattgttgatgatttctttgatgttgccggttctaaagaggaattggaaaacttagtcatgctggtcgaaatgtgggatgaacatcacaaagttgaattctattctgagcaggtcgaaatcatcttctcttccatctacgattctgtcaaccaattgggtgagaaggcctctttggttcaagacagatcaattacaaaacaccttgttgaaatatggttagacttgttaaagtccatgatgacggaagttgaatggagactgtcaaaatacgtgcctacagaaaaggaatacatgattaatgcctctcttatcttcggcctaggtccaatcgttttaccagctttgtatttcgttggtccaaagatttcagaaagtatagtaaaggacccagaatatgatgaattgttcaaactaatgtcaacatgtggtagattgttgaatgacgtgcaaacgttcgaaagagaatacaatgagggtaaactgaattctgtcagtctattggttcttcacggaggcccaatgtctatttcagacgcaaagaggaaattacaaaagcctattgatacgtgtagaagagatcttctttctttggtccttagagaagagtctgtagtaccaagaccatgtaaggaactattctggaaaatgtgtaaagtgtgctatttcttttactcaacaactgatgggttttctagtcaagtcgaaagagcaaaagaggtagacgctgtcataaatgagccactgaagttgcaaggttctcatacactggtatctgatgtttaa

SEQ ID NO: 48 - protein, 590 aa, Zea mays
MAMPVKLTPASLSLKAVCCRFSSGGHALRFGSSLPCWRRTPTQRSTSSSTTRPAAEVSSGKSKQHDQEASEATIRQQLQLVDVLENMGISRHFAAEIKCILDRTYRSWLQRHEEIMLDTMTCAMAFRILRLNGYNVSSDELYHVVEASGLHNSLGGYLNDTRTLLELHKASTVSISEDESILDSIGSRSRTLLREQLESGGALRKPSLFKEVEHALDGPFYTTLDRLHHRWNIENFNIIEQHMLETPYLSNQHTSRDILALSIRDFSSSQFTYQQELQHLESWVKECRLDQLQFARQKLAYFYLSAAGTMFSPELSDARTLWAKNGVLTTIVDDFFDVAGSKEELENLVMLVEMWDEHHKVEFYSEQVEIIFSSIYDSVNQLGEKASLVQDRSITKHLVEIWLDLLKSMMTEVEWRLSKYVPTEKEYMINASLIFGLGPIVLPALYFVGPKISESIVKDPEYDELFKLMSTCGRLLNDVQTFEREYNEGKLNSVSLLVLHGGPMSISDAKRKLQKPIDTCRRDLLSLVLREESVVPRPCKELFWKMCKVCYFFYSTTDGFSSQVERAKEVDAVINEPLKLQGSHTLVSDV

SEQ ID NO: 49 - DNA, 2232 bp, Artificial Sequence
  Codon-optimized KS
atgcagaacttccatggtacaaaggaaaggatcaaaaagatgtttgacaagattgaattgtccgtttcttcttatgatacagcctgggttgcaatggtcccatcccctgattgcccagaaacaccttgttttccagaatgtactaaatggatcctagaaaatcagttgggtgatggtagttggtcacttcctcatggcaatccacttctagttaaagatgcattatcttccactcttgcttgtattctggctcttaaaagatggggaatcggtgaggaacagattaacaaaggactgagattcatagaactcaactctgctagtgtaaccgataacgaacaacacaaaccaattggatttgacattatctttccaggtatgattgaatacgctatagacttagacctgaatctaccactaaaaccaactgacattaactccatgttgcatcgtagagcccttgaattgacatcaggtggaggcaaaaatctagaaggtagaagagcttacttggcctacgtctctgaaggaatcggtaagctgcaagattgggaaatggctatgaaataccaacgtaaaaacggatctctgttcaatagtccatcaacaactgcagctgcattcatccatatacaagatgctgaatgcctccactatattcgttctcttctccagaaatttggaaacgcagtccctacaatataccctctcgatatctatgccagactttcaatggtagatgccctggaacgtcttggtattgatagacatttcagaaaggagagaaagttcgttctggatgaaacatacagattttggttgcaaggagaagaggagattttctccgataacgcaacctgtgctttggccttcagaatattgagacttaatggttacgatgtctctcttgaagatcacttctctaactctctgggcggttacttaaaggactcaggagcagctttagaactgtacagagccctccaattgtcttacccagacgagtccctcctggaaaagcaaaattctagaacttcttacttcttaaaacaaggtttatccaatgtctccctctgtggtgacagattgcgtaaaaacataattggagaggtgcatgatgctttaaacttttccgaccacgctaacttacaaagattagctattcgtagaaggattaagcattacgctactgacgatacaaggattctaaaaacttcctacagatgctcaacaatcggtaaccaagattttctaaaacttgcagtggaagatttcaatatctgtcaatcaatacaaagagaggaattcaagcatattgaaagatgggtcgttgaaagacgtctagacaagttaaagttcgctagacaaaaagaggcctattgctatttctcagccgcagcaacattgtttgcccctgaattgtctgatgctagaatgtcttgggccaaaaatggtgtattgacaactgtggttgatgatttcttcgatgtcggaggctctgaagaggaattagttaacttgatagaattgatcgagcgttgggatgtgaatggcagtgcagatttttgtagtgaggaagttgagattatctattctgctatccactcaactatctctgaaataggtgataagtcatttggctggcaaggtagagatgtaaagtctcaagttatcaagatctggctggacttattgaaatcaatgttaactgaagctcaatggtcttcaaacaagtctgttcctaccctagatgagtatatgacaaccgcccatgtttcattcgcacttggtccaattgtacttccagccttatacttcgttggcccaaagttgtcagaagaggttgcaggtcatcctgaactactaaacctctacaaagtcacatctacttgtggcagactactgaatgattggagaagttttaagagagaatccgaggaaggtaagctcaacgctattagtttatacatgatccactccggtggtgcttctacagaagaggaaacaatcgaacatttcaaaggtttgattgattctcagagaaggcaactgttacaattggtgttgcaagagaaggatagtatcatacctagaccatgtaaagatctattttggaatatgattaagttattacacactttctacatgaaagatgatggcttcacctcaaatgagatgaggaatgtagttaaggcaatcattaacgaaccaatctcactggatgaattatga

SEQ ID NO: 50 - protein, 775 aa, Populus trichocarpa
MSCIRPWFCPSSISATLTDPASKLVTGEFKTTSLNFHGTKERIKKMFDKIELSVSSYDTAWVAMVPSPDCPETPCFPECTKWILENQLGDGSWSLPHGNPLLVKDALSSTLACILALKRWGIGEEQINKGLRFIELNSASVTDNEQHKPIGFDIIFPGMIEYAKDLDLNLPLKPTDINSMLHRRALELTSGGGKNLEGRRAYLAYVSEGIGKLQDWEMAMKYQRKNGSLFNSPSTTAAAFIHIQDAECLHYIRSLLQKFGNAVPTIYPLDIYARLSMVDALERLGIDRHFRKERKFVLDETYRFWLQGEEEIFSDNATCALAFRILRLNGYDVSLEDHFSNSLGGYLKDSGAALELYRALQLSYPDESLLEKQNSRTSYFLKQGLSNVSLCGDRLRKNIIGEVHDALNFPDHANLQRLAIRRRIKHYATDDTRILKTSYRCSTIGNQDFLKLAVEDFNICQSIQREEFKHIERWVVERRLDKLKFARQKEAYCYFSAAATLFAPELSDARMSWAKNGVLTTVVDDFFDVGGSEEELVNLIELIERWDVNGSADFCSEEVEIIYSAIHSTISEIGDKSFGWQGRDVKSHVIKIWLDLLKSMLTEAQWSSNKSVPTLDEYMTTAHVSFALGPIVLPALYFVGPKLSEEVAGHPELLNLYKVMSTCGRLLNDWRSFKRESEEGKLNAISLYMIHSGGASTEEETIEHFKGLIDSQRRQLLQLVLQEKDSIIPRPCKDLFWNMIKLLHTFYMKDDGFTSNEMRNVVKAIINEPISLDEL

SEQ ID NO: 51 - DNA, 2358 bp, Artificial Sequence
  Codon-optimized KS
atgtctatcaaccttcgctcctccggttgttcgtctccgatctcagctactttggaacgaggattggactcagaagtacagacaagagctaacaatgtgagctttgagcaaacaaaggagaagattaggaagatgttggagaaagtggagctttctgtttcggcctacgatactagttgggtagcaatggttccatcaccgagctcccaaaatgctccacttttcccacagtgtgtgaaatggttattggataatcaacatgaagatggatcttggggacttgataaccatgaccatcaatctcttaagaaggatgtgttatcatctacactggctagtatcctcgcgttaaagaagtggggaattggtgaaagacaaataaacaagggtctccagtttattgagctgaattctgcattagtcactgatgaaaccatacagaaaccaacagggtttgatattatatttcctgggatgattaaatatgctagagatttgaatctgacgattccattgggctcagaagtggtggatgacatgatacgaaaaagagatctggatcttaaatgtgatagtgaaaagttttcaaagggaagagaagcatatctggcctatgttttagaggggacaagaaacctaaaagattgggatttgatagtcaaatatcaaaggaaaaatgggtcactgtttgattctccagccacaacagcagctgcttttactcagtttgggaatgatggttgtctccgttatctctgttctctccttcagaaattcgaggctgcagttccttcagtttatccatttgatcaatatgcacgccttagtataattgtcactcttgaaagcttaggaattgatagagatttcaaaaccgaaatcaaaagcatattggatgaaacctatagatattggcttcgtggggatgaagaaatatgtttggacttggccacttgtgctttggctttccgattattgcttgctcatggctatgatgtgtcttacgatccgctaaaaccatttgcagaagaatctggtttctctgatactttggaaggatatgttaagaatacgttttctgtgttagaattatttaaggctgctcaaagttatccacatgaatcagctttgaagaagcagtgttgttggactaaacaatatctggagatggaattgtccagctgggttaagacctctgttcgagataaatacctcaagaaagaggtcgaggatgctcttgcttttccctcctatgcaagcctagaaagatcagatcacaggagaaaaatactcaatggttctgctgtggaaaacaccagagttacaaaaacctcatatcgtttgcacaatatttgcacctctgatatcctgaagttagctgtggatgacttcaatttctgccagtccatacaccgtgaagaaatggaacgtcttgataggtggattgtggagaatagattgcaggaactgaaatttgccagacagaagctggcttactgttatttctctggggctgcaactttattttctccagaactatctgatgctcgtatatcgtgggccaaaggtggagtacttacaacggttgtagacgacttctttgatgttggagggtccaaagaagaactggaaaacctcatacacttggtcgaaaagtgggatttgaacggtgttcctgagtacagctcagaacatgttgagatcatattctcagttctaagggacaccattctcgaaacaggagacaaagcattcacctatcaaggacgcaatgtgacacaccacattgtgaaaatttggttggatctgctcaagtctatgttgagagaagccgagtggtccagtgacaagtcaacaccaagcttggaggattacatggaaaatgcgtacatatcatttgcattaggaccaattgtcctcccagctacctatctgatcggacctccacttccagagaagacagtcgatagccaccaatataatcagctctacaagctcgtgagcactatgggtcgtcttctaaatgacatacaaggttttaagagagaaagcgcggaagggaagctgaatgcggtttcattgcacatgaaacacgagagagacaatcgcagcaaagaagtgatcatagaatcgatgaaaggtttagcagagagaaagagggaagaattgcataagctagttttggaggagaaaggaagtgtggttccaagggaatgcaaagaagcgttcttgaaaatgagcaaagtgttgaacttattttacaggaaggacgatggattcacatcaaatgatctgatgagtcttgttaaatcagtgatctacgagcctgttagcttacagaaagaatctttaacttga

SEQ ID NO: 52 - protein, 785 aa, Arabidopsis thaliana
MSINLRSSGCSSPISATLERGLDSEVQTRANNVSFEQTKEKIRKMLEKVELSVSAYDTSWVAMVPSPSSQNAPLFPQCVKWLLDNQHEDGSWGLDNHDHQSLKKDVLSSTLASILALKKWGIGERQINKGLQFIELNSALVTDETIQKPTGFDIIFPGMIKYARDLNLTIPLGSEVVDDMIRKRDLDLKCDSEKFSKGREAYLAYVLEGTRNLKDWDLIVKYQRKNGSLFDSPATTAAAFTQFGNDGCLRYLCSLLQKFEAAVPSVYPFDQYARLSIIVTLESLGIDRDFKTEIKSILDETYRYWLRGDEEICLDLATCALAFRLLLAHGYDVSYDPLKPFAEESGFSDTLEGYVKNTFSVLELFKAAQSYPHESALKKQCCWTKQYLEMELSSWVKTSVRDKYLKKEVEDALAFPSYASLERSDHRRKILNGSAVENTRVTKTSYRLHNICTSDILKLAVDDFNFCQSIHREEMERLDRWIVENRLQELKFARQKLAYCYFSGAATLFSPELSDARISWAKGGVLTTVVDDFFDVGGSKEELENLIHLVEKWDLNGVPEYSSEHVEIIFSVLRDTILETGDKAFTYQGRNVTHHIVKIWLDLLKSMLREAEWSSDKSTPSLEDYMENAYISFALGPIVLPATYLIGPPLPEKTVDSHQYNQLYKLVSTMGRLLNDIQGFKRESAEGKLNAVSLHMKHERDNRSKEVIIESMKGLAERKREELHKLVLEEKGSVVPRECKEAFLKMSKVLNLFYRKDDGFTSNDLMSLVKSVIYEPVSLQKESLT

SEQ ID NO: 53 - DNA, 2952 bp, Artificial Sequence
  Codon-optimized CDPS-KS
atggaatttgatgaaccattggttgacgaagcaagatctttagtgcagcgtactttacaagattatgatgacagatacggcttcggtactatgtcatgtgctgcttatgatacagcctgggtgtctttagttacaaaaacagtcgatgggagaaaacaatggcttttcccagagtgttttgaatttctactagaaacacaatctgatgccggaggatgggaaatcgggaattcagcaccaatcgacggtatattgaatacagctgcatccttacttgctctaaaacgtcacgttcaaactgagcaaatcatccaacctcaacatgaccataaggatctagcaggtagagctgaacgtgccgctgcatctttgagagcacaattggctgcattggatgtgtctacaactgaacacgtcggttttgagataattgttcctgcaatgctagacccattagaagccgaagatccatctctagttttcgattttccagctaggaaacctttgatgaagattcatgatgctaagatgagtagattcaggccagaatacttgtatggcaaacaaccaatgaccgccttacattcattagaggctttcataggcaaaatcgacttcgataaggtaagacaccaccgtacccatgggtctatgatgggttctccttcatctaccgcagcctacttaatgcacgcttcacaatgggatggtgactcagaggcttaccttagacacgtgattaaacacgcagcagggcagggaactggtgctgtaccatctgctttcccatcaacacattttgagtcatcttggattcttaccacattgtttagagctggattttcagcttctcatcttgcctgtgatgagttgaacaagttggtcgagatacttgagggctcattcgagaaggaaggtggggcaatcggttacgctccagggtttcaagcagatgttgatgatactgctaaaacaataagtacattagcagtccttggaagagatgctacaccaagacaaatgatcaaggtatttgaagctaatacacattttagaacataccctggtgaaagagatccttctttgacagctaattgtaatgctctatcagccttactacaccaaccagatgcagcaatgtatggatctcaaattcaaaagattaccaaatttgtctgtgactattggtggaagtctgatggtaagattaaagataagtggaacacttgctacttgtacccatctgtcttattagttgaggttttggttgatcttgttagtttattggagcagggtaaattgcctgatgttttggatcaagagcttcaatacagagtcgccatcacattgttccaagcatgtttaaggccattactagaccaagatgccgaaggatcatggaacaagtctatcgaagccacagcctacggcatccttatcctaactgaagctaggagagtttgtttcttcgacagattgtctgagccattgaatgaggcaatccgtagaggtatcgctttcgccgactctatgtctggaactgaagctcagttgaactacatttggatcgaaaaggttagttacgcacctgcattattgactaaatcctatttgttagcagcaagatgggctgctaagtctcctttaggcgcttccgtaggctcttctttgtggactccaccaagagaaggattggataagcatgtcagattattccatcaagctgagttattcagatcccttccagaatgggaattaagagcctccatgattgaagcagctttgttcacaccacttctaagagcacatagactagacgttttccctagacaagatgtaggtgaagacaaatatcttgatgtagttccattcttttggactgccgctaacaacagagatagaacttacgcttccactctattcctttacgatatgtgttttatcgcaatgttaaacttccagttagacgaattcatggaggccacagccggtatcttattcagagatcatatggatgatttgaggcaattgattcatgatcttttggcagagaaaacttccccaaagagttctggtagaagtagtcagggcacaaaagatgctgactcaggtatagaggaagacgtgtcaatgtccgattcagcttcagattcccaggatagaagtccagaatacgacttggttttcagtgcattgagtacctttacaaaacatgtcttgcaacacccatctatacaaagtgcctctgtatgggatagaaaactacttgctagagagatgaaggcttacttacttgctcatatccaacaagcagaagattcaactccattgtctgaattgaaagatgtgcctcaaaagactgatgtaacaagagtttctacatctactactaccttctttaactgggttagaacaacttccgcagaccatatatcctgcccatactccttccactttgtagcatgccatctaggcgcagcattgtcacctaaagggtctaacggtgattgctatccttcagctggtgagaagttcttggcagctgcagtctgcagacatttggccaccatgtgtagaatgtacaacgatcttggatcagctgaacgtgattctgatgaaggtaatttgaactccttggacttccctgaattcgccgattccgcaggaaacggagggatagaaattcagaaggccgctctattaaggttagctgagtttgagagagattcatacttagaggccttccgtcgtttacaagatgaatccaatagagttcacggtccagccggtggtgatgaagccagattgtccagaaggagaatggcaatccttgaattcttcgcccagcaggtagatttgtacggtcaagtatacgtcattagggatatttccgctcgtattcctaaaaacgaggttgagaaaaagagaaaattggatgatgctttcaattga

SEQ ID NO: 54 - protein, 983 aa, Phomopsis amygdali
MEFDEPLVDEARSLVQRTLQDYDDRYGFGTMSCAAYDTAWVSLVTKTVDGRKQWLFPECFEFLLETQSDAGGWEIGNSAPIDGILNTAASLLALKRHVQTEQIIQPQHDHKDLAGRAERAAASLRAQLAALDVSTTEHVGFEIIVPAMLDPLEAEDPSLVFDFPARKPLMKIHDAKMSRFRPEYLYGKQPMTALHSLEAFIGKIDFDKVRHHRTHGSMMGSPSSTAAYLMHASQWDGDSEAYLRHVIKHAAGQGTGAVPSAFPSTHFESSWILTTLFRAGFSASHLACDELNKLVEILEGSFEKEGGAIGYAPGFQADVDDTAKTISTLAVLGRDATPRQMIKVFEANTHFRTYPGERDPSLTANCNALSALLHQPDAAMYGSQIQKITKFVCDYWWKSDGKIKDKWNTCYLYPSVLLVEVLVDLVSLLEQGKLPDVLDQELQYRVAITLFQACLRPLLDQDAEGSWNKSIEATAYGILILTEARRVCFFDRLSEPLNEAIRRGIAFADSMSGTEAQLNYIWIEKVSYAPALLTKSYLLAARWAAKSPLGASVGSSLWTPPREGLDKHVRLFHQAELFRSLPEWELRASMIEAALFTPLLRAHRLDVFPRQDVGEDKYLDVVPFFWTAANNRDRTYASTLFLYDMCFIAMLNFQLDEFMEATAGILFRDHMDDLRQLIHDLLAEKTSPKSSGRSSQGTKDADSGIEEDVSMSDSASDSQDRSPEYDLVFSALSTFTKHVLQHPSIQSASVWDRKLLAREMKAYLLAHIQQAEDSTPLSELKDVPQKTDVTRVSTSTTTFFNWVRTTSADHISCPYSFHFVACHLGAALSPKGSNGDCYPSAGEKFLAAAVCRHLATMCRMYNDLGSAERDSDEGNLNSLDFPEFADSAGNGGIEIQKAALLRLAEFERDSYLEAFRRLQDESNRVHGPAGGDEARLSRRRMAILEFFAQQVDLYGQVYVIRDISARIPKNEVEKKRKLDDAFN

SEQ ID NO: 55 - DNA, 2646 bp, Artificial Sequence
  Codon-optimized CDPS-KS
atggcttctagtacacttatccaaaacagatcatgtggcgtcacatcatctatgtcaagttttcaaatcttcagaggtcaaccactaagatttcctggcactagaaccccagctgcagttcaatgcttgaaaaagaggagatgccttaggccaaccgaatccgtactagaatcatctcctggctctggttcatatagaatagtaactggcccttctggaattaaccctagttctaacgggcacttgcaagagggttccttgactcacaggttaccaataccaatggaaaaatctatcgataacttccaatctactctatatgtgtcagatatttggtctgaaacactacagagaactgaatgtttgctacaagtaactgaaaacgtccagatgaatgagtggattgaggaaattagaatgtactttagaaatatgactttaggtgaaatttccatgtccccttacgacactgcttgggtggctagagttccagcgttggacggttctcatgggcctcaattccacagatctttgcaatggattatcgacaaccaattaccagatggggactggggcgaaccttctcttttcttgggttacgatagagtttgtaatactttagcctgtgtgattgcgttgaaaacatggggtgttggggcacaaaacgttgaaagaggaattcagttcctacaatctaacatatacaagatggaggaagatgacgctaatcatatgccaataggattcgaaatcgtattccctgctatgatggaagatgccaaagcattaggtttggatttgccatacgatgctactattttgcaacagatttcagccgaaagagagaaaaagatgaaaaagatcccaatggcaatggtgtacaaatacccaaccactttacttcactccttagaaggcttgcatagagaagttgattggaataagttgttacaattacaatctgaaaatggtagttttctttattcacctgcttcaaccgcatgcgccttaatgtacactaaggacgttaaatgttttgattacttaaaccagttgttgatcaagttcgaccacgcatgcccaaatgtatatccagtcgatctattcgaaagattatggatggttgacagattgcagagattagggatctccagatactttgaaagagagattagagattgtttacaatacgtctacagatattggaaagattgtggaatcggatgggcttctaactcttccgtacaagatgttgatgatacagccatggcgtttagacttttaaggactcatggtttcgacgtaaaggaagattgctttagacagtttttcaaggacggagaattcttctgcttcgcaggccaatcatctcaagcagttacaggcatgtttaatctttcaagagccagtcaaacattgtttccaggagaatctttattgaaaaaggctagaaccttctctagaaacttcttgagaacaaagcatgagaacaacgaatgtttcgataaatggatcattactaaagatttggctggtgaagtcgagtataacttgaccttcccatggtatgcctctttgcctagattagaacataggacatacttagatcaatatggaatcgatgatatctggataggcaaatctttatacaaaatgcctgctgttaccaacgaagttttcctaaagttggcaaaggcagactttaacatgtgtcaagctctacacaaaaaggaattggaacaagtgataaagtggaacgcgtcctgtcaattcagagatcttgaattcgccagacaaaaatcagtagaatgctattttgctggtgcagccacaatgttcgaaccagaaatggttcaagctagattagtctgggcaagatgttgtgtattgacaactgtcttagacgattactttgaccacgggacacctgttgaggaacttagagtgtttgttcaagctgtcagaacatggaatccagagttgatcaacggtttgccagagcaagctaaaatcttgtttatgggcttatacaaaacagttaacacaattgcagaggaagcattcatggcacagaaaagagacgtccatcatcatttgaaacactattgggacaagttgataacaagtgccctaaaggaggccgaatgggcagagtcaggttacgtcccaacatttgatgaatacatggaagtagctgaaatttctgttgctctagaaccaattgtctgtagtaccttgttctttgcgggtcatagactagatgaggatgttctagatagttacgattaccatctagttatgcatttggtaaacagagtcggtagaatcttgaatgatatacaaggcatgaagagggaggcttcacaaggtaagatctcatcagttcaaatctacatggaggaacatccatctgttccatctgaggccatggcgatcgctcatcttcaagagttagttgataattcaatgcagcaattgacatacgaagttcttaggttcactgcggttccaaaaagttgtaagagaatccacttgaatatggctaaaatcatgcatgccttctacaaggatactgatggattctcatcccttactgcaatgacaggattcgtcaaaaaggttcttttcgaacctgtgcctgagtaa

SEQ ID NO: 56 - protein, 881 aa, Physcomitrella patens
MASSTLIQNRSCGVTSSMSSFQIFRGQPLRFPGTRTPAAVQCLKKRRCLRPTESVLESSPGSGSYRIVTGPSGINPSSNGHLQEGSLTHRLPIPMEKSIDNFQSTLYVSDIWSETLQRTECLLQVTENVQMNEWIEEIRMYFRNMTLGEISMSPYDTAWVARVPALDGSHGPQFHRSLQWIIDNQLPDGDWGEPSLFLGYDRVCNTLACVIALKTWGVGAQNVERGIQFLQSNIYKMEEDDANHMPIGFEIVFPAMMEDAKALGLDLPYDATILQQISAEREKKMKKIPMAMVYKYPTTLLHSLEGLHREVDWNKLLQLQSENGSFLYSPASTACALMYTKDVKCFDYLNQLLIKFDHACPNVYPVDLFERLWMVDRLQRLGISRYFEREIRDCLQYVYRYWKDCGIGWASNSSVQDVDDTAMAFRLLRTHGFDVKEDCFRQFFKDGEFFCFAGQSSQAVTGMFNLSRASQTLFPGESLLKKARTFSRNFLRTKHENNECFDKWIITKDLAGEVEYNLTFPWYASLPRLEHRTYLDQYGIDDIWIGKSLYKMPAVTNEVFLKLAKADFNMCQALHKKELEQVIKWNASCQFRDLEFARQKSVECYFAGAATMFEPEMVQARLVWARCCVLTTVLDDYFDHGTPVEELRVFVQAVRTWNPELINGLPEQAKILFMGLYKTVNTIAEEAFMAQKRDVHHHLKHYWDKLITSALKEAEWAESGYVPTFDEYMEVAEISVALEPIVCSTLFFAGHRLDEDVLDSYDYHLVMHLVNRVGRILNDIQGMKREASQGKISSVQIYMEEHPSVPSEAMAIAHLQELVDNSMQQLTYEVLRFTAVPKSCKRIHLNMAKIMHAFYKDTDGFSSLTAMTGFVKKVLFEPVPE

SEQ ID NO: 57 - DNA, 2859 bp, Artificial Sequence
  Codon-optimized CDPS-KS
atgcctggtaaaattgaaaatggtaccccaaaggacctcaagactggaaatgattttgtttctgctgctaagagtttactagatcgagctttcaaaagtcatcattcctactacggattatgctcaacttcatgtcaagtttatgatacagcttgggttgcaatgattccaaaaacaagagataatgtaaaacagtggttgtttccagaatgtttccattacctcttaaaaacacaagccgcagatggctcatggggttcattgcctacaacacagacagcgggtatcctagatacagcctcagctgtgctggcattattgtgccacgcacaagagcctttacaaatattggatgtatctccagatgaaatggggttgagaatagaacacggtgtcacatccttgaaacgtcaattagcagtttggaatgatgtggaggacaccaaccatattggcgtcgagtttatcataccagccttactttccatgctagaaaaggaattagatgttccatcttttgaatttccatgtaggtccatcttagagagaatgcacggggagaaattaggtcatttcgacctggaacaagtttacggcaagccaagctcattgttgcactcattggaagcatttctcggtaagctagattttgatcgactatcacatcacctataccacggcagtatgatggcatctccatcttcaacggctgcttatcttattggggctacaaaatgggatgacgaagccgaagattacctaagacatgtaatgcgtaatggtgcaggacatgggaatggaggtatttctggtacatttccaactactcatttcgaatgtagctggattatagcaacgttgttaaaggttggctttactttgaagcaaattgacggcgatggcttaagaggtttatcaaccatcttacttgaggcgcttcgtgatgagaatggtgtcataggctttgcccctagaacagcagatgtagatgacacagccaaagctctattggccttgtcattggtaaaccagccagtgtcacctgatatcatgattaaggtctttgagggcaaagaccattttaccacttttggttcagaaagagatccatcattgacttccaacctgcacgtccttttatctttacttaaacaatctaacttgtctcaataccatcctcaaatcctcaaaacaacattattcacttgtagatggtggtggggttccgatcattgtgtcaaagacaaatggaatttgagtcacctatatccaactatgttgttggttgaagccttcactgaagtgctccatctcattgacggtggtgaattgtctagtctgtttgatgaatcctttaagtgtaagattggtcttagcatctttcaagcggtacttagaataatcctcacccaagacaacgacggctcttggagaggatacagagaacagacgtgttacgcaatattggctttagttcaagcgagacatgtatgctttttcactcacatggttgacagactgcaatcatgtgttgatcgaggtttctcatggttgaaatcttgctcttttcattctcaagacctgacttggacctctaaaacagcttatgaagtgggtttcgtagctgaagcatataaactagctgctttacaatctgcttccctggaggttcctgctgccaccattggacattctgtcacgtctgccgttccatcaagtgatcttgaaaaatacatgagattggtgagaaaaactgcgttattctctccactggatgagtggggtctaatggcttctatcatcgaatcttcatttttcgtaccattactgcaggcacaaagagttgaaatataccctagagataatatcaaggtggacgaagataagtacttgtctattatcccattcacatgggtcggatgcaataataggtctagaactttcgcaagtaacagatggctatacgatatgatgtacctttcattactcggctatcaaaccgacgagtacatggaagctgtagctgggccagtgtttggggatgtttccttgttacatcaaacaattgataaggtgattgataatacaatgggtaaccttgcgagagccaatggaacagtacacagtggtaatggacatcagcacgaatctcctaatataggtcaagtcgaggacaccttgactcgtttcacaaattcagtcttgaatcacaaagacgtccttaactctagctcatctgatcaagatactttgagaagagagtttagaacattcatgcacgctcatataacacaaatcgaagataactcacgattcagtaagcaagcctcatccgatgcgttttcctctcctgaacaatcttactttcaatgggtgaactcaactggtggctcacatgtcgcttgcgcctattcatttgccttctctaattgcctcatgtctgcaaatttgttgcagggtaaagacgcatttccaagcggaacgcaaaagtacttaatctcctctgttatgagacatgccacaaacatgtgtagaatgtataacgactttggctctattgccagagacaacgctgagagaaatgttaatagtattcattttcctgagtttactctctgtaacggaacttctcaaaacctagatgaaaggaaggaaagacttctgaaaatcgcaacttacgaacaagggtatttggatagagcactagaggccttggaaagacagagtagagatgatgccggagacagagctggatctaaagatatgagaaagttgaaaatcgttaagttattctgtgatgttacggacttatacgatcagctctacgttatcaaagatttgtcatcctctatgaagtaa

SEQ ID NO: 58 - protein, 952 aa, Gibberella fujikuroi
MPGKIENGTPKDLKTGNDFVSAAKSLLDRAFKSHHSYYGLCSTSCQVYDTAWVAMIPKTRDNVKQWLFPECFHYLLKTQAADGSWGSLPTTQTAGILDTASAVLALLCHAQEPLQILDVSPDEMGLRIEHGVTSLKRQLAVWNDVEDTNHIGVEFIIPALLSMLEKELDVPSFEFPCRSILERMHGEKLGHFDLEQVYGKPSSLLHSLEAFLGKLDFDRLSHHLYHGSMMASPSSTAAYLIGATKWDDEAEDYLRHVMRNGAGHGNGGISGTFPTTHFECSWIIATLLKVGFTLKQIDGDGLRGLSTILLEALRDENGVIGFAPRTADVDDTAKALLALSLVNQPVSPDIMIKVFEGKDHFTTFGSERDPSLTSNLHVLLSLLKQSNLSQYHPQILKTTLFTCRWWWGSDHCVKDKWNLSHLYPTMLLVEAFTEVLHLIDGGELSSLFDESFKCKIGLSIFQAVLRIILTQDNDGSWRGYREQTCYAILALVQARHVCFFTHMVDRLQSCVDRGFSWLKSCSFHSQDLTWTSKTAYEVGFVAEAYKLAALQSASLEVPAATIGHSVTSAVPSSDLEKYMRLVRKTALFSPLDEWGLMASIIESSFFVPLLQAQRVEIYPRDNIKVDEDKYLSIIPFTWVGCNNRSRTFASNRWLYDMMYLSLLGYQTDEYMEAVAGPVFGDVSLLHQTIDKVIDNTMGNLARANGTVHSGNGHQHESPNIGQVEDTLTRFTNSVLNHKDVLNSSSSDQDTLRREFRTFMHAHITQIEDNSRFSKQASSDAFSSPEQSYFQWVNSTGGSHVACAYSFAFSNCLMSANLLQGKDAFPSGTQKYLISSVMRHATNMCRMYNDFGSIARDNAERNVNSIHFPEFTLCNGTSQNLDERKERLLKIATYEQGYLDRALEALERQSRDDAGDRAGSKDMRKLKIVKLFCDVTDLYDQLYVIKDLSSSMK

SEQ ID NO: 59 - DNA, 1542 bp, Artificial Sequence
  Codon-optimized KO
atggatgctgtgacgggtttgttaactgtcccagcaaccgctataactattggtggaactgctgtagcattggcggtagcgctaatcttttggtacctgaaatcctacacatcagctagaagatcccaatcaaatcatcttccaagagtgcctgaagtcccaggtgttccattgttaggaaatctgttacaattgaaggagaaaaagccatacatgacttttacgagatgggcagcgacatatggacctatctatagtatcaaaactggggctacaagtatggttgtggtatcatctaatgagatagccaaggaggcattggtgaccagattccaatccatatctacaaggaacttatctaaagccctgaaagtacttacagcagataagacaatggtcgcaatgtcagattatgatgattatcataaaacagttaagagacacatactgaccgccgtcttgggtcctaatgcacagaaaaagcatagaattcacagagatatcatgatggataacatatctactcaacttcatgaattcgtgaaaaacaacccagaacaggaagaggtagaccttagaaaaatctttcaatctgagttattcggcttagctatgagacaagccttaggaaaggatgttgaaagtttgtacgttgaagacctgaaaatcactatgaatagagacgaaatctttcaagtccttgttgttgatccaatgatgggagcaatcgatgttgattggagagacttctttccatacctaaagtgggtcccaaacaaaaagttcgaaaatactattcaacaaatgtacatcagaagagaagctgttatgaaatctttaatcaaagagcacaaaaagagaatagcgtcaggcgaaaagctaaatagttatatcgattaccttttatctgaagctcaaactttaaccgatcagcaactattgatgtccttgtgggaaccaatcattgaatcttcagatacaacaatggtcacaacagaatgggcaatgtacgaattagctaaaaaccctaaattgcaagataggttgtacagagacattaagtccgtctgtggatctgaaaagataaccgaagagcatctatcacagctgccttacattacagctattttccacgaaacactgagaagacactcaccagttcctatcattcctctaagacatgtacatgaagataccgttctaggcggctaccatgttcctgctggcacagaacttgccgttaacatctacggttgcaacatggacaaaaacgtttgggaaaatccagaggaatggaacccagaaagattcatgaaagagaatgagacaattgattttcaaaagacgatggccttcggtggtggtaagagagtttgtgctggttccttgcaagcccttttaactgcatctattgggattgggagaatggttcaagagttcgaatggaaactgaaggatatgactcaagaggaagtgaacacgataggcctaactacacaaatgttaagaccattgagagctattatcaaacctaggatctaa

SEQ ID NO: 60 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 61 - DNA, 1566 bp, Artificial Sequence
  Codon-optimized KO
aagcttactagtaaaatggacggtgtcatcgatatgcaaaccattccattgagaaccgctattgctattggtggtactgctgttgctttggttgttgcattatacttttggttcttgagatcctacgcttccccatctcatcattctaatcatttgccaccagtacctgaagttccaggtgttccagttttgggtaatttgttgcaattgaaagaaaaaaagccttacatgaccttcaccaagtgggctgaaatgtatggtccaatctactctattagaactggtgctacttccatggttgttgtctcttctaacgaaatcgccaaagaagttgttgttaccagattcccatctatctctaccagaaaattgtcttacgccttgaaggttttgaccgaagataagtctatggttgccatgtctgattatcacgattaccataagaccgtcaagagacatattttgactgctgttttgggtccaaacgcccaaaaaaagtttagagcacatagagacaccatgatggaaaacgtttccaatgaattgcatgccttcttcgaaaagaacccaaatcaagaagtcaacttgagaaagatcttccaatcccaattattcggtttggctatgaagcaagccttgggtaaagatgttgaatccatctacgttaaggatttggaaaccaccatgaagagagaagaaatcttcgaagttttggttgtcgatccaatgatgggtgctattgaagttgattggagagactttttcccatacttgaaatgggttccaaacaagtccttcgaaaacatcatccatagaatgtacactagaagagaagctgttatgaaggccttgatccaagaacacaagaaaagaattgcctccggtgaaaacttgaactcctacattgattacttgttgtctgaagcccaaaccttgaccgataagcaattattgatgtctttgtgggaacctattatcgaatcttctgataccactatggttactactgaatgggctatgtacgaattggctaagaatccaaacatgcaagacagattatacgaagaaatccaatccgtttgcggttccgaaaagattactgaagaaaacttgtcccaattgccatacttgtacgctgttttccaagaaactttgagaaagcactgtccagttcctattatgccattgagatatgttcacgaaaacaccgttttgggtggttatcatgttccagctggtactgaagttgctattaacatctacggttgcaacatggataagaaggtctgggaaaatccagaagaatggaatccagaaagattcttgtccgaaaaagaatccatggacttgtacaaaactatggcttttggtggtggtaaaagagtttgcgctggttctttacaagccatggttatttcttgcattggtatcggtagattggtccaagattttgaatggaagttgaaggatgatgccgaagaagatgttaacactttgggtttgactacccaaaagttgcatccattattggccttgattaacccaagaaagtaactcgagccgcgg

SEQ ID NO: 62 - protein, 512 aa, Lactuca sativa
MDGVIDMQTIPLRTAIAIGGTAVALVVALYFWFLRSYASPSHHSNHLPPVPEVPGVPVLGNLLQLKEKKPYMTFTKWAEMYGPIYSIRTGATSMVVVSSNEIAKEVVVTRFPSISTRKLSYALKVLTEDKSMVAMSDYHDYHKTVKRHILTAVLGPNAQKKFRAHRDTMMENVSNELHAFFEKNPNQEVNLRKIFQSQLFGLAMKQALGKDVESIYVKDLETTMKREEIFEVLVVDPMMGAIEVDWRDFFPYLKWVPNKSFENIIHRMYTRREAVMKALIQEHKKRIASGENLNSYIDYLLSEAQTLTDKQLLMSLWEPIIESSDTTMVTTEWAMYELAKNPNMQDRLYEEIQSVCGSEKITEENLSQLPYLYAVFQETLRKHCPVPIMPLRYVHENTVLGGYHVPAGTEVAINIYGCNMDKKVWENPEEWNPERFLSEKESMDLYKTMAFGGGKRVCAGSLQAMVISCIGIGRLVQDFEWKLKDDAEEDVNTLGLTTQKLHPLLALINPRK

SEQ ID NO: 63 - DNA, 1535 bp, Rubus suavissimus
atggccaccctccttgagcatttccaagctatgccctttgccatccctattgcactggctgctctgtcttggctgttcctcttttacatcaaagtttcattcttttccaacaagagtgctcaggctaagctccctcctgtgccagtggttcctgggctgccggtgattgggaatttactgcaactcaaggagaagaaaccctaccagacttttacaaggtgggctgaggagtatggaccaatctattctatcaggactggtgcttccaccatggtcgttctcaataccacccaagttgcaaaagaggccatggtgaccagatatttatccatctcaaccagaaagctatcaaacgcactaaagattcttactgctgataaatgtatggttgcaataagtgactacaacgattttcacaagatgataaagcgatacatactctcaaatgttcttggacctagtgctcagaagcgtcaccggagcaacagagataccttgagagctaatgtctgcagccgattgcattctcaagtaaagaactctcctcgagaagctgtgaatttcagaagagtttttgagtgggaactctttggaattgcattgaagcaagcctttggaaaggacatagaaaagcccatttatgtggaggaacttggcactacactgtcaagagatgagatctttaaggttctagtgcttgacataatggagggtgcaattgaggttgattggagagatttcttcccttacctgagatggattccgaatacgcgcatggaaacaaaaattcagcgactctatttccgcaggaaagcagtgatgactgccctgatcaacgagcagaagaagcgaattgcttcaggagaggaaatcaactgttatatcgacttcttgcttaaggaagggaagacactgacaatggaccaaataagtatgttgctttgggagacggttattgaaacagcagatactacaatggtaacgacagaatgggctatgtatgaagttgctaaagactcaaagcgtcaggatcgtctctatcaggaaatccaaaaggtttgtggatcggagatggttacagaggaatacttgtcccaactgccgtacctgaatgcagttttccatgaaacgctaaggaagcacagtccggctgcgttagttcctttaagatatgcacatgaagatacccaactaggaggttactacattccagctggaactgagattgctataaacatatacgggtgtaacatggacaagcatcaatgggaaagccctgaggaatggaaaccggagagatttttggacccgaaatttgatcctatggatttgtacaagaccatggcttttggggctggaaagagggtatgtgctggttctcttcaggcaatgttaatagcgtgcccgacgattggtaggctggtgcaggagtttgagtggaagctgagagatggagaagaagaaaatgtagatactgttgggctcaccactcacaaacgctatccaatgcatgcaatcctgaagccaagaagtta

SEQ ID NO: 64 - DNA, 1536 bp, Artificial Sequence
  Codon-optimized KO
atggctaccttgttggaacattttcaagctatgccattcgctattccaattgctttggctgctttgtcttggttgtttttgttctacatcaaggtttctttcttctccaacaaatccgctcaagctaaattgccaccagttccagttgttccaggtttgccagttattggtaatttgttgcaattgaaagaaaagaagccataccaaaccttcactagatgggctgaagaatatggtccaatctactctattagaactggtgcttctactatggttgtcttgaacactactcaagttgccaaagaagctatggttaccagatacttgtctatctctaccagaaagttgtccaacgccttgaaaattttgaccgctgataagtgcatggttgccatttctgattacaacgatttccacaagatgatcaagagatatatcttgtctaacgttttgggtccatctgcccaaaaaagacatagatctaacagagataccttgagagccaacgtttgttctagattgcattcccaagttaagaactctccaagagaagctgtcaactttagaagagttttcgaatgggaattattcggtatcgctttgaaacaagccttcggtaaggatattgaaaagccaatctacgtcgaagaattgggtactactttgtccagagatgaaatcttcaaggttttggtcttggacattatggaaggtgccattgaagttgattggagagattttttcccatacttgcgttggattccaaacaccagaatggaaactaagatccaaagattatactttagaagaaaggccgttatgaccgccttgattaacgaacaaaagaaaagaattgcctccggtgaagaaatcaactgctacatcgatttcttgttgaaagaaggtaagaccttgaccatggaccaaatctctatgttgttgtgggaaaccgttattgaaactgctgataccacaatggttactactgaatgggctatgtacgaagttgctaaggattctaaaagacaagacagattataccaagaaatccaaaaggtctgcggttctgaaatggttacagaagaatacttgtcccaattgccatacttgaatgctgttttccacgaaactttgagaaaacattctccagctgctttggttccattgagatatgctcatgaagatactcaattgggtggttattacattccagccggtactgaaattgccattaacatctacggttgcaacatggacaaacaccaatgggaatctccagaagaatggaagccagaaagatttttggatcctaagtttgacccaatggacttgtacaaaactatggcttttggtgctggtaaaagagtttgcgctggttctttacaagctatgttgattgcttgtccaaccatcggtagattggttcaagaatttgaatggaagttgagagatggtgaagaagaaaacgttgatactgttggtttgaccacccataagagatatccaatgcatgctattttgaagccaagatcttaa

SEQ ID NO: 65 - DNA, 1572 bp, Artificial Sequence
  Codon-optimized KO
aagcttactagtaaaatggcctccatcacccatttcttacaagattttcaagctactccattcgctactgcttttgctgttggtggtgtttctttgttgatattcttcttcttcatccgtggtttccactctactaagaaaaacgaatattacaagttgccaccagttccagttgttccaggtttgccagttgttggtaatttgttgcaattgaaagaaaagaagccatacaagactttcttgagatgggctgaaattcatggtccaatctactctattagaactggtgcttctaccatggttgttgttaactctactcatgttgccaaagaagctatggttaccagattctcttcaatctctaccagaaagttgtccaaggctttggaattattgacctccaacaaatctatggttgccacctctgattacaacgaatttcacaagatggtcaagaagtacatcttggccgaattattgggtgctaatgctcaaaagagacacagaattcatagagacaccttgatcgaaaacgtcttgaacaaattgcatgcccataccaagaattctccattgcaagctgttaacttcagaaagatcttcgaatctgaattattcggtttggctatgaagcaagccttgggttatgatgttgattccttgttcgttgaagaattgggtactaccttgtccagagaagaaatctacaacgttttggtcagtgacatgttgaagggtgctattgaagttgattggagagactttttcccatacttgaaatggatcccaaacaagtccttcgaaatgaagattcaaagattggcctctagaagacaagccgttatgaactctattgtcaaagaacaaaagaagtccattgcctctggtaagggtgaaaactgttacttgaattacttgttgtccgaagctaagactttgaccgaaaagcaaatttccattttggcctgggaaaccattattgaaactgctgatacaactgttgttaccactgaatgggctatgtacgaattggctaaaaacccaaagcaacaagacagattatacaacgaaatccaaaacgtctgcggtactgataagattaccgaagaacatttgtccaagttgccttacttgtctgctgtttttcacgaaaccttgagaaagtattctccatctccattggttccattgagatacgctcatgaagatactcaattgggtggttattatgttccagccggtactgaaattgctgttaatatctacggttgcaacatggacaagaatcaatgggaaactccagaagaatggaagccagaaagatttttggacgaaaagtacgatccaatggacatgtacaagactatgtcttttggttccggtaaaagagtttgcgctggttctttacaagctagtttgattgcttgtacctccatcggtagattggttcaagaatttgaatggagattgaaagacggtgaagttgaaaacgttgataccttgggtttgactacccataagttgtatccaatgcaagctatcttgcaacctagaaactgactcgagccgcgg

SEQ ID NO: 66 - protein, 514 aa, Castanea mollissima
MASITHFLQDFQATPFATAFAVGGVSLLIFFFFIRGFHSTKKNEYYKLPPVPVVPGLPVVGNLLQLKEKKPYKTFLRWAEIHGPIYSIRTGASTMVVVNSTHVAKEAMVTRFSSISTRKLSKALELLTSNKSMVATSDYNEFHKMVKKYILAELLGANAQKRHRIHRDTLIENVLNKLHAHTKNSPLQAVNFRKIFESELFGLAMKQALGYDVDSLFVEELGTTLSREEIYNVLVSDMLKGAIEVDWRDFFPYLKWIPNKSFEMKIQRLASRRQAVMNSIVKEQKKSIASGKGENCYLNYLLSEAKTLTEKQISILAWETIIETADTTVVTTEWAMYELAKNPKQQDRLYNEIQNVCGTDKITEEHLSKLPYLSAVFHETLRKYSPSPLVPLRYAHEDTQLGGYYVPAGTEIAVNIYGCNMDKNQWETPEEWKPERFLDEKYDPMDMYKTMSFGSGKRVCAGSLQASLIACTSIGRLVQEFEWRLKDGEVENVDTLGLTTHKLYPMQAILQPRN

SEQ ID NO: 67 - DNA, 1512 bp, Artificial Sequence
  Codon-optimized KO
atgatttccttgttgttgggttttgttgtctcctccttcttgtttatcttcttcttgaaaaaattgttgttcttcttcagtcgtcacaaaatgtccgaagtttctagattgccatctgttccagttccaggttttccattgattggtaacttgttgcaattgaaagaaaagaagccacacaagactttcaccaagtggtctgaattatatggtccaatctactctatcaagatgggttcctcttctttgatcgtcttgaactctattgaaaccgccaaagaagctatggtcagtagattctcttcaatctctaccagaaagttgtctaacgctttgactgttttgacctgcaacaaatctatggttgctacctctgattacgatgactttcataagttcgtcaagagatgcttgttgaacggtttgttgggtgctaatgctcaagaaagaaaaagacattacagagatgccttgatcgaaaacgttacctctaaattgcatgcccataccagaaatcatccacaagaaccagttaacttcagagccattttcgaacacgaattattcggtgttgctttgaaacaagccttcggtaaagatgtcgaatccatctatgtaaaagaattgggtgtcaccttgtccagagatgaaattttcaaggttttggtccacgacatgatggaaggtgctattgatgttgattggagagatttcttcccatacttgaaatggatcccaaacaactctttcgaagccagaattcaacaaaagcacaagagaagattggctgttatgaacgccttgatccaagacagattgaatcaaaacgattccgaatccgatgatgactgctacttgaatttcttgatgtctgaagctaagaccttgaccatggaacaaattgctattttggtttgggaaaccattatcgaaactgctgataccactttggttactactgaatgggctatgtacgaattggccaaacatcaatctgttcaagatagattattcaaagaaatccaatccgtctgcggtggtgaaaagatcaaagaagaacaattgccaagattgccttacgtcaatggtgtttttcacgaaaccttgagaaagtattctccagctccattggttccaattagatacgctcatgaagatacccaaattggtggttatcatattccagccggttctgaaattgccattaacatctacggttgcaacatggataagaagagatgggaaagacctgaagaatggtggccagaaagatttttggaagatagatacgaatcctccgacttgcataagactatggcttttggtgctggtaaaagagtttgtgctggtgctttacaagctagtttgatggctggtattgctatcggtagattggttcaagaattcgaatggaagttgagagatggtgaagaagaaaacgttgatacttacggtttgacctcccaaaagttgtatccattgatggccattatcaacccaagaagatcttaa

SEQ ID NO: 68 - protein, 506 aa, Thellungiella halophila
MASMISLLLGFVVSSFLFIFFLKKLLFFFSRHKMSEVSRLPSVPVPGFPLIGNLLQLKEKKPHKTFTKWSELYGPIYSIKMGSSSLIVLNSIETAKEAMVSRFSSISTRKLSNALTVLTCNKSMVATSDYDDFHKFVKRCLLNGLLGANAQERKRHYRDALIENVTSKLHAHTRNHPQEPVNFRAIFEHELFGVALKQAFGKDVESIYVKELGVTLSRDEIFKVLVHDMMEGAIDVDWRDFFPYLKWIPNNSFEARIQQKHKRRLAVMNALIQDRLNQNDSESDDDCYLNFLMSEAKTLTMEQIAILVWETIIETADTTLVTTEWAMYELAKHQSVQDRLFKEIQSVCGGEKIKEEQLPRLPYVNGVFHETLRKYSPAPLVPIRYAHEDTQIGGYHIPAGSEIAINIYGCNMDKKRWERPEEWWPERFLEDRYESSDLHKTMAFGAGKRVCAGALQASLMAGIAIGRLVQEFEWKLRDGEEENVDTYGLTSQKLYPLMAIINPRRS

SEQ ID NO: 69 - DNA, 1554 bp, Artificial Sequence
  Codon-optimized KO
aagcttactagtaaaatggacatgatgggtattgaagctgttccatttgctactgctgttgttttgggtggtatttccttggttgttttgatcttcatcagaagattcgtttccaacagaaagagatccgttgaaggtttgccaccagttccagatattccaggtttaccattgattggtaacttgttgcaattgaaagaaaagaagccacataagacctttgctagatgggctgaaacttacggtccaattttctctattagaactggtgcttctaccatgatcgtcttgaattcttctgaagttgccaaagaagctatggtcactagattctcttcaatctctaccagaaagttgtccaacgccttgaagattttgaccttcgataagtgtatggttgccacctctgattacaacgattttcacaaaatggtcaagggtttcatcttgagaaacgttttaggtgctccagcccaaaaaagacatagatgtcatagagataccttgatcgaaaacatctctaagtacttgcatgcccatgttaagacttctccattggaaccagttgtcttgaagaagattttcgaatccgaaattttcggtttggctttgaaacaagccttgggtaaggatatcgaatccatctatgttgaagaattgggtactaccttgtccagagaagaaatttttgccgttttggttgttgatccaatggctggtgctattgaagttgattggagagattttttcccatacttgtcctggattccaaacaagtctatggaaatgaagatccaaagaatggattttagaagaggtgctttgatgaaggccttgattggtgaacaaaagaaaagaatcggttccggtgaagaaaagaactcctacattgatttcttgttgtctgaagctaccactttgaccgaaaagcaaattgctatgttgatctgggaaaccatcatcgaaatttccgatacaactttggttacctctgaatgggctatgtacgaattggctaaagacccaaatagacaagaaatcttgtacagagaaatccacaaggtttgcggttctaacaagttgactgaagaaaacttgtccaagttgccatacttgaactctgttttccacgaaaccttgagaaagtattctccagctccaatggttccagttagatatgctcatgaagatactcaattgggtggttaccatattccagctggttctcaaattgccattaacatctacggttgcaacatgaacaaaaagcaatgggaaaatcctgaagaatggaagccagaaagattcttggacgaaaagtatgacttgatggacttgcataagactatggcttttggtggtggtaaaagagtttgtgctggtgctttacaagcaatgttgattgcttgcacttccatcggtagattcgttcaagaatttgaatggaagttgatgggtggtgaagaagaaaacgttgatactgttgctttgacctcccaaaaattgcatccaatgcaagccattattaaggccagagaatgactcgagccgcgg

SEQ ID NO: 70 - protein, 508 aa, Vitis vinifera
MDMMGIEAVPFATAVVLGGISLVVLIFIRRFVSNRKRSVEGLPPVPDIPGLPLIGNLLQLKEKKPHKTFARWAETYGPIFSIRTGASTMIVLNSSEVAKEAMVTRFSSISTRKLSNALKILTFDKCMVATSDYNDFHKMVKGFILRNVLGAPAQKRHRCHRDTLIENISKYLHAHVKTSPLEPVVLKKIFESEIFGLALKQALGKDIESIYVEELGTTLSREEIFAVLVVDPMAGAIEVDWRDFFPYLSWIPNKSMEMKIQRMDFRRGALMKALIGEQKKRIGSGEEKNSYIDFLLSEATTLTEKQIAMLIWETIIEISDTTLVTSEWAMYELAKDPNRQEILYREIHKVCGSNKLTEENLSKLPYLNSVFHETLRKYSPAPMVPVRYAHEDTQLGGYHIPAGSQIAINIYGCNMNKKQWENPEEWKPERFLDEKYDLMDLHKTMAFGGGKRVCAGALQAMLIACTSIGRFVQEFEWKLMGGEEENVDTVALTSQKLHPMQAIIKARE

SEQ ID NO: 71 - DNA, 1593 bp, Artificial Sequence
  Codon-optimized KO
aagcttaaaatgagtaagtctaatagtatgaattctacatcacacgaaaccctttttcaacaattggtcttgggtttggaccgtatgccattgatggatgttcactggttgatctacgttgctttcggcgcatggttatgttcttatgtgatacatgttttatcatcttcctctacagtaaaagtgccagttgttggatacaggtctgtattcgaacctacatggttgcttagacttagattcgtctgggaaggtggctctatcataggtcaagggtacaataagtttaaagactctattttccaagttaggaaattgggaactgatattgtcattataccacctaactatattgatgaagtgagaaaattgtcacaggacaagactagatcagttgaacctttcattaatgattttgcaggtcaatacacaagaggcatggttttcttgcaatctgacttacaaaaccgtgttatacaacaaagactaactccaaaattggtttccttgaccaaggtcatgaaggaagagttggattatgctttaacaaaagagatgcctgatatgaaaaatgacgaatgggtagaagtagatatcagtagtataatggtgagattgatttccaggatctccgccagagtctttctagggcctgaacactgtcgtaaccaggaatggttgactactacagcagaatattcagaatcacttttcattacagggtttatcttaagagttgtacctcatatcttaagaccattcatcgcccctctattaccttcatacaggactctacttagaaacgtttcaagtggtagaagagtcatcggtgacatcataagatctcagcaaggggatggtaacgaagatatactttcctggatgagagatgctgccacaggagaggaaaagcaaatcgataacattgctcagagaatgttaattctttctttagcatcaatccacactactgcgatgaccatgacacatgccatgtacgatctatgtgcttgccctgagtacattgaaccattaagagatgaagttaaatctgttgttggggcttctggctgggacaagacagcgttaaacagatttcataagttggactccttcctaaaagagtcacaaagattcaacccagtattcttattgacattcaatagaatctaccatcaatctatgaccttatcagatggcactaacattccatctggaacacgtattgctgttccatcacacgcaatgttgcaagattctgcacatgtcccaggtccaaccccacctactgaatttgatggattcagatatagtaagatacgttctgatagtaactacgcacaaaagtacctattctccatgaccgattcttcaaacatggctttcggatacggcaagtatgcttgtccaggtagattttacgcgtctaatgagatgaaactaacattagccattttgttgctacaatttgagttcaaactaccagatggtaaaggtcgtcctagaaatatcactatcgattctgatatgattccagacccaagagctagactttgcgtcagaaaaagatcacttagagatgaatgaccgcgg

SEQ ID NO: 72 - protein, 525 aa, Gibberella fujikuroi
MSKSNSMNSTSHETLFQQLVLGLDRMPLMDVHWLIYVAFGAWLCSYVIHVLSSSSTVKVPVVGYRSVFEPTWLLRLRFVWEGGSIIGQGYNKFKDSIFQVRKLGTDIVIIPPNYIDEVRKLSQDKTRSVEPFINDFAGQYTRGMVFLQSDLQNRVIQQRLTPKLVSLTKVMKEELDYALTKEMPDMKNDEWVEVDISSIMVRLISRISARVFLGPEHCRNQEWLTTTAEYSESLFITGFILRVVPHILRPFIAPLLPSYRTLLRNVSSGRRVIGDIIRSQQGDGNEDILSWMRDAATGEEKQIDNIAQRMLILSLASIHTTAMTMTHAMYDLCACPEYIEPLRDEVKSVVGASGWDKTALNRFHKLDSFLKESQRFNPVFLLTFNRIYHQSMTLSDGTNIPSGTRIAVPSHAMLQDSAHVPGPTPPTEFDGFRYSKIRSDSNYAQKYLFSMTDSSNMAFGYGKYACPGRFYASNEMKLTLAILLLQFEFKLPDGKGRPRNITIDSDMIPDPRARLCVRKRSLRDE

SEQ ID NO: 73 - DNA, 1515 bp, Artificial Sequence
  Codon-optimized KO
aagcttaaaatggaagatcctactgtcttatatgcttgtcttgccattgcagttgcaactttcgttgttagatggtacagagatccattgagatccatcccaacagttggtggttccgatttgcctattctatcttacatcggcgcactaagatggacaagacgtggcagagagatacttcaagagggatatgatggctacagaggatctacattcaaaatcgcgatgttagaccgttggatcgtgatcgcaaatggtcctaaactagctgatgaagtcagacgtagaccagatgaagagttaaactttatggacggattaggagcattcgtccaaactaagtacaccttaggtgaagctattcataacgatccataccatgtcgatatcataagagaaaaactaacaagaggccttccagccgtgcttcctgatgtcattgaagagttgacacttgcggttagacagtacattccaacagaaggtgatgaatgggtgtccgtaaactgttcaaaggccgcaagagatattgttgctagagcttctaatagagtctttgtaggtttgcctgcttgcagaaaccaaggttacttagatttggcaatagactttacattgtctgttgtcaaggatagagccatcatcaatatgtttccagaattgttgaagccaatagttggcagagttgtaggtaacgccaccagaaatgttcgtagagctgttccttttgttgctccattggtggaggaaagacgtagacttatggaagagtacggtgaagactggtctgaaaaacctaatgatatgttacagtggataatggatgaagctgcatccagagatagttcagtgaaggcaatcgcagagagattgttaatggtgaacttcgcggctattcatacctcatcaaacactatcactcatgctttgtaccaccttgccgaaatgcctgaaactttgcaaccacttagagaagagatcgaaccattagtcaaagaggagggctggaccaaggctgctatgggaaaaatgtggtggttagattcatttctaagagaatctcaaagatacaatggcattaacatcgtatctttaactagaatggctgacaaagatattacattgagtgatggcacatttttgccaaaaggtactctagtggccgttccagcgtattctactcatagagatgatgctgtctacgctgatgccttagtattcgatcctttcagattctcacgtatgagagcgagagaaggtgaaggtacaaagcaccagttcgttaatacttcagtcgagtacgttccatttggtcacggaaagcatgcttgtccaggaagattcttcgccgcaaacgaattgaaagcaatgttggcttacattgttctaaactatgatgtaaagttgcctggtgacggtaaacgtccattgaacatgtattggggtccaacagttttgcctgcaccagcaggccaagtattgttcagaaagagacaagttagtctataaccgcgg

SEQ ID NO: 74 - protein, 499 aa, Trametes versicolor
MEDPTVLYACLAIAVATFVVRWYRDPLRSIPTVGGSDLPILSYIGALRWTRRGREILQEGYDGYRGSTFKIAMLDRWIVIANGPKLADEVRRRPDEELNFMDGLGAFVQTKYTLGEAIHNDPYHVDIIREKLTRGLPAVLPDVIEELTLAVRQYIPTEGDEWVSVNCSKAARDIVARASNRVFVGLPACRNQGYLDLAIDFTLSVVKDRAIINMFPELLKPIVGRVVGNATRNVRRAVPFVAPLVEERRRLMEEYGEDWSEKPNDMLQWIMDEAASRDSSVKAIAERLLMVNFAAIHTSSNTITHALYHLAEMPETLQPLREEIEPLVKEEGWTKAAMGKMWWLDSFLRESQRYNGINIVSLTRMADKDITLSDGTFLPKGTLVAVPAYSTHRDDAVYADALVFDPFRFSRMRAREGEGTKHQFVNTSVEYVPFGHGKHACPGRFFAANELKAMLAYIVLNYDVKLPGDGKRPLNMYWGPTVLPAPAGQVLFRKRQVSL

SEQ ID NO: 75 - DNA, 1530 bp, Artificial Sequence
  Codon-optimized KO
atggcatttttctctatgatttcaattttgttgggatttgttatttcttctttcatcttcatctttttcttcaaaaagttacttagttttagtaggaaaaacatgtcagaagtttctactttgccaagtgttccagtagtgcctggttttccagttattgggaatttgttgcaactaaaggagaaaaagcctcataaaactttcactagatggtcagagatatatggacctatctactctataaagatgggttcttcatctcttattgtattgaacagtacagaaactgctaaggaagcaatggtcactagattttcatcaatatctaccagaaaattgtcaaacgccctaacagttctaacctgcgataagtctatggtcgccacttctgattatgatgacttccacaaattagttaagagatgtttgctaaatggacttcttggtgctaatgctcaaaagagaaaaagacactacagagatgctttgattgaaaatgtgagttccaagctacatgcacacgctagagatcatccacaagagccagttaactttagagcaattttcgaacacgaattgtttggtgtagcattaaagcaagccttcggtaaagacgtagaatccatatacgtcaaggagttaggcgtaacattatcaaaagatgaaatctttaaggtgcttgtacatgatatgatggagggtgcaattgatgtagattggagagatttcttcccatatttgaaatggatccctaataagtcttttgaagctaggatacaacaaaagcacaagagaagactagctgttatgaacgcacttatacaggacagattgaagcaaaatgggtctgaatcagatgatgattgttaccttaacttcttaatgtctgaggctaaaacattgactaaggaacagatcgcaatccttgtctgggaaacaatcattgaaacagcagatactaccttagtcacaactgaatgggccatatacgagctagccaaacatccatctgtgcaagataggttgtgtaaggagatccagaacgtgtgtggtggagagaaattcaaggaagagcagttgtcacaagttccttaccttaacggcgttttccatgaaaccttgagaaaatactcacctgcaccattagttcctattagatacgcccacgaagatacacaaatcggtggctaccatgttccagctgggtccgaaattgctataaacatctacgggtgcaacatggacaaaaagagatgggaaagaccagaagattggtggccagaaagattcttagatgatggcaaatatgaaacatctgatttgcataaaacaatggctttcggagctggcaaaagagtgtgtgccggtgctctacaagcctccctaatggctggtatcgctattggtagattggtccaagagttcgaatggaaacttagagatggtgaagaggaaaatgtcgatacttatgggttaacatctcaaaagttatacccactaatggcaatcatcaatcctagaagatcctaa

SEQ ID NO: 76 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 77 - DNA, 2133 bp, Artificial Sequence
  Codon-optimized CPR
atgcaatcagattcagtcaaagtctctccatttgatttggtttccgctgctatgaatggcaaggcaatggaaaagttgaacgctagtgaatctgaagatccaacaacattgcctgcactaaagatgctagttgaaaatagagaattgttgacactgttcacaacttccttcgcagttcttattgggtgtcttgtatttctaatgtggagacgttcatcctctaaaaagctggtacaagatccagttccacaagttatcgttgtaaagaagaaagagaaggagtcagaggttgatgacgggaaaaagaaagtttctattttctacggcacacaaacaggaactgccgaaggttttgctaaagcattagtcgaggaagcaaaagtgagatatgaaaagacctctttcaaggttatcgatctagatgactacgctgcagatgatgatgaatatgaggaaaaactgaaaaaggaatccttagccttcttcttcttggccacatacggtgatggtgaacctactgataatgctgctaacttctacaagtggttcacagaaggcgacgataaaggtgaatggctgaaaaagttacaatacggagtatttggtttaggtaacagacaatatgaacatttcaacaagatcgctattgtagttgatgataaacttactgaaatgggagccaaaagattagtaccagtaggattaggggatgatgatcagtgtatagaagatgacttcaccgcctggaaggaattggtatggccagaattggatcaacttttaagggacgaagatgatacttctgtgactaccccatacactgcagccgtattggagtacagagtggtttaccatgataaaccagcagactcatatgctgaagatcaaacccatacaaacggtcatgttgttcatgatgcacagcatccttcaagatctaatgtggctttcaaaaaggaactacacacctctcaatcagataggtcttgtactcacttagaattcgatatttctcacacaggactgtcttacgaaactggcgatcacgttggcgtttattccgagaacttgtccgaagttgtcgatgaagcactaaaactgttagggttatcaccagacacatacttctcagtccatgctgataaggaggatgggacacctatcggtggtgcttcactaccaccaccttttcctccttgcacattgagagacgctctaaccagatacgcagatgtcttatcctcacctaaaaaggtagctttgctggcattggctgctcatgctagtgatcctagtgaagccgataggttaaagttcctggcttcaccagccggaaaagatgaatatgcacaatggatcgtcgccaaccaacgttctttgctagaagtgatgcaaagttttccatctgccaagcctccattaggtgtgttcttcgcagcagtagctccacgtttacaaccaagatactactctatcagttcatctcctaagatgtctcctaacagaatacatgttacatgtgctttggtgtacgagactactccagcaggcagaattcacagaggattgtgttcaacctggatgaaaaatgctgtccctttaacagagtcacctgattgctctcaagcatccattttcgttagaacatcaaatttcagacttccagtggatccaaaagttccagtcattatgataggaccaggcactggtcttgccccattcaggggctttcttcaagagagattggccttgaaggaatctggtacagaattgggttcttctatctttttctttggttgccgtaatagaaaagttgactttatctacgaggacgagcttaacaattttgttgagacaggagcattgtcagaattgatcgtcgcattttcaagagaagggactgccaaagagtacgttcagcacaagatgagtcaaaaagcctccgatatatggaaacttctaagtgaaggtgcctatctttatgtctgtggcgatgcaaagggcatggccaaggatgtccatagaactctgcatacaattgttcaggaacaagggagtctggattcttccaaggctgaattgtacgtcaaaaacttacagatgtctggaagatacttaagagatgtttggtaa

SEQ ID NO: 78 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 79 - DNA, 2106 bp, Siraitia grosvenorii
atgaaggtcagtccattcgaattcatgtccgctattatcaagggtagaatggacccatctaactcctcatttgaatctactggtgaagttgcctccgttatctttgaaaacagagaattggttgccatcttgaccacttctattgctgttatgattggttgcttcgttgtcttgatgtggagaagagctggttctagaaaggttaagaatgtcgaattgccaaagccattgattgtccatgaaccagaacctgaagttgaagatggtaagaagaaggtttccatcttcttcggtactcaaactggtactgctgaaggttttgctaaggctttggctgatgaagctaaagctagatacgaaaaggctaccttcagagttgttgatttggatgattatgctgccgatgatgaccaatacgaagaaaaattgaagaacgaatccttcgccgttttcttgttggctacttatggtgatggtgaacctactgataatgctgctagattttacaagtggttcgccgaaggtaaagaaagaggtgaatggttgcaaaacttgcactatgctgtttttggtttgggtaacagacaatacgaacacttcaacaagattgctaaggttgccgacgaattattggaagctcaaggtggtaatagattggttaaggttggtttaggtgatgacgatcaatgcatcgaagatgatttttctgcttggagagaatctttgtggccagaattggatatgttgttgagagatgaagatgatgctactactgttactactccatatactgctgctgtcttggaatacagagttgtctttcatgattctgctgatgttgctgctgaagataagtcttggattaacgctaatggtcatgctgttcatgatgctcaacatccattcagatctaacgttgtcgtcagaaaagaattgcatacttctgcctctgatagatcctgttctcatttggaattcaacatttccggttccgctttgaattacgaaactggtgatcatgttggtgtctactgtgaaaacttgactgaaactgttgatgaagccttgaacttgttgggtttgtctccagaaacttacttctctatctacaccgataacgaagatggtactccattgggtggttcttcattgccaccaccatttccatcatgtactttgagaactgctttgaccagatacgctgatttgttgaactctccaaaaaagtctgctttgttggctttagctgctcatgcttctaatccagttgaagctgatagattgagatacttggcttctccagctggtaaagatgaatatgcccaatctgttatcggttcccaaaagtctttgttggaagttatggctgaattcccatctgctaaaccaccattaggtgttttttttgctgctgttgctccaagattgcaacctagattctactccatttcatcctctccaagaatggctccatctagaatccatgttacttgtgctttggtttacgataagatgccaactggtagaattcataagggtgtttgttctacctggatgaagaattctgttccaatggaaaagtcccatgaatgttcttgggctccaattttcgttagacaatccaattttaagttgccagccgaatccaaggttccaattatcatggttggtccaggtactggtttggctccttttagaggttttttacaagaaagattggccttgaaagaatccggtgttgaattgggtccatccattttgtttttcggttgcagaaacagaagaatggattacatctacgaagatgaattgaacaacttcgttgaaaccggtgctttgtccgaattggttattgctttttctagagaaggtcctaccaaagaatacgtccaacataagatggctgaaaaggcttctgatatctggaacttgatttctgaaggtgcttacttgtacgtttgtggtgatgctaaaggtatggctaaggatgttcatagaaccttgcataccatcatgcaagaacaaggttctttggattcttccaaagctgaatccatggtcaagaacttgcaaatgaatggtagatacttaagagatgtttggtaa

SEQ ID NO: 80 - protein, 701 aa, Siraitia grosvenorii
MKVSPFEFMSAIIKGRMDPSNSSFESTGEVASVIFENRELVAILTTSIAVMIGCFVVLMWRRAGSRKVKNVELPKPLIVHEPEPEVEDGKKKVSIFFGTQTGTAEGFAKALADEAKARYEKATFRVVDLDDYAADDDQYEEKLKNESFAVFLLATYGDGEPTDNAARFYKWFAEGKERGEWLQNLHYAVFGLGNRQYEHFNKIAKVADELLEAQGGNRLVKVGLGDDDQCIEDDFSAWRESLWPELDMLLRDEDDATTVTTPYTAAVLEYRVVFHDSADVAAEDKSWINANGHAVHDAQHPFRSNVVVRKELHTSASDRSCSHLEFNISGSALNYETGDHVGVYCENLTETVDEALNLLGLSPETYFSIYTDNEDGTPLGGSSLPPPFPSCTLRTALTRYADLLNSPKKSALLALAAHASNPVEADRLRYLASPAGKDEYAQSVIGSQKSLLEVMAEFPSAKPPLGVFFAAVAPRLQPRFYSISSSPRMAPSRIHVTCALVYDKMPTGRIHKGVCSTWMKNSVPMEKSHECSWAPIFVRQSNFKLPAESKVPIIMVGPGTGLAPFRGFLQERLALKESGVELGPSILFFGCRNRRMDYIYEDELNNFVETGALSELVIAFSREGPTKEYVQHKMAEKASDIWNLISEGAYLYVCGDAKGMAKDVHRTLHTIMQEQGSLDSSKAESMVKNLQMNGRYLRDVW

SEQ ID NO: 81 - DNA, 2142 bp, Artificial Sequence
  Codon-optimized CPR
atggcagaattagatacacttgatatagtagtattaggtgttatctttttgggtactgtggcatactttactaagggtaaattgtggggtgttaccaaggatccatacgctaacggattcgctgcaggtggtgcttccaagcctggcagaactagaaacatcgtcgaagctatggaggaatcaggtaaaaactgtgttgttttctacggcagtcaaacaggtacagcggaggattacgcatcaagacttgcaaaggaaggaaagtccagattcggtttgaacactatgatcgccgatctagaagattatgacttcgataacttagacactgttccatctgataacatcgttatgtttgtattggctacttacggtgaaggcgaaccaacagataacgccgtggatttctatgagttcattactggcgaagatgcctctttcaatgagggcaacgatcctccactaggtaacttgaattacgttgcgttcggtctgggcaacaatacctacgaacactacaactcaatggtcaggaacgttaacaaggctctagaaaagttaggagctcatagaattggagaagcaggtgagggtgacgacggagctggaactatggaagaggactttttagcttggaaagatccaatgtgggaagccttggctaaaaagatgggcttggaggaaagagaagctgtatatgaacctattttcgctatcaatgagagagatgatttgacccctgaagcgaatgaggtatacttgggagaacctaataagctacacttggaaggtacagcgaaaggtccattcaactcccacaacccatatatcgcaccaattgcagaatcatacgaacttttctcagctaaggatagaaattgtctgcatatggaaattgatatttctggtagtaatctaaagtatgaaacaggcgaccatatcgcgatctggcctaccaacccaggtgaagaggtcaacaaatttcttgacattctagatctgtctggtaagcaacattccgtcgtaacagtgaaagccttagaacctacagccaaagttccttttccaaatccaactacctacgatgctatattgagataccatctggaaatatgcgctccagtttctagacagtttgtctcaactttagcagcattcgcccctaatgatgatatcaaagctgagatgaaccgtttgggatcagacaaagattacttccacgaaaagacaggaccacattactacaatatcgctagatttttggcctcagtctctaaaggtgaaaaatggacaaagataccattttctgctttcatagaaggccttacaaaactacaaccaagatactattctatctcttcctctagtttagttcagcctaaaaagattagtattactgctgttgtcgaatctcagcaaattccaggtagagatgacccattcagaggtgtagcgactaactacttgttcgctttgaagcagaaacaaaacggtgatccaaatccagctccttttggccaatcatacgagttgacaggaccaaggaataagtatgatggtatacatgttccagtccatgtaagacattctaactttaagctaccatctgatccaggcaaacctattatcatgatcggtccaggtaccggtgttgccccttttagaggcttcgtccaagagagggcaaaacaagccagagatggtgtagaagttggtaaaacactgctgttctttggatgtagaaagagtacagaagatttcatgtatcaaaaagagtggcaagagtacaaggaagctcttggcgacaaattcgaaatgattacagctttttcaagagaaggatctaaaaaggtttatgttcaacacagactgaaggaaagatcaaaggaagtttctgatcttctatcccaaaaagcatacttctacgtttgcggagacgccgcacatatggcacgtgaagtgaacactgtgttagcacagatcatagcagaaggccgtggtgtatcagaagccaagggtgaggaaattgtcaaaaacatgagatcagcaaatcaataccaagtgtgttctgatttcgtaactttacactgtaaagagacaacatacgcgaattcagaattgcaagaggatgtctggagttaa

SEQ ID NO: 82 - protein, 713 aa, Gibberella fujikuroi
MAELDTLDIVVLGVIFLGTVAYFTKGKLWGVTKDPYANGFAAGGASKPGRTRNIVEAMEESGKNCVVFYGSQTGTAEDYASRLAKEGKSRFGLNTMIADLEDYDFDNLDTVPSDNIVMFVLATYGEGEPTDNAVDFYEFITGEDASFNEGNDPPLGNLNYVAFGLGNNTYEHYNSMVRNVNKALEKLGAHRIGEAGEGDDGAGTMEEDFLAWKDPMWEALAKKMGLEEREAVYEPIFAINERDDLTPEANEVYLGEPNKLHLEGTAKGPFNSHNPYIAPIAESYELFSAKDRNCLHMEIDISGSNLKYETGDHIAIWPTNPGEEVNKFLDILDLSGKQHSVVTVKALEPTAKVPFPNPTTYDAILRYHLEICAPVSRQFVSTLAAFAPNDDIKAEMNRLGSDKDYFHEKTGPHYYNIARFLASVSKGEKWTKIPFSAFIEGLTKLQPRYYSISSSSLVQPKKISITAVVESQQIPGRDDPFRGVATNYLFALKQKQNGDPNPAPFGQSYELTGPRNKYDGIHVPVHVRHSNFKLPSDPGKPIIMIGPGTGVAPFRGFVQERAKQARDGVEVGKTLLFFGCRKSTEDFMYQKEWQEYKEALGDKFEMITAFSREGSKKVYVQHRLKERSKEVSDLLSQKAYFYVCGDAAHMAREVNTVLAQIIAEGRGVSEAKGEEIVKNMRSANQYQVCSDFVTLHCKETTYANSELQEDVWS

SEQ ID NO: 83 - DNA, 2130 bp, Stevia rebaudiana
atgcaatcggaatccgttgaagcatcgacgattgatttgatgactgctgttttgaaggacacagtgatcgatacagcgaacgcatctgataacggagactcaaagatgccgccggcgttggcgatgatgttcgaaattcgtgatctgttgctgattttgactacgtcagttgctgttttggtcggatgtttcgttgttttggtgtggaagagatcgtccgggaagaagtccggcaaggaattggagccgccgaagatcgttgtgccgaagaggcggctggagcaggaggttgatgatggtaagaagaaggttacgattttcttcggaacacaaactggaacggctgaaggtttcgctaaggcacttttcgaagaagcgaaagcgcgatatgaaaaggcagcgtttaaagtgattgatttggatgattatgctgctgatttggatgagtatgcagagaagctgaagaaggaaacatatgctttcttcttcttggctacatatggagatggtgagccaactgataatgctgccaaattttataaatggtttactgagggagacgagaaaggcgtttggcttcaaaaacttcaatatggagtatttggtcttggcaacagacaatatgaacatttcaacaagattggaatagtggttgatgatggtctcaccgagcagggtgcaaaacgcattgttcccgttggtcttggagacgacgatcaatcaattgaagacgatttttcggcatggaaagagttagtgtggcccgaattggatctattgcttcgcgatgaagatgacaaagctgctgcaactccttacacagctgcaatccctgaataccgcgtcgtatttcatgacaaacccgatgcgttttctgatgatcatactcaaaccaatggtcatgctgttcatgatgctcaacatccatgcagatccaatgtggctgttaaaaaagagcttcatactcctgaatccgatcgttcatgcacacatcttgaatttgacatttctcacactggattatcttatgaaactggggatcatgttggtgtatactgtgaaaacctaattgaagtagtggaagaagctgggaaattgttaggattatcaacagatacttatttctcgttacatattgataacgaagatggttcaccacttggtggaccttcattacaacctccttttcctccttgtactttaagaaaagcattgactaattatgcagatctgttaagctctcccaaaaagtcaactttgcttgctctagctgctcatgcttccgatcccactgaagctgatcgtttaagatttcttgcatctcgcgagggcaaggatgaatatgctgaatgggttgttgcaaaccaaagaagtcttcttgaagtcatggaagctttcccgtcagctagaccgccacttggtgttttctttgcagcggttgcaccgcgtttacagcctcgttactactctatttcttcctccccaaagatggaaccaaacaggattcatgttacttgcgcgttggtttatgaaaaaactcccgcaggtcgtatccacaaaggaatctgctcaacctggatgaagaacgctgtacctttgaccgaaagtcaagattgcagttgggcaccgatttttgttagaacatcaaacttcagacttccaattgacccgaaagtcccggttatcatgattggtcctggaaccgggttggctccatttaggggttttcttcaagaaagattggctcttaaagaatccggaaccgaactcgggtcatctattttattcttcggttgtagaaaccgcaaagtggattacatatatgagaatgaactcaacaactttgttgaaaatggtgcgctttctgagcttgatgttgctttctcccgcgatggcccgacgaaagaatacgtgcaacataaaatgacccaaaaggcttctgaaatatggaatatgctttctgagggagcatatttatatgtatgtggtgatgctaaaggcatggctaaagatgtacaccgtacacttcacaccattgtgcaagaacagggaagtttggactcgtctaaagcggagttgtatgtgaagaatctacaaatgtcaggaagatacctccgtgatgtttggtaa

SEQ ID NO: 84 - protein, 709 aa, Stevia rebaudiana
MQSESVEASTIDLMTAVLKDTVIDTANASDNGDSKMPPALAMMFEIRDLLLILTTSVAVLVGCFVVLVWKRSSGKKSGKELEPPKIVVPKRRLEQEVDDGKKKVTIFFGTQTGTAEGFAKALFEEAKARYEKAAFKVIDLDDYAADLDEYAEKLKKETYAFFFLATYGDGEPTDNAAKFYKWFTEGDEKGVWLQKLQYGVFGLGNRQYEHFNKIGIVVDDGLTEQGAKRIVPVGLGDDDQSIEDDFSAWKELVWPELDLLLRDEDDKAAATPYTAAIPEYRVVFHDKPDAFSDDHTQTNGHAVHDAQHPCRSNVAVKKELHTPESDRSCTHLEFDISHTGLSYETGDHVGVYCENLIEVVEEAGKLLGLSTDTYFSLHIDNEDGSPLGGPSLQPPFPPCTLRKALTNYADLLSSPKKSTLLALAAHASDPTEADRLRFLASREGKDEYAEWVVANQRSLLEVMEAFPSARPPLGVFFAAVAPRLQPRYYSISSSPKMEPNRIHVTCALVYEKTPAGRIHKGICSTWMKNAVPLTESQDCSWAPIFVRTSNFRLPIDPKVPVIMIGPGTGLAPFRGFLQERLALKESGTELGSSILFFGCRNRKVDYIYENELNNFVENGALSELDVAFSRDGPTKEYVQHKMTQKASEIWNMLSEGAYLYVCGDAKGMAKDVHRTLHTIVQEQGSLDSSKAELYVKNLQMSGRYLRDVW

SEQ ID NO: 85 - DNA, 2124 bp, Artificial Sequence
  Codon-optimized CPR
atgcaatctaactccgtgaagatttcgccgcttgatctggtaactgcgctgtttagcggcaaggttttggacacatcgaacgcatcggaatcgggagaatctgctatgctgccgactatagcgatgattatggagaatcgtgagctgttgatgatactcacaacgtcggttgctgtattgatcggatgcgttgtcgttttggtgtggcggagatcgtctacgaagaagtcggcgttggagccaccggtgattgtggttccgaagagagtgcaagaggaggaagttgatgatggtaagaagaaagttacggttttcttcggcacccaaactggaacagctgaaggcttcgctaaggcacttgttgaggaagctaaagctcgatatgaaaaggctgtctttaaagtaattgatttggatgattatgctgctgatgacgatgagtatgaggagaaactaaagaaagaatctttggcctttttctttttggctacgtatggagatggtgagccaacagataatgctgccagattttataaatggtttactgagggagatgcgaaaggagaatggcttaataagcttcaatatggagtatttggtttgggtaacagacaatatgaacattttaacaagatcgcaaaagtggttgatgatggtcttgtagaacagggtgcaaagcgtcttgttcctgttggacttggagatgatgatcaatgtattgaagatgacttcaccgcatggaaagagttagtatggccggagttggatcaattacttcgtgatgaggatgacacaactgttgctactccatacacagctgctgttgcagaatatcgcgttgtttttcatgaaaaaccagacgcgctttctgaagattatagttatacaaatggccatgctgttcatgatgctcaacatccatgcagatccaacgtggctgtcaaaaaggaacttcatagtcctgaatctgaccggtcttgcactcatcttgaatttgacatctcgaacaccggactatcatatgaaactggggaccatgttggagtttactgtgaaaacttgagtgaagttgtgaatgatgctgaaagattagtaggattaccaccagacacttactcctccatccacactgatagtgaagacgggtcgccacttggcggagcctcattgccgcctcctttcccgccatgcactttaaggaaagcattgacgtgttatgctgatgttttgagttctcccaagaagtcggctttgcttgcactagctgctcatgccaccgatcccagtgaagctgatagattgaaatttcttgcatcccccgccggaaaggatgaatattctcaatggatagttgcaagccaaagaagtctccttgaagtcatggaagcattcccgtcagctaagccttcacttggtgttttctttgcatctgttgccccgcgcttacaaccaagatactactctatttcttcctcacccaagatggcaccggataggattcatgttacatgtgcattagtctatgagaaaacacctgcaggccgcatccacaaaggagtttgttcaacttggatgaagaacgcagtgcctatgaccgagagtcaagattgcagttgggccccaatatacgtccgaacatccaatttcagactaccatctgaccctaaggtcccggttatcatgattggacctggcactggtttggctccttttagaggtttccttcaagagcggttagctttaaaggaagccggaactgacctcggtttatccattttattcttcggatgtaggaatcgcaaagtggatttcatatatgaaaacgagcttaacaactttgtggagactggtgctctttctgagcttattgttgctttctcccgtgaaggcccgactaaggaatatgtgcaacacaagatgagtgagaaggcttcggatatctggaacttgctttctgaaggagcatatttatacgtatgtggtgatgccaaaggcatggccaaagatgtacatcgaaccctccacacaattgtgcaagaacagggatctcttgactcgtcaaaggcagaactctacgtgaagaatctacaaatgtcaggaagatacctccgtgacgtttggtaa

SEQ ID NO: 86 - protein, 707 aa, Stevia rebaudiana
MQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYSSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 87 - DNA, 2070 bp, Artificial Sequence
  Codon-optimized CPR
atgtcctccaactccgatttggtcagaagattggaatctgttttgggtgtttctttcggtggttctgttactgattccgttgttgttattgctaccacctctattgctttggttatcggtgttttggttttgttgtggagaagatcctctgacagatctagagaagttaagcaattggctgttccaaagccagttactatcgttgaagaagaagatgaattcgaagttgcttctggtaagaccagagtttctattttctacggtactcaaactggtactgctgaaggttttgctaaggctttggctgaagaaatcaaagccagatacgaaaaagctgccgttaaggttattgatttggatgattacacagccgaagatgacaaatacggtgaaaagttgaagaaagaaactatggccttcttcatgttggctacttatggtgatggtgaacctactgataatgctgctagattttacaagtggttcaccgaaggtactgatagaggtgtttggttggaacatttgagatacggtgtattcggtttgggtaacagacaatacgaacacttcaacaagattgccaaggttgttgatgatttgttggttgaacaaggtgccaagagattggttactgttggtttgggtgatgatgatcaatgcatcgaagatgatttctccgcttggaaagaagccttgtggccagaattggatcaattattgcaagatgataccaacaccgtttctactccatacactgctgttattccagaatacagagttgttatccacgatccatctgttacctcttatgaagatccatactctaacatggctaacggtaatgcctcttacgatattcatcatccatgtagagctaacgttgccgtccaaaaagaattgcataagccagaatctgacagaagttgcatccatttggaattcgatattttcgctactggtttgacttacgaaaccggtgatcatgttggtgtttacgctgataattgtgatgatactgtagaagaagccgctaagttgttgggtcaaccattggatttgttgttctccattcataccgataacaacgacggtacttctttgggttcttctttgccaccaccatttccaggtccatgtactttgagaactgctttggctagatatgccgatttgttgaatccaccaaaaaaggctgctttgattgctttagctgctcatgctgatgaaccatctgaagctgaaagattgaagttcttgtcatctccacaaggtaaggacgaatattctaaatgggttgtcggttcccaaagatccttggttgaagttatggctgaatttccatctgctaaaccaccattgggtgtattttttgctgctgttgttcctagattgcaacctagatattactccatctcttccagtccaagatttgctccacatagagttcatgttacttgcgctttggtttatggtccaactccaactggtagaattcacagaggtgtatgttcattctggatgaagaatgttgtcccattggaaaagtctcaaaactgttcttgggccccaattttcatcagacaatctaatttcaagttgccagccgatcattctgttccaatagttatggttggtccaggtactggtttagctccttttagaggtttcttacaagaaagattggccttgaaagaagaaggtgctcaagttggtcctgctttgttgttttttggttgcagaaacagacaaatggacttcatctacgaagtcgaattgaacaactttgtcgaacaaggtgctttgtccgaattgatcgttgctttttcaagagaaggtccatccaaagaatacgtccaacataagatggttgaaaaggcagcttacatgtggaacttgatttctcaaggtggttacttctacgtttgtggtgatgctaaaggtatggctagagatgttcatagaacattgcataccatcgtccaacaagaagaaaaggttgattctaccaaggccgaatccatcgttaagaaattgcaaatggacggtagatacttgagagatgtttggtga

SEQ ID NO: 88 - protein, 689 aa, Rubus suavissimus
MSSNSDLVRRLESVLGVSFGGSVTDSVVVIATTSIALVIGVLVLLWRRSSDRSREVKQLAVPKPVTIVEEEDEFEVASGKTRVSIFYGTQTGTAEGFAKALAEEIKARYEKAAVKVIDLDDYTAEDDKYGEKLKKETMAFFMLATYGDGEPTDNAARFYKWFTEGTDRGVWLEHLRYGVFGLGNRQYEHFNKIAKVVDDLLVEQGAKRLVTVGLGDDDQCIEDDFSAWKEALWPELDQLLQDDTNTVSTPYTAVIPEYRVVIHDPSVTSYEDPYSNMANGNASYDIHHPCRANVAVQKELHKPESDRSCIHLEFDIFATGLTYETGDHVGVYADNCDDTVEEAAKLLGQPLDLLFSIHTDNNDGTSLGSSLPPPFPGPCTLRTALARYADLLNPPKKAALIALAAHADEPSEAERLKFLSSPQGKDEYSKWVVGSQRSLVEVMAEFPSAKPPLGVFFAAVVPRLQPRYYSISSSPRFAPHRVHVTCALVYGPTPTGRIHRGVCSFWMKNVVPLEKSQNCSWAPIFIRQSNFKLPADHSVPIVMVGPGTGLAPFRGFLQERLALKEEGAQVGPALLFFGCRNRQMDFIYEVELNNFVEQGALSELIVAFSREGPSKEYVQHKMVEKAAYMWNLISQGGYFYVCGDAKGMARDVHRTLHTIVQQEEKVDSTKAESIVKKLQMDGRYLRDVW

SEQ ID NO: 89 - DNA, 2079 bp, Artificial Sequence
  Codon-optimized CPR
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 90 - protein, 692 aa, Arabidopsis thaliana [sequence omitted]

SEQ ID NO: 91 - DNA, 2139 bp, Artificial Sequence
  Codon-optimized CPR
atgtcttcctcttcctcttccagtacctctatgattgatttgatggctgctattattaaaggtgaaccagttatcgtctccgacccagcaaatgcctctgcttatgaatcagttgctgcagaattgtcttcaatgttgatcgaaaacagacaattcgccatgatcgtaactacatcaatcgctgttttgatcggttgtattgtcatgttggtatggagaagatccggtagtggtaattctaaaagagtcgaacctttgaaaccattagtaattaagccaagagaagaagaaatagatgacggtagaaagaaagttacaatatttttcggtacccaaactggtacagctgaaggttttgcaaaagccttaggtgaagaagctaaggcaagatacgaaaagactagattcaagatagtcgatttggatgactatgccgctgatgacgatgaatacgaagaaaagttgaagaaagaagatgttgcatttttctttttggcaacctatggtgacggtgaaccaactgacaatgcagccagattctacaaatggtttacagagggtaatgatcgtggtgaatggttgaaaaacttaaagtacggtgttttcggtttgggtaacagacaatacgaacatttcaacaaagttgcaaaggttgtcgacgatattttggtcgaacaaggtgctcaaagattagtccaagtaggtttgggtgacgatgaccaatgtatagaagatgactttactgcctggagagaagctttgtggcctgaattagacacaatcttgagagaagaaggtgacaccgccgttgctaccccatatactgctgcagtattagaatacagagtttccatccatgatagtgaagacgcaaagtttaatgatatcactttggccaatggtaacggttatacagttttcgatgcacaacacccttacaaagctaacgttgcagtcaagagagaattacatacaccagaatccgacagaagttgtatacacttggaatttgatatcgctggttccggtttaaccatgaagttgggtgaccatgtaggtgttttatgcgacaatttgtctgaaactgttgatgaagcattgagattgttggatatgtcccctgacacttattttagtttgcacgctgaaaaagaagatggtacaccaatttccagttctttaccacctccattccctccatgtaacttaagaacagccttgaccagatacgcttgcttgttatcatcccctaaaaagtccgccttggttgctttagccgctcatgctagtgatcctactgaagcagaaagattgaaacacttagcatctccagccggtaaagatgaatattcaaagtgggtagttgaatctcaaagatcattgttagaagttatggcagaatttccatctgccaagcctccattaggtgtcttctttgctggtgtagcacctagattgcaaccaagattctactcaatcagttcttcacctaagatcgctgaaactagaattcatgttacatgtgcattagtctacgaaaagatgccaaccggtagaattcacaagggtgtatgctctacttggatgaaaaatgctgttccttacgaaaaatcagaaaagttgttcttaggtagaccaatcttcgtaagacaatcaaacttcaagttgccttctgattcaaaggttccaataatcatgataggtcctggtacaggtttagccccattcagaggtttcttgcaagaaagattggctttagttgaatctggtgtcgaattaggtccttcagttttgttctttggttgtagaaacagaagaatggatttcatctatgaagaagaattgcaaagattcgtcgaatctggtgcattggccgaattatctgtagctttttcaagagaaggtccaactaaggaatacgttcaacataagatgatggataaggcatccgacatatggaacatgatcagtcaaggtgcttatttgtacgtttgcggtgacgcaaagggtatggccagagatgtccatagatctttgcacacaattgctcaagaacaaggttccatggatagtaccaaagctgaaggtttcgtaaagaacttacaaacttccggtagatacttgagagatgtctggtga

SEQ ID NO: 92 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 93 - DNA, 1503 bp, Artificial Sequence
  Codon-optimized KAH
atggaagcctcttacctatacatttctattttgcttttactggcatcatacctgttcaccactcaacttagaaggaagagcgctaatctaccaccaaccgtgtttccatcaataccaatcattggacacttatacttactcaaaaagcctctttatagaactttagcaaaaattgccgctaagtacggaccaatactgcaattacaactcggctacagacgtgttctggtgatttcctcaccatcagcagcagaagagtgctttaccaataacgatgtaatcttcgcaaatagacctaagacattgtttggcaaaatagtgggtggaacatcccttggcagtttatcctacggcgatcaatggcgtaatctaaggagagtagcttctatcgaaatcctatcagttcataggttgaacgaatttcatgatatcagagtggatgagaacagattgttaattagaaaacttagaagttcatcttctcctgttactcttataacagtcttttatgctctaacattgaacgtcattatgagaatgatctctggcaaaagatatttcgacagtggggatagagaattggaggaggaaggtaagagatttcgagaaatcttagacgaaacgttgcttctagccggtgcttctaatgttggcgactacttaccaatattgaactggttgggagttaagtctcttgaaaagaaattgatcgctttgcagaaaaagagagatgactttttccagggtttgattgaacaggttagaaaatctcgtggtgctaaagtaggcaaaggtagaaaaacgatgatcgaactcttattatctttgcaagagtcagaacctgagtactatacagatgctatgataagatcttttgtcctaggtctgctggctgcaggtagtgatacttcagcgggcactatggaatgggccatgagcttactggtcaatcacccacatgtattgaagaaagctcaagctgaaatcgatagagttatcggtaataacagattgattgacgagtcagacattggaaatatcccttacatcgggtgtattatcaatgaaactctaagactctatccagcagggccattgttgttcccacatgaaagttctgccgactgcgttatttccggttacaatatacctagaggtacaatgttaatcgtaaaccaatgggcgattcatcacgatcctaaagtctgggatgatcctgaaacctttaaacctgaaagatttcaaggattagaaggaactagagatggtttcaaacttatgccattcggttctgggagaagaggatgtccaggtgaaggtttggcaataaggctgttagggatgacactaggctcagtgatccaatgttttgattgggagagagtaggagatgagatggttgacatgacagaaggtttgggtgtcacacttcctaaggccgttccattagttgccaaatgtaagccacgttccgaaatgactaatctcctatccgaactttaa

SEQ ID NO: 94 - protein, 500 aa, Stevia rebaudiana
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 95 - DNA, 1572 bp, Rubus suavissimus
atggaagtaacagtagctagtagtgtagccctgagcctggtctttattagcatagtagtaagatgggcatggagtgtggtgaattgggtgtggtttaagccgaagaagctggaaagatttttgagggagcaaggccttaaaggcaattcctacaggtttttatatggagacatgaaggagaactctatcctgctcaaacaagcaagatccaaacccatgaacctctccacctcccatgacatagcacctcaagtcaccccttttgtcgaccaaaccgtgaaagcttacggtaagaactcttttaattgggttggccccataccaagggtgaacataatgaatccagaagatttgaaggacgtcttaacaaaaaatgttgactttgttaagccaatatcaaacccacttatcaagttgctagctacaggtattgcaatctatgaaggtgagaaatggactaaacacagaaggattatcaacccaacattccattcggagaggctaaagcgtatgttaccttcatttcaccaaagttgtaatgagatggtcaaggaatgggagagcttggtgtcaaaagagggttcatcatgtgagttggatgtctggccttttcttgaaaatatgtcggcagatgtgatctcgagaacagcatttggaactagctacaaaaaaggacagaaaatctttgaactcttgagagagcaagtaatatatgtaacgaaaggctttcaaagtttttacattccaggatggaggtttctcccaactaagatgaacaagaggatgaatgagattaacgaagaaataaaaggattaatcaggggtattataattgacagagagcaaatcattaaggcaggtgaagaaaccaacgatgacttattaggtgcacttatggagtcaaacttgaaggacattcgggaacatgggaaaaacaacaaaaatgttgggatgagtattgaagatgtaattcaggagtgtaagctgttttactttgctgggcaagaaaccacttcagtgttgctggcttggacaatggttttacttggtcaaaatcagaactggcaagatcgagcaagacaagaggttttgcaagtctttggaagcagcaagccagattttgatggtctagctcaccttaaagtcgtaaccatgattttgcttgaagttcttcgattatacccaccagtcattgaacttattcgaaccattcacaagaaaacacaacttgggaagctctcactaccagaaggagttgaagtccgcttaccaacactgctcattcaccatgacaaggaactgtggggtgatgatgcaaaccagttcaatccagagaggttttcggaaggagtttccaaagcaacaaagaaccgactctcattcttccccttcggagccggtccacgcatttgcattggacagaacttttctatgatggaagcaaagttggccttagcattgatcttgcaacacttcacctttgagctttctccatctcatgcacatgctccttcccatcgtataacccttcaaccacagtatggtgttcgtatcattttacatcgacgttag

SEQ ID NO: 96 - DNA, 1572 bp, Artificial Sequence
  Codon-optimized KAH
atggaagtcactgtcgcctcttctgtcgctttatccttagtcttcatttccattgtcgtcagatgggcttggtccgttgtcaactgggtttggttcaaaccaaagaagttggaaagattcttgagagagcaaggtttgaagggtaattcttatagattcttgtacggtgacatgaaggaaaattctattttgttgaagcaagccagatccaaaccaatgaacttgtctacctctcatgatattgctccacaagttactccattcgtcgatcaaactgttaaagcctacggtaagaactctttcaattgggttggtccaattcctagagttaacatcatgaacccagaagatttgaaggatgtcttgaccaagaacgttgacttcgttaagccaatttccaacccattgattaaattgttggctactggtattgccatttacgaaggtgaaaagtggactaagcatagaagaatcatcaaccctaccttccactctgaaagattgaagagaatgttaccatctttccatcaatcctgtaatgaaatggttaaggaatgggaatccttggtttctaaagaaggttcttcttgcgaattggatgtttggccattcttggaaaatatgtctgctgatgtcatttccagaaccgctttcggtacctcctacaagaagggtcaaaagattttcgaattgttgagagagcaagttatttacgttaccaagggtttccaatccttctacatcccaggttggagattcttgccaactaaaatgaacaagcgtatgaacgagatcaacgaagaaattaaaggtttgatcagaggtattattatcgacagagaacaaattattaaagctggtgaagaaaccaacgatgatttgttgggtgctttgatggagtccaacttgaaggatattagagaacatggtaagaacaacaagaatgttggtatgtctattgaagatgttattcaagaatgtaagttattctacttcgctggtcaagagaccacttctgttttgttagcctggactatggtcttgttaggtcaaaaccaaaattggcaagatagagctagacaagaagttttgcaagtcttcggttcttccaagccagactttgatggtttggcccacttgaaggttgttactatgattttgttagaagttttgagattgtacccaccagtcattgagttaatcagaaccattcataaaaagactcaattgggtaaattatctttgccagaaggtgttgaagtcagattaccaaccttgttgattcaccacgataaggaattatggggtgacgacgctaatcaatttaatccagaaagattttccgaaggtgtttccaaggctaccaaaaaccgtttgtccttcttcccatttggtgctggtccacgtatttgtatcggtcaaaacttttccatgatggaagccaagttggctttggctttaatcttgcaacacttcactttcgaattgtctccatcccatgcccacgctccttctcatagaatcactttacaaccacaatacggtgtcagaatcatcttacacagaagataa

SEQ ID NO: 97 - protein, 523 aa, Rubus suavissimus
MEVTVASSVALSLVFISIVVRWAWSVVNWVWFKPKKLERFLREQGLKGNSYRFLYGDMKENSILLKQARSKPMNLSTSHDIAPQVTPFVDQTVKAYGKNSFNWVGPIPRVNIMNPEDLKDVLTKNVDFVKPISNPLIKLLATGIAIYEGEKWTKHRRIINPTFHSERLKRMLPSFHQSCNEMVKEWESLVSKEGSSCELDVWPFLENMSADVISRTAFGTSYKKGQKIFELLREQVIYVTKGFQSFYIPGWRFLPTKMNKRMNEINEEIKGLIRGIIIDREQIIKAGEETNDDLLGALMESNLKDIREHGKNNKNVGMSIEDVIQECKLFYFAGQETTSVLLAWTMVLLGQNQNWQDRARQEVLQVFGSSKPDFDGLAHLKVVTMILLEVLRLYPPVIELIRTIHKKTQLGKLSLPEGVEVRLPTLLIHHDKELWGDDANQFNPERFSEGVSKATKNRLSFFPFGAGPRICIGQNFSMMEAKLALALILQHFTFELSPSHAHAPSHRITLQPQYGVRIILHRR

SEQ ID NO: 98 - DNA, 1566 bp, Prunus avium
atggaagcatcaagggctagttgtgttgcgctatgtgttgtttgggtgagcatagtaattacattggcatggagggtgctgaattgggtgtggttgaggccaaagaaactagaaagatgcttgagggagcaaggccttacaggcaattcttacaggcttttgtttggagacaccaaggatctctcgaagatgctggaacaaacacaatccaaacccatcaaactctccacctcccatgatatagcgccacgagtcaccccatttttccatcgaactgtgaactctaatggcaagaattcttttgtttggatgggccctataccaagagtgcacatcatgaatccagaagatttgaaagatgccttcaacagacatgatgattttcataagacagtaaaaaatcctatcatgaagtctccaccaccgggcattgtaggcattgaaggtgagcaatgggctaaacacagaaagattatcaacccagcattccatttagagaagctaaagggtatggtaccaatattttaccaaagttgtagcgagatgattaacaaatgggagagcttggtgtccaaagagagttcatgtgagttggatgtgtggccttatcttgaaaattttaccagcgatgtgatttcccgagctgcatttggaagtagctatgaagagggaaggaaaatatttcaactactaagagaggaagcaaaagtttattcggtagctctacgaagtgtttacattccaggatggaggtttctaccaaccaagcagaacaagaagacgaaggaaattcacaatgaaattaaaggcttacttaagggcattataaataaaagggaagaggcgatgaaggcaggggaagccactaaagatgacttactaggaatacttatggagtccaacttcagggaaattcaggaacatgggaacaacaaaaatgctggaatgagtattgaagatgtaattggagagtgtaagttgttttactttgctgggcaagagaccacttcggtgttgcttgtttggacaatgattttactaagccaaaatcaggattggcaagctcgtgcaagagaagaggtcttgaaagtctttggaagcaacatcccaacctatgaagagctaagtcacctaaaagttgtgaccatgattttacttgaagttcttcgattatacccatcagtcgttgcgcttcctcgaaccactcacaagaaaacacagcttggaaaattatcattaccagctggagtggaagtctccttgcccatactgcttgttcaccatgacaaagagttgtggggtgaggatgcaaatgagttcaagccagagaggttttcagagggagtttcaaaggcaacaaagaacaaatttacatacttacctttcggagggggtccaaggatttgcattggacaaaactttgccatggtggaagctaaattggccttggccctgattttacaacactttgcctttgagctttctccatcctatgctcatgctccttctgcagttataacccttcaacctcaatttggtgctcatatcattttgcataaacgttga

SEQ ID NO: 99 - DNA, 1567 bp, Artificial Sequence
  Codon-optimized KAH
atggaagcttctagagcatcttgtgttgctttgtgtgttgtttgggtttccatcgttattactttggcttggagagttttgaattgggtctggttaagaccaaaaaagttggaaagatgcttgagagaacaaggtttgactggtaactcttacagattgttgttcggtgataccaaggacttgtctaagatgttggaacaaactcaatccaagcctatcaagttgtctacctctcatgatattgctccaagagttactccattcttccatagaactgttaactccaacggtaagaactcttttgtttggatgggtccaattccaagagtccatattatgaaccctgaagatttgaaggacgctttcaacagacatgatgatttccataagaccgtcaagaacccaattatgaagtctccaccaccaggtatagttggtattgaaggtgaacaatgggccaaacatagaaagattattaacccagccttccacttggaaaagttgaaaggtatggttccaatcttctaccaatcctgctctgaaatgattaacaagtgggaatccttggtttccaaagaatcttcctgtgaattggatgtctggccatatttggaaaacttcacctccgatgttatttccagagctgcttttggttcttcttacgaagaaggtagaaagatcttccaattattgagagaagaagccaaggtttactccgttgctttgagatctgtttacattccaggttggagattcttgccaactaagcaaaacaaaaagaccaaagaaatccacaacgaaatcaagggtttgttgaagggtatcatcaacaagagagaagaagctatgaaggctggtgaagctacaaaagatgatttgttgggtatcttgatggaatccaacttcagagaaatccaagaacacggtaacaacaagaatgccggtatgtctattgaagatgttatcggtgaatgcaagttgttctactttgctggtcaagaaactacctccgttttgttggtttggaccatgattttgttgtcccaaaatcaagattggcaagctagagctagagaagaagtcttgaaagttttcggttctaacatcccaacctacgaagaattgtctcacttgaaggttgtcactatgatcttgttggaagtattgagattatacccatccgttgttgcattgccaagaactactcataagaaaactcaattgggtaaattgtccttgccagctggtgttgaagtttctttgccaattttgttagtccaccacgacaaagaattgtggggtgaagatgctaatgaattcaagccagaaagattctccgaaggtgtttctaaagctaccaagaacaagttcacttacttgccatttggtggtggtccaagaatatgtattggtcaaaatttcgctatggtcgaagctaaattggctttggctttgatcttgcaacatttcgctttcgaattgtcaccatcttatgctcatgctccatctgctgttattacattgcaaccacaatttggtgcccatatcatcttgcataagagataac

SEQ ID NO: 100 - protein, 521 aa, Prunus avium
MEASRASCVALCVVWVSIVITLAWRVLNWVWLRPKKLERCLREQGLTGNSYRLLFGDTKDLSKMLEQTQSKPIKLSTSHDIAPRVTPFFHRTVNSNGKNSFVWMGPIPRVHIMNPEDLKDAFNRHDDFHKTVKNPIMKSPPPGIVGIEGEQWAKHRKIINPAFHLEKLKGMVPIFYQSCSEMINKWESLVSKESSCELDVWPYLENFTSDVISRAAFGSSYEEGRKIFQLLREEAKVYSVALRSVYIPGWRFLPTKQNKKTKEIHNEIKGLLKGIINKREEAMKAGEATKDDLLGILMESNFREIQEHGNNKNAGMSIEDVIGECKLFYFAGQETTSVLLVWTMILLSQNQDWQARAREEVLKVFGSNIPTYEELSHLKVVTMILLEVLRLYPSVVALPRTTHKKTQLGKLSLPAGVEVSLPILLVHHDKELWGEDANEFKPERFSEGVSKATKNKFTYLPFGGGPRICIGQNFAMVEAKLALALILQHFAFELSPSYAHAPSAVITLQPQFGAHIILHKR

SEQ ID NO: 101 - protein, 517 aa, Prunus mume
ASWVAVLSVVWVSMVIAWAWRVLNWVWLRPKKLEKCLREQGLAGNSYRLLFGDTKDLSKMLEQTQSKPIKLSTSHDIAPHVTPFFHQTVNSYGKNSFVWMGPIPRVHIMNPEDLKDTFNRHDDFHKVVKNPIMKSLPQGIVGIEGEQWAKHRKIINPAFHLEKLKGMVPIFYRSCSEMINKWESLVSKESSCELDVWPYLENFTSDVISRAAFGSSYEEGRKIFQLLREEAKIYTVAMRSVYIPGWRFLPTKQNKKAKEIHNEIKGLLKGIINKREEAMKAGEATKDDLLGILMESNFREIQEHGNNKNAGMSIEDVIGECKLFYFAGQETTSVLLVWTMVLLSQNQDWQARAREEVLQVFGSNIPTYEELSQLKVVTMILLEVLRLYPSVVALPRTTHKKTQLGKLSLPAGVEVSLPILLVHHDKELWGEDANEFKPERFSEGVSKATKNQFTYFPFGGGPRICIGQNFAMMEAKLALSLILRHFALELSPLYAHAPSVTITLQPQYGAHIILHKR

SEQ ID NO: 102 - protein, 521 aa, Prunus mume
MEASRPSCVALSVVLVSIVIAWAWRVLNWVWLRPNKLERCLREQGLTGNSYRLLFGDTKEISMMVEQAQSKPIKLSTTHDIAPRVIPFSHQIVYTYGRNSFVWMGPTPRVTIMNPEDLKDAFNKSDEFQRAISNPIVKSISQGLSSLEGEKWAKHRKIINPAFHLEKLKGMLPTFYQSCSEMINKWESLVFKEGSREMDVWPYLENLTSDVISRAAFGSSYEEGRKIFQLLREEAKFYTIAARSVYIPGWRFLPTKQNKRMKEIHKEVRGLLKGIINKREDAIKAGEAAKGNLLGILMESNFREIQEHGNNKNAGMSIEDVIGECKLFYFAGQETTSVLLVWTLVLLSQNQDWQARAREEVLQVFGTNIPTYDQLSHLKVVTMILLEVLRLYPAVVELPRTTYKKTQLGKFLLPAGVEVSLHIMLAHHDKELWGEDAKEFKPERFSEGVSKATKNQFTYFPFGAGPRICIGQNFAMLEAKLALSLILQHFTFELSPSYAHAPSVTITLHPQFGAHFILHKR

SEQ ID NO: 103 - protein, 514 aa, Prunus mume
CVALSVVLVSIVIAWAWRVLNWVWLRPNKLERCLREQGLTGNSYRLLFGDTKEISMMVEQAQSKPIKLSTTHDIAPRVIPFSHQIVYTYGRNSFVWMGPTPRVTIMNPEDLKDAFNKSDEFQRAISNPIVKSISQGLSSLEGEKWAKHRKIINPAFHLEKLKGMLPTFYQSCSEMINKWESLVFKEGSREMDVWPYLENLTSDVISRAAFGSSYEEGRKIFQLLREEAKFYTIAARSVYIPGWRFLPTKQNKRMKEIHKEVRGLLKGIINKREDAIKAGEAAKGNLLGILMESNFREIQEHGNNKNAGMSIEDVIGECKLFYFAGQETTSVLLVWTLVLLSQNQDWQARAREEVLQVFGTNIPTYDQLSHLKVVTMILLEVLRLYPAVVELPRTTYKKTQLGKFLLPAGVEVSLHIMLAHHDKELWGEDAKEFKPERFSEGVSKATKNQFTYFPFGAGPRICIGQNFAMLEAKLALSLILQHFTFELSPSYAHAPSVTITLHPQFGAHFILHKR

SEQ ID NO: 104 - protein, 418 aa, Prunus persica
MGPIPRVHIMNPEDLKDTFNRHDDFHKVVKNPIMKSLPQGIVGIEGDQWAKHRKIINPAFHLEKLKGMVPIFYQSCSEMINIWKSLVSKESSCELDVWPYLENFTSDVISRAAFGSSYEEGRKIFQLLREEAKVYTVAVRSVYIPGWRFLPTKQNKKTKEIHNEIKGLLKGIINKREEAMKAGEATKDDLLGILMESNFREIQEHGNNKNAGMSIEDVIGECKLFYFAGQETTSVLLVWTMVLLSQNQDWQARAREEVLQVFGSNIPTYEELSHLKVVTMILLEVLRLYPSVVALPRTTHKKTQLGKLSLPAGVEVSLPILLVHHDKELWGEDANEFKPERFSEGVSKATKNQFTYFPFGGGPRICIGQNFAMMEAKLALSLILQHFTFELSPQYSHAPSVTITLQPQYGAHLILHKR

SEQ ID NO: 105 - DNA, 1578 bp, Artificial Sequence
  Codon-optimized KAH
atgggtttgttcccattagaggattcctacgcgctggtctttgaaggactagcaataacactggctttgtactatctactgtctttcatctacaaaacatctaaaaagacatgtacacctcctaaagcatctggtgaaatcattccaattacaggaatcatattgaatctgctatctggctcaagtggtctacctattatcttagcacttgcctctttagcagacagatgtggtcctattttcaccattaggctgggtattaggagagtgctagtagtatcaaattgggaaatcgctaaggagattttcactacccacgatttgatagtttctaatagaccaaaatacttagccgctaagattcttggtttcaattatgtttcattctctttcgctccatacggcccatattgggtcggaatcagaaagattattgctacaaaactaatgtcttcttccagacttcagaagttgcaatttgtaagagtttttgaactagaaaactctatgaaatctatcagagaatcatggaaggagaaaaaggatgaagagggaaaggtattagttgagatgaaaaagtggttctgggaactgaatatgaacatagtgttaaggacagttgctggtaaacaatacactggtacagttgatgatgccgatgcaaagcgtatctccgagttattcagagaatggtttcactacactggcagatttgtcgttggagacgcttttccttttctaggttggttggacctgggcggatacaaaaagacaatggaattagttgctagtagattggactcaatggtcagtaaatggttagatgagcatcgtaaaaagcaagctaacgatgacaaaaaggaggatatggatttcatggatatcatgatctccatgacagaagcaaattcaccacttgaaggatacggcactgatactattatcaagaccacatgtatgactttgattgtttcaggagttgatacaacctcaatcgtacttacttgggccttatcacttttgttaaacaacagagatactttgaaaaaggcacaagaggaattagatatgtgcgtaggtaaaggaagacaagtcaacgagtctgatcttgttaacttgatatacttggaagcagtgcttaaagaggctttaagactttacccagcagcgttcttaggcggaccaagagcattcttggaagattgtactgttgctggttatagaattccaaagggcacctgcttgttgattaacatgtggaaactgcatagagatccaaacatttggagtgatccttgcgaattcaagccagaaagatttttgacacctaatcaaaaggatgttgatgtgatcggtatggatttcgaattgataccatttggtgccggcagaagatattgtccaggtactagattggctttacagatgttgcatatcgtattagcgacattgctgcaaaacttcgaaatgtcaacaccaaacgatgcgccagtcgatatgactgcttctgttggcatgacaaatgccaaagcatcacctttagaagtcttgctatcacctcgtgttaaatggtcctaa

SEQ ID NO: 106 - protein, 522 aa, Stevia rebaudiana
MGLFPLEDSYALVFEGLAITLALYYLLSFIYKTSKKTCTPPKASGEHPITGHLNLLSGSSGLPHLALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 107 - DNA, 1431 bp, Artificial Sequence
  Codon-optimized KAH
atgatacaagttttaactccaattctactcttcctcatcttcttcgttttctggaaagtctacaaacatcaaaagactaaaatcaatctaccaccaggttccttcggctggccatttttgggtgaaaccttagccttacttagagcaggctgggattctgagccagaaagattcgtaagagagcgtatcaaaaagcatggatctccacttgttttcaagacatcactatttggagacagattcgctgttctttgcggtccagctggtaataagtttttgttctgcaacgaaaacaaattagtggcatcttggtggccagtccctgtaaggaagttgttcggtaaaagtttactcacaataagaggagatgaagcaaaatggatgagaaaaatgctattgtcttacttgggtccagatgcatttgccacacattatgccgttactatggatgttgtaacacgtagacatattgatgtccattggaggggcaaggaggaagttaatgtatttcaaacagttaagttgtacgcattcgaattagcttgtagattattcatgaacctagatgacccaaaccacatcgcgaaactcggtagtcttttcaacattttcctcaaagggatcatcgagcttcctatagacgttcctggaactagattttactccagtaaaaaggccgcagctgccattagaattgaattgaaaaagctcattaaagctagaaaactcgaattgaaggagggtaaggcgtcttcttcacaggacttgctttctcatctattaacatcacctgatgagaatgggatgttcttgacagaagaggaaatagtcgataacattctacttttgttattcgctggtcacgatacctctgcactatcaataacacttttgatgaaaaccttaggtgaacacagtgatgtgtacgacaaggttttgaaggaacaattagaaatttccaaaacaaaggaggcttgggaatcactaaagtgggaagatatccagaagatgaagtactcatggtcagtaatctgtgaagtcatgagattgaatcctcctgtcatagggacatacagagaggcgttggttgatatcgactatgctggttacactatcccaaaaggatggaagttgcattggtcagctgtttctactcaaagagacgaagccaatttcgaagatgtaactagattcgatccatccagatttgaaggggcaggccctactccattcacatttgtgcctttcggtggaggtcctagaatgtgtttaggcaaagagtttgccaggttagaagtgttagcatttctccacaacattgttaccaactttaagtgggatcttctaatccctgatgagaagatcgaatatgatccaatggctactccagctaagggcttgccaattagacttcatccacaccaagtctaa

SEQ ID NO: 108 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 109 - DNA, 1578 bp, Artificial Sequence
  Codon-optimized KAH
atggagtctttagtggttcatacagtaaatgctatctggtgtattgtaatcgtcgggattttctcagttggttatcacgtttacggtagagctgtggtcgaacaatggagaatgagaagatcactgaagctacaaggtgttaaaggcccaccaccatccatcttcaatggtaacgtctcagaaatgcaacgtatccaatccgaagctaaacactgctctggcgataacattatctcacatgattattcttcttcattattcccacacttcgatcactggagaaaacagtacggcagaatctacacatactctactggattaaagcaacacttgtacatcaatcatccagaaatggtgaaggagctatctcagactaacacattgaacttgggtagaatcacccatataaccaaaagattgaatcctatcttaggtaacggaatcataacctctaatggtcctcattgggcccatcagcgtagaattatcgcctacgagtttactcatgataagatcaagggtatggttggtttgatggttgagtctgctatgcctatgttgaataagtgggaggagatggtaaagagaggcggagaaatgggatgcgacataagagttgatgaggacttgaaagatgtttcagcagatgtgattgcaaaagcctgtttcggatcctcattttctaaaggtaaggctattttctctatgataagagatttgcttacagctatcacaaagagaagtgttctattcagattcaacggattcactgatatggtctttgggagtaaaaagcatggtgacgttgatatagacgctttagaaatggaattggaatcatccatttgggaaactgtcaaggaacgtgaaatagaatgtaaagatactcacaaaaaggatctgatgcaattgattttggaaggggcaatgcgttcatgtgacggtaacctttgggataaatcagcatatagaagatttgttgtagataattgtaaatctatctacttcgcagggcatgatagtacagctgtctcagtgtcatggtgtttgatgttactggccctaaacccatcatggcaagttaagatccgtgatgaaattctgtcttcttgcaaaaatggtattccagatgccgaaagtatcccaaaccttaaaacagtgactatggttattcaagagacaatgagattataccctccagcaccaatcgtcgggagagaagcctctaaagatatcagattgggcgatctagttgttcctaaaggcgtctgtatatggacactaataccagctttacacagagatcctgagatttggggaccagatgcaaacgatttcaaaccagaaagattttctgaaggaatttcaaaggcttgtaagtatcctcaaagttacattccatttggtctgggtcctagaacatgcgttggtaaaaactttggcatgatggaagtaaaggttcttgtttccctgattgtctccaagttctctttcactctatctcctacctaccaacatagtcctagtcacaaacttttagtagaaccacaacatggggtggtaattagagtggtttaa

SEQ ID NO: 110 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 111 - DNA, 1590 bp, Artificial Sequence
  Codon-optimized KAH
atgtacttcctactacaatacctcaacatcacaaccgttggtgtctttgccacattgtttctctcttattgtttacttctctggagaagtagagcgggtaacaaaaagattgccccagaagctgccgctgcatggcctattatcggccacctccacttacttgcaggtggatcccatcaactaccacatattacattgggtaacatggcagataagtacggtcctgtattcacaatcagaataggcttgcatagagctgtagttgtctcatcttgggaaatggcaaaggaatgttcaacagctaatgatcaagtgtcttcttcaagacctgaactattagcttctaagttgttgggttataactacgccatgtttggtttttcaccatacggttcatactggagagaaatgagaaagatcatctctctcgaattactatctaattccagattggaactattgaaagatgttagagcctcagaagttgtcacatctattaaggaactatacaaattgtgggcggaaaagaagaatgagtcaggattggtttctgtcgagatgaaacaatggttcggagatttgactttaaacgtgatcttgagaatggtggctggtaaaagatacttctccgcgagtgacgcttcagaaaacaaacaggcccagcgttgtagaagagtcttcagagaattcttccatctctccggcttgtttgtggttgctgatgctataccttttcttggatggctcgattggggaagacacgagaagaccttgaaaaagaccgccatagaaatggattccatcgcccaggagtggcttgaggaacatagacgtagaaaagattctggagatgataattctacccaagatttcatggacgttatgcaatctgtgctagatggcaaaaatctaggcggatacgatgctgatacgattaacaaggctacatgcttaactcttatatcaggtggcagtgatactactgtagtttctttgacatgggctcttagtcttgtgttaaacaatagagatactttgaaaaaggcacaggaagagttagacatccaagtcggtaaggaaagattggttaacgagcaagacatcagtaagttagtttacttgcaagcaatagtaaaagagacactcagactttatccaccaggtcctttgggtggtttgagacaattcactgaagattgtacactaggtggctatcacgtttcaaaaggaactagattaatcatgaacttatccaagattcaaaaagatccacgtatttggtctgatcctactgaattccaaccagagagattccttacgactcataaagatgtcgatccacgtggtaaacactttgaattcattccattcggtgcaggaagacgtgcatgtcctggtatcacattcggattacaagtactacatctaacattggcatctttcttgcatgcgtttgaattttcaacaccatcaaatgagcaggttaacatgagagaatcattaggtcttacgaatatgaaatctaccccattagaagttttgatttctccaagactatcccttaattgcttcaaccttatgaaaatttga

SEQ ID NO: 112 - protein, 526 aa, Vitis vinifera
MYFLLQYLNITTVGVFATLFLSYCLLLWRSRAGNKKIAPEAAAAWPIIGHLHLLAGGSHQLPHITLGNMADKYGPVFTIRIGLHRAVVVSSWEMAKECSTANDQVSSSRPELLASKLLGYNYAMFGFSPYGSYWREMRKIISLELLSNSRLELLKDVRASEVVTSIKELYKLWAEKKNESGLVSVEMKQWFGDLTLNVILRMVAGKRYFSASDASENKQAQRCRRVFREFFHLSGLFVVADAIPFLGWLDWGRHEKTLKKTAIEMDSIAQEWLEEHRRRKDSGDDNSTQDFMDVMQSVLDGKNLGGYDADTINKATCLTLISGGSDTTVVSLTWALSLVLNNRDTLKKAQEELDIQVGKERLVNEQDISKLVYLQAIVKETLRLYPPGPLGGLRQFTEDCTLGGYHVSKGTRLIMNLSKIQKDPRIWSDPTEFQPERFLTTHKDVDPRGKHFEFIPFGAGRRACPGITFGLQVLHLTLASFLHAFEFSTPSNEQVNMRESLGLTNMKSTPLEVLISPRLSSCSLYN

SEQ ID NO: 113 - DNA, 1440 bp, Artificial Sequence
  Codon-optimized KAH
atggaacctaacttttacttgtcattactattgttgttcgtgaccttcatttctttaagtctgtttttcatcttttacaaacaaaagtccccattgaatttgccaccagggaaaatgggttaccctatcataggtgaaagtttagaattcctatccacaggctggaagggacatcctgaaaagttcatatttgatagaatgcgtaagtacagtagtgagttattcaagacttctattgtaggcgaatccacagttgtttgctgtggggcagctagtaacaaattcctattctctaacgaaaacaaactggtaactgcctggtggccagattctgttaacaaaatcttcccaacaacttcactggattctaatttgaaggaggaatctataaagatgagaaagttgctgccacagttcttcaaaccagaagcacttcaaagatacgtcggcgttatggatgtaatcgcacaaagacattttgtcactcactgggacaacaaaaatgagatcacagtttatccacttgctaaaagatacactttcttgcttgcgtgtagactgttcatgtctgttgaggatgaaaatcatgtggcgaaattctcagacccattccaactaatcgctgcaggcatcatttcacttcctatcgatcttcctggtactccattcaacaaggccataaaggcttcaaatttcattagaaaagagctgataaagattatcaaacaaagacgtgttgatctggcagagggtacagcatctccaacccaggatatcttgtcacatatgctattaacatctgatgaaaacggtaaatctatgaacgagttgaacattgccgacaagattcttggactattgataggaggccacgatacagcttcagtagcttgcacatttctagtgaagtacttaggagaattaccacatatctacgataaagtctaccaagagcaaatggaaattgccaagtccaaacctgctggggaattgttgaattgggatgacttgaaaaagatgaagtattcatggaatgtggcatgtgaggtaatgagattgtcaccacctttacaaggtggttttagagaggctataactgactttatgtttaacggtttctctattccaaaagggtggaagttatactggtccgccaactctacacacaaaaatgcagaatgtttcccaatgcctgagaaattcgatcctaccagatttgaaggtaatggtccagcgccttatacatttgtaccattcggtggaggccctagaatgtgtcctggaaaggaatacgctagattagaaatcttggttttcatgcataatctggtcaaacgttttaagtgggaaaaggttattccagacgaaaagattattgtcgatccattcccaatcccagctaaagatcttccaatccgtttgtatcctcacaaagcttaa

SEQ ID NO: 114 - protein, 479 aa, Medicago truncatula
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 115 - DNA, 1116 bp, Artificial Sequence
  Codon-optimized GGPPS
atggcctctgttactttgggttcctggatcgtcgtccaccaccataaccatcaccatccatcatctatcctaactaaatctcgttcaagatcctgtcctattacactaaccaaaccaatctcttttcgttcaaagagaacagtttcctctagtagttctatcgtgtcctctagtgtcgtcactaaggaagacaatctgagacagtctgaaccttcttcctttgatttcatgtcatatatcattactaaggcagaactagtgaataaggctcttgattcagcagttccattaagagagccattgaaaatccatgaagcaatgagatactctcttctagctggcgggaagagagtcagacctgtactctgcatagcagcgtgcgaattagttggtggcgaggaatcaaccgctatgcctgccgcttgtgctgtagaaatgattcatacaatgtcactgatacacgatgatttgccatgtatggataacgatgatctgagaaggggtaagccaactaaccataaggttttcggcgaagatgttgccgtcttagctggtgatgctttgttatctttcgcgttcgaacatttggcatccgcaacatcaagtgatgttgtgtcaccagtaagagtagttagagcagttggagaactggctaaagctattggaactgagggtttagttgcaggtcaagtcgtcgatatctcttccgaaggtcttgatttgaatgatgtaggtcttgaacatctcgaattcatccatcttcacaagacagctgcacttttagaagccagtgcggttctcggcgcaattgttggcggagggagtgatgacgaaattgagagattgaggaagtttgctagatgtataggattactgttccaagtagtagacgatatactagatgtgacaaagtcttccaaagagttgggaaaaacagctggtaaagatttgattgccgacaaattgacctaccctaagattatggggctagaaaaatcaagagaatttgccgagaaactcaatagagaggcgcgtgatcaactgttgggtttcgattctgataaagttgcaccactcttagccttagccaactacatcgcttacagacaaaactaa

SEQ ID NO: 116 - protein, 371 aa, Arabidopsis thaliana
MASVTLGSWIVVHHHNHHHPSSILTKSRSRSCPITLTKPISFRSKRTVSSSSSIVSSSVVTKEDNLRQSEPSSFDFMSYIITKAELVNKALDSAVPLREPLKIHEAMRYSLLAGGKRVRPVLCIAACELVGGEESTAMPAACAVEMIHTMSLIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 117 - protein, 511 aa, Rubus suavissimus
MATLLEHFQAMPFAIPIALAALSWLFLFYIKVSFFSNKSAQAKLPPVPVVPGLPVIGNLLQLKEKKPYQTFTRWAEEYGPIYSIRTGASTMVVLNTTQVAKEAMVTRYLSISTRKLSNALKILTADKCMVAISDYNDFHKMIKRYILSNVLGPSAQKRHRSNRDTLRANVCSRLHSQVKNSPREAVNFRRVFEWELFGIALKQAFGKDIEKPIYVEELGTTLSRDEIFKVLVLDIMEGAIEVDWRDFFPYLRWIPNTRMETKIQRLYFRRKAVMTALINEQKKRIASGEEINCYIDFLLKEGKTLTMDQISMLLWETVIETADTTMVTTEWAMYEVAKDSKRQDRLYQEIQKVCGSEMVTEEYLSQLPYLNAVFHETLRKHSPAALVPLRYAHEDTQLGGYYIPAGTEIAINIYGCNMDKHQWESPEEWKPERFLDPKFDPMDLYKTMAFGAGKRVCAGSLQAMLIACPTIGRLVQEFEWKLRDGEEENVDTVGLTTHKRYPMHAILKPRS

SEQ ID NO: 118 - DNA, 1449 bp, Rubus suavissimus
atggattccaaggccttgaagaagccacatgcagtttgtgtaccattccctactcaaggtcacataaacccgttgctaaaattggcaaaactccttcactacagaggcttccatataacctttgtcaacacagagtacaaccataagcgcctgcttcaatcccgaggtaccaactctcttgatggcctgccgtcctttaggttcaaatcgattcccgatggcctccctcctactgatgccaacgccacccaagaagtaacatccctagtcgattccactagaaaaaactgcttagcacccttcaggtgccttctttcagaactcaacacatccccaaattcccctcctgttactctcatagtttctgatggtggcatgttcttcactcttgatgcagcccaagaacttggtcttccagaagttatattcaagccaacttgtgcttcttcattcatgtgctaccatcattgtgcccatgtcattgaaaagggtctcgttcctcttaaagatgccagttatgtgacaaatggctatttggacactgtcatagattggataccaggcataagcagtatccgtttgagggatatgccgagcttcattagaaccacagacccaaatgacatcttgctgaaatttatgatggcagagacggaaagaacccaaagagcgtctgcaatcattattaacacatttgatgcgttggagcatgaagttttagatgcactttcaacttttctaccacctatttattccattggacctctacatctacaactcagtcagatcccagaggatgaggactcaaggtcgattgggtcaaatctgtggagagaagaaccagagtgtcttgaatggctcgactctaaagcaccaaattctgtggtttatgtaaacttcggaagtatcacagtcatgacaaacgagcagctgattgagtttgcttggggtcttgcaaacagtaacatgacctttttatgggtgattaggcccgaccttgttgctgggaaatcagctgttgttcctccagagtttgcggaagtgaccaagggaaggagtttattggcgagttggtgccctcaagaacaagtgttgaaccacccggctgtaggaggattcttgacacacagtggatggaactctacaattgaaagtatatgcggtggagttcccatgatctgttggcccttctttgctgatcaacagacaaattgtaggttctgttgcaaagaatggggtataggcctggagatagagggtgatgttaaaaggaataacatagaagaccttgtaagaaagttaatggaaggaaaggaaggccaagaaatgaggacgaaagccttggaatggaagaagttggcagaggaggctactactgctcccaatggttcatctttcttgaatttggacaagatggttaaccaggtgcttcttcgtcctaagaattag

SEQ ID NO: 119 - protein, 482 aa, Rubus suavissimus
MDSKALKKPHAVCVPFPTQGHINPLLKLAKLLHYRGFHITFVNTEYNHKRLLQSRGTNSLDGLPSFRFKSIPDGLPPTDANATQEVTSLVDSTRKNCLAPFRCLLSELNTSPNSPPVTLIVSDGGMFFTLDAAQELGLPEVIFKPTCASSFMCYHHCAHVIEKGLVPLKDASYVTNGYLDTVIDWIPGISSIRLRDMPSFIRTTDPNDILLKFMMAETERTQRASAIIINTFDALEHEVLDALSTFLPPIYSIGPLHLQLSQIPEDEDSRSIGSNLWREEPECLEWLDSKAPNSVVYVNFGSITVMTNEQLIEFAWGLANSNMTFLWVIRPDLVAGKSAVVPPEFAEVTKGRSLLASWCPQEQVLNHPAVGGFLTHSGWNSTIESICGGVPMICWPFFADQQTNCRFCCKEWGIGLEIEGDVKRNNIEDLVRKLMEGKEGQEMRTKALEWKKLAEEATTAPNGSSFLNLDKMVNQVLLRPKN

(19) United States
(12) Patent Application Publication
(10) Pub. No.: US 2019/0062796 A1
(43) Pub. Date: Feb. 28, 2019

(54) PRODUCTION OF STEVIOL GLYCOSIDES IN RECOMBINANT HOSTS

(71) Applicant: EVOLVA SA, Reinach (CH)

(72) Inventors: Jane Dannow Dyekjaer, Reinach (CH); Jens Houghton-Larsen, Birkeroed (DK); Veronique Douchin, Frederiksberg (DK)

(21) Appl. No.: 15/778,739

(22) PCT Filed: Dec. 9, 2016
(86) PCT No.: PCT/EP2016/080516

§ 371 Date: May 24, 2018

Related U.S. Application Data
(60) Provisional application No. 62/265,895, filed on Dec. 10, 2015.

(51) Int. Cl. C12P 19/56; C12N 15/52; A23L 27/30; A23L 2/60

(52) U.S. Cl. CPC C12P 19/56; C12N 15/52; A23V 2002/00; A23L 2/60; A23L 27/36

(57) ABSTRACT

The invention relates to recombinant microorganisms and methods for producing steviol glycosides and steviol glycoside precursors.

### BACKGROUND OF THE INVENTION

### Field of the Invention

This disclosure relates to recombinant production of steviol glycosides and steviol glycoside precursors in recombinant hosts. In particular, this disclosure relates to production of steviol glycosides comprising Steviol-13-O-Glucoside (13-SMG), Steviol-19-O-Glucoside (19-SMG), Steviol-1,2-Bioside, Steviol-1,3-Bioside, 1,2-Stevioside, 1,3-Stevioside, Rubusoside (Rubu), Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, tri-glucosylated steviol glycosides, tetra-glycosylated steviol glycosides, penta-glucosylated steviol glycosides, hexa-glucosylated steviol glycosides hepta-glucosylated steviol glycosides, or isomers thereof in recombinant hosts.

### Description of Related Art

Sweeteners are well known as ingredients used most commonly in the food, beverage, or confectionary industries. The sweetener can either be incorporated into a final food product during production or for stand-alone use, when appropriately diluted, as a tabletop sweetener or an at-home replacement for sugars in baking. Sweeteners include natural sweeteners such as sucrose, high fructose corn syrup, molasses, maple syrup, and honey and artificial sweeteners such as aspartame, saccharine, and sucralose. Stevia extract is a natural sweetener that can be isolated and extracted from a perennial shrub, Stevia rebaudiana. Stevia is commonly grown in South America and Asia for commercial production of stevia extract. Stevia extract, purified to various degrees, is used commercially as a high intensity sweetener in foods and in blends or alone as a tabletop sweetener.

Chemical structures for several steviol glycosides are shown in FIG. 2, including the diterpene steviol and various steviol glycosides. Extracts of the Stevia plant generally comprise steviol glycosides that contribute to the sweet flavor, although the amount of each steviol glycoside often varies, inter alia, among different production batches.

As recovery and purification of steviol glycosides from the Stevia plant have proven to be labor intensive and inefficient, there remains a need for a recombinant production system that can accumulate high yields of desired steviol glycosides, such as RebD and RebM. There also remains a need for improved production of steviol glycosides in recombinant hosts for commercial uses. As well, there remains a need for identifying enzymes selective towards particular substrates to produce one or more specific steviol glycosides. In some aspects, there remains a need to increase the catalytic capability of enzymes with 19-0 glycosylation activity in order to produce higher yields of steviol glycosides.

### SUMMARY OF THE INVENTION

It is against the above background that the present invention provides certain advantages and advancements over the prior art.

Although this invention as disclosed herein is not limited to specific advantages or functionalities, the invention provides a recombinant host cell comprising a recombinant gene encoding a polypeptide capable of glycosylating steviol or a steviol glycoside at its C-19 carboxyl group and having at least 50% sequence identity to the amino acid sequence set forth in SEQ ID NO:2 or 119, wherein the recombinant host cell is capable of producing the steviol glycoside or a steviol glycoside composition.

In some aspects, the recombinant host cell further comprises:
(a) a gene encoding a polypeptide capable of synthesizing geranylgeranyl pyrophosphate (GGPP) from farnesyl diphosphate (FPP) and isopentenyl diphosphate (IPP);
(b) a gene encoding a polypeptide capable of synthesizing ent-copalyl diphosphate from GGPP;
(c) a gene encoding an a polypeptide capable of synthesizing ent-kaurene from ent-copalyl pyrophosphate;
(d) a gene encoding a polypeptide capable of synthesizing ent-kaurenoic acid from ent-kaurene;
(e) a gene encoding a polypeptide capable of reducing cytochrome P450 complex; and
(f) a gene encoding a polypeptide capable of synthesizing steviol from ent-kaurenoic acid;
(g) a gene encoding a polypeptide capable of glycosylating steviol or a steviol glycoside at its C-13 hydroxyl group;
(h) a gene encoding a polypeptide capable of beta 1,3 glycosylation of the C3′ of the 13-O-glucose, 19-O-glucose, or both 13-O-glucose and 19-O-glucose of a steviol glycoside;
(i) a gene encoding a polypeptide capable of glycosylating steviol or a steviol glycoside at its C-19 carboxyl group;
(j) a gene encoding a polypeptide capable of beta 1,2 glycosylation of the C2′ of the 13-O-glucose, 19-O-glucose, or both 13-O-glucose and 19-O-glucose of a steviol glycoside; and/or
(k) a gene encoding a bifunctional polypeptide capable of synthesizing ent-copalyl diphosphate from GGPP and synthesizing ent-kaurene from ent-copalyl pyrophosphate;

wherein at least one of the genes is a recombinant gene.

In some aspects of the recombinant host cell disclosed herein:
(a) the polypeptide capable of synthesizing GGPP comprises a polypeptide having at least 70% identity to the amino acid sequence set forth in SEQ ID NO:20, SEQ ID NO:22, SEQ ID NO:24, SEQ ID NO:26, SEQ ID NO:28, SEQ ID NO:30, SEQ ID NO:32, or SEQ ID NO:116;
(b) the polypeptide capable of synthesizing ent-copalyl diphosphate comprises a polypeptide having at least 70% identity to the amino acid sequence set forth in SEQ ID NO:34, SEQ ID NO:36, SEQ ID NO:38, SEQ ID NO:40, or SEQ ID NO:42;
(c) the polypeptide capable of synthesizing ent-kaurene comprises a polypeptide having at least 70% identity to the amino acid sequence set forth in SEQ ID NO:44, SEQ ID NO:46, SEQ ID NO:48, SEQ ID NO:50, or SEQ ID NO:52;
(d) the polypeptide capable of synthesizing ent-kaurenoic acid comprises a polypeptide having at least 70% identity to the amino acid sequence set forth in SEQ ID NO:60, SEQ ID NO:62, SEQ ID NO:66, SEQ ID NO:68, SEQ ID NO:70, SEQ ID NO:72, SEQ ID NO:74, SEQ ID NO:76, or SEQ ID NO:117;
(e) the polypeptide capable of reducing cytochrome P450 complex comprises a polypeptide having at least 70% identity to the amino acid sequence set forth in SEQ ID NO:78, SEQ ID SEQ ID NO:82, SEQ ID NO:84, SEQ ID NO:86, SEQ ID NO:88, SEQ ID NO:90, SEQ ID NO:92;
(f) the polypeptide capable of synthesizing steviol comprises a polypeptide having at least 70% identity to the amino acid sequence set forth in SEQ ID NO:94, SEQ ID NO:97, SEQ ID NO:100, SEQ ID NO:101, SEQ ID NO:102, SEQ ID NO:103, SEQ ID NO:104, SEQ ID NO:106, SEQ ID NO:108, SEQ ID NO:110, SEQ ID NO:112, or SEQ ID NO:114;
(g) the polypeptide capable of glycosylating steviol or a steviol glycoside at its C-13 hydroxyl group thereof comprises a polypeptide having at least 55% identity to the amino acid sequence set forth in SEQ ID NO:7;
(h) the polypeptide capable of beta 1,3 glycosylation of the C3′ of the 13-O-glucose, 19-O-glucose, or both 13-O-glucose and 19-O-glucose of a steviol glycoside comprises a polypeptide having at least 50% identity to the amino acid sequence set forth in SEQ ID NO:9;
(i) the polypeptide capable of glycosylating steviol or a steviol glycoside at its C-19 carboxyl group thereof comprises a polypeptide having at least 55% identity to the amino acid sequence set forth in SEQ ID NO:4;
(j) the polypeptide capable of beta 1,2 glycosylation of the C2′ of the 13-O-glucose, 19-O-glucose, or both 13-O-glucose and 19-O-glucose of a steviol glycoside comprises a polypeptide having 80% or greater identity to the amino acid sequence set forth in SEQ ID NO:11; a polypeptide having 80% or greater identity to the amino acid sequence set forth in SEQ ID NO:13; or a polypeptide having at least 65% identity to the amino acid sequence set forth in SEQ ID NO:16; and
(k) the bifunctional polypeptide capable of synthesizing ent-copalyl diphosphate from GGPP and synthesizing ent-kaurene from ent-copalyl pyrophosphate comprises a polypeptide having at least 50% identity to the amino acid sequence set forth in SEQ ID NO:54, SEQ ID NO:56, or SEQ ID NO:58.

In some aspects, the recombinant host cell disclosed herein comprises a plant cell, a mammalian cell, an insect cell, a fungal cell, an algal cell, an archeal cell or a bacterial cell.

In some aspects, the bacterial cell comprises Escherichia bacteria cells, Lactobacillus cells, Lactococcus, Cornebacterium cells, Acetobacter cells, Acinetobacter cells, or Pseudomonas cells.

In some aspects, the fungal cell comprises a yeast cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In some aspects the yeast cell is a Saccharomycete.

In some aspects, the yeast cell is a cell from the Saccharomyces cerevisiae species.

The invention further provides a method of producing a steviol glycoside or a steviol glycoside composition, comprising growing the recombinant host cell disclosed herein in a cell culture medium, under conditions in which the genes are expressed, wherein the steviol glycoside or the steviol glycoside composition is produced by the recombinant host cell.

The invention further provides a method for producing a steviol glycoside or a steviol glycoside composition, comprising whole-cell bioconversion of a plant-derived or synthetic steviol and/or steviol glycosides in a cell culture medium using a recombinant polypeptide capable of glycosylating steviol or a steviol glycoside at its C-19 carboxyl group and having at least 50% sequence identity to the amino acid sequence set forth in SEQ IN NO:2 or 119 produced by the recombinant host cell disclosed herein, and synthesizing the steviol glycoside thereby.

In some aspects, the methods disclosed herein further comprises isolating the steviol glycoside or the steviol glycoside composition.

In some aspects of the methods disclosed herein, the isolating step comprises:
(a) providing the cell culture medium comprising the steviol glycoside or the steviol glycoside composition;
(b) separating a liquid phase of the cell culture medium from a solid phase of the cell culture to obtain a supernatant comprising the steviol glycoside or the steviol glycoside composition;
(c) providing one or more adsorbent resins, comprising providing the adsorbent resins in a packed column; and
(d) contacting the supernatant of step (b) with the one or more adsorbent resins in order to obtain at least a portion of the steviol glycoside or the steviol glycoside composition, thereby isolating the steviol glycoside or the steviol glycoside composition.

In some aspects of the methods disclosed herein, the isolating step comprises:
(a) providing the cell culture comprising the steviol glycoside or the steviol glycoside composition;
(b) separating a liquid phase of the cell culture from a solid phase of the cell culture to obtain a supernatant comprising the steviol glycoside or the steviol glycoside composition;
(c) providing one or more ion exchange or ion exchange or reversed-phase chromatography columns; and
(d) contacting the supernatant of step (b) with the one or more ion exchange or ion exchange or reversed-phase chromatography columns in order to obtain at least a portion of the steviol glycoside or the steviol glycoside composition.

In some aspects of the methods disclosed herein, the isolating step comprises:
(a) providing the cell culture comprising the steviol glycoside or the steviol glycoside composition;
(b) separating a liquid phase of the cell culture from a solid phase of the cell culture to obtain a supernatant comprising the steviol glycoside or the steviol glycoside composition;
(c) crystallizing or extracting the steviol glycoside, thereby isolating the steviol glycoside or the steviol glycoside composition.

In some aspects, the methods disclosed herein further comprise recovering the steviol glycoside alone or as a composition comprising the steviol glycoside.

In some aspects of the methods disclosed herein, the recovered composition is enriched for the steviol glycoside, relative to a glycoside composition from a Stevia plant and has a reduced level of Stevia plant-derived components relative to a plant-derived Stevia extract.

In some aspects of the method disclosed herein, the recovered composition has a reduced level of Stevia plant-derived components relative to a plant-derived Stevia extract.

In some aspects of the methods disclosed herein, the cell culture medium comprises:
(a) the steviol glycoside or the steviol glycoside composition produced by the recombinant host cell disclosed herein or whole-cell bioconversion of a plant-derived or synthetic steviol and/or steviol glycosides,
(b) glucose, fructose, and/or sucrose; and/or
(c) supplemental nutrients comprising trace metals, vitamins, salts, yeast nitrogen base (YNB), and/or amino acids.

In some aspects of the method disclosed herein, the steviol glycoside is produced in a permeabilized recombinant host cell which has been transformed with the gene encoding the polypeptide capable of glycosylating steviol or a steviol glycoside at its C-19 carboxyl group.

In some aspects of the methods disclosed herein, the recombinant host comprises a plant cell, a mammalian cell, an insect cell, a fungal cell, an algal cell or a bacterial cell.

In some aspects of the methods disclosed herein, the bacterial cell comprises Escherichia cells, Lactobacillus cells, Lactococcus cells, Cornebacterium cells, Acetobacter cells, Acinetobacter cells, or Pseudomonas cells.

In some aspects of the methods disclosed herein, the fungal cell comprises a yeast cell.

In some aspects of the methods disclosed herein, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In some aspects of the methods disclosed herein, the yeast cell is a Saccharomycete.

In some aspects of the methods disclosed herein, the yeast cell is a cell from the Saccharomyces cerevisiae species.

The invention further provides in vitro method for producing steviol-19-O-glucoside (19-SMG), comprising adding a recombinant polypeptide capable of glycosylating steviol or a steviol glycoside at its C-19 carboxyl group and having at least 50% sequence identity to the amino acid sequence set forth in SEQ IN NO:2 or 119 and a plant-derived or synthetic steviol to a reaction mixture; and synthesizing 19-SMG thereby.

In some aspects of the methods disclosure herein, the reaction mixture comprises:
(a) one or more 19-SMG;
(b) the recombinant polypeptide;
(c) glucose, fructose, and/or sucrose, uridine diphosphate (UDP)-glucose, UDP-rhamnose, UDP-xylose, and/or N-acetyl-glucosamine; and/or
(d) reaction buffer and/or salts.

In some aspects of the methods disclosed herein, the steviol glycoside comprises steviol-13-O-glucoside (13-SMG), steviol-1,2-bioside, steviol-1,3-bioside, steviol-19-O-glucoside (19-SMG), stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, or isomers thereof.

The invention further provides a cell culture medium, comprising:
(a) the recombinant host cells disclosed herein; and
(b) one or more steviol glycosides produced by the recombinant host cells disclosed herein;

wherein one or more steviol glycosides is present at a concentration of at least 0.1 mg/liter of the cell culture medium.

The invention further provides a cell culture medium, comprising:
(a) the recombinant host cells disclosed herein; and
(b) one or more steviol glycosides produced by the recombinant host cells disclosed herein;

wherein one or more steviol glycosides is present at a concentration of at least 0.1 mg/liter of the cell culture medium and the cell culture medium further comprises glucose, sucrose, UDP-glucose, UDP-rhamnose, UDP-xylose, N-acetyl-glucosamine, and/or YNB.

The invention further provides a cell culture medium, comprising:
(a) the recombinant host cells disclosed herein; and
(b) one or more steviol glycosides produced by the recombinant host cells disclosed herein;
wherein one or more steviol glycosides is present at a concentration of at least 0.1 mg/liter of the culture medium and the cell culture medium further comprises glucose, sucrose, UDP-glucose, UDP-rhamnose, UDP-xylose, N-acetyl-glucosamine, and/or YNB; and
wherein the cell culture medium has a reduced level of Stevia plant-derived components relative to a plant-derived Stevia extract.

The invention further provides a cell culture medium, comprising:
(a) the recombinant host cells disclosed herein; and
(b) one or more steviol glycosides produced by the recombinant host cells disclosed herein;
wherein one or more steviol glycosides is present at a concentration of at least 0.1 mg/liter of the cell culture medium and the cell culture medium further comprises glucose, sucrose, UDP-glucose, UDP-rhamnose, UDP-xylose, N-acetyl-glucosamine, and/or YNB; and
wherein the cell culture medium is enriched for 19-SMG relative to a steviol glycoside extract from Stevia plant and has a reduced level of Stevia plant-derived components relative to a plant-derived Stevia extract.

The invention further provides a cell lysate comprising one or more steviol glycosides produced by the recombinant host cells disclosed herein, and the cell lysate further comprises glucose, sucrose, UDP-glucose, UDP-rhamnose, UDP-xylose, N-acetyl-glucosamine, and/or YNB.

The invention further provides a steviol glycoside produced by the recombinant host cells disclosed herein.

The invention further provides a steviol glycoside produced by the methods disclosed herein.

The invention further provides a sweetener composition, comprising a steviol glycoside produced by the recombinant host cells or the method disclosed herein.

The invention further provides a food product comprising the sweetener composition disclosed herein.

The invention further provides a beverage or a beverage concentration comprising the sweetener composition disclosed herein.

The invention further provides a composition of steviol glycosides produced by a recombinant host disclosed herein, wherein the relative levels of steviol glycosides in the composition correspond to the relative levels of steviol glycosides in the recombinant host.

In some aspects, the composition further comprises an increased level of 19-SMG relative to a composition of steviol glycosides produced by a corresponding recombinant host cell lacking the gene encoding a polypeptide capable of glycosylating steviol or a steviol glycoside at its C-19 carboxyl group and having at least 50% sequence identity to the amino acid sequence set forth in SEQ IN NO:2 or 119.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic of an engineered biosynthetic pathway for producing steviol in yeast from geranylgeranyl diphosphate using a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a polypeptide capable of synthesizing ent-copalyl diphosphate from GGPP (e.g., ent-copalyl diphosphate synthase (CDPS)); a polypeptide capable of synthesizing ent-kaurene from ent-copalyl pyrophosphate (e.g., kaurene synthase (KS)); a polypeptide capable of synthesizing ent-kaurenoic acid from ent-kaurene (e.g., kaurene oxidase (KO)); a polypeptide capable of reducing cytochrome P450 complex (e.g., cytochrome P450 reductase (CPR)) (not shown); and a polypeptide capable of synthesizing steviol from ent-kaurenoic acid (e.g., steviol synthase (KAH)).

FIG. 2 shows representative steviol glycoside glycosylation reactions catalyzed by suitable uridine 5′-diphospho (UDP) glycosyl transferases (UGT) enzymes and chemical structures for several steviol glycoside compounds.

FIG. 3 shows 19-SMG accumulation (in area-under-the-curve; AUC) by a steviol-fed S. cerevisiae strain expressing UGT33942 (SEQ ID NO:1, SEQ ID NO:2).

FIG. 4 shows liquid chromatography-mass spectrometry (LC-MS) selected-ion recording (SIR) traces, corresponding to the mass of 19-SMG, of samples from a steviol-fed S. cerevisiae control strain (top) and a steviol-fed S. cerevisiae strain expressing UGT33786 (SEQ ID NO:118, SEQ ID NO:119) (bottom).

### DETAILED DESCRIPTION OF THE INVENTION

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent dearee of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide”, “nucleotide”, “oligonucleotide”, and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the terms “microorganism,” “microorganism host,” “microorganism host cell,” “host,” and “host cell” can be used interchangeably. As used herein, the terms “recombinant host” or “recombinant microorganism” are intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in S. cerevisiae.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous yeast gene is deleted. See, e.g., Giaever & Nislow, 2014, Genetics 197(2):451-65. As used herein, the terms “deletion,” “deleted,” “knockout,” and “knocked out” can be used interchangeably to refer to an endogenous gene that has been manipulated to no longer be expressed in an organism, including, but not limited to, S. cerevisiae.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the term “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

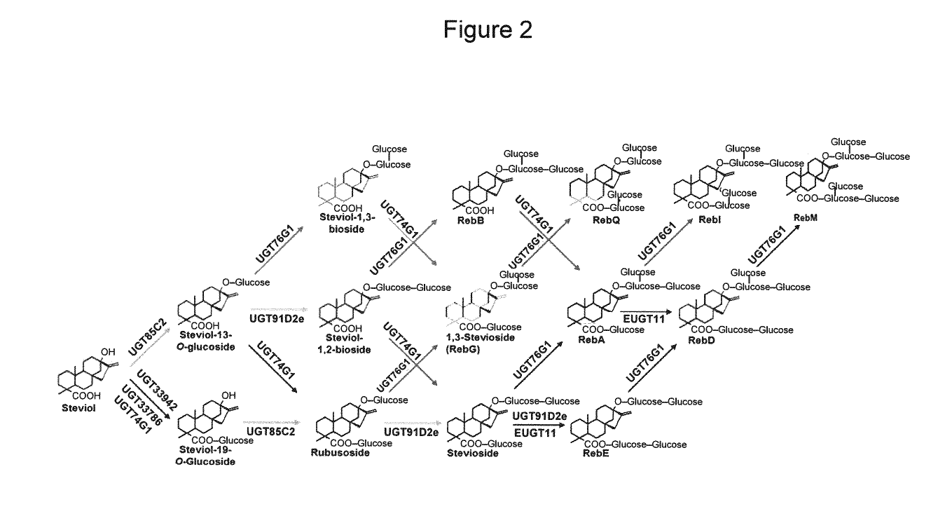

As used herein, the term “steviol glycoside” refers to Rebaudioside A (RebA) (CAS #58543-16-1), Rebaudioside B (RebB) (CAS #58543-17-2), Rebaudioside C (RebC) (CAS #63550-99-2), Rebaudioside D (RebD) (CAS #63279-13-0), Rebaudioside E (RebE) (CAS #63279-14-1), Rebaudioside F (RebF) (CAS #438045-89-7), Rebaudioside M (RebM) (CAS #1220616-44-3), Rubusoside (CAS #63849-39-4), Dulcoside A (CAS #64432-06-0), Rebaudioside I (RebI) (MassBank Record: FU000332), Rebaudioside Q (RebQ), 1,2-Stevioside (CAS #57817-89-7), 1,3-Stevioside (RebG), 1,2-bioside (MassBank Record: FU000299), 1,3-bioside, Steviol-13-O-glucoside (13-SMG), Steviol-19-O-glucoside (19-SMG), a tri-glucosylated steviol glycoside, a tetra-glycosylated steviol glycoside, a penta-glucosylated steviol glycoside, a hexa-glucosylated steviol glycoside, a hepta-glucosylated steviol glycoside, and isomers thereof. See FIG. 2; see also, Steviol Glycosides Chemical and Technical Assessment 69th JECFA, 2007, prepared by Harriet Wallin, Food Agric. Org.

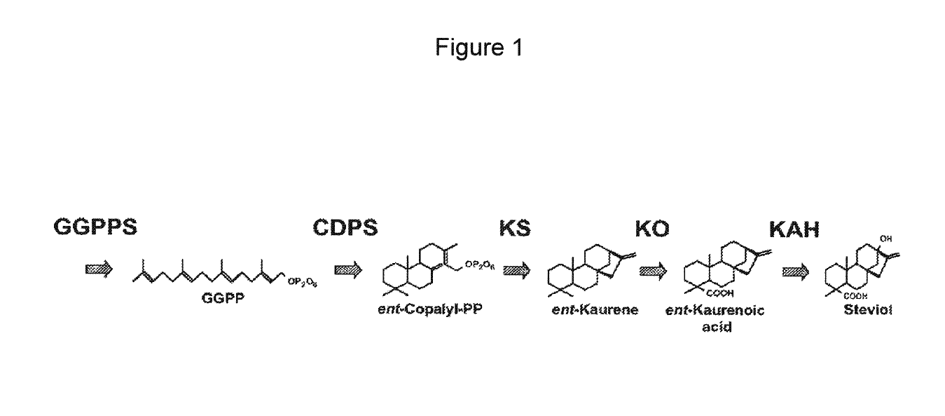

As used herein, the terms “steviol glycoside precursor” and “steviol glycoside precursor compound” are used to refer to intermediate compounds in the steviol glycoside biosynthetic pathway. Steviol glycoside precursors include, but are not limited to, geranylgeranyl diphosphate (GGPP), ent-copalyl-diphosphate, ent-kaurene, ent-kaurenol, ent-kaurenal, ent-kaurenoic acid, and steviol. See FIG. 1. In some embodiments, steviol glycoside precursors are themselves steviol glycoside compounds. For example, 19-SMG, rubusoside, stevioside, and RebE are steviol glycoside precursors of RebM. See FIG. 2. Steviol glycosides and/or steviol glycoside precursors can be produced in vivo (i.e., in a recombinant host), in vitro (i.e., enzymatically), or by whole cell bioconversion. As used herein, the terms “produce” and “accumulate” can be used interchangeably to describe synthesis of steviol glycosides and steviol glycoside precursors in vivo, in vitro, or by whole cell bioconversion.

Recombinant steviol glycoside-producing Saccharomyces cerevisiae (S. cerevisiae) strains are described in WO 2011/153378, WO 2013/022989, WO 2014/122227, and WO 2014/122328. Methods of producing steviol glycosides in recombinant hosts, by whole cell bioconversion, and in vitro are also described in WO 2011/153378, WO 2013/022989, WO 2014/122227, and WO 2014/122328. All of these publications are hereby incorporated herein by reference in their entirety.

In some embodiments, steviol glycosides are produced in vivo through expression of one or more enzymes involved in the steviol glycoside biosynthetic pathway in a recombinant host. For example, a steviol-producing recombinant host expressing a recombinant gene encoding a polypeptide capable of glycosylating steviol or a steviol glycoside at its C-19 carboxyl group and having at least 50% sequence identity to the amino acid sequence set forth in SEQ IN NO:2 or 119 (UDP-glycosyltransferase (UGT) polypeptide structurally similar to members of the UGT85 family capable of glycosyiating steviol or a steviol glycoside at its C-19 carboxyl group) can produce a steviol glycoside in vivo.

In some embodiments, a recombinant host comprising a gene encoding a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a gene encoding a polypeptide capable of synthesizing ent-copalyl diphosphate from GGPP (e.g., ent-copalyl diphosphate synthase (CDPS)); a gene encoding a polypeptide capable of synthesizing ent-kaurene from ent-copalyl pyrophosphate (e.g., kaurene synthase (KS)); a gene encoding a polypeptide capable of synthesizing ent-kaurenoic acid from ent-kaurene (e.g., kaurene oxidase (KO)); a gene encoding a polypeptide capable of reducing cytochrome P450 complex (e.g., cytochrome P450 reductase (CPR); for example, but not limited to a polypeptide capable of electron transfer from NADPH to cytochrome P450 complex during conversion of NADPH to NADP⁺, which is utilized as a cofactor for terpenoid biosynthesis); and a gene encoding a polypeptide capable of synthesizing steviol from ent-kaurenoic acid (e.g., steviol synthase (KAH)); and/or a gene encoding a bifunctional polypeptide capable of synthesizing ent-copalyl diphosphate from GGPP and synthesizing ent-kaurene from ent-copalyl pyrophosphate (e.g., an ent-copalyl diphosphate synthase (CDPS)—ent-kaurene synthase (KS) polypeptide) can produce steviol in vivo. See, e.g., FIG. 1. The skilled worker will appreciate that one or more of these genes can be endogenous to the host provided that at least one (and in some embodiments, all) of these genes is a recombinant gene introduced into the recombinant host.

In some embodiments, steviol glycosides and/or steviol glycoside precursors are produced in vivo through expression of a recombinant gene encoding a polypeptide capable of glycosylating steviol or a steviol glycoside at its C-19 carboxyl group and having at least 50% sequence identity to the amino acid sequence set forth in SEQ IN NO:2 or 119 and one or more enzymes involved in the steviol glycoside biosynthetic pathway in a recombinant host. For example, a steviol-producing recombinant host comprising a gene encoding a polypeptide capable of glycosylating steviol or a steviol glycoside at its C-19 carboxyl group and having at least 50% sequence identity to the amino acid sequence set forth in SEQ IN NO:2 or 119, a gene encoding a polypeptide capable of synthesizing geranylgeranyl pyrophosphate (GGPP) from farnesyl diphosphate (FPP) and isopentenyl diphosphate (IPP); a gene encoding a polypeptide capable of synthesizing ent-copalyl diphosphate from GGPP; a gene encoding a polypeptide capable of synthesizing ent-kaurene from ent-copalyl pyrophosphate; a gene encoding a polypeptide capable of synthesizing ent-kaurenoic acid from ent-kaurene; a gene encoding a polypeptide capable of reducing cytochrome P450 complex; and/or a gene encoding a bifunctional polypeptide capable of synthesizing ent-copalyl diphosphate from GGPP and synthesizing ent-kaurene from ent-copalyl pyrophosphate; a gene encoding a polypeptide capable of glycosylating steviol or a steviol glycoside at its C-13 hydroxyl group (e.g., UGT85C2 polypeptide); a gene encoding a polypeptide capable of beta 1,3 glycosylation of the C3′ of the 13-O-glucose, 19-O-glucose, or both 13-O-glucose and 19-O-glucose of a steviol glycoside (e.g., UGT76G1 polypeptide); a gene encoding a polypeptide capable of glycosylating steviol or a steviol glycoside at its C-19 carboxyl group (e.g., UGT74G1 polypeptide); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., UGT91D2 and EUGT11 polypeptide) can produce a steviol glycoside and/or a steviol glycoside precursor in vivo. See, e.g., FIGS. 1 and 2. The skilled worker will appreciate that one or more of these genes can be endogenous to the host provided that at least one (and in some embodiments, all) of these genes is a recombinant gene introduced into the recombinant host.

In some aspects, the polypeptide capable of synthesizing geranylgeranyl pyrophosphate (GGPP) from farnesyl diphosphate (FPP) and isopentenyl diphosphate (IPP) comprises a polypeptide having the amino acid sequence set forth in SEQ ID NO:20 (which can be encoded by the nucleotide sequence set forth in SEQ ID NO:19), SEQ ID NO:22 (encoded by the nucleotide sequence set forth in SEQ ID NO:21), SEQ ID NO:24 (encoded by the nucleotide sequence set forth in SEQ ID NO:23), SEQ ID NO:26 (encoded by the nucleotide sequence set forth in SEQ ID NO:25), SEQ ID NO:28 (encoded by the nucleotide sequence set forth in SEQ ID NO:27), SEQ ID NO:30 (encoded by the nucleotide sequence set forth in SEQ ID NO:29), SEQ ID NO:32 (encoded by the nucleotide sequence set forth in SEQ ID NO:31), or SEQ ID NO:116 (encoded by the nucleotide sequence set forth in SEQ ID NO:115).

In some aspects, the polypeptide capable of synthesizing ent-copalyl diphosphate from GGPP comprises a polypeptide having the amino acid sequence set forth in SEQ ID NO:34 (which can be encoded by the nucleotide sequence set forth in SEQ ID NO:33), SEQ ID NO:36 (encoded by the nucleotide sequence set forth in SEQ ID NO:35), SEQ ID NO:38 (encoded by the nucleotide sequence set forth in SEQ ID NO:37), SEQ ID NO:40 (encoded by the nucleotide sequence set forth in SEQ ID NO:39), or SEQ ID NO:42 (encoded by the nucleotide sequence set forth in SEQ ID NO:41). In some embodiments, the polypeptide capable of synthesizing ent-copalyl diphosphate from GGPP lacks a chloroplast transit peptide.

In some aspects, the polypeptide capable of synthesizing ent-kaurene from ent-copalyl pyrophosphate comprises a polypeptide having the amino acid sequence set forth in SEQ ID NO:44 (which can be encoded by the nucleotide sequence set forth in SEQ ID NO:43), SEQ ID NO:46 (encoded by the nucleotide sequence set forth in SEQ ID NO:45), SEQ ID NO:48 (encoded by the nucleotide sequence set forth in SEQ ID NO:47), SEQ ID NO:50 (encoded by the nucleotide sequence set forth in SEQ ID NO:49), or SEQ ID NO:52 (encoded by the nucleotide sequence set forth in SEQ ID NO:51).

In some embodiments, a recombinant host comprises a gene encoding a bifunctional polypeptide capable of synthesizing ent-copalyl diphosphate from GGPP and synthesizing ent-kaurene from ent-copalyl pyrophosphate. In some aspects, the bifunctional polypeptide comprises a polypeptide having the amino acid sequence set forth in SEQ ID NO:54 (which can be encoded by the nucleotide sequence set forth in SEQ ID NO:53), SEQ ID NO:56 (encoded by the nucleotide sequence set forth in SEQ ID NO:55), or SEQ ID NO:58 (encoded by the nucleotide sequence set forth in SEQ ID NO:57).

In some aspects, the polypeptide capable of synthesizing ent-kaurenoic acid from ent-kaurene comprises a polypeptide having the amino acid sequence set forth in SEQ ID NO:60 (which can be encoded by the nucleotide sequence set forth in SEQ ID NO:59), SEQ ID NO:62 (encoded by the nucleotide sequence set forth in SEQ ID NO:61), SEQ ID NO:66 (encoded by the nucleotide sequence set forth in SEQ ID NO:65), SEQ ID NO:68 (encoded by the nucleotide sequence set forth in SEQ ID NO:67), SEQ ID NO:70 (encoded by the nucleotide sequence set forth in SEQ ID NO:69), SEQ ID NO:72 (encoded by the nucleotide sequence set forth in SEQ ID NO:71), SEQ ID NO:74 (encoded by the nucleotide sequence set forth in SEQ ID NO:73), SEQ ID NO:76 (encoded by the nucleotide sequence set forth in SEQ ID NO:75), or SEQ ID NO:117 (encoded by the nucleotide sequence set forth in SEQ ID NO:63 or SEQ ID NO:64).

In some aspects, the polypeptide capable of reducing cytochrome P450 complex comprises a polypeptide having the amino acid sequence set forth in SEQ ID NO:78 (which can be encoded by the nucleotide sequence set forth in SEQ ID NO:77), SEQ ID NO:80 (encoded by the nucleotide sequence set forth in SEQ ID NO:79), SEQ ID NO:82 (encoded by the nucleotide sequence set forth in SEQ ID NO:81), SEQ ID NO:84 (encoded by the nucleotide sequence set forth in SEQ ID NO:83), SEQ ID NO:86 (encoded by the nucleotide sequence set forth in SEQ ID NO:85), SEQ ID NO:88 (encoded by the nucleotide sequence set forth in SEQ ID NO:87), SEQ ID NO:90 (encoded by the nucleotide sequence set forth in SEQ ID NO:89), or SEQ ID NO:92 (encoded by the nucleotide sequence set forth in SEQ ID NO:91).

In some aspects, the polypeptide capable of synthesizing steviol from ent-kaurenoic acid comprises a polypeptide having the amino acid sequence set forth in SEQ ID NO:94 (which can be encoded by the nucleotide sequence set forth in SEQ ID NO:93), SEQ ID NO:97 (encoded by the nucleotide sequence set forth in SEQ ID NO:95 or SEQ ID NO:96), SEQ ID NO:100 (encoded by the nucleotide sequence set forth in SEQ ID NO:98 or SEQ ID NO:99), SEQ ID NO:101, SEQ ID NO:102, SEQ ID NO:103, SEQ ID NO:104, SEQ ID NO:106 (encoded by the nucleotide sequence set forth in SEQ ID NO:105), SEQ ID NO:108 (encoded by the nucleotide sequence set forth in SEQ ID NO:107), SEQ ID NO:110 (encoded by the nucleotide sequence set forth in SEQ ID NO:109), SEQ ID NO:112 (encoded by the nucleotide sequence set forth in SEQ ID NO:111), or SEQ ID NO:114 (encoded by the nucleotide sequence set forth in SEQ ID NO:113).

In some embodiments, a recombinant host comprises a nucleic acid encoding a polypeptide capable of glycosylating steviol or a steviol glycoside at its C-13 hydroxyl group (e.g., UGT85C2 polypeptide) (SEQ ID NO:7), a nucleic acid encoding a polypeptide capable of beta 1,3 glycosylation of the C3′ of the 13-O-glucose, 19-O-glucose, or both 13-O-glucose and 19-O-glucose of a steviol glycoside (e.g., UGT76G1 polypeptide) (SEQ ID NO:9), a nucleic acid encoding a polypeptide capable of glycosylating steviol or a steviol glycoside at its C-19 carboxyl group (e.g., UGT74G1 polypeptide) (SEQ ID NO:4), a nucleic acid encoding a polypeptide capable of beta 1,2 glycosylation of the C2′ of the 13-O-glucose, 19-O-glucose, or both 13-O-glucose and 19-O-glucose of a steviol glycoside (e.g., EUGT11 polypeptide) (SEQ ID NO:16), a nucleic acid encoding a polypeptide capable of glycosylating steviol or a steviol glycoside at its C-19 carboxyl group (e.g., UGT33942 polypeptide) (SEQ ID NO:2), and/or a nucleic acid encoding a polypeptide capable of glycosylating steviol or a steviol glycoside at its C-19 carboxyl group (e.g., UGT33786 polypeptide) (SEQ ID NO:119). In some aspects, the polypeptide capable of beta 1,2 glycosylation of the C2′ of the 13-O-glucose, 19-O-glucose, or both 13-O-glucose and 19-O-glucose of a steviol glycoside (e.g., UGT91D2 polypeptide) can be a UGT91D2e polypeptide (SEQ ID NO:11) or a UGT91D2e-b polypeptide (SEQ ID NO:13).

In some aspects, the polypeptide capable of glycosylating steviol or a steviol glycoside at its C-13 hydroxyl group is encoded by the nucleotide sequence set forth in SEQ ID NO:5 or SEQ ID NO:6, the polypeptide capable of beta 1,3 glycosylation of the C3′ of the 13-O-glucose, 19-O-glucose, or both 13-O-glucose and 19-O-glucose of a steviol glycoside is encoded by the nucleotide sequence set forth in SEQ ID NO:8, the polypeptide capable of glycosylating steviol or a steviol glycoside at its C-19 carboxyl group is encoded by the nucleotide sequence set forth in SEQ ID NO:3, the polypeptide capable of beta 1,2 glycosylation of the C2′ of the 13-O-glucose, 19-O-glucose, or both 13-O-glucose and 19-O-glucose of a steviol glycoside is encoded by the nucleotide sequence set forth in SEQ ID NO:10,12,14 or 15, the UGT33942 polypeptide is encoded by the nucleotide sequence set forth in SEQ ID NO:1, and the UGT33786 polypeptide is encoded by the nucleotide sequence set forth in SEQ ID NO:118. The skilled worker will appreciate that expression of these genes may be necessary to produce a particular steviol glycoside but that one or more of these genes can be endogenous to the host provided that at least one (and in some embodiments, all) of these genes is a recombinant gene introduced into the recombinant host.

In a particular embodiment, a steviol-producing recombinant microorganism comprises exogenous nucleic acids encoding a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, or a polypeptide capable of beta 1,2 glycosylation of the C2′ of the 13-O-glucose, 19-0-glucose, or both 13-O-glucose and 19-O-glucose of a steviol glycoside polypeptides.

In another particular embodiment, a steviol-producing recombinant microorganism comprises exogenous nucleic acids encoding a polypeptide capable of glycosylating steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside.

In another particular embodiment, a steviol-producing recombinant microorganism comprises exogenous nucleic acids encoding a polypeptide capable of glycosylating steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside and/or UGT33942 polypeptide.

In yet another particular embodiment, a steviol-producing recombinant microorganism comprises exogenous nucleic acids encoding a polypeptide capable of glycosylating steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside, and/or UGT33786 polypeptides.

In yet another particular embodiment, a steviol-producing recombinant microorganism comprises exogenous nucleic acids encoding a polypeptide capable of glycosylating steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside, and/or UGT33942 polypeptide and UGT33786 polypeptides.

In some embodiments, steviol glycosides and/or steviol glycoside precursors are produced in vivo through expression in a recombinant host of one or more enzymes capable of reactions found in the steviol glycoside biosynthetic pathway. For example, a steviol-producing recombinant host expressing one or more of a gene encoding a polypeptide capable of synthesizing geranylgeranyl pyrophosphate (GGPP) from farnesyl diphosphate (FPP) and isopentenyl diphosphate (IPP), a gene encoding a polypeptide capable of synthesizing ent-copalyl diphosphate from GGPP, a gene encoding a polypeptide capable of synthesizing ent-kaurene from ent-copalyl pyrophosphate, a gene encoding a polypeptide capable of synthesizing ent-kaurenoic acid from ent-kaurene, a gene encoding a polypeptide capable of synthesizing steviol from ent-kaurenoic acid, a gene encoding a polypeptide capable of reducing cytochrome P450 complex, and/or a gene encoding a bifunctional polypeptide capable of synthesizing ent-copalyl diphosphate from GGPP and synthesizing ent-kaurene from ent-copalyl pyrophosphate, and one or more of a gene encoding a polypeptide capable of beta 1,3 glycosylation of the C3′ of the 13-O-glucose, 19-O-glucose, or both 13-O-glucose and 19-O-glucose of a steviol glycoside, a polypeptide capable of beta 1,2 glycosylation of the C2′ of the 13-O-glucose, 19-O-glucose, or both 13-O-glucose and 19-O-glucose of a steviol glycoside, and a polypeptide capable of glycosylating steviol or a steviol glycoside at its C-13 hydroxyl group or a polypeptide capable of glycosylating steviol or a steviol glycoside at its C-19 carboxyl group, can produce a steviol glycoside and/or steviol glycoside precursors in vivo. The skilled worker will appreciate that one or more of these genes can be endogenous to the host provided that at least one (and in some embodiments, all) of these genes is a recombinant gene introduced into the recombinant host.

In some embodiments, steviol glycosides and/or steviol glycoside precursors are produced in vivo through expression in a recombinant host of a recombinant gene encoding a UDP-glycosyltransferase (UGT) polypeptide structurally similar to members of the UGT85 family capable of glycosylating steviol or a steviol glycoside at its C-19 carboxyl group (e.g., UGT33942 and UGT33786), and one or more enzymes capable of reactions found in the steviol glycoside biosynthetic pathway. For example, a steviol-producing recombinant host expressing a UDP-glycosyltransferase (UGT) polypeptide structurally similar to members of the UGT85 family capable of glycosylating steviol or a steviol glycoside at its C-19 carboxyl group (e.g., UGT33942 and UGT33786), and one or more of a gene encoding a polypeptide capable of synthesizing geranylgeranyl pyrophosphate (GGPP) from farnesyl diphosphate (FPP) and isopentenyl diphosphate (IPP), a gene encoding a polypeptide capable of synthesizing ent-copalyl diphosphate from GGPP, a gene encoding a polypeptide capable of synthesizing ent-kaurene from ent-copalyl pyrophosphate, a gene encoding a polypeptide capable of synthesizing ent-kaurenoic acid from ent-kaurene, a gene encoding a polypeptide capable of synthesizing steviol from ent-kaurenoic acid, a gene encoding a polypeptide capable of reducing cytochrome P450 complex, and/or a gene encoding a bifunctional polypeptide capable of synthesizing ent-copalyl diphosphate from GGPP and synthesizing ent-kaurene from ent-copalyl pyrophosphate, and one or more of a gene encoding a polypeptide capable of beta 1,3 glycosylation of the C3′ of the 13-O-glucose, 19-O-glucose, or both 13-O-glucose and 19-O-glucose of a steviol glycoside, a polypeptide capable of beta 1,2 glycosylation of the C2′ of the 13-O-glucose, 19-O-glucose, or both 13-O-glucose and 19-O-glucose of a steviol glycoside, and a polypeptide capable of glycosylating steviol or a steviol glycoside at its C-13 hydroxyl group or a polypeptide capable of glycosylating steviol or a steviol glycoside at its C-19 carboxyl group, can produce a steviol glycoside in vivo. The skilled worker will appreciate that one or more of these genes can be endogenous to the host provided that at least one (and in some embodiments, all) of these genes is a recombinant gene introduced into the recombinant host.

In another example, a recombinant host expressing a gene encoding a polypeptide capable of synthesizing geranylgeranyl pyrophosphate (GGPP) from farnesyl diphosphate (FPP) and isopentenyl diphosphate (IPP), a gene encoding a polypeptide capable of synthesizing ent-copalyl diphosphate from GGPP, a gene encoding a polypeptide capable of synthesizing ent-kaurene from ent-copalyl pyrophosphate, a gene encoding a polypeptide capable of synthesizing ent-kaurenoic acid from ent-kaurene, a gene encoding a polypeptide capable of synthesizing steviol from ent-kaurenoic acid, a gene encoding a polypeptide capable of reducing cytochrome P450 complex, and/or a gene encoding a bifunctional polypeptide capable of synthesizing ent-copalyl diphosphate from GGPP and synthesizing ent-kaurene from ent-copalyl pyrophosphate can produce steviol in vivo. The skilled worker will appreciate that one or more of these genes can be endogenous to the host provided that at least one (and in some embodiments, all) of these genes is a recombinant gene introduced into the recombinant host.

In another example, a recombinant host expressing a recombinant gene encoding a UDP-glycosyltransferase (UGT) polypeptide structurally similar to members of the UGT85 family capable of glycosylating steviol or a steviol glycoside at its C-19 carboxyl group (e.g., UGT33942 and UGT33786), a gene encoding a polypeptide capable of synthesizing geranylgeranyl pyrophosphate (GGPP) from farnesyl diphosphate (FPP) and isopentenyl diphosphate (IPP), a gene encoding a polypeptide capable of synthesizing ent-copalyl diphosphate from GGPP, a gene encoding a polypeptide capable of synthesizing ent-kaurene from ent-copalyl pyrophosphate, a gene encoding a polypeptide capable of synthesizing ent-kaurenoic acid from ent-kaurene, a gene encoding a polypeptide capable of synthesizing steviol from ent-kaurenoic acid, a gene encoding a polypeptide capable of reducing cytochrome P450 complex, and/or a gene encoding a bifunctional polypeptide capable of synthesizing ent-copalyl diphosphate from GGPP and synthesizing ent-kaurene from ent-copalyl pyrophosphate can produce a steviol glycoside in vivo. The skilled worker will appreciate that one or more of these genes can be endogenous to the host provided that at least one (and in some embodiments, all) of these genes is a recombinant gene introduced into the recombinant host.

In another example, a recombinant host expressing a gene encoding a polypeptide capable of synthesizing geranylgeranyl pyrophosphate (GGPP) from farnesyl diphosphate (FPP) and isopentenyl diphosphate (IPP), a gene encoding a polypeptide capable of synthesizing ent-copalyl diphosphate from GGPP, a gene encoding a polypeptide capable of synthesizing ent-kaurene from ent-copalyl pyrophosphate, a gene encoding a polypeptide capable of synthesizing ent-kaurenoic acid from ent-kaurene, a gene encoding a polypeptide capable of synthesizing steviol from ent-kaurenoic acid, a gene encoding a polypeptide capable of reducing cytochrome P450 complex, and/or a bifunctional polypeptide capable of synthesizing ent-copalyl diphosphate from GGPP and synthesizing ent-kaurene from ent-copalyl pyrophosphate and one or more of a gene encoding a polypeptide capable of beta 1,3 glycosylation of the C3′ of the 13-O-glucose, 19-O-glucose, or both 13-O-glucose and 19-O-glucose of a steviol glycoside, a polypeptide capable of beta 1,2 glycosylation of the C2′ of the 13-O-glucose, 19-O-glucose, or both 13-O-glucose and 19-O-glucose of a steviol glycoside, and a polypeptide capable of glycosylating steviol or a steviol glycoside at its C-13 hydroxyl group or a polypeptide capable of glycosylating steviol or a steviol glycoside at its C-19 carboxyl group, can produce a steviol glycoside in vivo. The skilled worker will appreciate that one or more of these genes can be endogenous to the host provided that at least one (and in some embodiments, all) of these genes is a recombinant gene introduced into the recombinant host.

In another example, a recombinant host expressing a recombinant gene encoding a UDP-glycosyltransferase (UGT) polypeptide structurally similar to members of the UGT85 family capable of glycosylating steviol or a steviol glycoside at its C-19 carboxyl group (e.g., UGT33942 and UGT33786), a gene encoding a polypeptide capable of synthesizing geranylgeranyl pyrophosphate (GGPP) from farnesyl diphosphate (FPP) and isopentenyl diphosphate (IPP), a gene encoding a polypeptide capable of synthesizing ent-copalyl diphosphate from GGPP, a gene encoding a polypeptide capable of synthesizing ent-kaurene from ent-copalyl pyrophosphate, a gene encoding a polypeptide capable of synthesizing ent-kaurenoic acid from ent-kaurene, a gene encoding a polypeptide capable of synthesizing steviol from ent-kaurenoic acid, a gene encoding polypeptide a capable of reducing cytochrome P450 complex, and/or a bifunctional polypeptide capable of synthesizing ent-copalyl diphosphate from GGPP and synthesizing ent-kaurene from ent-copalyl pyrophosphate and one or more of a gene encoding a polypeptide capable of beta 1,3 glycosylation of the C3′ of the 13-O-glucose, 19-O-glucose, or both 13-O-glucose and 19-O-glucose of a steviol glycoside, a polypeptide capable of beta 1,2 glycosylation of the C2′ of the 13-O-glucose, 19-O-glucose, or both 13-O-glucose and 19-O-glucose of a steviol glycoside, and a polypeptide capable of glycosylating steviol or a steviol glycoside at its C-13 hydroxyl group or a polypeptide capable of glycosylating steviol or a steviol glycoside at its C-19 carboxyl group, can produce a steviol glycoside in vivo. The skilled worker will appreciate that one or more of these genes can be endogenous to the host provided that at least one (and in some embodiments, all) of these genes is a recombinant gene introduced into the recombinant host.

In some embodiments, the steviol glycoside comprises steviol-13-O-glucoside (13-SMG), steviol-1,2-bioside, steviol-1,3-bioside, steviol-19-O-glucoside (19-SMG), stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, or isomers thereof.

In some aspects, the polypeptide capable of synthesizing geranylgeranyl pyrophosphate (GGPP) from farnesyl diphosphate (FPP) and isopentenyl diphosphate (IPP) comprises a polypeptide having the amino acid sequence set forth in SEQ ID NO:20 (which can be encoded by the nucleotide sequence set forth in SEQ ID NO:19), SEQ ID NO:22 (encoded by the nucleotide sequence set forth in SEQ ID NO:21), SEQ ID NO:24 (encoded by the nucleotide sequence set forth in SEQ ID NO:23), SEQ ID NO:26 (encoded by the nucleotide sequence set forth in SEQ ID NO:25), SEQ ID NO:28 (encoded by the nucleotide sequence set forth in SEQ ID NO:27), SEQ ID NO:30 (encoded by the nucleotide sequence set forth in SEQ ID NO:29), SEQ ID NO:32 (encoded by the nucleotide sequence set forth in SEQ ID NO:31), or SEQ ID NO:116 (encoded by the nucleotide sequence set forth in SEQ ID NO:115).

In some aspects, the polypeptide capable of synthesizing ent-copalyl diphosphate from GGPP comprises a polypeptide having the amino acid sequence set forth in SEQ ID NO:34 (which can be encoded by the nucleotide sequence set forth in SEQ ID NO:33), SEQ ID NO:36 (encoded by the nucleotide sequence set forth in SEQ ID NO:35), SEQ ID NO:38 (encoded by the nucleotide sequence set forth in SEQ ID NO:37), SEQ ID NO:40 (encoded by the nucleotide sequence set forth in SEQ ID NO:39), or SEQ ID NO:42 (encoded by the nucleotide sequence set forth in SEQ ID NO:41). In some embodiments, the polypeptide capable of synthesizing ent-copalyl diphosphate from GGPP lacks a chloroplast transit peptide.

In some aspects, the polypeptide capable of synthesizing ent-kaurene from ent-copalyl pyrophosphate comprises a polypeptide having the amino acid sequence set forth in SEQ ID NO:44 (which can be encoded by the nucleotide sequence set forth in SEQ ID NO:43), SEQ ID NO:46 (encoded by the nucleotide sequence set forth in SEQ ID NO:45), SEQ ID NO:48 (encoded by the nucleotide sequence set forth in SEQ ID NO:47), SEQ ID NO:50 (encoded by the nucleotide sequence set forth in SEQ ID NO:49), or SEQ ID NO:52 (encoded by the nucleotide sequence set forth in SEQ ID NO:51).

In some aspects, the polypeptide capable of synthesizing ent-kaurenoic acid from ent-kaurene comprises a polypeptide having the amino acid sequence set forth in SEQ ID NO:60 (which can be encoded by the nucleotide sequence set forth in SEQ ID NO:59), SEQ ID NO:62 (encoded by the nucleotide sequence set forth in SEQ ID NO:61), SEQ ID NO:66 (encoded by the nucleotide sequence set forth in SEQ ID NO:65), SEQ ID NO:68 (encoded by the nucleotide sequence set forth in SEQ ID NO:67), SEQ ID NO:70 (encoded by the nucleotide sequence set forth in SEQ ID NO:69), SEQ ID NO:72 (encoded by the nucleotide sequence set forth in SEQ ID NO:71), SEQ ID NO:74 (encoded by the nucleotide sequence set forth in SEQ ID NO:73), SEQ ID NO:76 (encoded by the nucleotide sequence set forth in SEQ ID NO:75), or SEQ ID NO:117 (encoded by the nucleotide sequence set forth in SEQ ID NO:63 or SEQ ID NO:64).

In some aspects, the polypeptide capable of reducing cytochrome P450 complex comprises a polypeptide having the amino acid sequence set forth in SEQ ID NO:78 (which can be encoded by the nucleotide sequence set forth in SEQ ID NO:77), SEQ ID NO:80 (encoded by the nucleotide sequence set forth in SEQ ID NO:79), SEQ ID NO:82 (encoded by the nucleotide sequence set forth in SEQ ID NO:81), SEQ ID NO:84 (encoded by the nucleotide sequence set forth in SEQ ID NO:83), SEQ ID NO:86 (encoded by the nucleotide sequence set forth in SEQ ID NO:85), SEQ ID NO:88 (encoded by the nucleotide sequence set forth in SEQ ID NO:87), SEQ ID NO:90 (encoded by the nucleotide sequence set forth in SEQ ID NO:89), or SEQ ID NO:92 (encoded by the nucleotide sequence set forth in SEQ ID NO:91).

In some aspects, the polypeptide capable of synthesizing steviol from ent-kaurenoic acid comprises a polypeptide having the amino acid sequence set forth in SEQ ID NO:94 (which can be encoded by the nucleotide sequence set forth in SEQ ID NO:93), SEQ ID NO:97 (encoded by the nucleotide sequence set forth in SEQ ID NO:95 or SEQ ID NO:96), SEQ ID NO:100 (encoded by the nucleotide sequence set forth in SEQ ID NO:98 or SEQ ID NO:99), SEQ ID NO:101, SEQ ID NO:102, SEQ ID NO:103, SEQ ID NO:104, SEQ ID NO:106 (encoded by the nucleotide sequence set forth in SEQ ID NO:105), SEQ ID NO:108 (encoded by the nucleotide sequence set forth in SEQ ID NO:107), SEQ ID NO:110 (encoded by the nucleotide sequence set forth in SEQ ID NO:109), SEQ ID NO:112 (encoded by the nucleotide sequence set forth in SEQ ID NO:111), or SEQ ID NO:114 (encoded by the nucleotide sequence set forth in SEQ ID NO:113).

In some aspects, the bifunctional polypeptide capable of synthesizing ent-copalyl diphosphate from GGPP and synthesizing ent-kaurene from ent-copalyl pyrophosphate comprises a polypeptide having at least 50% identity to the amino acid sequence set forth in SEQ ID NO:54, SEQ ID NO:56, or SEQ ID NO:58.

In some aspects, the polypeptide capable of glycosylating steviol or a steviol glycoside at its C-13 hydroxyl group comprises a polypeptide having the amino acid sequence set forth in SEQ ID NO:7 (which can be encoded by the nucleotides sequence set forth in SEQ ID NO:5 or SEQ ID NO:6). In some aspects, the polypeptide capable of beta 1,3 glycosylation of the C3′ of the 13-O-glucose, 19-O-glucose, or both 13-O-glucose and 19-O-glucose of a steviol glycoside comprises a polypeptide having a sequence set forth in SEQ ID NO:9 (encoded by the nucleotide sequence set forth in SEQ ID NO:8). In some aspects, the polypeptide capable of glycosylating steviol or a steviol glycoside at its C-19 carboxyl group comprises a polypeptide having a sequence set forth in SEQ ID NO:2, SEQ ID NO:4, or SEQ ID NO:119 (encoded by the nucleotide sequence set forth in SEQ ID NO:1, SEQ ID NO:3, or SEQ ID NO:118). In some aspects, the polypeptide capable of beta 1,2 glycosylation of the C2′ of the 13-O-glucose, 19-O-glucose, or both 13-O-glucose and 19-O-glucose of a steviol glycoside comprises a polypeptide having the amino acid sequence set forth in SEQ ID NO:16, SEQ ID NO:11, or SEQ ID NO:13 (encoded by the nucleotide sequence set forth in SEQ ID NO:10, SEQ ID NO:12, SEQ ID NO:14, or SEQ ID NO:15).

In some embodiments, the polypeptide capable of synthesizing geranylgeranyl pyrophosphate (GGPP) from farnesyl diphosphate (FPP) and isopentenyl diphosphate (IPP) is a geranylgeranyl diphosphate synthase (GGPPS) polypeptide. In some embodiments, the polypeptide capable of synthesizing ent-copalyl diphosphate from GGPP is an ent-copalyl diphosphate synthase (CDPS) polypeptide. In some embodiments, the polypeptide capable of synthesizing ent-kaurene from ent-copalyl pyrophosphate is an ent-kaurene synthase (KS) polypeptide. In some embodiments, the polypeptide capable of synthesizing ent-kaurenoic acid from ent-kaurene is an ent-kaurene oxidase (KO) polypeptide. In some embodiments, the polypeptide capable of synthesizing steviol from ent-kaurenoic acid is an ent-kaurenoic acid hydroxylase (KAH) polypeptide. In some embodiments, the polypeptide capable of reducing cytochrome P450 complex is a cytochrome P450 reductase (CPR) polypeptide. In some embodiments, the polypeptide capable of beta 1,3 glycosylation of the C3′ of the 13-O-glucose, 19-O-glucose, or both 13-O-glucose and 19-O-glucose of a steviol glycoside is a polypeptide capable of beta 1,3 glycosylation of the C3′ of the 13-O-glucose, 19-O-glucose, or both 13-O-glucose and 19-O-glucose of a steviol glycoside. In some embodiments, the polypeptide capable of glycosylating steviol or a steviol glycoside at its C-13 hydroxyl group thereof is a polypeptide capable of glycosylating steviol or a steviol glycoside at its C-13 hydroxyl group. In some embodiments, the bifunctional polypeptide capable of synthesizing ent-copalyl diphosphate from GGPP and synthesizing ent-kaurene from ent-copalyl pyrophosphate is an ent-copalyl diphosphate synthase (CDPS)—ent-kaurene synthase (KS) polypeptide. In some embodiments, the polypeptide capable of glycosylating steviol or a steviol glycoside at its C-19 carboxyl group is a UGT74G1 polypeptide, a UGT33942 polypeptide, or a UGT33786 polypeptide. In some embodiments, the polypeptide capable of beta 1,2 glycosylation of the C2′ of the 13-O-glucose, 19-O-glucose, or both 13-O-glucose and 19-O-glucose of a steviol glycoside is a UGT91D2 polypeptide such as UGT91D2e or UGT91D2e-b, or a EUGT11 polypeptide.

In certain embodiments, the steviol glycoside is RebA, RebB, RebD, and/or RebM. RebA can be synthesized, for example, in a steviol-producing recombinant microorganism expressing a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group, ii) UGT33942, and/or iii) UGT33786. RebB can be synthesized, for example, in a steviol-producing recombinant microorganism expressing a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, and polypeptide capable of beta 1,2 glycosylation of the C2′ of the 13-O-glucose, 19-O-glucose, or both 13-O-glucose and 19-O-glucose of a steviol glycoside. RebD can be synthesized, for example, in a steviol-producing recombinant microorganism expressing a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, polypeptide capable of beta 1,2 glycosylation of the C2′ of the 13-0-glucose, 19-O-glucose, or both 13-O-glucose and 19-O-glucose of a steviol glycoside, and i) a polypeptide capable of glycosylating steviol or a steviol glycoside at its C-19 carboxyl group, ii) UGT33942, and/or iii) UGT33786. RebM can be synthesized, for example, in a steviol-producing recombinant microorganism expressing a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group, ii) UGT33942, and/or iii) UGT33786 (see FIG. 2).

In certain embodiments, the steviol glycoside is RebA, RebB, RebD, and/or RebM. RebA can be synthesized in a steviol-producing recombinant microorganism expressing a polypeptide capable of glycosylating steviol or a steviol glycoside at its C-13 hydroxyl group, a polypeptide capable of beta 1,3 glycosylation of the 03′ of the 13-O-glucose, 19-O-glucose, or both 13-O-glucose and 19-O-glucose of a steviol glycoside, polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group, ii) UGT33942, and/or iii) UGT33786; RebB can be synthesized in a steviol-producing recombinant microorganism expressing a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, and polypeptide capable of beta 1,2 glycosylation of the C2′ of the 13-O-glucose, 19-O-glucose, or both 13-O-glucose and 19-O-glucose of a steviol glycoside; RebD can be synthesized in a steviol-producing recombinant microorganism expressing a polypeptide capable of glycosylating steviol or a steviol glycoside at its C-13 hydroxyl group; a polypeptide capable of beta 1,3 glycosylation of the C3′ of the 13-0-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group, ii) UGT33942, and/or iii) UGT33786; RebM can be synthesized in a steviol-producing recombinant microorganism expressing a polypeptide capable of glycosylating steviol or a steviol glycoside at its C-13 hydroxyl group; a polypeptide capable of beta 1,3 glycosylation of the C3′ of the 13-0-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group, ii) UGT33942, and/or iii) UGT33786; wherein the steviol-producing host expresses one or more of a gene encoding a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a gene encoding a polypeptide capable of synthesizing ent-copalyl diphosphate from GGPP (e.g., ent-copaiyl diphosphate synthase (CDPS)); a gene encoding a polypeptide capable of synthesizing ent-kaurene from ent-copalyl pyrophosphate (e.g., kaurene synthase (KS)); a gene encoding a polypeptide capable of synthesizing ent-kaurenoic acid from ent-kaurene (e.g., kaurene oxidase (KO)); a gene encoding a polypeptide capable of reducing cytochrome P450 complex (e.g., cytochrome P450 reductase (CPR)); and a gene encoding a polypeptide capable of synthesizing steviol from ent-kaurenoic acid (e.g., steviol synthase (KAH)); and/or a gene encoding a bifunctional polypeptide capable of synthesizing ent-copalyl diphosphate from GGPP and synthesizing ent-kaurene from ent-copalyl pyrophosphate (e.g., an ent-copalyl diphosphate synthase (CDPS)—ent-kaurene synthase (KS) polypeptide), and a gene encoding a UGT polypeptide can produce a steviol glycoside and/or steviol glycoside precursors in vivo.

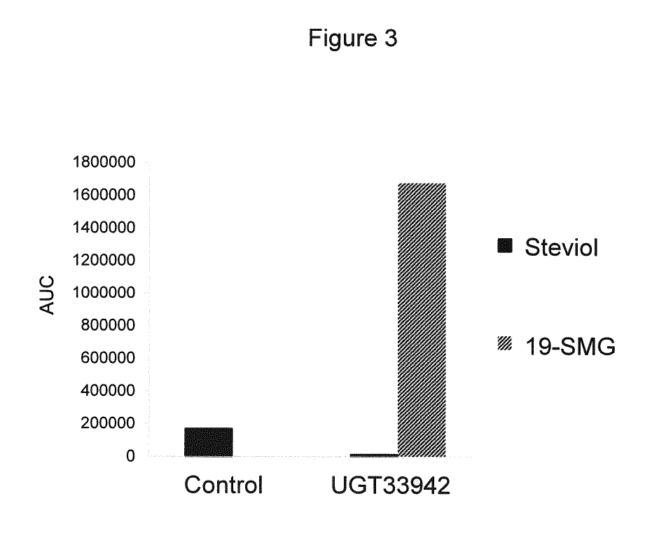

In some embodiments, an S. cerevisiae strain expressing a gene encoding a UGT33942 polypeptide (SEQ ID NO:1, SEQ ID NO:2) converts steviol to 19-SMG. UGT33942 is a UGT polypeptide with high structural similarity to members of the UGT85 family and catalyzes glycosylation of the 19-0 position of steviol and steviol glycosides. See Example 1 and FIG. 3. In some embodiments, UGT33942 catalyzes conversion of i) steviol-1,2-bioside to stevioside, ii) steviol-1,3-bioside to 1,3-stevioside, iii) 13-SMG to rubusoside, and/or iv) RebB to RebA.

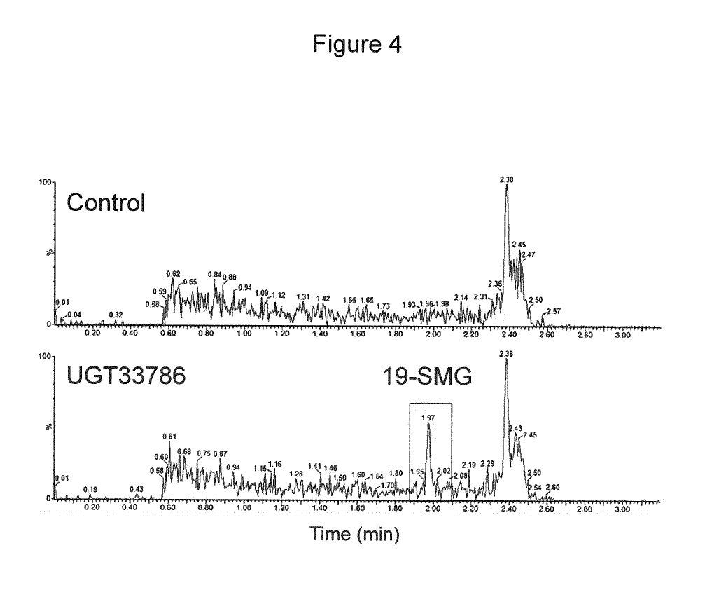

In some embodiments, an S. cerevisiae strain expressing a gene encoding a UGT33786 polypeptide (SEQ ID NO:118, SEQ ID NO:119) converts steviol to 19-SMG. UGT33786 is a UGT polypeptide with high structural similarity to members of the UGT85 family and catalyzes glycosylation of the 19-0 position of steviol and steviol glycosides. See Example 2 and FIG. 4. In some embodiments, UGT33786 catalyzes conversion of i) steviol-1,2-bioside to stevioside, ii) steviol-1,3-bioside to 1,3-stevioside, iii) 13-SMG to rubusoside, and/or iv) RebB to RebA.

In some embodiments, steviol glycosides and/or steviol glycoside precursors are produced through contact of a steviol glycoside precursor with one or more enzymes involved in the steviol glycoside pathway in vitro. For example, contacting steviol with a UGT polypeptide can result in production of a steviol glycoside in vitro. In some embodiments, a steviol glycoside precursor is produced through contact of an upstream steviol glycoside precursor with one or more enzymes involved in the steviol glycoside pathway in vitro. For example, contacting ent-kaurenoic acid with a polypeptide capable of synthesizing steviol from ent-kaurenoic acid can result in production of steviol in vitro.

In some embodiments, steviol glycosides are produced in vitro through contact of a steviol glycoside and/or a steviol glycoside precursor with a polypeptide capable of glycosylating steviol or a steviol glycoside at its C-19 carboxyl group. For example, contacting steviol with a polypeptide capable of glycosylating steviol or a steviol glycoside at its C-19 carboxyl group can result in production of a steviol glycoside in vitro. In some embodiments, the polypeptide capable of glycosylating steviol or a steviol glycoside at its C-19 carboxyl group is a UGT33786 polypeptide. In some embodiments, the UGT33942 polypeptide comprises a polypeptide having at least 50% identity to the amino acid sequence set forth in SEQ ID NO:2. In some embodiments, the UGT33786 polypeptide comprises a polypeptide having at least 50% identity to the amino acid sequence set forth in SEQ ID NO:119. In some embodiments, the steviol glycoside produced is 19-SMG. In some embodiments, the contact occurs in a reaction mixture comprising 19-SMG, a UDP-glycosyltransferase (UGT) polypeptide structurally similar to members of the UGT85 family capable of glycosylating steviol or a steviol glycoside at its C-19 carboxyl group (e.g., UGT33942 and UGT33786), uridine diphosphate (UDP)-glucose, UDP-rhamnose, UDP-xylose, and/or N-acetyl-glucosamine, and/or reaction buffer and/or salts.

In some embodiments, a steviol glycoside or steviol glycoside precursor is produced by whole cell bioconversion. For whole cell bioconversion to occur, a host cell expressing one or more enzymes involved in the steviol glycoside pathway takes up and modifies the steviol glycoside or steviol glycoside precursor in the cell; following modification in vivo, the steviol glycoside or steviol glycoside precursor remains in the cell and/or is excreted into the cell culture medium. For example, a recombinant host cell expressing a gene encoding a UGT polypeptide can take up steviol and glycosylate steviol in the cell; following glycosylation in vivo, a steviol glycoside can be excreted into the cell culture medium. In some embodiments, the cell is permeabilized to take up a substrate to be modified or to excrete a modified product. In another example, a recombinant host cell expressing a gene encoding a UGT polypeptide can take up steviol and glycosylate steviol in the cell; following glycosylation in vivo, a steviol glycoside can be excreted into the cell culture medium. A permeabilized recombinant host cell can then be added to the cell culture medium to take up the excreted steviol glycoside to be further modified and to excrete a further modified product.

In some embodiments, the UGT polypeptide can be a polypeptide capable of glycosylating steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group and/or ii) a UDP-glycosyltransferase (UGT) polypeptide structurally similar to members of the UGT85 family capable of glycosylating steviol or a steviol glycoside at its C-19 carboxyl group (e.g., UGT33942 and UGT33786).

In another example, a steviol-producing recombinant host expressing one or more of a gene encoding a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a gene encoding a polypeptide capable of synthesizing ent-copalyl diphosphate from GGPP (e.g., ent-copalyl diphosphate synthase (CDPS)); a gene encoding a polypeptide capable of synthesizing ent-kaurene from ent-copalyl pyrophosphate (e.g., kaurene synthase (KS)); a gene encoding a polypeptide capable of synthesizing ent-kaurenoic acid from ent-kaurene (e.g., kaurene oxidase (KO)); a gene encoding a polypeptide capable of reducing cytochrome P450 complex (e.g., cytochrome P450 reductase (CPR)); and a gene encoding a polypeptide capable of synthesizing steviol from ent-kaurenoic acid (e.g., steviol synthase (KAH)); and/or a gene encoding a bifunctional polypeptide capable of synthesizing ent-copalyl diphosphate from GGPP and synthesizing ent-kaurene from ent-copalyl pyrophosphate (e.g., an ent-copalyl diphosphate synthase (CDPS)—ent-kaurene synthase (KS) polypeptide) can produce steviol in the cell; following production in vivo, steviol can be excreted into the cell culture medium. A permeabilized recombinant host cell expressing a gene encoding a UGT polypeptide capable of glycosylating steviol or a steviol glycoside in vivo can then be added to the cell culture medium to take up the excreted steviol to be modified and to excrete a modified product.

In some embodiments, the UGT polypeptide can be a polypeptide capable of glycosylating steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating steviol or a steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside, and i) a polypeptide capable of glycosylating steviol or a steviol glycoside at its C-19 carboxyl group and/or ii) a UDP-glycosyltransferase (UGT) polypeptide structurally similar to members of the UGT85 family capable of glycosylating steviol or a steviol glycoside at its C-19 carboxyl group (e.g., UGT33942 and UGT33786).

In some embodiments, a steviol glycoside or steviol glycoside precursor is produced by whole cell bioconversion in a host cell expressing of one or more enzymes involved in the steviol glycoside biosynthetic pathway in a recombinant host. For example, a host cell expressing a recombinant gene encoding a polypeptide capable of glycosylating steviol or a steviol glycoside at its C-19 carboxyl group can modify steviol glycosides and/or steviol glycoside precursors in vivo.

In some embodiments, a polypeptide capable of glycosylating steviol or a steviol glycoside at its C-19 carboxyl group can be displayed on the surface of the recombinant host cells disclosed herein by fusing it with the anchoring motifs. The polypeptide capable of glycosylating steviol or a steviol glycoside at its C-19 carboxyl group to be displayed—the passenger protein—can be fused to an anchoring motif—the carrier protein—by N-terminal fusion, C-terminal fusion or sandwich fusion. Such cell-surface display can be used for production of steviol glycosides and steviol glycoside precursors by whole cell bioconversion.

In some embodiments, the polypeptide capable of glycosylating steviol or a steviol glycoside at its C-19 carboxyl group is a UGT33942 polypeptide. In some embodiments, the polypeptide capable of glycosylating steviol or a steviol glycoside at its C-19 carboxyl group is a UGT33786 polypeptide. In some embodiments, the UGT33942 polypeptide comprises a polypeptide having at least 50% identity to the amino acid sequence set forth in SEQ ID NO:2. In some embodiments, the UGT33786 polypeptide comprises a polypeptide having at least 50% identity to the amino acid sequence set forth in SEQ ID NO:119.

In some embodiments, a steviol glycoside or steviol glycoside precursor is produced by whole cell bioconversion in a host cell expressing of one or more enzymes involved in the steviol glycoside biosynthetic pathway in a recombinant host. For example, a steviol-producing recombinant host expressing one or more of a gene encoding a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a gene encoding a polypeptide capable of synthesizing ent-copalyl diphosphate from GGPP (e.g., ent-copalyl diphosphate synthase (CDPS)); a gene encoding a polypeptide capable of synthesizing ent-kaurene from ent-copalyl pyrophosphate (e.g., kaurene synthase (KS)); a gene encoding a polypeptide capable of synthesizing ent-kaurenoic acid from ent-kaurene (e.g., kaurene oxidase (KO)); a gene encoding a polypeptide capable of reducing cytochrome P450 complex (e.g., cytochrome P450 reductase (CPR)); and a gene encoding a polypeptide capable of synthesizing steviol from ent-kauienoic acid (e.g., steviol synthase (KAH)); and/or a gene encoding a bifunctional polypeptide capable of synthesizing ent-copalyl diphosphate from GGPP and synthesizing ent-kaurene from ent-copalyl pyrophosphate (e.g., an ent-copalyl diphosphate synthase (CDPS)—ent-kaurene synthase (KS) polypeptide), and a gene encoding a UGT can modify steviol glycosides and/or steviol glycoside precursors in vivo. See, e.g., FIGS. 1 and 2. The skilled worker will appreciate that one or more of these genes can be endogenous to the host provided that at least one (and in some embodiments, all) of these genes is a recombinant gene introduced into the recombinant host.

In some embodiments, a steviol glycoside or steviol glycoside precursor is produced by whole cell bioconversion in a host cell expressing a polypeptide capable of glycosylating steviol or a steviol glycoside at its C-19 carboxyl group (e.g., UGT33942 and UGT33786) and one or more enzymes involved in the steviol glycoside biosynthetic pathway in a recombinant host. For example, a steviol-producing recombinant host expressing a UDP-glycosyltransferase (UGT) polypeptide structurally similar to members of the UGT85 family capable of glycosylating steviol or a steviol glycoside at its C-19 carboxyl group (e.g., UGT33942 and UGT33786) and one or more of a gene encoding a polypeptide capable of synthesizing geranylgeranyl pyrophosphate (GGPP) from farnesyl diphosphate (FPP) and isopentenyl diphosphate (IPP) (e.g., geranylgeranyl diphosphate synthase (GGPPS)); a gene encoding a polypeptide capable of synthesizing ent-copalyl diphosphate from GGPP (e.g., ent-copalyl diphosphate synthase (CDPS)); a gene encoding a polypeptide capable of synthesizing ent-kaurene from ent-copalyl pyrophosphate (e.g., kaurene synthase (KS)); a gene encoding a polypeptide capable of synthesizing ent-kaurenoic acid from ent-kaurene (e.g., kaurene oxidase (KO)); a gene encoding a polypeptide capable of reducing cytochrome P450 complex (e.g., cytochrome P450 reductase (CPR)); and a gene encoding a polypeptide capable of synthesizing steviol from ent-kaurenoic acid (e.g., steviol synthase (KAH)); and/or a gene encoding a bifunctional polypeptide capable of synthesizing ent-copalyl diphosphate from GGPP and synthesizing ent-kaurene from ent-copalyl pyrophosphate (e.g., an ent-copalyl diphosphate synthase (CDPS)—ent-kaurene synthase (KS) polypeptide), and a gene encoding a UGT polypeptide can modify steviol glycosides and/or steviol glycoside precursors in vivo.

In some embodiments, the steviol glycoside comprises steviol-13-O-glucoside (13-SMG), steviol-1,2-bioside, steviol-1,3-bioside, steviol-19-O-glucoside (19-SMG), stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, or isomers thereof.

In some embodiments, steviol, one or more steviol glycoside precursors, and/or one or more steviol glycosides are produced by co-culturing of two or more hosts. In some embodiments, one or more hosts, each expressing one or more enzymes involved in the steviol glycoside pathway, produce steviol, one or more steviol glycoside precursors, and/or one or more steviol glycosides. For example, a host comprising a polypeptide capable of synthesizing geranylgeranyl pyrophosphate (GGPP) from farnesyl diphosphate (FPP) and isopentenyl diphosphate (IPP), a polypeptide capable of synthesizing ent-copalyl diphosphate from GGPP, a polypeptide capable of synthesizing ent-kaurenoic acid from ent-kaurene, a polypeptide capable of synthesizing ent-kaurene from ent-copalyl pyrophosphate, a polypeptide capable of synthesizing steviol from ent-kaurenoic acid, and/or a polypeptide capable of reducing cytochrome P450 complex and a host comprising one or more UGTs produce one or more steviol glycosides.

In some embodiments, the steviol glycoside comprises steviol-13-O-glucoside (13-SMG), steviol-1,2-bioside, steviol-1,3-bioside, steviol-19-O-glucoside (19-SMG), stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, or isomers thereof.

In some embodiments, a steviol glycoside or steviol glycoside precursor composition produced in vivo, in vitro, or by whole cell bioconversion does not comprise or comprises a reduced amount or reduced level of plant-derived components than a Stevia extract from, inter alia, a Stevia plant. Plant-derived components can contribute to off-flavors and include pigments, lipids, proteins, phenolics, saccharides, spathulenol and other sesquiterpenes, labdane diterpenes, monoterpenes, decanoic acid, 8,11,14-eicosatrienoic acid, 2-methyloctadecane, pentacosane, octacosane, tetracosane, octadecanol, stigmasterol, β-sitosterol, α- and β-amyrin, lupeol, β-amryin acetate, pentacyclic triterpenes, centauredin, quercitin, epi-alpha-cadinol, carophyllenes and derivatives, beta-pinene, beta-sitosterol, and gibberellin. In some embodiments, the plant-derived components referred to herein are non-glycoside compounds.

As used herein, the terms “detectable amount,” “detectable concentration,” “measurable amount,” and “measurable concentration” refer to a level of steviol glycosides measured in AUC, μM/OD₆₀₀, mg/L, μM, or mM. Steviol glycoside production (i.e., total, supernatant, and/or intracellular steviol glycoside levels) can be detected and/or analyzed by techniques generally available to one skilled in the art, for example, but not limited to, liquid chromatography-mass spectrometry (LC-MS), thin layer chromatography (TLC), high-performance liquid chromatography (HPLC), ultraviolet-visible spectroscopy/spectrophotometry (UV-Vis), mass spectrometry (MS), and nuclear magnetic resonance spectroscopy (NMR).

As used herein, the term “undetectable concentration” refers to a level of a compound that is too low to be measured and/or analyzed by techniques such as TLC, HPLC, UV-Vis, MS, or NMR. In some embodiments, a compound of an “undetectable concentration” is not present in a steviol glycoside or steviol glycoside precursor composition.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to production of steviol glycosides and/or steviol glycoside precursors. In some embodiments, “and/or” is used to refer to production of steviol glycosides, wherein one or more steviol glycosides are produced. In some embodiments, “and/or” is used to refer to production of steviol glycosides, wherein one or more steviol glycosides are produced through one or more of the following steps: culturing a recombinant microorganism, synthesizing one or more steviol glycosides in a recombinant microorganism, and/or isolating one or more steviol glycosides.

### Functional Homologs

Functional homologs of the polypeptides described above are also suitable for use in producing steviol glycosides in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of steviol glycoside biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a steviol glycoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in steviol glycoside biosynthesis polypeptides, e.g., conserved functional domains. In some embodiments, nucleic acids and polypeptides are identified from transcriptome data based on expression levels rather than by using BLAST analysis.

Conserved regions can be identified by locating a region within the primary amino acid sequence of a steviol glycoside biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al. Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing steviol in a recombinant host include functional homologs of UGTs.

Methods to modify the substrate specificity of, for example, a UGT, are known to those skilled in the art, and include without limitation site-directed/rational mutagenesis approaches, random directed evolution approaches and combinations in which random mutagenesis/saturation techniques are performed near the active site of the enzyme. For example see Osmani et al., 2009, Phytochemistry 70: 325-347.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A % identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program ClustalW (version 1.83, default parameters), which allows alignments of nucleic acid or polypeptide sequences to be carried out across their entire length (global alignment). Chenna et al., 2003, Nucleic Acids Res. 31(13):3497-500.

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method:% age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine % identity of a candidate nucleic acid or amino acid sequence to a reference sequence, the sequences are aligned using ClustalW, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that functional UGT proteins (e.g., a polypeptide capable of glycosylating steviol or a steviol glycoside at its C-19 carboxyl group) can include additional amino acids that are not involved in the enzymatic activities carried out by the enzymes. In some embodiments, UGT proteins are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. In some embodiments, a nucleic acid sequence encoding a UGT polypeptide (e.g., a polypeptide capable of glycosylating steviol or a steviol glycoside at its C-19 carboxyl group) can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, Conn.). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, a UGT polypeptide (e.g., a polypeptide capable of glycosylating steviol or a steviol glycoside at its C-19 carboxyl group) is altered by domain swapping.

In some embodiments, a fusion protein is a protein altered by circular permutation, which consists in the covalent attachment of the ends of a protein that would be opened elsewhere afterwards. Thus, the order of the sequence is altered without causing changes in the amino acids of the protein. In some embodiments, a targeted circular permutation can be produced, for example but not limited to, by designing a spacer to join the ends of the original protein. Once the spacer has been defined, there are several possibilities to generate permutations through generally accepted molecular biology techniques, for example but not limited to, by producing concatemers by means of PCR and subsequent amplification of specific permutations inside the concatemer or by amplifying discrete fragments of the protein to exchange to join them in a different order. The step of generating permutations can be followed by creating a circular gene by binding the fragment ends and cutting back at random, thus forming collections of permutations from a unique construct. In some embodiments, a UGT polypeptide (e.g., a polypeptide capable of glycosylating steviol or a steviol glycoside at its C-19 carboxyl group) is altered by circular permutation.

In some embodiments, the polypeptide capable of glycosylating steviol or a steviol glycoside at its C-19 carboxyl group is a UGT33942 polypeptide. In some embodiments, the polypeptide capable of glycosylating steviol or a steviol glycoside at its C-19 carboxyl group is a UGT33786 polypeptide. In some embodiments, the UGT33942 polypeptide comprises a polypeptide having at least 50% identity to the amino acid sequence set forth in SEQ ID NO:2. In some embodiments, the UGT33786 polypeptide comprises a polypeptide having at least 50% identity to the amino acid sequence set forth in SEQ ID NO:119.

### Steviol and Steviol Glycoside Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of steviol and/or steviol glycoside production. Combining a plurality of genes in a module, particularly a polycistronic module, facilitates the use of the module in a variety of species. For example, a steviol biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for steviol or steviol glycoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards steviol or steviol glycoside biosynthesis. For example, it may be desirable to downregulate synthesis of sterols in a yeast strain in order to further increase steviol or steviol glycoside production, e.g., by downregulating squalene epoxidase. As another example, it may be desirable to inhibit degradative functions of certain endogenous gene products, e.g., glycohydrolases that remove glucose moieties from secondary metabolites or phosphatases as discussed herein. In such cases, a nucleic acid that overexpresses the polypeptide or gene product may be included in a recombinant construct that is transformed into the strain. Alternatively, mutagenesis can be used to generate mutants in genes for which it is desired to increase or enhance function.

### Host Microorganisms

Recombinant hosts can be used to express polypeptides for producing steviol glycosides, including mammalian, insect, plant, and algal cells. A number of prokaryotes and eukaryotes are also suitable for use in constructing the recombinant microorganisms described herein, e.g., gram-negative bacteria, yeast, and fungi. A species and strain selected for use as a steviol glycoside production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

Typically, the recombinant microorganism is grown in a fermenter at a temperature(s) for a period of time, wherein the temperature and period of time facilitate the production of a steviol glycoside. The constructed and genetically engineered microorganisms provided by the invention can be cultivated using conventional fermentation processes, including, inter alia, chemostat, batch, fed-batch cultivations, semi-continuous fermentations such as draw and fill, continuous perfusion fermentation, and continuous perfusion cell culture. Depending on the particular microorganism used in the method, other recombinant genes such as isopentenyl biosynthesis genes and terpene synthase and cyclase genes may also be present and expressed. Levels of substrates and intermediates, e.g., isopentenyl diphosphate, dimethylallyl diphosphate, GGPP, ent-kaurene and ent-kaurenoic acid, can be determined by extracting samples from culture media for analysis according to published methods.

Carbon sources of use in the instant method include any molecule that can be metabolized by the recombinant host cell to facilitate growth and/or production of the steviol glycosides. Examples of suitable carbon sources include, but are not limited to, sucrose (e.g., as found in molasses), fructose, xylose, ethanol, glycerol, glucose, cellulose, starch, cellobiose or other glucose-comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

After the recombinant microorganism has been grown in culture for the period of time, wherein the temperature and period of time facilitate the production of a steviol glycoside, steviol and/or one or more steviol glycosides can then be recovered from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC. See also, WO 2009/140394.

It will be appreciated that the various genes and modules discussed herein can be present in two or more recombinant hosts rather than a single host. When a plurality of recombinant hosts is used, they can be grown in a mixed culture to accumulate steviol and/or steviol glycosides.

Alternatively, the two or more hosts each can be grown in a separate culture medium and the product of the first culture medium, e.g., steviol, can be introduced into second culture medium to be converted into a subsequent intermediate, or into an end product such as, for example, RebA. The product produced by the second, or final host is then recovered. It will also be appreciated that in some embodiments, a recombinant host is grown using nutrient sources other than a culture medium and utilizing a system other than a fermenter.

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

In some embodiments of the recombinant hosts as otherwise described herein, a microorganism can be a prokaryote such as Escherichia bacteria cells, for example, Escherichia coli cells; Lactobacillus bacteria cells; Lactococcus bacteria cells; Cornebacterium bacteria cells; Acetobacter bacteria cells; Acinetobacter bacteria cells; or Pseudomonas bacterial cells.

In some embodiments of the recombinant hosts as otherwise described herein, a microorganism can be an Ascomycete such as Gibberella fujikuroi, Kluyveromyces lactis, Schizosaccharomyces pombe, Aspergillus niger, Yarrowia lipolytica, Ashbya gossypii, or S. cerevisiae.

In some embodiments of the recombinant hosts as otherwise described herein, a microorganism can be an algal cell such as Blakeslea trispora, Dunaliella salina, Haematococcus pluvialis, Chlorella sp., Undaria pinnatifida, Sargassum, Laminaria japonica, Scenedesmus almeriensis species.

In some embodiments of the recombinant hosts as otherwise described herein, a microorganism can be a cyanobacterial cell such as Blakeslea trispora, Dunaliella salina, Haematococcus pluvialis, Chlorella sp., Undaria pinnatifida, Sargassum, Laminaria japonica, Scenedesmus almeriensis.

### Saccharomyces spp.

Saccharomyces is a widely used chassis organism in synthetic biology, and can be used as the recombinant microorganism platform of a recombinant host as otherwise described herein. For example, there are libraries of mutants, plasmids, detailed computer models of metabolism and other information available for S. cerevisiae, allowing for rational design of various modules to enhance product yield. Methods are known for making recombinant microorganisms.

### Aspergillus spp.

Aspergillus species such as A. oryzae, A. niger and A. sojae are widely used microorganisms in food production and can also be used as the recombinant microorganism platform of a recombinant host as otherwise described herein. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for producing steviol glycosides.

E. coli

E. coli, another widely used platform organism in synthetic biology, can also be used as the recombinant microorganism platform of a recombinant host as otherwise described herein. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of steviol glycosides are already produced by endogenous genes. Thus, modules comprising recombinant genes for steviol glycoside biosynthesis polypeptides can be introduced into species from such genera without the necessity of introducing mevalonate or MEP pathway genes. Accordingly, Agaricus, Gibberella, and Phanerochaete spp. can be used as the recombinant microorganism platform of a recombinant host as otherwise described herein.

Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples. Accordingly, Arxula adeninivorans can be used as the recombinant microorganism platform of a recombinant host as otherwise described herein.

Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Bankar et al., 2009, Appl Microbiol Biotechnol. 84(5):847-65. Accordingly, Yarrowia lipolytica can be used as the recombinant microorganism platform of a recombinant host as otherwise described herein.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7). Accordingly, Rhodotorula can be used as the recombinant microorganism platform of a recombinant host as otherwise described herein.

Rhodosporidium toruloides

Rhodosporidium toruloides is oleaginous yeast and useful for engineering lipid-production pathways (See e.g. Zhu et al., 2013, Nature Commun. 3:1112; Ageitos et al., 2011, Applied Microbiology and Biotechnology 90(4):1219-27). Accordingly, Rhodosporidium toruloides can be used as the recombinant microorganism platform of a recombinant host as otherwise described herein.

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38. Accordingly, Candida boidinii can be used as the recombinant microorganism platform of a recombinant host as otherwise described herein.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9. Accordingly, Hansenula polymorpha can be used as the recombinant microorganism platform of a recombinant host as otherwise described herein.

Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92. Accordingly, Kluyveromyces lactis can be used as the recombinant microorganism platform of a recombinant host as otherwise described herein.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7. Accordingly, Pichia pastoris can be used as the recombinant microorganism platform of a recombinant host as otherwise described herein.

### Physcomitrella spp.

Physcomitrella mosses, when grown in suspension culture, have characteristics similar to yeast or other fungal cultures. This genera can be used for producing plant secondary metabolites, which can be difficult to produce in other types of cells. Accordingly, Physcomitrella mosses can be used as the recombinant microorganism platform of a recombinant host as otherwise described herein.

### Steviol Glycoside Compositions

Steviol glycosides do not necessarily have equivalent performance in different food systems. It is therefore desirable to have the ability to direct the synthesis to steviol glycoside compositions of choice. Recombinant hosts described herein can produce compositions that are selectively enriched for specific steviol glycosides (e.g., 19-SMG, RebD or RebM) and have a consistent taste profile. As used herein, the term “enriched” is used to describe a steviol glycoside composition with an increased proportion of a particular steviol glycoside, compared to a steviol glycoside composition (extract) from a Stevia plant. Thus, the recombinant hosts described herein can facilitate the production of compositions that are tailored to meet the sweetening profile desired for a given food product and that have a proportion of each steviol glycoside that is consistent from batch to batch. In some embodiments, hosts described herein do not produce or produce a reduced amount of undesired plant-derived components found in Stevia extracts. Thus, steviol glycoside compositions produced by the recombinant hosts described herein are distinguishable from compositions derived from Stevia plants.

The amount of an individual steviol glycoside (e.g., RebA, RebB, RebD, or RebM) accumulated can be from about 1 to about 7,000 mg/L, e.g., about 1 to about 10 mg/L, about 3 to about 10 mg/L, about 5 to about 20 mg/L, about 10 to about 50 mg/L, about 10 to about 100 mg/L, about 25 to about 500 mg/L, about 100 to about 1,500 mg/L, or about 200 to about 1,000 mg/L, at least about 1,000 mg/L, at least about 1,200 mg/L, at least about at least 1,400 mg/L, at least about 1,600 mg/L, at least about 1,800 mg/L, at least about 2,800 mg/L, or at least about 7,000 mg/L. In some aspects, the amount of an individual steviol glycoside can exceed 7,000 mg/L. The amount of a combination of steviol glycosides (e.g., RebA, RebB, RebD, or RebM) accumulated can be from about 1 mg/L to about 7,000 mg/L, e.g., about 200 to about 1,500, at least about 2,000 mg/L, at least about 3,000 mg/L, at least about 4,000 mg/L, at least about 5,000 mg/L, at least about 6,000 mg/L, or at least about 7,000 mg/L. In some aspects, the amount of a combination of steviol glycosides can exceed 7,000 mg/L. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

It will be appreciated that the various genes and modules discussed herein can be present in two or more recombinant microorganisms rather than a single microorganism. When a plurality of recombinant microorganisms is used, they can be grown in a mixed culture to produce steviol and/or steviol glycosides. For example, a first microorganism can comprise one or more biosynthesis genes for producing a steviol glycoside precursor, while a second microorganism comprises steviol glycoside biosynthesis genes. The product produced by the second, or final microorganism is then recovered. It will also be appreciated that in some embodiments, a recombinant microorganism is grown using nutrient sources other than a culture medium and utilizing a system other than a fermenter.

Alternatively, the two or more microorganisms each can be grown in a separate culture medium and the product of the first culture medium, e.g., steviol, can be introduced into second culture medium to be converted into a subsequent intermediate, or into an end product such as RebA. The product produced by the second, or final microorganism is then recovered. It will also be appreciated that in some embodiments, a recombinant microorganism is grown using nutrient sources other than a culture medium and utilizing a system other than a fermenter.

Steviol glycosides and compositions obtained by the methods disclosed herein can be used to make food products, dietary supplements and sweetener compositions. See, e.g., WO 2011/153378, WO 2013/022989, WO 2014/122227, and WO 2014/122328. These publications are hereby incorporated herein by reference in their entirety.

For example, substantially pure steviol or steviol glycoside such as RebM or RebD can be included in food products such as ice cream, carbonated beverages, fruit juices, yogurts, baked goods, chewing gums, hard and soft candies, and sauces. Substantially pure steviol or steviol glycoside can also be included in non-food products such as pharmaceutical products, medicinal products, dietary supplements and nutritional supplements. Substantially pure steviol or steviol glycosides may also be included in animal feed products for both the agriculture industry and the companion animal industry. Alternatively, a mixture of steviol and/or steviol glycosides can be made by culturing recombinant microorganisms separately, each producing a specific steviol or steviol glycoside, recovering the steviol or steviol glycoside in substantially pure form from each microorganism and then combining the compounds to obtain a mixture comprising each compound in the desired proportion. The recombinant microorganisms described herein permit more precise and consistent mixtures to be obtained compared to current Stevia products.

In another alternative, a substantially pure steviol or steviol glycoside can be incorporated into a food product along with other sweeteners, e.g. saccharin, dextrose, sucrose, fructose, erythritol, aspartame, sucralose, monatin, or acesulfame potassium. The weight ratio of steviol or steviol glycoside relative to other sweeteners can be varied as desired to achieve a satisfactory taste in the final food product. See, e.g., U.S. 2007/0128311. In some embodiments, the steviol or steviol glycoside may be provided with a flavor (e.g., citrus) as a flavor modulator.

Compositions produced by a recombinant microorganism described herein can be incorporated into food products. For example, a steviol glycoside composition produced by a recombinant microorganism can be incorporated into a food product in an amount ranging from about 20 mg steviol glycoside/kg food product to about 1800 mg steviol glycoside/kg food product on a dry weight basis, depending on the type of steviol glycoside and food product. For example, a steviol glycoside composition produced by a recombinant microorganism can be incorporated into a dessert, cold confectionary (e.g., ice cream), dairy product (e.g., yogurt), or beverage (e.g., a carbonated beverage) such that the food product has a maximum of 500 mg steviol glycoside/kg food on a dry weight basis. A steviol glycoside composition produced by a recombinant microorganism can be incorporated into a baked good (e.g., a biscuit) such that the food product has a maximum of 300 mg steviol glycoside/kg food on a dry weight basis. A steviol glycoside composition produced by a recombinant microorganism can be incorporated into a sauce (e.g., chocolate syrup) or vegetable product (e.g., pickles) such that the food product has a maximum of 1000 mg steviol glycoside/kg food on a dry weight basis. A steviol glycoside composition produced by a recombinant microorganism can be incorporated into bread such that the food product has a maximum of 160 mg steviol glycoside/kg food on a dry weight basis. A steviol glycoside composition produced by a recombinant microorganism, plant, or plant cell can be incorporated into a hard or soft candy such that the food product has a maximum of 1600 mg steviol glycoside/kg food on a dry weight basis. A steviol glycoside composition produced by a recombinant microorganism, plant, or plant cell can be incorporated into a processed fruit product (e.g., fruit juices, fruit filling, jams, and jellies) such that the food product has a maximum of 1000 mg steviol glycoside/kg food on a dry weight basis. In some embodiments, a steviol glycoside composition produced herein is a component of a pharmaceutical composition. See, e.g., Steviol Glycosides Chemical and Technical Assessment 69th JECFA, 2007, prepared by Harriet Wallin, Food Agric. Org.; EFSA Panel on Food Additives and Nutrient Sources added to Food (ANS), “Scientific Opinion on the safety of steviol glycosides for the proposed uses as a food additive,” 2010, EFSA Journal 8(4):1537; U.S. Food and Drug Administration GRAS Notice 323; U.S Food and Drug Administration GRAS Notice 329; WO 2011/037959; WO 2010/146463; WO 2011/046423; and WO 2011/056834.

For example, such a steviol glycoside composition can have from 90-99 weight % RebA and an undetectable amount of Stevia plant-derived components relative to a plant-derived Stevia extract, and be incorporated into a food product at from 25-1600 mg/kg, e.g., 100-500 mg/kg, 25-100 mg/kg, 250-1000 mg/kg, 50-500 mg/kg or 500-1000 mg/kg on a dry weight basis.

Such a steviol glycoside composition can be a RebB-enriched composition having greater than 3 weight % RebB and be incorporated into the food product such that the amount of RebB in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebB-enriched composition has an undetectable amount of Stevia plant-derived components relative to a plant-derived Stevia extract.

Such a steviol glycoside composition can be a RebD-enriched composition having greater than 3 weight % RebD and be incorporated into the food product such that the amount of RebD in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebD-enriched composition has an undetectable amount of Stevia plant-derived components relative to a plant-derived Stevia extract.

Such a steviol glycoside composition can be a RebE-enriched composition having greater than 3 weight % RebE and be incorporated into the food product such that the amount of RebE in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebE-enriched composition has an undetectable amount of Stevia plant-derived components relative to a plant-derived Stevia extract.

Such a steviol glycoside composition can be a RebM-enriched composition having greater than 3 weight % RebM and be incorporated into the food product such that the amount of RebM in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebM-enriched composition has an undetectable amount of Stevia plant-derived components relative to a plant-derived Stevia extract.

In some embodiments, a substantially pure steviol or steviol glycoside is incorporated into a tabletop sweetener or “cup-for-cup” product. Such products typically are diluted to the appropriate sweetness level with one or more bulking agents, e.g., maltodextrins, known to those skilled in the art. Steviol glycoside compositions enriched for RebA, RebB, RebD, RebE, or RebM, can be package in a sachet, for example, at from 10,000 to 30,000 mg steviol glycoside/kg product on a dry weight basis, for tabletop use. In some embodiments, a steviol glycoside produced in vitro, in vivo, or by whole cell bioconversion

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1. Engineering of S. cerevisiae Strain Comprising UGT33942

UGT33942 was cloned and overexpressed in an S. cerevisiae strain. The S. cerevisiae strain transformed with a UGT33942 expression plasmid was grown for 24 h in synthetic complete medium (SC medium) lacking uracil to select for plasmid being there at 30′C. After 24 h of growth, steviol was added to the culture to a final concentration of 40 μM. Samples for LC-MS were prepared 24 h after addition of steviol by mixing 1 volume of culture with one volume of DMSO, placing them at 80° C. for 10 min, and centrifuging in an Eppendorff centrifuge 5415D at maximum speed. An aliquot of the resulting supernatant was then transferred to a new vial and subjected to LC-MS analysis.

LC-MS analysis was performed on Waters ACQUITY UPLC (Waters Corporation, Milford, Mass.) with coupled to a Waters ACQUITY ESI (electrospray ionization)-TQD triple quadropole mass spectrometer. Compound separation was achieved on Waters ACQUITY UPLC® BEH C18 column (2.1×50 mm, 1.7 μm particles, 130 Å pore size) equipped with ACQUITY UPLC BEH C18 VanGuard pre-column (130 Å, 1.7 μm, 2.1 mm×5 mm) by using a gradient of the two mobile phases: A (H₂O with 0.1% formic acid) and B (Acetonitrile with 0.1% formic acid), increasing B from 20% to 50% between 0.3 to 2.0 min and up to 100% at 2.01 min, holding at 100% for 0.6 min, and re-equilibrating for 0.6 min. The flow rate was 0.6 mL/min, and the column temperature was 55° C. The MS acquisition was in negative ion-mode using Single Ion Monitoring (SIM) mode. Steviol glycoside identification was done by comparison with known standards.

Area-under-the-curve (AUC) values for LC-MS derived peaks corresponding to steviol and 19-SMG were determined for the S. cerevisiae strain overexpressing UGT33942 and for a control S. cerevisiae strain comprising an empty vector. Results are shown in FIG. 3. The majority of the steviol fed to the S. cerevisiae strain overexpressing UGT33942 was converted to 19-SMG, while the control yeast strain did not convert the fed steviol. Thus, UGT33942 glucosylates steviol on the 19-O-position resulting in production of 19-SMG. In S. rebaudiana, a UGT of the UGT74G family catalyzes 19-0 glycosylation; however, UGT33942 is a UGT polypeptide with high structural similarity to members of the UGT85 family. The activity of UGT33942 is surprising and unexpected because members of the UGT85 family have not been previously characterized as having predominantly 19-0 glycosylation activity.

### Example 2. Engineering of S. cerevisiae Strain Comprising UGT33786

UGT33786 was cloned and overexpressed in an S. cerevisiae strain. The S. cerevisiae strain transformed with a UGT33786 expression plasmid was grown for 24 h in synthetic complete medium (SC medium) lacking uracil to select for plasmid being there and to maintain the plasmid at 30° C. After 24 h of growth, steviol was added to the culture to a final concentration of 40 μM. Samples for LC-MS were prepared 24 h after addition of steviol by mixing 1 volume of culture with one volume of DMSO, placing them at 80° C. for 10 min, and centrifuging in an Eppendorff centrifuge 5415D at maximum speed. An aliquot of the resulting supernatant was then transferred to a new vial and subjected to LC-MS analysis.

LC-MS analysis was performed according to the method described in Example 1. Area-under-the-curve (AUC) values for LC-MS derived peaks corresponding to steviol and 19-SMG were determined for the S. cerevisiae strain overexpressing UGT33786 and for a control S. cerevisiae strain comprising an empty vector. Results are shown in FIG. 4. Portion of the steviol fed to the S. cerevisiae strain overexpressing UGT33786 was converted to 19-SMG, while the control yeast strain did not convert the fed steviol. Thus, UGT33786 glucosylates steviol on the 19-O-position, resulting in production of 19-SMG. As for UGT33944, UGT33786 is a UGT polypeptide with high structural similarity to members of the UGT85 family. Thus, the 19-0 glycosylation activity of UGT33944 is also surprising and unexpected.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 1
Sequences disclosed herein.

SEQ ID NO: 1

| | |
|---|---|
| atggcttgca cttccaccgg aagcacaaag cctcatgttg tttgtactaa tgttgcttct | 60 |
| caaagccaca ttaaggcaat gcttaaatta gccaaaatcc ttcaccatag aggctttcac | 120 |
| gtaacctttg ttaacacaga atccatccac aagcgttttc ttgaatctca aggacccaat | 180 |
| tccctcaacg gcttagctga ttttcgcttc gaaacccttc cagattcaga cgaaaatgcc | 240 |
| cccagaaacc atctgttggc tccatttcgt gacctattga tgaaactcaa cgatactagt | 300 |
| cctccggtga cttgcattgt ttcaaatggt ttcatgtcca cattcacaat cactgctgca | 360 |
| gaagaactaa gtgtccctat tgcattgttc tacagttttt ctgcttgcag ctttatggga | 420 |
| ttaaagcaat tccgcacttt acgagaaaaa ggccttacac cactcaaaga tgagagctgt | 480 |
| ttgacaaatg gatttttgga taaagttata gattgggttc caggaatgaa gggtatctgt | 540 |
| ttaaagcatc tcccaacctt ctttagaact acaaatcctg atgataaatt gttcaacatc | 600 |
| agcatggaaa caacagaagc agtagacaaa gcttcagcag ttgttcttct cacctttgat | 660 |
| gctttggaaa aagatgtttt ggcagctctc tccgcttcta tttctccacc tgtttataca | 720 |
| attggtcccc tccaattact tctcaaccaa ataccagaag accctctgga ggctatggga | 780 |
| tacagtattt tgaaagaaga atcagaatgt ctccaatggc tgaactccaa acctccaaat | 840 |
| tcagttgttt atgtcaactt tggcagcata gcggtcttga cagcagaaca gcttctggaa | 900 |
| tttggatggg gacttgcaaa tactaagctt cccttcttct gggttattag gcctgatttg | 960 |
| gttgttggca agtcggcggt tttgcctcca gagttcgaag ctgaaaccaa agaccggggt | 1020 |
| ctaatcgcaa gttggtgccc ccaagaacaa gtcctaaacc acccatcagt tggagggttt | 1080 |
| ctgacacata gtggttggaa ttcaaccatt gaaagcgtga cagctggagt gccgatgctg | 1140 |
| tgttggccat tttttgcaga ccagccaaca aacagttact acacttgcaa taaatgggga | 1200 |
| attggcatgg agatcaacaa tgatgtcaag agaaatgatg tagaaaagct tgtaaaggag | 1260 |
| ttaatggagg gagagaaggg taagaaaatg aaaagcaagg tcttggagtg gaagaaactt | 1320 |
| gcagaagaag caactgttcc acatggttct tcatcgatta atttagataa tctagtgaat | 1380 |
| caagttctac acagtgtaag acaaagctag | 1410 |
| SEQ ID NO: 2 | |
| MACTSTGSTK PHVVCTNVAS QSHIKAMLKL AKILHHRGFH VTFVNTESIH KRFLESQGPN | 60 |
| SLNGLADFRF ETLPDSDENA PRNHLLAPFR DLLMKLNDTS PPVTCIVSNG FMSTFTITAA | 120 |
| EELSVPIALF YSFSACSFMG LKQFRTLREK GLTPLKDESC LTNGFLDKVI DWVPGMKGIC | 180 |
| LKHLPTFFRT TNPDDKLFNI SMETTEAVDK ASAVVLLTFD ALEKDVLAAL SASISPPVYT | 240 |
| IGPLQLLLNQ IPEDPLEAMG YSILKEESEC LQWLNSKPPN SVVYVNFGSI AVLTAEQLLE | 300 |
| FGWGLANTKL PFFWVIRPDL VVGKSAVLPP EFEAETKDRG LIASWCPQEQ VLNHPSVGGF | 360 |
| LTHSGWNSTI ESVTAGVPML CWPFFADQPT NSYYTCNKWG IGMEINNDVK RNDVEKLVKE | 420 |
| LMEGEKGKKM KSKVLEWKKL AEEATVPHGS SSINLDNLVN QVLHSVRQS | 469 |
| SEQ ID NO: 3 | |
| atggcagagc aacaaaagat caaaaagtca cctcacgtct tacttattcc atttcctctg | 60 |
| caaggacata tcaacccatt catacaattt gggaaaagat tgattagtaa gggtgtaaag | 120 |
| acaacactgg taaccactat ccacactttg aattctactc tgaaccactc aaatactact | 180 |
| actacaagta tagaaattca agctatatca gacggatgcg atgagggtgg ctttatgtct | 240 |
| gccggtgaat cttacttgga aacattcaag caagtgggat ccaagtctct ggccgatcta | 300 |
| atcaaaaagt tacagagtga aggcaccaca attgacgcca taatctacga ttctatgaca | 360 |
| gagtgggttt tagacgttgc tatcgaattt ggtattgatg gaggttcctt tttcacacaa | 420 |
| gcatgtgttg tgaattctct atactaccat gtgcataaag ggttaatctc tttaccattg | 480 |
| ggtgaaactg tttcagttcc aggttttcca gtgttacaac gttgggaaac cccattgatc | 540 |
| ttacaaaatc atgaacaaat acaatcacct tggtcccaga tgttgtttgg tcaattcgct | 600 |
| aacatcgatc aagcaagatg ggtctttact aattcattct ataagttaga ggaagaggta | 660 |
| attgaatgga ctaggaagat ctggaatttg aaagtcattg gtccaacatt gccatcaatg | 720 |
| tatttggaca aaagacttga tgatgataaa gataatggtt tcaatttgta caaggctaat | 780 |
| catcacgaat gtatgaattg gctggatgac aaaccaaagg aatcagttgt atatgttgct | 840 |
| ttcggctctc ttgttaaaca tggtccagaa caagttgagg agattacaag agcacttata | 900 |
| gactctgacg taaacttttt gtgggtcatt aagcacaaag aggaggggaa actgccagaa | 960 |
| aacctttctg aagtgataaa gaccggaaaa ggtctaatcg ttgcttggtg taaacaattg | 1020 |
| gatgttttag ctcatgaatc tgtaggctgt tttgtaacac attgcggatt caactctaca | 1080 |
| ctagaagcca tttccttagg cgtacctgtc gttgcaatgc ctcagttctc cgatcagaca | 1140 |
| accaacgcta aacttttgga cgaaatacta ggggtgggtg tcagagttaa agcagacgag | 1200 |
| aatggtatcg tcagaagagg gaacctagct tcatgtatca aaatgatcat ggaagaggaa | 1260 |
| agaggagtta tcataaggaa aaacgcagtt aagtggaagg atcttgcaaa ggttgccgtc | 1320 |
| catgaaggcg gctcttcaga taatgatatt gttgaatttg tgtccgaact aatcaaagcc | 1380 |
| taa | 1383 |
| SEQ ID NO: 4 | |
| MAEQQKIKKS PHVLLIPFPL QGHINPFIQF GKRLISKGVK TTLVTTIHTL NSTLNHSNTT | 60 |
| TTSIEIQAIS DGCDEGGFMS AGESYLETFK QVGSKSLADL IKKLQSEGTT IDAIIYDSMT | 120 |
| EWVLDVAIEF GIDGGSFFTQ ACVVNSLYYH VHKGLISLPL GETVSVPGFP VLQRWETPLI | 180 |
| LQNHEQIQSP WSQMLFGQFA NIDQARWVFT NSFYKLEEEV IEWTRKIWNL KVIGPTLPSM | 240 |
| YLDKRLDDDK DNGFNLYKAN HHECMNWLDD KPKESVVYVA FGSLVKHGPE QVEEITRALI | 300 |
| DSDVNFLWVI KHKEEGKLPE NLSEVIKTGK GLIVAWCKQL DVLAHESVGC FVTHCGFNST | 360 |
| LEAISLGVPV VAMPQFSDQT TNAKLLDEIL GVGVRVKADE NGIVRRGNLA SCIKMIMEEE | 420 |
| RGVIIRKNAV KWKDLAKVAV HEGGSSDNDI VEFVSELIKA | 460 |
| SEQ ID NO: 5 | |
| atggatgcaa tggctacaac tgagaagaaa ccacacgtca tcttcatacc atttccagca | 60 |
| caaagccaca ttaaagccat gctcaaacta gcacaacttc tccaccacaa aggactccag | 120 |
| ataaccttcg tcaacaccga cttcatccac aaccagtttc ttgaatcatc gggcccacat | 180 |
| tgtctagacg gtgcaccggg tttccggttc gaaaccattc cggatggtgt ttctcacagt | 240 |
| ccggaagcga gcatcccaat cagagaatca ctcttgagat ccattgaaac caacttcttg | 300 |
| gatcgtttca ttgatcttgt aaccaaactt ccggatcctc cgacttgtat tatctcagat | 360 |
| gggttcttgt cggttttcac aattgacgct gcaaaaaagc ttggaattcc ggtcatgatg | 420 |
| tattggacac ttgctgcctg tgggttcatg ggtttttacc atattcattc tctcattgag | 480 |
| aaaggatttg caccacttaa agatgcaagt tacttgacaa atgggtattt ggacaccgtc | 540 |
| attgattggg ttccgggaat ggaaggcatc cgtctcaagg atttcccgct ggactggagc | 600 |
| actgacctca atgacaaagt tttgatgttc actacggaag ctcctcaaag gtcacacaag | 660 |
| gtttcacatc atattttcca cacgttcgat gagttggagc ctagtattat aaaaactttg | 720 |
| tcattgaggt ataatcacat ttacaccatc ggcccactgc aattacttct tgatcaaata | 780 |
| cccgaagaga aaaagcaaac tggaattacg agtatccatg gatacagttt agtaaaagaa | 840 |
| gaaccagagt gtttccagtg gcttcagtct aaagaaccaa attccgtcgt ttatgtaaat | 900 |
| tttggaagta ctacagtaat gtctttagaa gacatgacgg aatttggttg gggacttgct | 960 |
| aatagcaacc attatttcct ttggatcatc cgatcaaact tggtgatagg ggaaaatgca | 1020 |
| gttttgcccc ctgaacttga ggaacatata aagaaaagag gctttattgc tagctggtgt | 1080 |
| tcacaagaaa aggtcttgaa gcacccttcg gttggagggt tcttgactca ttgtgggtgg | 1140 |
| ggatcgacca tcgagagctt gtctgctggg gtgccaatga taLgctggcc ttattcgtgg | 1200 |
| gaccagctga ccaactgtag gtatatatgc aaagaatggg aggttgggct cgagatggga | 1260 |
| accaaagtga aacgagatga agtcaagagg cttgtacaag agttgatggg agaaggaggt | 1320 |
| cacaaaatga ggaacaaggc taaagattgg aaagaaaagg ctcgcattgc aatagctcct | 1380 |
| aacggttcat cttctttgaa catagacaaa atggtcaagg aaatcaccgt gctagcaaga | 1440 |
| aactagttac aaagttgttt cacattgtgc tttctattta agatgtaact ttgttctaat | 1500 |
| ttaatattgt ctagatgtat tgaaccataa gtttagttgg tctcaggaat tgatttttaa | 1560 |
| tgaaataatg gtcattaggg gtgagt | 1586 |
| SEQ ID NO: 6 | |
| atggatgcaa tggcaactac tgagaaaaag cctcatgtga tcttcattcc atttcctgca | 60 |
| caatctcaca taaaggcaat gctaaagtta gcacaactat tacaccataa gggattacag | 120 |
| ataactttcg tgaataccga cttcatccat aatcaatttc tggaatctag tggccctcat | 180 |
| tgtttggacg gagccccagg gtttagattc gaaacaattc ctgacggtgt ttcacattcc | 240 |
| ccagaggcct ccatcccaat aagagagagt ttactgaggt caatagaaac caactttttg | 300 |
| gatcgtttca ttgacttggt cacaaaactt ccagacccac caacttgcat aatctctgat | 360 |
| ggctttctgt cagtgtttac tatcgacgct gccaaaaagt tgggtatccc agttatgatg | 420 |
| tactggactc ttgctgcatg cggtttcatg ggtttctatc acatccattc tcttatcgaa | 480 |
| aagggttttg ctccactgaa agatgcatca tacttaacca acggctacct ggatactgtt | 540 |
| attgactggg taccaggtat ggaaggtata agacttaaag attttccttt ggattggtct | 600 |
| acagacctta atgataaagt attgatgttt actacagaag ctccacaaag atctcataag | 660 |
| gtttcacatc atatctttca cacctttgat gaattggaac catcaatcat caaaaccttg | 720 |
| tctctaagat acaatcatat ctacactatt ggtccattac aattacttct agatcaaatt | 780 |
| cctgaagaga aaaagcaaac tggtattaca tccttacacg gctactcttt agtgaaagag | 840 |
| gaaccagaat gttttcaatg gctacaaagt aaagagccta attctgtggt ctacgtcaac | 900 |
| ttcggaagta caacagtcat gtccttggaa gatatgactg aatttggttg gggccttgct | 960 |
| aattcaaatc attactttct atggattatc aggtccaatt tggtaatagg ggaaaacgcc | 1020 |
| gtattacctc cagaattgga ggaacacatc aaaaagagag gtttcattgc ttcctggtgt | 1080 |
| tctcaggaaa aggtattgaa acatccttct gttggtggtt tccttactca ttgcggttgg | 1140 |
| ggctctacaa tcgaatcact aagtgcagga gttccaatga tttgttggcc atattcatgg | 1200 |
| gaccaactta caaattgtag gtatatctgt aaagagtggg aagttggatt agaaatggga | 1260 |
| acaaaggtta aacgtgatga agtgaaaaga ttggttcagg agttgatggg ggaaggtggc | 1320 |
| cacaagatga gaaacaaggc caaagattgg aaggaaaaag ccagaattgc tattgctcct | 1380 |
| aacgggtcat cctctctaaa cattgataag atggtcaaag agattacagt cttagccaga | 1440 |
| aactaa | 1446 |
| SEQ ID NO: 7 | |
| MDAMATTEKK PHVIFIPFPA QSHIKAMLKL AQLLHHKGLQ ITFVNTDFIH NQFLESSGPH | 60 |
| CLDGAPGFRF ETIPDGVSHS PEASIPIRES LLRSIETNFL DRFIDLVTKL PDPPTCIISD | 120 |
| GFLSVFTIDA AKKLGIPVMM YWTLAACCFM GFYHIHSLIE KGFAPLKDAS YLTNGYLDTV | 180 |
| IDWVPGMEGI RLKDEPLOWS TDLNDKVLMF TTEAPQRSHK VSHHIFHTFD ELEPSIIKTL | 240 |
| SLRYNHIYTI GPLQLLLDQI PEEKKQTGIT SLHGYSLVKE EPECFQWLQS KEPNSVVYVN | 300 |
| FGSTTVMSLE DMTEFGWGLA NSNHYFLWII RSNLVIGENA VLPPELEEHI KKRGFIASWC | 360 |
| SQEKVLKHPS VGGFLTHCGW GSTIESLSAG VPMICWPYSW DQLTNCRYIC KEWEVGLEMG | 420 |
| TKVKRDEVKR LVQELMGEGG HKMRNKAKDW KEKARIAIAP NGSSSLNIDK MVKE1TVLAR | 480 |
| N | 481 |
| SEQ ID NO: 8 | |
| atggaaaaca agaccgaaac aacagttaga cgtaggcgta gaatcattct gtttccagta | 60 |
| ccttttcaag ggcacatcaa tccaatacta caactagcca acgttttgta ctctaaaggt | 120 |
| ttttctatta caatctttca caccaatttc aacaaaccaa aaacatccaa ttacccacat | 180 |
| ttcacattca gattcatact tgataatgat ccacaagatg aacgtatttc aaacttacct | 240 |
| acccacggtc ctttagctgg aatgagaatt ccaatcatca atgaacatgg tgccgatgag | 300 |
| cttagaagag aattagagtt acttatgttg gcatccgaag aggacgagga agtctcttgt | 360 |
| ctgattactg acgctctatg gtactttgcc caatctgtgg ctgatagttt gaatttgagg | 420 |
| agattggtac taatgacatc cagtctgttt aactttcacg ctcatgttag tttaccacaa | 480 |
| tttgacgaat tgggatactt ggaccctgat gacaagacta ggttagagga acaggcctct | 540 |
| ggttttccta tgttgaaagt caaagatatc aagtctgcct attctaattg gcaaatcttg | 600 |
| aaagagatct taggaaagat gatcaaacag acaaaggctt catctggagt gatttggaac | 660 |
| agtttcaaag agttagaaga gtctgaattg gagactgtaa tcagagaaat tccagcacct | 720 |
| tcattcctga taccattacc aaaacatttg actgcttcct cttcctcttt gttggatcat | 780 |
| gacagaacag tttttcaatg gttggaccaa caaccaccta gttctgtttt gtacgtgtca | 840 |
| tttggtagta cttctgaagt cgatgaaaag gacttccttg aaatcgcaag aggcttagtc | 900 |
| gatagtaagc agtcattcct ttgggtcgtg cgtccaggtt tcgtgaaagg ctcaacatgg | 960 |
| gtcgaaccac ttccagatgg ttttctaggc gaaagaggta gaatagtcaa atgggttcct | 1020 |
| caacaggaag ttttagctca tggcgctatt ggggcattct ggactcattc cggatggaat | 1080 |
| tcaactttag aatcagtatg cgaaggggta cctatgatct tttcagattt tggtcttgat | 1140 |
| caaccactga acgcaagata catgtctgat gttttgaaag tgggtgtata tctagaaaat | 1200 |
| ggctgggaaa ggggtgaaat agctaatgca ataagacgtg ttatggttga tgaagagggg | 1260 |
| gagtatatca gacaaaacgc aagagtgctg aagcaaaagg ccgacgtttc tctaatgaag | 1320 |
| ggaggctctt catacgaatc cttagaatct cttgtttcct acatttcatc actgtaa | 1377 |
| SEQ ID NO: 9 | |
| MENKTETTVR RRRRIILFPV PFQGHINPIL QLANVLYSKG FSITIFHTNF NKPKTSNYPH | 60 |
| FTFRFILDND PQDERISNLP THGPLAGMRI PIINEHGADE LRRELELLML ASEEDEEVSC | 120 |
| LITDALWYFA QSVADSLNLR RLVLMTSSLF NFHAHVSLPQ FDELGYLDPD DKIRLEEQAS | 180 |
| GFPMLKVKDI KSAYSNWQIL KEILGKMIKQ TKASSGVIWN SFKELEESEL ETVIREIPAP | 240 |
| SFLIPLPKHL TASSSSLLDH DRTVFQWLDQ QPPSSVLYVS FGSTSEVDEK DFLEIARGLV | 300 |
| DSKQSFLWVV RPGFVKGSTW VEPLPDGFLG ERGRIVKWVP QQEVLAHGAI GAFWTHSGWN | 360 |
| STLESVCEGV PMIFSDFGLD QPLNARYMSD VLKVGVYLEN GWERGEIANA IRRVMVDEEG | 420 |
| EYIRQNARVL KQKADVSLMK GGSSYESLES LVSYISSL | 458 |
| SEQ ID NO: 10 | |
| atggctacat ctgattctat tgttgatgac aggaagcagt tgcatgtggc tactttccct | 60 |
| tggcttgctt tcggtcatat actgccttac ctacaactat caaaactgat agctgaaaaa | 120 |
| ggacataaag tgtcattcct ttcaacaact agaaacattc aaagattatc ttcccacata | 180 |
| tcaccattga ttaacgtcgt tcaattgaca cttccaagag tacaggaatt accagaagat | 240 |
| gctgaagcta caacagatgt gcatcctgaa gatatccctt acttgaaaaa ggcatccgat | 300 |
| ggattacagc ctgaggtcac tagattcctt gagcaacaca gtccagattg gatcatatac | 360 |
| gactacactc actattggtt gccttcaatt gcagcatcac taggcatttc tagggcacat | 420 |
| ttcagtgtaa ccacaccttg ggccattgct tacatgggtc catccgctga tgctatgatt | 480 |
| aacggcagtg atggtagaac taccgttgaa gatttgacaa ccccaccaaa gtggtttcca | 540 |
| tttccaacta aagtctgttg gagaaaacac gacttagcaa gactggttcc atacaaggca | 600 |
| ccaggaatct cagacggcta tagaatgggt ttagtcctta aagggtctga ctgcctattg | 660 |
| tctaagtgtt accatgagtt tgggacacaa tggctaccac ttttggaaac attacaccaa | 720 |
| gttcctgtcg taccagttgg tctattacct ccagaaatcc ctggtgatga gaaggacgag | 780 |
| acttgggttt caatcaaaaa gtggttagac gggaagcaaa aaggctcagt ggtatatgtg | 840 |
| gcactgggtt ccgaagtttt agtatctcaa acagaagttg tggaacttgc cttaggtttg | 900 |
| gaactatctg gattgccatt tgtctgggcc tacagaaaac caaaaggccc tgcaaagtcc | 960 |
| gattcagttg aattgccaga cggctttgtc gagagaacta gagatagagg gttggtatgg | 1020 |
| acttcatggg ctccacaatt gagaatcctg agtcacgaat ctgtgtgcgg tttcctaaca | 1080 |
| cattgtggtt ctggttctat agttgaagga ctgatgtttg gtcatccact tatcatgttg | 1140 |
| ccaatctttg gtgaccagcc tttgaatgca cgtctgttag aagataaaca agttggaatt | 1200 |
| gaaatcccac gtaatgagga agatggatgt ttaaccaagg agtctgtggc cagatcatta | 1260 |
| cgttccgttg tcgttgaaaa ggaaggcgaa atctacaagg ccaatgcccg tgaactttca | 1320 |
| aagatctaca atgacacaaa agtagagaag gaatatgttt ctcaatttgt agattaccta | 1380 |
| gagaaaaacg ctagagccgt agctattgat catgaatcct aa | 1422 |
| SEQ ID NO: 11 | |
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTIPPKWFP | 180 |
| FPTKVCWRKH DLARLVPYKA PGISDGYRMG LVLKGSDCLL SKCYHEFGTQ WLPLLETLHQ | 240 |
| VPVVPVGLLP PEIPGDEKDE TWVSIKKWLD GKQKGSVVYV ALGSEVLVSQ TEVVELALGL | 300 |
| ELSGLPFVWA YRKPKGPAKS DSVELPDGEV ERTRDRGLVW TSWAPQLRIL SHESVCGFLT | 360 |
| HCGSGSIVEG LMFGHPLIML PIFGDQPLNA RLLEDKQVGI EIPRNEEDGC LTKESVARSL | 420 |
| RSVVVEKEGE IYKANARELS KIYNDTKVEK EYVSQFVDYL EKNARAVAID HES | 473 |
| SEQ ID NO: 12 | |
| atggctactt ctgattccat cgttgacgat agaaagcaat tgcatgttgc tacttttcca | 60 |
| tggttggctt tcggtcatat tttgccatac ttgcaattgt ccaagttgat tgctgaaaag | 120 |
| ggtcacaagg tttcattctt gtctaccacc agaaacatcc aaagattgtc ctctcatatc | 180 |
| tccccattga tcaacgttgt tcaattgact ttgccaagag tccaagaatt gccagaagat | 240 |
| gctgaagcta ctactgatgt tcatccagaa gatatccctt acttgaaaaa ggcttccgat | 300 |
| ggtttacaac cagaagttac tagattcttg gaacaacatt ccccagattg gatcatctac | 360 |
| gattatactc attactggtt gccatccatt gctgcttcat tgggtatttc tagagcccat | 420 |
| ttctctgtta ctactccatg ggctattgct tatatgggtc catctgctga tgctatgatt | 480 |
| aacggttctg atggtagaac taccgttgaa gatttgacta ctccaccaaa gtggtttcca | 540 |
| tttccaacaa aagtctgttg gagaaaacac gatttggcta gattggttcc atacaaagct | 600 |
| ccaggtattt ctgatggtta cagaatgggt atggttttga aaggttccga ttgcttgttg | 660 |
| tctaagtgct atcatgaatt cggtactcaa tggttgcctt tgttggaaac attgcatcaa | 720 |
| gttccagttg ttccagtagg tttgttgcca ccagaaattc caggtgacga aaaagacgaa | 780 |
| acttgggttt ccatcaaaaa gtggttggat ggtaagcaaa agggttctgt tgtttatgtt | 840 |
| gctttgggtt ccgaagcttt ggtttctcaa accgaagttg ttgaattggc tttgggtttg | 900 |
| gaattgtctg gtttgccatt tgtttgggct tacagaaaac ctaaaggtcc agctaagtct | 960 |
| gattctgttg aattgccaga tggtttcgtt gaaagaacta gagatagagg tttggtttgg | 1020 |
| acttcttggg ctccacaatt gagaattttg tctcatgaat ccgtctgtgg tttcttgact | 1080 |
| cattgtggtt ctggttctat cgttgaaggt ttgatgtttg gtcacccatt gattatgttg | 1140 |
| ccaatctttg gtgaccaacc attgaacgct agattattgg aagataagca agtcggtatc | 1200 |
| gaaatcccaa gaaatgaaga agatggttgc ttgaccaaag aatctgttgc tagatctttg | 1260 |
| agatccgttg tcgttgaaaa agaaggtgaa atctacaagg ctaacgctag agaattgtcc | 1320 |
| aagatctaca acgataccaa ggtcgaaaaa gaatacgttt cccaattcgt tgactacttg | 1380 |
| gaaaagaatg ctagagctgt tgccattgat catgaatctt ga | 1422 |
| SEQ ID NO: 13 | |
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG MVLKGSDCLL SKCYHEFGTQ WLPLLETLHQ | 240 |
| VPVVPVGLLP PEIPGDEKDE TWVSIKKWLD GKQKGSVVYV ALGSEALVSQ TEVVELALGL | 300 |
| ELSGLPFVWA YRKPKGPAKS DSVELPDGFV ERTRDRGLVW TSWAPQLRIL SHESVCGFLT | 360 |
| HCGSGSIVEG LMFGHPLIML PIFGDQPLNA RLLEDKQVGI EIPRNEEDGC LTKESVARSL | 420 |
| RSVVVEKEGE IYKANARELS KIYNDTKVEK EYVSQFVDYL EKNARAVAID HES | 473 |
| SEQ ID NO: 14 | |
| atggactccg gctactcctc ctcctacgcc gccgccgccg ggatgcacgt cgtgatctgc | 60 |
| ccgtggctcg ccttcggcca cctgctcccg tgcctcgacc tcgcccagcg cctcgcgtcg | 120 |
| cggggccacc gcgtgtcgtt cgtctccacg ccgcggaaca tatcccgcct cccgccggtg | 180 |
| cgccccgcgc tcgcgccgct cgtcgccttc gtggcgctgc cgctcccgcg cgtcgagggg | 240 |
| ctccccgagg gcgccgagtc caccaacgac gtcccccacg acaggccgga catggtcgag | 300 |
| ctccaccgga gggccttcga cgggctcgcc gcgcccttct cggagttctt gggcaccgcg | 360 |
| tgcgccgact gggtcatcgt cgacgtcttc caccactggg ccgcagccgc cgctctcgag | 420 |
| cacaaggtgc catgtgcaat gatgttgttg ggctctgcac atatgatcgc ttccatagca | 480 |
| gacagacggc tcgagcgcgc ggagacagag tcgcctgcgg ctgccgggca gggacgccca | 540 |
| gcggcggcgc caacgttcga ggtggcgagg atgaagttga tacgaaccaa aggctcatcg | 600 |
| ggaatgtccc tcgccgagcg cttctccttg acgctctcga ggagcagcct cgtcgtcggg | 660 |
| cggagctgcg tggagttcga gccggagacc gtcccgctcc tgtcgacgct ccgcggtaag | 720 |
| cctattacct tccttggcct tatgccgccg ttgcatgaag gccgccgcga ggacggcgag | 780 |
| gatgccaccg tccgctggct cgacgcgcag ccggccaagt ccgtcgtgta cgtcgcgcta | 840 |
| ggcagcgagg tgccactggg agtggagaag gtccacgagc tcgcgctcgg gctggagctc | 900 |
| gccgggacgc gcttcctctg ggctcttagg aagcccactg gcgtctccga cgccgacctc | 960 |
| ctccccgccg gcttcgagga gcgcacgcgc ggccgcggcg tcgtggcgac gagatgggtt | 1020 |
| cctcagatga gcatactggc gcacgccgcc gtgggcgcgt tcctgaccca ctgcggctgg | 1080 |
| aactcgacca tcgaggggct catgttcggc cacccgctta tcatgctgcc gatcttcggc | 1140 |
| gaccagggac cgaacgcgcg gctaatcgag gcgaagaacg ccggattgca ggtggcaaga | 1200 |
| aacgacggcg atggatcgtt cgaccgagaa ggcgtcgcgg cggcgattcg tgcagtcgcg | 1260 |
| gtggaggaag aaagcagcaa agtgtttcaa gccaaagcca agaagctgca ggagatcgtc | 1320 |
| gcggacatgg cctgccatga gaggtacatc gacggattca ttcagcaatt gagatcttac | 1380 |
| aaggattga | 1389 |
| SEQ ID NO: 15 | |
| atggatagtg gctactcctc atcttatgct gctgccgctg gtatgcacgt tgtgatctgc | 60 |
| ccttggttgg cctttggtca cctgttacca tgtctggatt tagcccaaag actggcctca | 120 |
| agaggccata gagtatcatt tgtgtctact cctagaaata tctctcgttt accaccagtc | 180 |
| agacctgctc tagctcctct agttgcattc gttgctcttc cacttccaag agtagaagga | 240 |
| ttgccagacg gcgctgaatc tactaatgac gtaccacatg atagacctga catggtcgaa | 300 |
| ttgcatagaa gagcctttga tggattggca gctccatttt ctgagttcct gggcacagca | 360 |
| tgtgcagact gggttatagt cgatgtattt catcactggg ctgctgcagc cgcattggaa | 420 |
| cataaggtgc cttgtgctat gatgttgtta gggtcagcac acatgatcgc atccatagct | 480 |
| gatagaagat tggaaagagc tgaaacagaa tccccagccg cagcaggaca aggtaggcca | 540 |
| gctgccgccc caacctttga agtggctaga atgaaattga ttcgtactaa aggtagttca | 600 |
| gggatgagtc ttgctgaaag gttttctctg acattatcta gatcatcatt agttgtaggt | 660 |
| agatcctgcg tcgagttcga acctgaaaca gtacctttac tatctacttt gagaggcaaa | 720 |
| cctattactt tccttggtct aatgcctcca ttacatgaag gaaggagaga agatggtgaa | 780 |
| gatgctactg ttaggtggtt agatgcccaa cctgctaagt ctgttgttta cgttgcattg | 840 |
| ggttctgagg taccactagg ggtggaaaag gtgcatgaat tagcattagg acttgagctg | 900 |
| gccggaacaa gattcctttg ggctttgaga aaaccaaccg gtgtttctga cgccgacttg | 960 |
| ctaccagctg ggttcgaaga gagaacaaga ggccgtggtg tcgttgctac tagatgggtc | 1020 |
| ccacaaatga gtattctagc tcatgcagct gtaggggcct ttctaaccca ttgcggttgg | 1080 |
| aactcaacaa tagaaggact gatgtttggt catccactta ttatgttacc aatctttggc | 1140 |
| gatcagggac ctaacgcaag attgattgag gcaaagaacg caggtctgca ggttgcacgt | 1200 |
| aatgatggtg atggttcctt tgatagagaa ggcgttgcag ctgccatcag agcagtcgcc | 1260 |
| gttgaggaag agtcatctaa agttttccaa gctaaggcca aaaaattaca agagattgtg | 1320 |
| gctgacatgg cttgtcacga aagatacatc gatggtttca tccaacaatt gagaagttat | 1380 |
| aaagactaa | 1389 |
| SEQ ID NO: 16 | |
| MDSGYSSSYA AAAGMHVVIC PWLAFGHLLP CLDLAQRLAS RGHRVSFVST PRNISRLPPV | 60 |
| RPALAPLVAF VALPLPRVEG LPDGAESTND VPHDRPDMVE LHRRAFDGLA APFSEFLGTA | 120 |
| CADWVIVDVF HHWAAPAPLE HKVPCAMMLL GSAHMIASIA DRRLERAETE SPAAAGQGRP | 180 |
| AAAPTFEVAR MKLIRTKGSS GMSLAERFSL TLSRSSLVVG RSCVEFEPET VPLLSTLRGK | 240 |
| PITFLGLMPP LHEGRREDGE DATVRWLDAQ PAKSVVYVAL GSEVPLGVEK VHELALGLEL | 300 |
| AGTRFLWALR KPTGVSDADL LPAGFEERTR GRGVVATRWV PQMSILAHAA VGAFLTHCGW | 360 |
| NSTIEGLMEG HPLIMLPIFG DQGPNARLIE AKNAGLQVAR NDGDGSFDRE GVAAAIRAVA | 420 |
| VEEESSKVFQ AKAKKLQEIV ADMACHERYI DGFIQQLRSY KD | 462 |
| SEQ ID NO: 17 | |
| MDSGYSSSYA AAAGMHVVIC PWLAFGHLLP CLDLAQRLAS RGHRVSFVST PRNISRLPPV | 60 |
| RPALAPLVAF VALPLPRVEG LPDGAESTND VPHDRPDMVE LHRRAFDGLA APFSEFLGTA | 120 |
| CADWVIVDVF HHWAAAAALE HKVPCAMMLL GSAHMIASIA DRRLERAETE SPAAAGQGRP | 180 |
| AAAPTFEVAR MKLIRTKGSS GMSLAERFSL TLSRSSLVVG RSCVEFEPET VPLLSTLRGK | 240 |
| PITFLGLLPP EIPGDEKDET WVSIKKWLDG KQKGSVVYVA LGSEALVSQT EVVELALGLE | 300 |
| LSGLPFVWAY RKPKGPAKSD SVELPDGFVE RTRDRGLVWT SWAPQLRILS HESVCGFLTH | 360 |
| CGSGSIVEGL MFGHPLIMLP IFGDQPLNAR LLEDKQVGIE IARNDGDGSF DREGVAAAIR | 420 |
| AVAVEEESSK VFQAKAKKLQ EIVADMACHE RYIDGFIQQL RSYKD | 465 |
| SEQ ID NO: 18 | |
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG MVLKGSDCLL SKCYHEFGTQ WLPLLETLHQ | 240 |
| VPVVPVGLMP PLHEGRREDG EDATVRWLDA QPAKSVVYVA LGSEVPLGVE KVHELALGLE | 300 |
| LAGTRFLWAL RKPTGVSDAD LLPAGFEERT RGRGVVATRW VPQMSILAHA AVGAFLTHCG | 360 |
| WNSTIEGLMF GHPLIMLPIF GDQGPNARLI EAKNAGLQVP RNEEDGCLTK ESVARSLRSV | 420 |
| VVEKEGEIYK ANARELSKIY NDTKVEKEYV SQFVDYLEKN ARAVAIDHES | 470 |
| SEQ ID NO: 19 | |
| atggctttgg taaacccaac cgctcttttc tatggtacct ctatcagaac aagacctaca | 60 |
| aacttactaa atccaactca aaagctaaga ccagtttcat catcttcctt accttctttc | 120 |
| tcatcagtta gtgcgattct tactgaaaaa catcaatcta atccttctga gaacaacaat | 180 |
| ttgcaaactc atctagaaac tcctttcaac tttgatagtt atatgttgga aaaagtcaac | 240 |
| atggttaacg aggcgcttga tgcatctgtc ccactaaaag acccaatcaa aatccatgaa | 300 |
| tccatgagat actctttatt ggcaggcggt aagagaatca gaccaatgat gtgtattgca | 360 |
| gcctgcgaaa tagtcggagg taatatcctt aacgccatgc cagccgcatg tgccgtggaa | 420 |
| atgattcata ctatgtcttt ggtgcatgac gatcttccat gtatggataa tgatgacttc | 480 |
| agaagaggta aacctatttc acacaaggtc tacggggagg aaatggcagt attgaccggc | 540 |
| gatgctttac taagtttatc tttcgaacat atagctactg ctacaaaggg tgtatcaaag | 600 |
| gatagaatcg tcagagctat aggggagttg gcccgttcag ttggctccga aggtttagtg | 660 |
| gctggacaag ttgtagatat cttgtcagag ggtgctgatg ttggattaga tcacctagaa | 720 |
| tacattcaca tccacaaaac agcaatgttg cttgagtcct cagtagttat tggcgctatc | 780 |
| atgggaggag gatctgatca gcagatcgaa aagttgagaa aattcgctag atctattggt | 840 |
| ctactattcc aagttgtgga tgacattttg gatgttacaa aatctaccga agagttgggg | 900 |
| aaaacagctg gtaaggattt gttgacagat aagacaactt acccaaagtt gttaggtata | 960 |
| gaaaagtcca gagaatttgc cgaaaaactt aacaaggaag cacaagagca attaagtggc | 1020 |
| tttgatagac gtaaggcagc tcctttgatc gcgttagcca actacaatgc gtaccgtcaa | 1080 |
| aattga | 1086 |
| SEQ ID NO: 20 | |
| MALVNPTALF YGTSIRTRPT NLLNPTQKLR PVSSSSLPSF SSVSAILTEK HQSNPSENNN | 60 |
| LQTHLETPFN FDSYMLEKVN MVNEALDASV PLKDPIKIHE SMRYSLLAGG KRIRPMMCIA | 120 |
| ACEIVGGNIL NAMPAACAVE MIHTMSLVHD DLPCMDNDDF RRGKPISHKV YGEEMAVLTG | 180 |
| DALLSLSFEH IATATKGVSK DRIVRAIGEL ARSVGSEGLV AGQVVDILSE GADVGLDHLE | 240 |
| YIHIHKTAML LESSVVIGAI MGGGSDQQIE KLRKFARSIG LLFQVVDDIL DVTKSTEELG | 300 |
| KTAGKDLLTD KTTYPKLLGI EKSREFAEKL NKEAQEQLSG FDRRKAAPLI ALANYNAYRQ | 360 |
| N | 361 |
| SEQ ID NO: 21 | |
| atggctgagc aacaaatatc taacttgctg tctatgtttg atgcttcaca tgctagtcag | 60 |
| aaattagaaa ttactgtcca aatgatggac acataccatt acagagaaac gcctccagat | 120 |
| tcctcatctt ctgaaggcgg ttcattgtct agatacgacg agagaagagt ctctttgcct | 180 |
| ctcagtcata atgctgcctc tccagatatt gtatcacaac tatgtttttc cactgcaatg | 240 |
| tcttcagagt tgaatcacag atggaaatct caaagattaa aggtggccga ttctccttac | 300 |
| aactatatcc taacattacc atcaaaagga attagaggtg cctttatcga ttccctgaac | 360 |
| gtatggttgg aggttccaga ggatgaaaca tcagtcatca aggaagttat tggtatgctc | 420 |
| cacaactctt cattaatcat tgatgacttc caagataatt ctccacttag aagaggaaag | 480 |
| ccatctaccc atacagtctt cggccctgcc caggctatca atactgctac ttacgttata | 540 |
| gttaaagcaa tcgaaaagat acaagacata gtgggacacg atgcattggc agatgttacg | 600 |
| ggtactatta caactatttt ccaaggtcag gccatggact tgtggtggac agcaaatgca | 660 |
| atcgttccat caatacagga atacttactt atggtaaacg ataaaaccgg tgctctcttt | 720 |
| agactgagtt tggagttgtt agctctgaat tccgaagcca gtatttctga ctctgcttta | 780 |
| gaaagtttat ctagtgctgt ttccttgcta ggtcaatact tccaaatcag agacgactat | 840 |
| atgaacttga tcgataacaa gtatacagat cagaaaggct tctgcgaaga tcttgatgaa | 900 |
| ggcaagtact cactaacact tattcatgcc ctccaaactg attcatccga tctactgacc | 960 |
| aacatccttt caatgagaag agtgcaagga aagttaacgg cacaaaagag atgttggttc | 1020 |
| tggaaatga | 1029 |
| SEQ ID NO: 22 | |
| MAEQQISNLL SMFDASHASQ KLEITVQMMD TYHYRETPPD SSSSEGGSLS RYDERRVSLP | 60 |
| LSHNAASPDI VSQLCFSTAM SSELNHRWKS QRLKVADSPY NYILTLPSKG IRGAFIDSLN | 120 |
| VWLEVPEDET SVIKEVIGML HNSSLIIDDF QDNSPLRRGK PSTHTVFGPA QAINTATYVI | 180 |
| VKAIEKIQDI VGHDALADVT GTITTIFQGQ AMDLWWTANA IVPSIQEYLL MVNDKTGALF | 240 |
| RLSLELLALN SEASISDSAL ESLSSAVSLL GQYFQIRDDY MNLIDNKYTD QKGFCEDLDE | 300 |
| GKYSLTLIHA LQTDSSDLLT NILSMRRVQG KLTAQKRCWF WK | 342 |
| SEQ ID NO: 23 | |
| atggaaaaga ctaaggagaa agcagaacgt atcttgctgg agccatacag atacttatta | 60 |
| caactaccag gaaagcaagt ccgttctaaa ctatcacaag cgttcaatca ctggttaaaa | 120 |
| gttcctgaag ataagttaca aatcattatt gaagtcacag aaatgctaca caatgcttct | 180 |
| ttactgatcg atgatataga ggattcttcc aaactgagaa gaggttttcc tgtcgctcat | 240 |
| tccatatacg gggtaccaag tgtaatcaac tcagctaatt acgtctactt cttgggattg | 300 |
| gaaaaagtat tgacattaga tcatccagac gctgtaaagc tattcaccag acaacttctt | 360 |
| gaattgcatc aaggtcaagg tttggatatc tattggagag acacttatac ttgcccaaca | 420 |
| gaagaggagt acaaagcaat ggttctacaa aagactggcg gtttgttcgg acttgccgtt | 480 |
| ggtctgatgc aacttttctc tgattacaag gaggacttaa agcctctgtt ggataccttg | 540 |
| ggcttgtttt tccagattag agatgactac gctaacttac attcaaagga atattcagaa | 600 |
| aacaaatcat tctgtgaaga tttgactgaa gggaagttta gttttccaac aatccacgcc | 660 |
| atttggtcaa gaccagaatc tactcaagtg caaaacattc tgcgtcagag aacagagaat | 720 |
| attgacatca aaaagtattg tgttcagtac ttggaagatg ttggttcttt tgcttacaca | 780 |
| agacatacac ttagagaatt agaggcaaaa gcatacaagc aaatagaagc ctgtggaggc | 840 |
| aatccttctc tagtggcatt ggttaaacat ttgtccaaaa tgttcaccga ggaaaacaag | 900 |
| taa | 903 |
| SEQ ID NO: 24 | |
| MEKTKEKAER ILLEPYRYLL QLPGKQVRSK LSQAFNHWLK VPEDKLQIII EVTEMLHNAS | 60 |
| LLIDDIEDSS KLRRGFPVAH SIYGVPSVIN SANYVYFLGL EKVLTLDHPD AVKLFTRQLL | 120 |
| ELHQGQGLDI YWRDTYTCPT EEEYKAMVLQ KTGGLFGLAV GLMQLFSDYK EDLKPLLDTL | 180 |
| GLFFQIRDDY ANLHSKEYSE NKSFCEDLTE GKESEPTIHA IWSRPESTQV QNILRQRTEN | 240 |
| IDIKKYCVQY LEDVGSFAYT RHTLRELEAK AYKQIEACGG NPSLVALVKH LSKMFTEENK | 300 |
| SEQ ID NO: 25 | |
| atggcaagat tctattttct taacgcacta ttgatggtta tctcattaca atcaactaca | 60 |
| gccttcactc cagctaaact tgcttatcca acaacaacaa cagctctaaa tgtcgcctcc | 120 |
| gccgaaactt ctttcagtct agatgaatac ttggcctcta agataggacc tatagagtct | 180 |
| gccttggaag catcagtcaa atccagaatt ccacagaccg ataagatctg cgaatctatg | 240 |
| gcctactctt tgatggcagg aggcaagaga attagaccag tgttgtgtat cgctgcatgt | 300 |
| gagatgttcg gtggatccca agatgtcgct atgcctactg ctgtggcatt agaaatgata | 360 |
| cacacaatgt ctttgattca tgatgatttg ccatccatgg ataacgatga cttgagaaga | 420 |
| ggtaaaccaa caaaccatgt cgttttcggc gaagatgtag ctattcttgc aggtgactct | 480 |
| ttattgtcaa cttccttcga gcacgtcgct agagaaacaa aaggagtgtc agcagaaaag | 540 |
| atcgtggatg ttatcgctag attaggcaaa tctgttggtg ccgagggcct tgctggcggt | 600 |
| caagttatgg acttagaatg tgaagctaaa ccaggtacca cattagacga cttgaaatgg | 660 |
| attcatatcc ataaaaccgc tacattgtta caagttgctg tagcttctgg tgcagttcta | 720 |
| ggtggtgcaa ctcctgaaga ggttgctgca tgcgagttgt ttgctatgaa tataggtctt | 780 |
| gcctttcaag ttgccgacga tatccttgat gtaaccgctt catcagaaga tttgggtaaa | 840 |
| actgcaggca aagatgaagc tactgataag acaacttacc caaagttatt aggattagaa | 900 |
| gagagtaagg catacgcaag acaactaatc gatgaagcca aggaaagttt ggctcctttt | 960 |
| ggagatagag ctgccccttt attggccatt gcagatttca ttattgatag aaagaattga | 1020 |
| SEQ ID NO: 26 | |
| MAREYELNAL LMVISLQSTT AFTPAKLAYP TTTTALNVAS AETSFSLDEY LASKIGPIES | 60 |
| ALEASVKSRI PQTDKICESM AYSLMAGGKR IRPVLCIAAC EMFGGSQDVA MPTAVALEMI | 120 |
| HTMSLIHDDL PSMDNDDLRR GKPINHVVEG EDVAILAGDS LLSTSFEHVA RETKGVSAEK | 180 |
| IVDVIARLGK SVGAEGLAGG QVMDLECEAK PGTTLDDLKW IHIHKTATLL QVAVASGAVL | 240 |
| GGATPEEVAA CELFAMNIGL AFQVADDILD VTASSEDLGK TAGKDEATDK TTYPKLLGLE | 300 |
| ESKAYARQLI DEAKESLAPF GDRAAPLLAI ADFIIDRKN | 339 |
| SEQ ID NO: 27 | |
| atgcacttag caccacgtag agtccctaga ggtagaagat caccacctga cagagttcct | 60 |
| gaaagacaag gtgccttggg tagaagacgt ggagctggct ctactggctg tgcccgtgct | 120 |
| gctgctggtg ttcaccgtag aagaggagga ggcgaggctg atccatcagc tgctgtgcat | 180 |
| agaggctggc aagccggtgg tggcaccggt ttgcctgatg aggtggtgtc taccgcagcc | 240 |
| gccttagaaa tgtttcatgc ttttgcttta atccatgatg atatcatgga tgatagtgca | 300 |
| actagaagag gctccccaac tgttcacaga gccctagctg atcgtttagg cgctgctctg | 360 |
| gacccagatc aggccggtca actaggagtt tctactgcta tcttggttgg agatctggct | 420 |
| ttgacatggt ccgatgaatt gttatacgct ccattgactc cacatagact ggcagcagta | 480 |
| ctaccattgg taacagctat gagagctgaa accgttcatg gccaatatct tgatataact | 540 |
| agtgctagaa gacctgggac cgatacttct cttgcattga gaatagccag atataagaca | 600 |
| gcagcttaca caatggaacg tccactgcac attggtgcag ccctggctgg ggcaagacca | 660 |
| gaactattag cagggctttc agcatacgcc ttgccagctg gagaagcctt ccaattggca | 720 |
| gatgacctgc taggcgtctt cggtgatcca agacgtacag ggaaacctga cctagatgat | 780 |
| cttagaggtg gaaagcatac tgtcttagtc gccttggcaa gagaacatgc cactccagaa | 840 |
| cagagacaca cattggatac attattgggt acaccaggtc ttgatagaca aggcgcttca | 900 |
| agactaagat gcgtattggt agcaactggt gcaagagccg aagccgaaag acttattaca | 960 |
| gagagaagag atcaagcatt aactgcattg aacgcattaa cactgccacc tcctttagct | 1020 |
| gaggcattag caagattgac attagggtct acagctcatc ctgcctaa | 1068 |
| SEQ ID NO: 28 | |
| MHLAPRRVPR GRRSPPDRVP ERQGALGRRR GAGSTGCARA AAGVHRRRGG GEADPSAAVH | 60 |
| RGWQAGGGTG LPDEVVSTAA ALEMFHAFAL IHDDIMDDSA TRRGSPTVHR ALADRLGAAL | 120 |
| DPDQAGQLGV STAILVGDLA LTWSDELLYA PLTPHRLAAV LPLVTAMRAE TVHGQYLDIT | 180 |
| SARRPGTDTS LALRIARYKT AAYTMERPLH IGAALAGARP ELLAGLSAYA LPAGEAFQLA | 240 |
| DDLLGVFGDP RRTGKPDLDD LRGGKHTVLV ALAREHATPE QRHTLDTLLG TPGLDRQGAS | 300 |
| RLRCVLVATG ARAEAERLIT ERRDQALTAL NALTLPPPLA EALARLTLGS TAHPA | 355 |
| SEQ ID NO: 29 | |
| atgtcatatt tcgataacta cttcaatgag atagttaatt ccgtgaacga catcattaag | 60 |
| tcttacatct ctggcgacgt accaaaacta tacgaagcct cctaccattt gtttacatca | 120 |
| ggaggaaaga gactaagacc attgatcctt acaatttctt ctgatctttt cggtggacag | 180 |
| agagaaagag catactatgc tggcgcagca atcgaagttt tgcacacatt cactttggtt | 240 |
| cacgatgata tcatggatca agataacatt cgtagaggtc ttcctactgt acatgtcaag | 300 |
| tatggcctac ctttggccat tttagctggt gacttattgc atgcaaaagc ctttcaattg | 360 |
| ttgactcagg cattgagagg tctaccatct gaaactatca tcaaggcgtt tgatatcttt | 420 |
| acaagatcta tcattatcat atcagaaggt caagctgtcg atatggaatt cgaagataga | 480 |
| attgatatca aggaacaaga gtatttggat atgatatctc gtaaaaccgc tgccttattc | 540 |
| tcagcttctt cttccattgg ggcgttgata gctggagcta atgataacga tgtgagatta | 600 |
| atgtccgatt tcggtacaaa tcttgggatc gcatttcaaa ttgtagatga tatacttggt | 660 |
| ttaacagctg atgaaaaaga gctaggaaaa cctgttttca gtgatatcag agaaggtaaa | 720 |
| aagaccatat tagtcattaa gactttagaa ttgtgtaagg aagacgagaa aaagattgtg | 780 |
| ttaaaagcgc taggcaacaa gtcagcatca aaggaagagt tgatgagttc tgctgacata | 840 |
| atcaaaaagt actcattgga ttacgcctac aacttagctg agaaatacta caaaaacgcc | 900 |
| atcgattctc taaatcaagt ttcaagtaaa agtgatattc cagggaaggc attgaaatat | 960 |
| cttgctgaat tcaccatcag aagacgtaag taa | 993 |
| SEQ ID NO: 30 | |
| MSYFDNYFNE IVNSVNDIIK SYISGDVPKL YEASYHLFTS GGKRLRPLIL TISSDLEGGQ | 60 |
| RERAYYAGAA IEVLHTFTLV HDDIMDQDNI RRGLPTVHVK YGLPLAILAG DLLHAKAFQL | 120 |
| LTQALRGLPS ETIIKAFDIF TRSIIIISEG QAVDMEFEDR IDIKEQEYLD MISRKTAALF | 180 |
| SASSSIGALI AGANDNDVRL MSDFGTNLGI AFQIVDDILG LTADEKELGK PVFSDIREGK | 240 |
| KTILVIKTLE LCKEDEKKIV LKALGNKSAS KEELMSSADI IKKYSLDYAY NLAEKYYKNA | 300 |
| IDSLNQVSSK SDIPGKALKY LAEFTIRRRK | 330 |
| SEQ ID NO: 31 | |
| atggtcgcac aaactttcaa cctggatacc tacttatccc aaagacaaca acaagttgaa | 60 |
| gaggccctaa gtgctgctct tgtgccagct tatcctgaga gaatatacga agctatgaga | 120 |
| tactccctcc tggcaggtgg caaaagatta agacctatct tatgtttagc tgcttgcgaa | 180 |
| ttggcaggtg gttctgttga acaagccatg ccaactgcgt gtgcacttga aatgatccat | 240 |
| acaatgtcac taattcatga tgacctgcca gccatggata acgatgattt cagaagagga | 300 |
| aagccaacta atcacaaggt gttcggggaa gatatagcca tcttagcggg tgatgcgctt | 360 |
| ttagcttacg cttttgaaca tattgcttct caaacaagag gagtaccacc tcaattggtg | 420 |
| ctacaagtta ttgctagaat cggacacgcc gttgctgcaa caggcctcgt tggaggccaa | 480 |
| gtcgtagacc ttgaatctga aggtaaagct atttccttag aaacattgga gtatattcac | 540 |
| tcacataaga ctggagcctt gctggaagca tcagttgtct caggcggtat tctcgcaggg | 600 |
| gcagatgaag agcttttggc cagattgtct cattacgcta gagatatagg cttggctttt | 660 |
| caaatcgtcg atgatatcct ggatgttact gctacatctg aacagttggg gaaaaccgct | 720 |
| ggtaaagacc aggcagccgc aaaggcaact tatccaagtc tattgggttt agaagcctct | 780 |
| agacagaaag cggaagagtt gattcaatct gctaaggaag ccttaagacc ttacggttca | 840 |
| caagcagagc cactcctagc gctggcagac ttcatcacac gtcgtcagca ttaa | 894 |
| SEQ ID NO: 32 | |
| MVAQTFNLDT YLSQRQQQVE EALSAALVPA YPERIYEAMR YSLLAGGKRL RPILCLAACE | 60 |
| LAGGSVEQAM PTACALEMIH TMSLIHDDLP AMDNDDFRRG KPTNHKVFGE DIAILAGDAL | 120 |
| LAYAFEHIAS QTRGVPPQLV LQVIARIGHA VAATGLVGGQ VVDLESEGKA ISLETLEYIH | 180 |
| SHKTGALLEA SVVSGGILAG ADEELLARLS HYARDIGLAF QIVDDILDVT ATSEQLGKTA | 240 |
| GKDQAAAKAT YPSLLGLEAS RQKAEELIQS AKEALRPYGS QAEPLLALAD FITRRQH | 297 |
| SEQ ID NO: 33 | |
| atgaaaaccg ggtttatctc accagcaaca gtatttcatc acagaatctc accagcgacc | 60 |
| actttcagac atcacttatc acctgctact acaaactcta caggcattgt cgccttaaga | 120 |
| gacatcaact tcagatgtaa agcagtttct aaagagtact ctgatctgtt gcagaaagat | 180 |
| gaggcttctt tcacaaaatg ggacgatgac aaggtgaaag atcatcttga taccaacaaa | 240 |
| aacttatacc caaatgatga gattaaggaa tttgttgaat cagtaaaggc tatgttcggt | 300 |
| agtatgaatg acggggagat aaacgtctct gcatacgata ctgcatgggt tgctttggtt | 360 |
| caagatgtcg atggatcagg tagtcctcag ttcccttctt ctttagaatg gattgccaac | 420 |
| aatcaattgt cagatggatc atggggagat catttgctgt tctcagctca cgatagaatc | 480 |
| atcaacacat tagcatgcgt tattgcactt acaagttgga atgttcatcc ttctaagtgt | 540 |
| gaaaaaggtt tgaattttct gagagaaaac atttgcaaat tagaagatga aaacgcagaa | 600 |
| catatgccaa ttggttttga agtaacattc ccatcactaa ttgatatcgc gaaaaagttg | 660 |
| aacattgaag tacctgagga tactccagca cttaaagaga tctacgcacg tagagatatc | 720 |
| aagttaacta agatcccaat ggaagttctt cacaaggtac ctactacttt gttacattct | 780 |
| ttggaaggaa tgcctgattt ggagtgggaa aaactgttaa agctacaatg taaagatggt | 840 |
| agtttcttgt tttccccatc tagtaccgca ttcgccctaa tgcaaacaaa agatgagaaa | 900 |
| tgcttacagt atctaacaaa tatcgtcact aagttcaacg gtggcgtgcc taatgtgtac | 960 |
| ccagtcgatt tgtttgaaca tatttgggtt gttgatagac tgcagagatt ggggattgcc | 1020 |
| agatacttca aatcagagat aaaagattgt gtagagtata tcaataagta ctggaccaaa | 1080 |
| aatggaattt gttgggctag aaatactcac gttcaagata tcgatgatac agccatggga | 1140 |
| ttcagagtgt tgagagcgca cggttatgac gtcactccag atgtttttag acaatttgaa | 1200 |
| aaagatggta aattcgtttg ctttgcaggg caatcaacac aagccgtgac aggaatgttt | 1260 |
| aacgtttaca gagcctctca aatgttgttc ccaggggaga gaattttgga agatgccaaa | 1320 |
| aagttctctt acaattactt aaaggaaaag caaagtacca acgaattgct ggataaatgg | 1380 |
| ataatcgcta aagatctacc tggtgaagtt ggttatgctc tggatatccc atggtatgct | 1440 |
| tccttaccaa gattggaaac tcgttattac cttgaacaat acggcggtga agatgatgtc | 1500 |
| tggataggca agacattata cagaatgggt tacgtgtcca ataacacata tctagaaatg | 1560 |
| gcaaagctgg attacaataa ctatgttgca gtccttcaat tagaatggta cacaatacaa | 1620 |
| caatggtacg tcgatattgg tatagagaag ttcgaatctg acaacatcaa gtcagtcctg | 1680 |
| SEQ ID NO: 34 | |
| MKTGFISPAT VFHHRISPAT TFRHHLSPAT TNSTGIVALR DINFRCKAVS KEYSDLLQKD | 60 |
| EASFTKWDDD KVKDHLDTNK NLYPNDEIKE FVESVKAMFG SMNDGEINVS AYDTAWVALV | 120 |
| QDVDGSGSPQ FPSSLEWIAN NQLSDGSWGD HLLFSAHDRI INTLACVIAL TSWNVHPSKC | 180 |
| EKGLNFLREN ICKLEDENAE HMPIGFEVTF PSLIDIAKKL NIEVPEDTPA LKEIYARRDI | 240 |
| KLTKIPMEVL HKVPTTLLHS LEGMPDLEWE KLLKLQCKDG SFLFSPSSTA FALMQTKDEK | 300 |
| CLQYLTNIVT KFNGGVPNVY PVDLFEHIWV VDRLQRLGIA RYFKSEIKDC VEYINKYWTK | 360 |
| NGICWARNTH VQDIDDTAMG FRVLRAHGYD VTPDVFRQFE KDGKFVCFAG QSTQAVTGMF | 420 |
| NVYRASQMLF PGERILEDAK KFSYNYLKEK QSTNELLDKW IIAKDLPGEV GYALDIPWYA | 480 |
| SLPRLETRYY LEQYGGEDDV WIGKTLYRMG YVSNNTYLEM AKLDYNNYVA VLQLEWYTIQ | 540 |
| QWYVDIGIEK FESDNIKSVL VSYYLAAASI FEPERSKERI AWAKTTILVD KITSIFDSSQ | 600 |
| SSKEDITAFI DKFRNKSSSK KHSINGEPWH EVMVALKKTL HGFALDALMT HSQDIHPQLH | 660 |
| QAWEMWLTKL QDGVDVTAEL MVQMINMTAG RWVSKELLTH PQYQRLSTVT NSVCHDITKL | 720 |
| HNFKENSTTV DSKVQELVQL VFSDTPDDLD QDMKQTFLTV MKTFYYKAWC DPNTINDHIS | 780 |
| KVFEIVI | 787 |
| SEQ ID NO: 35 | |
| atgcctgatg cacacgatgc tccacctcca caaataagac agagaacact agtagatgag | 60 |
| gctacccaac tgctaactga gtccgcagaa gatgcatggg gtgaagtcag tgtgtcagaa | 120 |
| tacgaaacag caaggctagt tgcccatgct acatggttag gtggacacgc cacaagagtg | 180 |
| gccttccttc tggagagaca acacgaagac gggtcatggg gtccaccagg tggatatagg | 240 |
| ttagtcccta cattatctgc tgttcacgca ttattgacat gtcttgcctc tcctgctcag | 300 |
| gatcatggcg ttccacatga tagactttta agagctgttg acgcaggctt gactgccttg | 360 |
| agaagattgg ggacatctga ctccccacct gatactatag cagttgagct ggttatccca | 420 |
| tctttgctag agggcattca acacttactg gaccctgctc atcctcatag tagaccagcc | 480 |
| ttctctcaac atagaggctc tcttgtttgt cctggtggac tagatgggag aactctagga | 540 |
| gctttgagat cacacgccgc agcaggtaca ccagtaccag gaaaagtctg gcacgcttcc | 600 |
| gagactttgg gcttgagtac cgaagctgct tctcacttgc aaccagccca aggtataatc | 660 |
| ggtggctctg ctgctgccac agcaacatgg ctaaccaggg ttgcaccatc tcaacagtca | 720 |
| gattctgcca gaagatacct tgaggaatta caacacagat actctggccc agttccttcc | 780 |
| attaccccta tcacatactt cgaaagagca tggttattga acaattttgc agcagccggt | 840 |
| gttccttgtg aggctccagc tgctttgttg gattccttag aagcagcact tacaccacaa | 900 |
| ggtgctcctg ctggagcagg attgcctcca gatgctgatg atacagccgc tgtgttgctt | 960 |
| gcattggcaa cacatgggag aggtagaaga ccagaagtac tgatggatta caggactgac | 1020 |
| gggtatttcc aatgctttat tggggaaagg actccatcaa tttcaacaaa cgctcacgta | 1080 |
| ttggaaacat tagggcatca tgtggcccaa catccacaag atagagccag atacggatca | 1140 |
| gccatggata ccgcatcagc ttggctgctg gcagctcaaa agcaagatgg ctcttggtta | 1200 |
| gataaatggc atgcctcacc atactacgct actgtttgtt gcacacaagc cctagccgct | 1260 |
| catgcaagtc ctgcaactgc accagctaga cagagagctg tcagatgggt tttagccaca | 1320 |
| caaagatccg atggcggttg gggtctatgg cattcaactg ttgaagagac tgcttatgcc | 1380 |
| ttacagatct tggccccacc ttctggtggt ggcaatatcc cagtccaaca agcacttact | 1440 |
| agaggcagag caagattgtg tggagccttg ccactgactc ctttatggca tgataaggat | 1500 |
| ttgtatactc cagtaagagt agtcagagct gccagagctg ctgctctgta cactaccaga | 1560 |
| gatctattgt taccaccatt gtaa | 1584 |
| SEQ ID NO: 36 | |
| MPDAHDAPPP QIRQRTLVDE ATQLLTESAE DAWGEVSVSE YETARLVAHA TWLGGHATRV | 60 |
| AFLLERQHED GSWGPPGGYR LVPTLSAVHA LLTCLASPAQ DHGVPHDRLL RAVDAGLTAL | 120 |
| RRLGTSDSPP DTIAVELVIP SLLEGIQHLL DPAHPHSRPA FSQHRGSLVC PGGLDGRTLG | 180 |
| ALRSHAAAGT PVPGKVWHAS ETLGLSTEAA SHLQPAQGII GGSAAATATW LTRVAPSQQS | 240 |
| DSARRYLEEL QHRYSGPVPS ITPITYFERA WLLNNFAAAG VPCEAPAALL DSLEAALTPQ | 300 |
| GAPAGAGLPP DADDTAAVLL ALATHGRGRR PEVLMDYRTD GYFQCFIGER TPSISTNAHV | 360 |
| LETLGHHVAQ HPQDRARYGS AMDTASAWLL AAQKQDGSWL DKWHASPYYA TVCCTQALAA | 420 |
| HASPATAPAR QRAVRWVLAT QRSDGGWGLW HSTVEETAYA LQILAPPSGG GNIPVQQALT | 480 |
| RGRARLCGAL PLTPLWHDKD LYTPVRVVRA ARAAALYTTR DLLLPPL | 527 |
| SEQ ID NO: 37 | |
| atgaacgccc tatccgaaca cattttgtct gaattgagaa gattattgtc tgaaatgagt | 60 |
| gatggcggat ctgttggtcc atctgtgtat gatacggccc aggccctaag attccacggt | 120 |
| aacgtaacag gtagacaaga tgcatatgct tggttgatcg cccagcaaca agcagatgga | 180 |
| ggttggggct ctgccgactt tccactcttt agacatgctc caacatgggc tgcacttctc | 240 |
| gcattacaaa gagctgatcc acttcctggc gcagcagacg cagttcagac cgcaacaaga | 300 |
| ttcttgcaaa gacaaccaga tccatacgct catgccgttc ctgaggatgc ccctattggt | 360 |
| gctgaactga tcttgcctca gttttgtgga gaggctgctt ggttgttggg aggtgtggcc | 420 |
| ttccctagac acccagccct attaccatta agacaggctt gtttagtcaa actgggtgca | 480 |
| gtcgccatgt tgccttcagg acacccattg ctccactcct gggaggcatg gggtacttct | 540 |
| ccaacaacag cctgtccaga cgatgatggt tctataggta tctcaccagc agctacagcc | 600 |
| gcctggagag cccaggctgt gaccagaggc tcaactcctc aagtgggcag agctgacgca | 660 |
| tacttacaaa tggcttcaag agcaacgaga tcaggcatag aaggagtctt ccctaatgtt | 720 |
| tggcctataa acgtattcga accatgctgg tcactgtaca ctctccatct tgccggtctg | 780 |
| ttcgcccatc cagcactggc tgaggctgta agagttatcg ttgctcaact tgaagcaaga | 840 |
| ttgggagtgc atggcctcgg accagcttta cattttgctg ccgacgctga tgatactgca | 900 |
| gttgccttat gcgttctgca tttggctggc agagatcctg cagttgacgc attgagacat | 960 |
| tttgaaattg gtgagctctt tgttacattc ccaggagaga gaaatgctag tgtctctacg | 1020 |
| aacattcacg ctcttcatgc tttgagattg ttaggtaaac cagctgccgg agcaagtgca | 1080 |
| tacgtcgaag caaatagaaa tccacatggt ttgtgggaca acgaaaaatg gcacgtttca | 1140 |
| tggctttatc caactgcaca cgccgttgca gctctagctc aaggcaagcc tcaatggaga | 1200 |
| gatgaaagag cactagccgc tctactacaa gctcaaagag atgatggtgg ttggggagct | 1260 |
| ggtagaggat ccactttcga ggaaaccgcc tacgctcttt tcgctttaca cgttatggac | 1320 |
| ggatctgagg aagccacagg cagaagaaga atcgctcaag tcgtcgcaag agccttagaa | 1380 |
| tggatgctag ctagacatgc cgcacatgga ttaccacaaa caccactctg gattggtaag | 1440 |
| gaattgtact gtcctactag agtcgtaaga gtagctgagc tagctggcct gtggttagca | 1500 |
| ttaagatggg gtagaagagt attagctgaa ggtgctggtg ctgcacctta a | 1551 |
| SEQ ID NO: 38 | |
| MNALSEHILS ELRRLLSEMS DGGSVGPSVY DTAQALRFHG NVTGRQDAYA WLIAQQQADG | 60 |
| GWGSADFPLF RHAPTWAALL ALQRADPLPG AADAVQTATR FLQRQPDPYA HAVPEDAPIG | 120 |
| AELILPQFCG EAAWLLGGVA FPRHPALLPL RQACLVKLGA VAMLPSGHPL LHSWEAWGTS | 180 |
| PTTACPDDDG SIGISPAATA AWRAQAVTRG STPQVGRADA YLQMASRATR SGIEGVFPNV | 240 |
| WPINVFEPCW SLYTLHLAGL FAHPALAEAV RVIVAQLEAR LGVHGLGPAL HFAADADDTA | 300 |
| VALCVLHLAG RDPAVDALRH FEIGELFVTF PGERNASVST NIHALHALRL LGKPAAGASA | 360 |
| YVEANRNPHG LWDNEKWHVS WLYPTAHAVA ALAQGKPQWR DERALAALLQ AQRDDGGWGA | 420 |
| GRGSTFEETA YALFALHVMD GSEEATGRRR IAQVVARALE WMLARHAAHG LPQTPLWIGK | 480 |
| ELYCPTRVVR VAELAGLWLA LRWGRRVLAE GAGAAP | 516 |
| SEQ ID NO: 39 | |
| atggttttgt cttcttcttg tactacagta ccacacttat cttcattagc tgtcgtgcaa | 60 |
| cttggtcctt ggagcagtag gattaaaaag aaaaccgata ctgttgcagt accagccgct | 120 |
| gcaggaaggt ggagaagggc cttggctaga gcacagcaca catcagaatc cgcagctgtc | 180 |
| gcaaagggca gcagtttgac ccctatagtg agaactgacg ctgagtcaag gagaacaaga | 240 |
| tggccaaccg atgacgatga cgccgaacct ttagtggatg agatcagggc aatgcttact | 300 |
| tccatgtctg atggtgacat ttccgtgagc gcatacgata cagcctgggt cggattggtt | 360 |
| ccaagattag acggcggtga aggtcctcaa tttccagcag ctgtgagatg gataagaaat | 420 |
| aaccagttgc ctgacggaag ttggggcgat gccgcattat tctctgccta tgacaggctt | 480 |
| atcaataccc ttgcctgcgt tgtaactttg acaaggtggt ccctagaacc agagatgaga | 540 |
| ggtagaggac tatctttttt gggtaggaac atgtggaaat tagcaactga agatgaagag | 600 |
| tcaatgccta ttggcttcga attagcattt ccatctttga tagagcttgc taagagccta | 660 |
| ggtgtccatg acttccctta tgatcaccag gccctacaag gaatctactc ttcaagagag | 720 |
| atcaaaatga agaggattcc aaaagaagtg atgcataccg ttccaacatc aatattgcac | 780 |
| agtttggagg gtatgcctgg cctagattgg gctaaactac ttaaactaca gagcagcgac | 840 |
| ggaagttttt tgttctcacc agctgccact gcatatgctt taatgaatac cggagatgac | 900 |
| aggtgtttta gctacatcga tagaacagta aagaaattca acggcggcgt ccctaatgtt | 960 |
| tatccagtgg atctatttga acatatttgg gccgttgata gacttgaaag attaggaatc | 1020 |
| tccaggtact tccaaaagga gatcgaacaa tgcatggatt atgtaaacag gcattggact | 1080 |
| gaggacggta tttgttgggc aaggaactct gatgtcaaag aggtggacga cacagctatg | 1140 |
| gcctttagac ttcttaggtt gcacggctac agcgtcagtc ctgatgtgtt taaaaacttc | 1200 |
| gaaaaggacg gtgaattttt cgcatttgtc ggacagtcta atcaagctgt taccggtatg | 1260 |
| tacaacttaa acagagcaag ccagatatcc ttcccaggcg aggatgtgct tcatagagct | 1320 |
| ggtgccttct catatgagtt cttgaggaga aaagaagcag agggagcttt gagggacaag | 1380 |
| tggatcattt ctaaagatct acctggtgaa gttgtgtata ctttggattt tccatggtac | 1440 |
| ggcaacttac ctagagtcga ggccagagac tacctagagc aatacggagg tggtgatgac | 1500 |
| gtttggattg gcaagacatt gtataggatg ccacttgtaa acaatgatgt atatttggaa | 1560 |
| ttggcaagaa tggatttcaa ccactgccag gctttgcatc agttagagtg gcaaggacta | 1620 |
| aaaagatggt atactgaaaa taggttgatg gactttggtg tcgcccaaga agatgccctt | 1680 |
| agagcttatt ttcttgcagc cgcatctgtt tacgagcctt gtagagctgc cgagaggctt | 1740 |
| gcatgggcta gagccgcaat actagctaac gccgtgagca cccacttaag aaatagccca | 1800 |
| tcattcagag aaaggttaga gcattctctt aggtgtagac ctagtgaaga gacagatggc | 1860 |
| tcctggttta actcctcaag tggctctgat gcagttttag taaaggctgt cttaagactt | 1920 |
| actgattcat tagccaggga agcacagcca atccatggag gtgacccaga agatattata | 1980 |
| cacaagttgt taagatctgc ttgggccgag tgggttaggg aaaaggcaga cgctgccgat | 2040 |
| agcgtgtgca atggtagttc tgcagtagaa caagagggat caagaatggt ccatgataaa | 2100 |
| cagacctgtc tattattggc tagaatgatc gaaatttctg ccggtagggc agctggtgaa | 2160 |
| gcagccagtg aggacggcga tagaagaata attcaattaa caggctccat ctgcgacagt | 2220 |
| cttaagcaaa aaatgctagt ttcacaggac cctgaaaaaa atgaagagat gatgtctcac | 2280 |
| gtggatgacg aattgaagtt gaggattaga gagttcgttc aatatttgct tagactaggt | 2340 |
| gaaaaaaaga ctggatctag cgaaaccagg caaacatttt taagtatagt gaaatcatgt | 2400 |
| tactatgctg ctcattgccc acctcatgtc gttgatagac acattagtag agtgattttc | 2460 |
| gagccagtaa gtgccgcaaa gtaaccgcgg | 2490 |
| SEQ ID NO: 40 | |
| MVLSSSCTTV PHLSSLAVVQ LGPWSSRIKK KTDTVAVPAA AGRWRRALAR AQHTSESAAV | 60 |
| AKGSSLTPIV RTDAESRRTR WPTDDDDAEP LVDEIRAMLT SMSDGDISVS AYDTAWVGLV | 120 |
| PRLDGGEGPQ FPAAVRWIRN NQLPDGSWGD AALFSAYDRL INTLACVVTL TRWSLEPEMR | 180 |
| GRGLSFLGRN MWKLATEDEE SMPIGFELAF PSLIELAKSL GVHDEPYDHQ ALQGIYSSRE | 240 |
| IKMKRIPKEV MHTVPTSILH SLEGMPGLDW AKLLKLQSSD GSFLFSPAAT AYALMNTGDD | 300 |
| RCFSYIDRTV KKFNGGVPNV YPVDLFEHIW AVDRLERLGI SRYFQKEIEQ CMDYVNRHWT | 360 |
| EDGICWARNS DVKEVDDTAM AFRLLRLHGY SVSPDVFKNF EKDGEFFAFV GQSNQAVTGM | 420 |
| YNLNRASQIS FPGEDVLHRA GAFSYEFLRR KEAEGALRDK WIISKDLPGE VVYTLDFPWY | 480 |
| GNLPRVEARD YLEQYGGGDD VWIGKTLYRM PLVNNDVYLE LARMDFNHCQ ALHQLEWQGL | 540 |
| KRWYTENRLM DFGVAQEDAL RAYFLAAASV YEPCRAAERL AWARAAILAN AVSTHLRNSP | 600 |
| SFRERLEHSL RCRPSEETDG SWFNSSSGSD AVLVKAVLRL TDSLAREAQP IHGGDPEDII | 660 |
| HKLLRSAWAE WVREKADAAD SVCNGSSAVE QEGSRMVHDK QTCLLLARMI EISAGRAAGE | 720 |
| AASEDGDRRI IQLTGSICDS LKQKMLVSQD PEKNEEMMSH VDDELKLRIR EFVQYLLRLG | 780 |
| EKKTGSSETR QTFLSIVKSC YYAAHCPPHV VDRHISRVIF EPVSAAK | 827 |
| SEQ ID NO: 41 | |
| cttcttcact aaatacttag acagagaaaa cagagctttt taaagccatg tctcttcagt | 60 |
| atcatgttct aaactccatt ccaagtacaa cctttctcag ttctactaaa acaacaatat | 120 |
| cttcttcttt ccttaccatc tcaggatctc ctctcaatgt cgctagagac aaatccagaa | 180 |
| gcggttccat acattgttca aagcttcgaa ctcaagaata cattaattct caagaggttc | 240 |
| aacatgattt gcctctaata catgagtggc aacagcttca aggagaagat gctcctcaga | 300 |
| ttagtgttgg aagtaatagt aatgcattca aagaagcagt gaagagtgtg aaaacgatct | 360 |
| tgagaaacct aacggacggg gaaattacga tatcggctta cgatacagct tgggttgcat | 420 |
| tgatcgatgc cggagataaa actccggcgt ttccctccgc cgtgaaatgg atcgccgaga | 480 |
| accaactttc cgatggttct tggggagatg cgtatctctt ctcttatcat gatcgtctca | 540 |
| tcaataccct tgcatgcgtc gttgctctaa gatcatggaa tctctttcct catcaatgca | 600 |
| acaaaggaat cacgtttttc cgggaaaata ttgggaagct agaagacgaa aatgatgagc | 660 |
| atatgccaat cggattcgaa gtagcattcc catcgttgct tgagatagct cgaggaataa | 720 |
| acattgatgt accgtacgat tctccggtct taaaagatat atacgccaag aaagagctaa | 780 |
| agcttacaag gataccaaaa gagataatgc acaagatacc aacaacattg ttgcatagtt | 840 |
| tggaggggat gcgtgattta gattgggaaa agctcttgaa acttcaatct caagacggat | 900 |
| ctttcctctt ctctccttcc tctaccgctt ttgcattcat gcagacccga gacagtaact | 960 |
| gcctcgagta tttgcgaaat gccgtcaaac gtttcaatgg aggagttccc aatgtctttc | 1020 |
| ccgtggatct tttcgagcac atatggatag tggatcggtt acaacgttta gggatatcga | 1080 |
| gatactttga agaagagatt aaagagtgtc ttgactatgt ccacagatat tggaccgaca | 1140 |
| atggcatatg ttgggctaga tgttcccatg tccaagacat cgatgataca gccatggcat | 1200 |
| ttaggctctt aagacaacat ggataccaag tgtccgcaga tgtattcaag aactttgaga | 1260 |
| aagagggaga gtttttctgc tttgtggggc aatcaaacca agcagtaacc ggtatgttca | 1320 |
| acctataccg ggcatcacaa ttggcgtttc caagggaaga gatattgaaa aacgccaaag | 1380 |
| agttttctta taattatctg ctagaaaaac gggagagaga ggagttgatt gataagtgga | 1440 |
| ttataatgaa agacttacct ggcgagattg ggtttgcgtt agagattcca tggtacgcaa | 1500 |
| gcttgcctcg agtagagacg agattctata ttgatcaata tggtggagaa aacgacgttt | 1560 |
| ggattggcaa gactctttat aggatgccat acgtgaacaa taatggatat ctggaattag | 1620 |
| caaaacaaga ttacaacaat tgccaagctc agcatcagct cgaatgggac atattccaaa | 1680 |
| agtggtatga agaaaatagg ttaagtgagt ggggtgtgcg cagaagtgag cttctcgagt | 1740 |
| gttactactt agcggctgca actatatttg aatcagaaag gtcacatgag agaatggttt | 1800 |
| gggctaagtc aagtgtattg gttaaagcca tttcttcttc ttttggggaa tcctctgact | 1860 |
| ccagaagaag cttctccgat cagtttcatg aatacattgc caatgctcga cgaagtgatc | 1920 |
| atcactttaa tgacaggaac atgagattgg accgaccagg atcggttcag gccagtcggc | 1980 |
| ttgccggagt gttaatcggg actttgaatc aaatgtcttt tgaccttttc atgtctcatg | 2040 |
| gccgtgacgt taacaatctc ctctatctat cgtggggaga ttggatggaa aaatggaaac | 2100 |
| tatatggaga tgaaggagaa ggagagctca tggtgaagat gataattcta atgaagaaca | 2160 |
| atgacctaac taacttcttc acccacactc acttcgttcg tctcgcggaa atcatcaatc | 2220 |
| gaatctgtct tcctcgccaa tacttaaagg caaggagaaa cgatgagaag gagaagacaa | 2280 |
| taaagagtat ggagaaggag atggggaaaa tggttgagtt agcattgtcg gagagtgaca | 2340 |
| catttcgtga cgtcagcatc acgtttcttg atgtagcaaa agcattttac tactttgctt | 2400 |
| tatgtggcga tcatctccaa actcacatct ccaaagtctt gtttcaaaaa gtctagtaac | 2460 |
| ctcatcatca tcatcgatcc attaacaatc agtggatcga tgtatccata gatgcgtgaa | 2520 |
| taatatttca tgtagagaag gagaacaaat tagatcatgt agggttatca | 2570 |
| SEQ ID NO: 42 | |
| MSLQYHVLNS IPSTTFLSST KTTISSSFLT ISGSPLNVAR DKSRSGSIHC SKLRTQEYIN | 60 |
| SQEVQHDLPL IHEWQQLQGE DAPQISVGSN SNAFKEAVKS VKTILRNLTD GEITISAYDT | 120 |
| AWVALIDAGD KTPAFPSAVK WIAENQLSDG SWGDAYLFSY HDRLINTLAC VVALRSWNLF | 180 |
| PHQCNKGITF FRENIGKLED ENDEHMPIGF EVAFPSLLEI ARGINIDVPY DSPVLKDIYA | 240 |
| KKELKLTRIP KEIMHKIPTT LLHSLEGMRD LDWEKLLKLQ SQDGSFLFSP SSTAFAFMQT | 300 |
| RDSNCLEYLR NAVKRFNGGV PNVFPVDLFE HIWIVDRLQR LGISRYFEEE IKECLDYVHR | 360 |
| YWTDNGICWA RCSHVQDIDD TAMAFRLLRQ HGYQVSADVF KNFEKEGEFF CFVGQSNQAV | 420 |
| TGMFNLYRAS QLAFPREEIL KNAKEFSYNY LLEKREREEL IDKWIIMKDL PGEIGFALEI | 480 |
| PWYASLPRVE TRFYIDQYGG ENDVWIGKTL YRMPYVNNNG YLELAKQDYN NCQAQHQLEW | 540 |
| DIFQKWYEEN RLSEWGVRRS ELLECYYLAA ATIFESERSH ERMVWAKSSV LVKAISSSFG | 600 |
| ESSDSRRSFS DQFHEYIANA RRSDHHFNDR NMRLDRPGSV QASRLAGVLI GTLNQMSFDL | 660 |
| FMSHGRDVNN LLYLSWGDWM EKWKLYGDEG EGELMVKMII LMKNNDLTNF FTHTHFVRLA | 720 |
| EIINRICLPR QYLKARRNDE KEKTIKSMEK EMGKMVELAL SESDTFRDVS ITFLDVAKAF | 780 |
| YYFALCGDHL QTHISKVLFQ KV | 802 |
| SEQ ID NO: 43 | |
| atgaatttga gtttgtgtat agcatctcca ctattgacca aatctaatag accagctgct | 60 |
| ttatcagcaa ttcatacagc tagtacatcc catggtggcc aaaccaaccc tacgaatctg | 120 |
| ataatcgata cgaccaagga gagaatacaa aaacaattca aaaatgttga aatttcagtt | 180 |
| tcttcttatg atactgcgtg ggttgccatg gttccatcac ctaattctcc aaagtctcca | 240 |
| tgtttcccag aatgtttgaa ttggctgatt aacaaccagt tgaatgatgg atcttggggt | 300 |
| ttagtcaatc acacgcacaa tcacaaccat ccacttttga aagattcttt atcctcaact | 360 |
| ttggcttgca tcgtggccct aaagagatgg aacgtaggtg aggatcagat taacaagggg | 420 |
| cttagtttca ttgaatctaa cttggcttcc gcgactgaaa aatctcaacc atctccaata | 480 |
| ggattcgata tcatctttcc aggtctgtta gagtacgcca aaaatctaga tatcaactta | 540 |
| ctgtctaagc aaactgattt ctcactaatg ttacacaaga gagaattaga acaaaagaga | 600 |
| tgtcattcaa acgaaatgga tggttaccta gcttatatct ctgaaggtct tggtaatctt | 660 |
| tacgattgga atatggtgaa aaagtaccag atgaaaaatg gctcagtttt caattcccct | 720 |
| tctgcaactg cggcagcatt cattaaccat caaaatccag gatgcctgaa ctatttgaat | 780 |
| tcactactag acaaattcgg caacgcagtt ccaactgtat accctcacga tttgtttatc | 840 |
| agattgagta tggtggatac aattgaaaga cttggtatat cccaccactt tagagtcgag | 900 |
| atcaaaaatg ttttggatga gacataccgt tgttgggtgg agagagatga acaaatcttt | 960 |
| atggatgttg tgacgtgcgc gttggccttt agattgttgc gtattaacgg ttacgaagtt | 1020 |
| agtccagatc cacttgccga aattacaaac gaattagctt taaaggatga atacgccgct | 1080 |
| cttgaaacat atcatgcgtc acatatcctt taccaagagg acttatcatc tggaaaacaa | 1140 |
| attcttaaat ctgctgattt cctgaaggaa atcatatcca ctgatagtaa tagactgtcc | 1200 |
| aaactgatcc ataaagaggt tgaaaatgca cttaagttcc ctattaacac cggcttagaa | 1260 |
| cgtattaaca caagacgtaa catccagctt tacaacgtag acaatactag aatcttgaaa | 1320 |
| accacttacc attcttccaa catatcaaac actgattacc taagattagc tgttgaagat | 1380 |
| ttctacacat gtcagtctat ctatagagaa gagctgaaag gattagagag atgggtcgtt | 1440 |
| gagaataagc tagatcaatt gaaatttgcc agacaaaaga cagcttattg ttacttctca | 1500 |
| gttgccgcca ctttatcaag tccagaattg tcagatgcac gtatttcttg ggctaaaaac | 1560 |
| ggaattttga caactgttgt tgatgatttc tttgatattg gcgggacaat cgacgaattg | 1620 |
| acaaacctga ttcaatgcgt tgaaaagtgg aatgtcgatg tcgataaaga ctgttgctca | 1680 |
| gaacatgtta gaatactgtt cttggctctg aaagatgcta tctgttggat cggggatgag | 1740 |
| gctttcaaat ggcaagctag agatgtgacg tctcacgtca ttcaaacctg gctagaactg | 1800 |
| atgaactcta tgttgagaga agcaatttgg actagagatg catacgttcc tacattaaac | 1860 |
| gagtatatgg aaaacgctta tgtctccttt gctttgggtc ctatcgttaa gcctgccata | 1920 |
| tactttgtag gaccaaagct atccgaggaa atcgtcgaat catcagaata ccataacttg | 1980 |
| ttcaagttaa tgtccacaca aggcagatta cttaatgata ttcattcttt caaaagagag | 2040 |
| tttaaggaag gaaagttaaa tgctgttgct ctgcatcttt ctaatggcga aagtggtaaa | 2100 |
| gtcgaagagg aagtagttga ggaaatgatg atgatgatca aaaacaagag aaaggagttg | 2160 |
| atgaaactaa tcttcgaaga gaacggttca attgttccta gagcatgtaa ggatgcattt | 2220 |
| tggaacatgt gtcatgtgct aaactttttc tacgcaaacg acgatggttt tactgggaac | 2280 |
| acaatactag atacagtaaa agacatcata tacaaccctt tggtcttagt aaacgaaaac | 2340 |
| gaggagcaaa gataa | 2355 |
| SEQ ID NO: 44 | |
| MNLSLCIASP LLTKSNRPAA LSAIHTASTS HGGQTNPTNL IIDTTKERIQ KQFKNVEISV | 60 |
| SSYDTAWVAM VPSPNSPKSP CFPECLNWLI NNQLNDGSWG LVNHTHNHNH PLLKDSLSST | 120 |
| LACIVALKRW NVGEDQINKG LSFIESNLAS ATEKSQPSPI GFDIIFPGLL EYAKNLDINL | 180 |
| LSKQTDFSLM LHKRELEQKR CHSNEMDGYL AYISEGLGNL YDWNMVKKYQ MKNGSVFNSP | 240 |
| SATAAAFINH QNPGCLNYLN SLLDKFGNAV PTVYPHDLEI RLSMVDTIER LGISHHFRVE | 300 |
| IKNVLDETYR CWVERDEQIF MDVVTCALAF RLLRINGYEV SPDPLAEITN ELALKDEYAA | 360 |
| LETYHASHIL YQEDLSSGKQ ILKSADFLKE IISTDSNRLS KLIHKEVENA LKFPINTGLE | 420 |
| RINTRRNIQL YNVDNTRILK TTYHSSNISN TDYLRLAVED FYTCQSIYRE ELKGLERWVV | 480 |
| ENKLDQLKFA RQKTAYCYFS VAATLSSPEL SDARISWAKN GILTTVVDDF FDIGGTIDEL | 540 |
| TNLIQCVEKW NVDVDKDCCS EHVRILFLAL KDAICWIGDE AFKWQARDVT SHVIQTWLEL | 600 |
| MNSMLREAIW TRDAYVPTLN EYMENAYVSF ALGPIVKPAI YFVGPKLSEE IVESSEYHNL | 660 |
| FKLMSTQGRL LNDIHSFKRE FKEGKLNAVA LHLSNGESGK VEEEVVEEMM MMIKNKRKEL | 720 |
| MKLIFEENGS IVPRACKDAF WNMCHVLNFF YANDDGFTGN TILDTVKDII YNPLVLVNEN | 780 |
| EEQR | 784 |
| SEQ ID NO: 45 | |
| atgaatctgt ccctttgtat agctagtcca ctgttgacaa aatcttctag accaactgct | 60 |
| ctttctgcaa ttcatactgc cagtactagt catggaggtc aaacaaaccc aacaaatttg | 120 |
| ataatcgata ctactaagga gagaatccaa aagctattca aaaatgttga aatctcagta | 180 |
| tcatcttatg acaccgcatg ggttgcaatg gtgccatcac ctaattcccc aaaaagtcca | 240 |
| tgttttccag agtgcttgaa ttggttaatc aataatcagt taaacgatgg ttcttggggt | 300 |
| ttagtcaacc acactcataa ccacaatcat ccattattga aggactcttt atcatcaaca | 360 |
| ttagcctgta ttgttgcatt gaaaagatgg aatgtaggtg aagatcaaat caacaagggt | 420 |
| ttatcattca tagaatccaa tctagcttct gctaccgaca aatcacaacc atctccaatc | 480 |
| gggttcgaca taatcttccc tggtttgctg gagtatgcca aaaaccttga tatcaactta | 540 |
| ctgtctaaac aaacagattt ctctttgatg ctacacaaaa gagagttaga gcagaaaaga | 600 |
| tgccattcta acgaaattga cgggtactta gcatatatct cagaaggttt gggtaatttg | 660 |
| tatgactgga acatggtcaa aaagtatcag atgaaaaatg gatccgtatt caattctcct | 720 |
| tctgcaactg ccgcagcatt cattaatcat caaaaccctg ggtgtcttaa ctacttgaac | 780 |
| tcactattag ataagtttgg aaatgcagtt ccaacagtct atcctttgga cttgtacatc | 840 |
| agattatcta tggttgacac tatagagaga ttaggtattt ctcatcattt cagagttgag | 900 |
| atcaaaaatg ttttggacga gacatacaga tgttgggtcg aaagagatga gcaaatcttt | 960 |
| atggatgtcg tgacctgcgc tctggctttt agattgctaa ggatacacgg atacaaagta | 1020 |
| tctcctgatc aactggctga gattacaaac gaactggctt tcaaagacga atacgccgca | 1080 |
| ttagaaacat accatgcatc ccaaatactt taccaggaag acctaagttc aggaaaacaa | 1140 |
| atcttgaagt ctgcagattt cctgaaaggc attctgtcta cagatagtaa taggttgtct | 1200 |
| aaattgatac acaaggaagt agaaaacgca ctaaagtttc ctattaacac tggtttagag | 1260 |
| agaatcaata ctaggagaaa cattcagctg tacaacgtag ataatacaag gattcttaag | 1320 |
| accacctacc atagttcaaa catttccaac acctattact taagattagc tgtcgaagac | 1380 |
| ttttacactt gtcaatcaat ctacagagag gagttaaagg gcctagaaag atgggtagtt | 1440 |
| caaaacaagt tggatcaact gaagtttgct agacagaaga cagcatactg ttatttctct | 1500 |
| gttgctgcta ccctttcatc cccagaattg tctgatgcca gaataagttg ggccaaaaat | 1560 |
| ggtattctta caactgtagt cgatgatttc tttgatattg gaggtactat tgatgaactg | 1620 |
| acaaatctta ttcaatgtgt tgaaaagtgg aacgtggatg tagataagga ttgctgcagt | 1680 |
| gaacatgtga gaatactttt cctggctcta aaagatgcaa tatgttggat tggcgacgag | 1740 |
| gccttcaagt ggcaagctag agatgttaca tctcatgtca tccaaacttg gcttgaactg | 1800 |
| atgaactcaa tgctaagaga agcaatctgg acaagagatg catacgttcc aacattgaac | 1860 |
| gaatacatgg aaaacgctta cgtctcattt gccttgggtc ctattgttaa gccagccata | 1920 |
| tactttgttg ggccaaagtt atccgaagag attgttgagt cttccgaata tcataaccta | 1980 |
| ttcaagttaa tgtcaacaca aggcagactt ctgaacgata tccactcctt caaaagagaa | 2040 |
| ttcaaggaag gtaagctaaa cgctgttgct ttgcacttgt ctaatggtga atctggcaaa | 2100 |
| gtggaagagg aagtcgttga ggaaatgatg atgatgatca aaaacaagag aaaggaattg | 2160 |
| atgaaattga ttttcgagga aaatggttca atcgtaccta gagcttgtaa agatgctttt | 2220 |
| tggaatatgt gccatgttct taacttcttt tacgctaatg atgatggctt cactggaaat | 2280 |
| acaatattgg atacagttaa agatatcatc tacaacccac ttgttttggt caatgagaac | 2340 |
| gaggaacaaa gataa | 2355 |
| SEQ ID NO: 46 | |
| MNLSLCIASP LLTKSSRPTA LSAIHTASTS HGGQTNPTNL IIDTTKERIQ KLFKNVEISV | 60 |
| SSYDTAWVAM VPSPNSPKSP CFPECLNWLI NNQLNDGSWG LVNHTHNHNH PLLKDSLSST | 120 |
| LACIVALKRW NVGEDQINKG LSFIESNLAS ATDKSQPSPI GFDIIFPGLL EYAKNLDINL | 180 |
| LSKQTDFSLM LHKRELEQKR CHSNEIDGYL AYISEGLGNL YDWNMVKKYQ MKNGSVFNSP | 240 |
| SATAAAFINH QNPGCLNYLN SLLDKFGNAV PTVYPLDLYI RLSMVDTIER LGISHHFRVE | 300 |
| IKNVLDETYR CWVERDEQIF MDVVTCALAF RLLRIHGYKV SPDQLAEITN ELAFKDEYAA | 360 |
| LETYHASQIL YQEDLSSGKQ ILKSADFLKG ILSTDSNRLS KLIHKEVENA LKFPINTGLE | 420 |
| RINTRRNIQL YNVDNTRILK TTYHSSNISN TYYLRLAVED FYTCQSIYRE ELKGLERWVV | 480 |
| QNKLDQLKFA RQKTAYCYFS VAATLSSPEL SDARISWAKN GILTTVVDDF FDIGGTIDEL | 540 |
| TNLIQCVEKW NVDVDKDCCS EHVRILFLAL KDAICWIGDE AFKWQARDVT SHVIQTWLEL | 600 |
| MNSMLREAIW TRDAYVPTLN EYMENAYVSF ALGPIVKPAI YFVGPKLSEE IVESSEYHNL | 660 |
| FKLMSTQGRL LNDIHSFKRE FKEGKLNAVA LHLSNGESGK VEEEVVEEMM MMIKNKRKEL | 720 |
| MKLIFEENGS IVPRACKDAF WNMCHVLNFF YANDDGFTGN TILDTVKDII YNPLVLVNEN | 780 |
| EEQR | 784 |
| SEQ ID NO: 47 | |
| atggctatgc cagtgaagct aacacctgcg tcattatcct taaaagctgt gtgctgcaga | 60 |
| ttctcatccg gtggccatgc tttgagattc gggagtagtc tgccatgttg gagaaggacc | 120 |
| cctacccaaa gatctacttc ttcctctact actagaccag ctgccgaagt gtcatcaggt | 180 |
| aagagtaaac aacatgatca ggaagctagt gaagcgacta tcagacaaca attacaactt | 240 |
| gtggatgtcc tggagaatat gggaatatcc agacattttg ctgcagagat aaagtgcata | 300 |
| ctagacagaa cttacagatc ttggttacaa agacacgagg aaatcatgct ggacactatg | 360 |
| acatgtgcta tggcttttag aatcctaaga ttgaacggat acaacgtttc atcagatgaa | 420 |
| ctataccacg ttgtagaggc atctggtctg cataattctt tgggtgggta tcttaacgat | 480 |
| accagaacac tacttgaatt acacaaggct tcaacagtta gtatctctga ggatgaatct | 540 |
| atcttagatt caattggctc tagatccaga acattgctta gagaacaatt ggagtctggt | 600 |
| ggcgcactga gaaagccttc tttattcaaa gaggttgaac atgcactgga tggacctttt | 660 |
| tacaccacac ttgatagact tcatcatagg tggaatattg aaaacttcaa cattattgag | 720 |
| caacacatgt tggagactcc atacttatct aaccagcata catcaaggga tatcctagca | 780 |
| ttgtcaatta gagatttttc ctcctcacaa ttcacttatc aacaagagct acagcatctg | 840 |
| gagagttggg ttaaggaatg tagattagat caactacagt tcgcaagaca gaaattagcg | 900 |
| tacttttacc tatcagccgc aggcaccatg ttttctcctg agctttctga tgcgagaaca | 960 |
| ttatgggcca aaaacggggt gttgacaact attgttgatg atttctttga tgttgccggt | 1020 |
| tctaaagagg aattggaaaa cttagtcatg ctggtcgaaa tgtgggatga acatcacaaa | 1080 |
| gttgaattct attctgagca ggtcgaaatc atcttctctt ccatctacga ttctgtcaac | 1140 |
| caattgggtg agaaggcctc tttggttcaa gacagatcaa ttacaaaaca ccttgttgaa | 1200 |
| atatggttag acttgttaaa gtccatgatg acggaagttg aatggagact gtcaaaatac | 1260 |
| gtgcctacag aaaaggaata catgattaat gcctctctta tcttcggcct aggtccaatc | 1320 |
| gttttaccag ctttgtattt cgttggtcca aagatttcag aaagtatagt aaaggaccca | 1380 |
| gaatatgatg aattgttcaa actaatgtca acatgtggta gattgttgaa tgacgtgcaa | 1440 |
| acgttcgaaa gagaatacaa tgagggtaaa ctgaattctg tcagtctatt ggttcttcac | 1500 |
| ggaggcccaa tgtctatttc agacgcaaag aggaaattac aaaagcctat tgatacgtgt | 1560 |
| agaagagatc ttctttcttt ggtccttaga gaagagtctg tagtaccaag accatgtaag | 1620 |
| gaactattct ggaaaatgtg taaagtgtgc tatttctttt actcaacaac tgatgggttt | 1680 |
| tctagtcaag tcgaaagagc aaaagaggta gacgctgtca taaatgagcc actgaagttg | 1740 |
| caaggttctc atacactggt atctgatgtt taa | 1773 |
| SEQ ID NO: 48 | |
| MAMPVKLTPA SLSLKAVCCR FSSGGHALRF GSSLPCWRRT PTQRSTSSST TRPAAEVSSG | 60 |
| KSKQHDQEAS EATIRQQLQL VDVLENMGIS RHFAAEIKCI LDRTYRSWLQ RHEEIMLDTM | 120 |
| TCAMAFRILR LNGYNVSSDE LYHVVEASGL HNSLGGYLND TRTLLELHKA STVSISEDES | 180 |
| ILDSIGSRSR TLLREQLESG GALRKPSLFK EVEHALDGPF YTTLDRLHHR WNIENFNIIE | 240 |
| QHMLETPYLS NQHTSRDILA LSIRDFSSSQ FTYQQELQHL ESWVKECRLD QLQFARQKLA | 300 |
| YFYLSAAGTM FSPELSDART LWAKNGVLTT IVDDFFDVAG SKEELENLVM LVEMWDEHHK | 360 |
| VEFYSEQVEI IFSSIYDSVN QLGEKASLVQ DRSITKHLVE IWLDLLKSMM TEVEWRLSKY | 420 |
| VPTEKEYMIN ASLIFGLGPI VLPALYFVGP KISESIVKDP EYDELFKLMS TCGRLLNDVQ | 480 |
| TFEREYNEGK LNSVSLLVLH GGPMSISDAK RKLQKPIDTC RRDLLSLVLR EESVVPRPCK | 540 |
| ELFWKMCKVC YFFYSTTDGF SSQVERAKEV DAVINEPLKL QGSHTLVSDV | 590 |
| SEQ ID NO: 49 | |
| atgcagaact tccatggtac aaaggaaagg atcaaaaaga tgtttgacaa gattgaattg | 60 |
| tccgtttctt cttatgatac agcctgggtt gcaatggtcc catcccctga ttgcccagaa | 120 |
| acaccttgtt ttccagaatg tactaaatgg atcctagaaa atcagttggg tgatggtagt | 180 |
| tggtcacttc ctcatggcaa tccacttcta gttaaagatg cattatcttc cactcttgct | 240 |
| tgtattctgg ctcttaaaag atggggaatc ggtgaggaac agattaacaa aggactgaga | 300 |
| ttcatagaac tcaactctgc tagtgtaacc gataacgaac aacacaaacc aattggattt | 360 |
| gacattatct ttccaggtat gattgaatac gctatagact tagacctgaa tctaccacta | 420 |
| aaaccaactg acattaactc catgttgcat cgtagagccc ttgaattgac atcaggtgga | 480 |
| ggcaaaaatc tagaagytag aagagcttac ttggcctacg tctctgaagg aatcggtaag | 540 |
| ctgcaagatt gggaaatggc tatgaaatac caacgtaaaa acggatctct gttcaatagt | 600 |
| ccatcaacaa ctgcagctgc attcatccat atacaagatg ctgaatgcct ccactatatt | 660 |
| cgttctcttc tccagaaatt tggaaacgca gtccctacaa tataccctct cgatatctat | 720 |
| gccagacttt caatggtaga tgccctggaa cgtcttggta ttgatagaca tttcagaaag | 780 |
| gagagaaagt tcgttctgga tgaaacatac agattttggt tgcaaggaga agaggagatt | 840 |
| ttctccgata acgcaacctg tgctttggcc ttcagaatat tgagacttaa tggttacgat | 900 |
| gtctctcttg aagatcactt ctctaactct ctgggcggtt acttaaagga ctcaggagca | 960 |
| gctttagaac tgtacagagc cctccaattg tcttacccag acgagtccct cctggaaaag | 1020 |
| caaaattcta gaacttctta cttcttaaaa caaggtttat ccaatgtctc cctctgtggt | 1080 |
| gacagattgc gtaaaaacat aattggagag gtgcatgatg ctttaaactt ttccgaccac | 1140 |
| gctaacttac aaagattagc tattcgtaga aggattaagc attacgctac tgacgataca | 1200 |
| aggattctaa aaacttccta cagatgctca acaatcggta accaagattt tctaaaactt | 1260 |
| gcagtggaag atttcaatat ctgtcaatca atacaaagag aggaattcaa gcatattgaa | 1320 |
| agatgggtcg ttgaaagacg tctagacaag ttaaagttcg ctagacaaaa agaggcctat | 1380 |
| tgctatttct cagccgcagc aacattgttt gcccctgaat tgtctgatgc tagaatgtct | 1440 |
| tgggccaaaa atggtgtatt gacaactgtg gttgatgatt tcttcgatgt cggaggctct | 1500 |
| gaagaggaat tagttaactt gatagaattg atcgagcgtt gggatgtgaa tggcagtgca | 1560 |
| gatttttgta gtgaggaagt tgagattatc tattctgcta tccactcaac tatctctgaa | 1620 |
| ataggtgata agtcatttgg ctggcaaggt agagatgtaa agtctcaagt tatcaagatc | 1680 |
| tggctggact tattgaaatc aatgttaact gaagctcaat ggtcttcaaa caagtctgtt | 1740 |
| cctaccctag atgagtatat gacaaccgcc catgtttcat tcgcacttgg tccaattgta | 1800 |
| cttccagcct tatacttcgt tggcccaaag ttgtcagaag aggttgcagg tcatcctgaa | 1860 |
| ctactaaacc tctacaaagt cacatctact tgtggcagac tactgaatga ttggagaagt | 1920 |
| tttaagagag aatccgagga aggtaagctc aacgctatta gtttatacat gatccactcc | 1980 |
| ggtggtgctt ctacagaaga ggaaacaatc gaacatttca aaggtttgat tgattctcag | 2040 |
| agaaggcaac tgttacaatt ggtgttgcaa gagaaggata gtatcatacc tagaccatgt | 2100 |
| aaagatctat tttggaatat gattaagtta ttacacactt tctacatgaa agatgatggc | 2160 |
| ttcacctcaa atgagatgag gaatgtagtt aaggcaatca ttaacgaacc aatctcactg | 2220 |
| gatgaattat ga | 2232 |
| SEQ ID NO: 50 | |
| MSCIRPWFCP SSISATLTDP ASKLVTGEFK TTSLNFHGTK ERIKKMFDKI ELSVSSYDTA | 60 |
| WVAMVPSPDC PETPCFPECT KWILENQLGD GSWSLPHGNP LLVKDALSST LACILALKRW | 120 |
| GIGEEQINKG LRFIELNSAS VTDNEQHKPI GFDIIFPGMI EYAKDLDLNL PLKPTDINSM | 180 |
| LHRRALELTS GGGKNLEGRR AYLAYVSEGI GKLQDWEMAM KYQRKNGSLF NSPSTTAAAF | 240 |
| IHIQDAECLH YIRSLLQKFG NAVPTIYPLD IYARLSMVDA LERLGIDRHF RKERKFVLDE | 300 |
| TYREWLQGEE EIFSDNATCA LAFRILRLNG YDVSLEDHFS NSLGGYLKDS GAALELYRAL | 360 |
| QLSYPDESLL EKQNSRTSYF LKQGLSNVSL CGDRLRKNII GEVHDALNFP DHANLQRLAI | 420 |
| RRRIKHYATD DTRILKTSYR CSTIGNQDFL KLAVEDFNIC QSIQREEFKH IERWVVERRL | 480 |
| DKLKFARQKE AYCYFSAAAT LEAPELSDAR MSWAKNGVLT TVVDDFFDVG GSEEELVNLI | 540 |
| ELIERWDVNG SADFCSEEVE IIYSAIHSTI SEIGDKSFGW QGRDVKSHVI KIWLDLLKSM | 600 |
| LTEAQWSSNK SVPTLDEYMT TAHVSFALGP IVLPALYFVG PKLSEEVAGH PELLNLYKVM | 660 |
| STCGRLLNDW RSFKRESEEG KLNAISLYMI HSGGASTEEE TIEHFKGLID SQRRQLLQLV | 720 |
| LQEKDSIIPR PCKDLFWNMI KLLHTFYMKD DGFTSNEMRN VVKAIINEPI SLDEL | 775 |
| SEQ ID NO: 51 | |
| atgtctatca accttcgctc ctccggttgt tcgtctccga tctcagctac tttggaacga | 60 |
| ggattggact cagaagtaca gacaagagct aacaatgtga gctttgagca aacaaaggag | 120 |
| aagattagga agatgttgga gaaagtggag ctttctgttt cggcctacga tactagttgg | 180 |
| gtagcaatgg ttccatcacc gagctcccaa aatgctccac ttttcccaca gtgtgtgaaa | 240 |
| tggttattgg ataatcaaca tgaagatgga tcttggggac ttgataacca tgaccatcaa | 300 |
| tctcttaaga aggatgtgtt atcatctaca ctggctagta tcctcgcgtt aaagaagtgg | 360 |
| ggaattggtg aaagacaaat aaacaagggt ctccagttta ttgagctgaa ttctgcatta | 420 |
| gtcactgatg aaaccataca gaaaccaaca gggtttgata ttatatttcc tgggatgatt | 480 |
| aaatatgcta gagatttgaa tctgacgatt ccattgggct cagaagtggt ggatgacatg | 540 |
| atacgaaaaa gagatctgga tcttaaatgt gatagtgaaa agttttcaaa gggaagagaa | 600 |
| gcatatctgg cctatgtttt agaggggaca agaaacctaa aagattggga tttgatagtc | 660 |
| aaatatcaaa ggaaaaatgg gtcactgttt gattctccag ccacaacagc agctgctttt | 720 |
| actcagtttg ggaatgatgg ttgtctccgt tatctctgtt ctctccttca gaaattcgag | 780 |
| gctgcagttc cttcagttta tccatttgat caatatgcac gccttagtat aattgtcact | 840 |
| cttgaaagct taggaattga tagagatttc aaaaccgaaa tcaaaagcat attggatgaa | 900 |
| acctatagat attggcttcg tggggatgaa gaaatatgtt tggacttggc cacttgtgct | 960 |
| ttggctttcc gattattgct tgctcatggc tatgatgtgt cttacgatcc gctaaaacca | 1020 |
| tttgcagaag aatctggttt ctctgatact ttggaaggat atgttaagaa tacgttttct | 1080 |
| gtgttagaat tatttaaggc tgctcaaagt tatccacatg aatcagcttt gaagaagcag | 1140 |
| tgttgttgga ctaaacaata tctggagatg gaattgtcca gctgggttaa gacctctgtt | 1200 |
| cgagataaat acctcaagaa agaggtcgag gatgctcttg cttttccctc ctatgcaagc | 1260 |
| ctagaaagat cagatcacag gagaaaaata ctcaatggtt ctgctgtgga aaacaccaga | 1320 |
| gttacaaaaa cctcatatcg tttgcacaat atttgcacct ctgatatcct gaagttagct | 1380 |
| gtggatgact tcaatttctg ccagtccata caccgtgaag aaatggaacg tcttgatagg | 1440 |
| tggattgtgg agaatagatt gcaggaactg aaatttgcca gacagaagct ggcttactgt | 1500 |
| tatttctctg gggctgcaac tttattttct ccagaactat ctgatgctcg tatatcgtgg | 1560 |
| gccaaaggtg gagtacttac aacggttgta gacgacttct ttgatgttgg agggtccaaa | 1620 |
| gaagaactgg aaaacctcat acacttggtc gaaaagtggg atttgaacgg tgttcctgag | 1680 |
| tacagctcag aacatgttga gatcatattc tcagttctaa gggacaccat tctcgaaaca | 1740 |
| ggagacaaag cattcaccta tcaaggacgc aatgtgacac accacattgt gaaaatttgg | 1800 |
| ttggatctgc tcaagtctat gttgagagaa gccgagtggt ccagtgacaa gtcaacacca | 1860 |
| agcttggagg attacatgga aaatgcgtac atatcatttg cattaggacc aattgtcctc | 1920 |
| ccagctacct atctgatcgg acctccactt ccagagaaga cagtcgatag ccaccaatat | 1980 |
| aatcagctct acaagctcgt gagcactatg ggtcgtcttc taaatgacat acaaggtttt | 2040 |
| aagagagaaa gcgcggaagg gaagctgaat gcggtttcat tgcacatgaa acacgagaga | 2100 |
| gacaatcgca gcaaagaagt gatcatagaa tcgatgaaag gtttagcaga gagaaagagg | 2160 |
| gaagaattgc ataagctagt tttggaggag aaaggaagtg tggttccaag ggaatgcaaa | 2220 |
| gaagcgttct tgaaaatgag caaagtgttg aacttatttt acaggaagga cgatggattc | 2280 |
| acatcaaatg atctgatgag tcttgttaaa tcagtgatct acgagcctgt tagcttacag | 2340 |
| aaagaatctt taacttga | 2358 |
| SEQ ID NO: 52 | |
| MSINLRSSGC SSPISATLER GLDSEVQTRA NNVSFEQTKE KIRKMLEKVE LSVSAYDTSW | 60 |
| VAMVPSPSSQ NAPLFPQCVK WLLDNQHEDG SWGLDNHDHQ SLKKDVLSST LASILALKKW | 120 |
| GIGERQINKG LQFIELNSAL VTDETIQKPT GFDIIFPGMI KYARDLNLTI PLGSEVVDDM | 180 |
| IRKRDLDLKC DSEKFSKGRE AYLAYVLEGT RNLKDWDLIV KYQRKNGSLF DSPATTAAAF | 240 |
| TQFGNDGCLR YLCSLLQKFE AAVPSVYPFD QYARLSIIVT LESLGIDRDF KTEIKSILDE | 300 |
| TYRYWLRGDE EICLDLATCA LAFRLLLAHG YDVSYDPLKP FAEESGFSDT LEGYVKNTFS | 360 |
| VLELFKAAQS YPHESALKKQ CCWIKQYLEM ELSSWVKTSV RDKYLKKEVE DALAFPSYAS | 420 |
| LERSDHRRKI LNGSAVENTR VIKTSYRLHN ICTSDILKLA VDDFNFCQSI HREEMERLDR | 480 |
| WIVENRLQEL KFARQKLAYC YFSGAATLFS PELSDARISW AKGGVLTTVV DDFFDVGGSK | 540 |
| EELENLIHLV EKWDLNGVPE YSSEHVEIIF SVLRDTILET GDKAFTYQGR NVTHHIVKIW | 600 |
| LDLLKSMLRE AEWSSDKSTP SLEDYMENAY ISFALGPIVL PATYLIGPPL PEKTVDSHQY | 660 |
| NQLYKLVSTM GRLLNDIQGF KRESAEGKLN AVSLHMKHER DNRSKEVIIE SMKGLAERKR | 720 |
| EELHKLVLEE KGSVVPRECK EAFLKMSKVL NLFYRKDDGF TSNDLMSLVK SVIYEPVSLQ | 780 |
| KESLT | 785 |
| SEQ ID NO: 53 | |
| atggaatttg atgaaccatt ggttgacgaa gcaagatctt tagtgcagcg tactttacaa | 60 |
| gattatgatg acagatacgg cttcggtact atgtcatgtg ctgcttatga tacagcctgg | 120 |
| gtgtctttag ttacaaaaac agtcgatggg agaaaacaat ggcttttccc agagtgtttt | 180 |
| gaatttctac tagaaacaca atctgatgcc ggaggatggg aaatcgggaa ttcagcacca | 240 |
| atcgacggta tattgaatac agctgcatcc ttacttgctc taaaacgtca cgttcaaact | 300 |
| gagcaaatca tccaacctca acatgaccat aaggatctag caggtagagc tgaacgtgcc | 360 |
| gctgcatctt tgagagcaca attggctgca ttggatgtgt ctacaactga acacgtcggt | 420 |
| tttgagataa Ltgttcctgc aatgctagac ccattagaag ccgaagatcc atctctagtt | 480 |
| ttcgattttc cagctaggaa acctttgatg aagattcatg atgctaagat gagtagattc | 540 |
| aggccagaat acttgtatgg caaacaacca atgaccgcct tacattcatt agaggctttc | 600 |
| ataggcaaaa tcgacttcga taaggtaaga caccaccgta cccatgggtc tatgatgggt | 660 |
| tctccttcat ctaccgcagc ctacttaatg cacgcttcac aatgggatgg tgactcagag | 720 |
| gcttacctta gacacgtgat taaacacgca gcagggcagg gaactggtgc tgtaccatct | 780 |
| gctttcccat caacacattt tgagtcatct tggattctta ccacattgtt tagagctgga | 840 |
| ttttcagctt ctcatcttgc ctgtgatgag ttgaacaagt tggtcgagat acttgagggc | 900 |
| tcattcgaga aggaaggtgg ggcaatcggt tacgctccag ggtttcaagc agatgttgat | 960 |
| gatactgcta aaacaataag tacattagca gtccttggaa gagatgctac accaagacaa | 1020 |
| atgatcaagg tatttgaagc taatacacat tttagaacat accctggtga aagagatcct | 1080 |
| tctttgacag ctaattgtaa tgctctatca gccttactac accaaccaga tgcagcaatg | 1140 |
| tatggatctc aaattcaaaa gattaccaaa tttgtctgtg actattggtg gaagtctgat | 1200 |
| ggtaagatta aagataagtg gaacacttgc tacttgtacc catctgtctt attagttgag | 1260 |
| gttttggttg atcttgttag tttattggag cagggtaaat tgcctgatgt tttggatcaa | 1320 |
| gagcttcaat acagagtcgc catcacattg ttccaagcat gtttaaggcc attactagac | 1380 |
| caagatgccg aaggatcatg gaacaagtct atcgaagcca cagcctacgg catccttatc | 1440 |
| ctaactgaag ctaggagagt ttgtttcttc gacagattgt ctgagccatt gaatgaggca | 1500 |
| atccgtagag gtatcgcttt cgccgactct atgtctggaa ctgaagctca gttgaactac | 1560 |
| atttggatcg aaaaggttag ttacgcacct gcattattga ctaaatccta tttgttagca | 1620 |
| gcaagatggg ctgctaagtc tcctttaggc gcttccgtag gctcttcttt gtggactcca | 1680 |
| ccaagagaag gattggataa gcatgtcaga ttattccatc aagctgagtt attcagatcc | 1740 |
| cttccagaat gggaattaag agcctccatg attgaagcag ctttgttcac accacttcta | 1800 |
| agagcacata gactagacgt tttccctaga caagatgtag gtgaagacaa atatcttgat | 1860 |
| gtagttccat tcttttggac tgccgctaac aacagagata gaacttacgc ttccactcta | 1920 |
| ttcctttacg atatgtgttt tatcgcaatg ttaaacttcc agttagacga attcatggag | 1980 |
| gccacagccg gtatcttatt cagagatcat atggatgatt tgaggcaatt gattcatgat | 2040 |
| cttttggcag agaaaacttc cccaaagagt tctggtagaa gtagtcaggg cacaaaagat | 2100 |
| gctgactcag gtatagagga agacgtgtca atgtccgatt cagcttcaga ttcccaggat | 2160 |
| agaagtccag aatacgactt ggttttcagt gcattgagta cctttacaaa acatgtcttg | 2220 |
| caacacccat ctatacaaag tgcctctgta tgggatagaa aactacttgc tagagagatg | 2280 |
| aaggcttact tacttgctca tatccaacaa gcagaagatt caactccatt gtctgaattg | 2340 |
| aaagatgtgc ctcaaaagac tgatgtaaca agagtttcta catctactac taccttcttt | 2400 |
| aactgggtta gaacaacttc cgcagaccat atatcctgcc catactcctt ccactttgta | 2460 |
| gcatgccatc taggcgcagc attgtcacct aaagggtcta acggtgattg ctatccttca | 2520 |
| gctggtgaga agttcttggc agctgcagtc tgcagacatt tggccaccat gtgtagaatg | 2580 |
| tacaacgatc ttggatcagc tgaacgtgat tctgatgaag gtaatttgaa ctccttggac | 2640 |
| ttccctgaat tcgccgattc cgcaggaaac ggagggatag aaattcagaa ggccgctcta | 2700 |
| ttaaggttag ctgagtttga gagagattca tacttagagg ccttccgtcg tttacaagat | 2760 |
| gaatccaata gagttcacgg tccagccggt ggtgatgaag ccagattgtc cagaaggaga | 2820 |
| atggcaatcc ttgaattctt cgcccagcag gtagatttgt acggtcaagt atacgtcatt | 2880 |
| agggatattt ccgctcgtat tcctaaaaac gaggttgaga aaaagagaaa attggatgat | 2940 |
| gctttcaatt ga | 2952 |
| SEQ ID NO: 54 | |
| MEFDEPLVDE ARSLVQRTLQ DYDDRYGFGT MSCAAYDTAW VSLVTKTVDG RKQWLEPECF | 60 |
| EFLLETQSDA GGWEIGNSAP IDGILNTAAS LLALKRHVQT EQIIQPQHDH KDLAGRAERA | 120 |
| AASLRAQLAA LDVSTTEHVG FEIIVPAMLD PLEAEDPSLV FDFPARKPLM KIHDAKMSRF | 180 |
| RPEYLYGKQP MTALHSLEAF IGKIDFDKVR HHRTHGSMMG SPSSTAAYLM HASQWDGDSE | 240 |
| AYLRHVIKHA AGQGTGAVPS AFPSTHFESS WILTTLFRAG FSASHLACDE LNKLVEILEG | 300 |
| SFEKEGGAIG YAPGFQADVD DTAKTISTLA VLGRDATPRQ MIKVFEANTH FRTYPGERDP | 360 |
| SLTANCNALS ALLHQPDAAM YGSQIQKITK FVCDYWWKSD GKIKDKWNTC YLYPSVLLVE | 420 |
| VLVDLVSLLE QGKLPDVLDQ ELQYRVAITL FQACLRPLLD QDAEGSWNKS IEATAYGILI | 480 |
| LTEARRVCFF DRLSEPLNEA IRRGIAFADS MSGTEAQLNY IWIEKVSYAP ALLTKSYLLA | 540 |
| ARWAAKSPLG ASVGSSLWTP PREGLDKHVR LFHQAELFRS LPEWELRASM IEAALFTPLL | 600 |
| RAHRLDVFPR QDVGEDKYLD VVPFFWTAAN NRDRTYASTL FLYDMCFIAM LNFQLDEFME | 660 |
| ATAGILFRDH MDDLRQLIHD LLAEKTSPKS SGRSSQGTKD ADSGIEEDVS MSDSASDSQD | 720 |
| RSPEYDLVFS ALSTFTKHVL QHPSIQSASV WDRKLLAREM KAYLLAHIQQ AEDSTPLSEL | 780 |
| KDVPQKTDVT RVSTSTTTFF NWVRTTSADH ISCPYSFHFV ACHLGAALSP KGSNGDCYPS | 840 |
| AGEKFLAAAV CRHLATMCRM YNDLGSAERD SDEGNLNSLD FPEFADSAGN GGIEIQKAAL | 900 |
| LRLAEFERDS YLEAFRRLQD ESNRVHGPAG GDEARLSRRR MAILEFFAQQ VDLYGQVYVI | 960 |
| RDISARIPKN EVEKKRKLDD AFN | 983 |
| SEQ ID NO: 55 | |
| atggcttcta gtacacttat ccaaaacaga tcatgtggcg tcacatcatc tatgtcaagt | 60 |
| tttcaaatct tcagaggtca accactaaga tttcctggca ctagaacccc agctgcagtt | 120 |
| caatgcttga aaaagaggag atgccttagg ccaaccgaat ccgtactaga atcatctcct | 180 |
| ggctctggtt catatagaat agtaactggc ccttctggaa ttaaccctag ttctaacggg | 240 |
| cacttgcaag agggttcctt gactcacagg ttaccaatac caatggaaaa atctatcgat | 300 |
| aacttccaat ctactctata tgtgtcagat atttggtctg aaacactaca gagaactgaa | 360 |
| tgtttgctac aagtaactga aaacgtccag atgaatgagt ggattgagga aattagaatg | 420 |
| tactttagaa atatgacttt aggtgaaatt tccatgtccc cttacgacac tgcttgggtg | 480 |
| gctagagttc cagcgttgga cggttctcat gggcctcaat tccacagatc tttgcaatgg | 540 |
| attatcgaca accaattacc agatggggac tggggcgaac cttctctttt cttgggttac | 600 |
| gatagagttt gtaatacttt agcctgtgtg attgcgttga aaacatgggg tgttggggca | 660 |
| caaaacgttg aaagaggaat tcagttccta caatctaaca tatacaagat ggaggaagat | 720 |
| gacgctaatc atatgccaat aggattcgaa atcgtattcc ctgctatgat ggaagatgcc | 780 |
| aaagcattag gtttggattt gccatacgat gctactattt tgcaacagat ttcagccgaa | 840 |
| agagagaaaa agatgaaaaa gatcccaatg gcaatggtgt acaaataccc aaccacttta | 900 |
| cttcactcct tagaaggctt gcatagagaa gttgattgga ataagttgtt acaattacaa | 960 |
| tctgaaaatg gtagttttct ttattcacct gcttcaaccg catgcgcctt aatgtacact | 1020 |
| aaggacgtta aatgttttga ttacttaaac cagttgttga tcaagttcga ccacgcatgc | 1080 |
| ccaaatgtat atccagtcga tctattcgaa agattatgga tggttgacag attgcagaga | 1140 |
| ttagggatct ccagatactt tgaaagagag attagagatt gtttacaata cgtctacaga | 1200 |
| tattggaaag attgtggaat cggatgggct tctaactctt ccgtacaaga tgttgatgat | 1260 |
| acagccatgg cgtttagact tttaaggact catggtttcg acgtaaagga agattgcttt | 1320 |
| agacagtttt tcaaggacgg agaattcttc tgcttcgcag gccaatcatc tcaagcagtt | 1380 |
| acaggcatgt ttaatctttc aagagccagt caaacattgt ttccaggaga atctttattg | 1440 |
| aaaaaggcta gaaccttctc tagaaacttc ttgagaacaa agcatgagaa caacgaatgt | 1500 |
| ttcgataaat ggatcattac taaagatttg gctggtgaag tcgagtataa cttgaccttc | 1560 |
| ccatggtatg cctctttgcc tagattagaa cataggacat acttagatca atatggaatc | 1620 |
| gatgatatct ggataggcaa atctttatac aaaatgcctg ctgttaccaa cgaagttttc | 1680 |
| ctaaagttgg caaaggcaga ctttaacatg tgtcaagctc tacacaaaaa ggaattggaa | 1740 |
| caagtgataa agtggaacgc gtcctgtcaa ttcagagatc ttgaattcgc cagacaaaaa | 1800 |
| tcagtagaat gctattttgc tggtgcagcc acaatgttcg aaccagaaat ggttcaagct | 1860 |
| agattagtct gggcaagatg ttgtgtattg acaactgtct tagacgatta ctttgaccac | 1920 |
| gggacacctg ttgaggaact tagagtgttt gttcaagctg tcagaacatg gaatccagag | 1980 |
| ttgatcaacg gtttgccaga gcaagctaaa atcttgttta tgggcttata caaaacagtt | 2040 |
| aacacaattg cagaggaagc attcatggca cagaaaagag acgtccatca tcatttgaaa | 2100 |
| cactattggg acaagttgat aacaagtgcc ctaaaggagg ccgaatgggc agagtcaggt | 2160 |
| tacgtcccaa catttgatga atacatggaa gtagctgaaa tttctgttgc tctagaacca | 2220 |
| attgtctgta gtaccttgtt ctttgcgggt catagactag atgaggatgt tctagatagt | 2280 |
| tacgattacc atctagttat gcatttggta aacagagtcg gtagaatctt gaatgatata | 2340 |
| caaggcatga agagggaggc ttcacaaggt aagatctcat cagttcaaat ctacatggag | 2400 |
| gaacatccat ctgttccatc tgaggccatg gcgatcgctc atcttcaaga gttagttgat | 2460 |
| aattcaatgc agcaattgac atacgaagtt cttaggttca ctgcggttcc aaaaagttgt | 2520 |
| aagagaatcc acttgaatat ggctaaaatc atgcatgcct tctacaagga tactgatgga | 2580 |
| ttctcatccc ttactgcaat gacaggattc gtcaaaaagg ttcttttcga acctgtgcct | 2640 |
| gagtaa | 2646 |
| SEQ ID NO: 56 | |
| MASSTLIQNR SCGVTSSMSS FQIFRGQPLR FPGTRTPAAV QCLKKRRCLR PTESVLESSP | 60 |
| GSGSYRIVTG PSGINPSSNG HLQEGSLTHR LPIPMEKSID NFQSTLYVSD IWSETLQRTE | 120 |
| CLLQVTENVQ MNEWIEEIRM YFRNMTLGEI SMSPYDTAWV ARVPALuGSH GPQFHRSLQW | 180 |
| IIDNQLPDGD WGEPSLFLGY DRVCNTLACV IALKTWGVGA QNVERGIQFL QSNIYKMEED | 240 |
| DANHMPIGFE IVFPAMMEDA KALGLDLPYD ATILQQISAE REKKMKKIPM AMVYKYPTTL | 300 |
| LHSLEGLHRE VDWNKLLQLQ SENGSFLYSP ASTACALMYT KDVKCFDYLN QLLIKFDHAC | 360 |
| PNVYPVDLFE RLWMVDRLQR LGISRYFERE IRDCLQYVYR YWKDCGIGWA SNSSVQDVDD | 420 |
| TAMAFRLLRT HGFDVKEDCF RQFFKDGEFF CFAGQSSQAV TGMFNLSRAS QTLFPGESLL | 480 |
| KKARTFSRNF LRTKHENNEC FDKWIITKDL AGEVEYNLTF PWYASLPRLE HRTYLDQYGI | 540 |
| DDIWIGKSLY KMPAVTNEVF LKLAKADFNM CQALHKKELE QVIKWNASCQ FRDLEFARQK | 600 |
| SVECYFAGAA TMFEPEMVQA RLVWARCCVL TTVLDDYFDH GTPVEELRVF VQAVRTWNPE | 660 |
| LINGLPEQAK ILFMGLYKTV NTIAEEAFMA QKRDVHHHLK HYWDKLITSA LKEAEWAESG | 720 |
| YVPTFDEYME VAEISVALEP IVCSTLFFAG HRLDEDVLDS YDYHLVMHLV NRVGRILNDI | 780 |
| QGMKREASQG KISSVQIYME EHPSVPSEAM AIAHLQELVD NSMQQLTYEV LRFTAVPKSC | 840 |
| KRIHLNMAKI MHAFYKDTDG FSSLTAMTGF VKKVLFEPVP E | 881 |
| SEQ ID NO: 57 | |
| atgcctggta aaattgaaaa tggtacccca aaggacctca agactggaaa tgattttgtt | 60 |
| tctgctgcta agagtttact agatcgagct ttcaaaagtc atcattccta ctacggatta | 120 |
| tgctcaactt catgtcaagt ttatgataca gcttgggttg caatgattcc aaaaacaaga | 180 |
| gataatgtaa aacagtggtt gtttccagaa tgtttccatt acctcttaaa aacacaagcc | 240 |
| gcagatggct catggggttc attgcctaca acacagacag cgggtatcct agatacagcc | 300 |
| tcagctgtgc tggcattatt gtgccacgca caagagcctt tacaaatatt ggatgtatct | 360 |
| ccagatgaaa tggggttgag aatagaacac ggtgtcacat ccttgaaacg tcaattagca | 420 |
| gtttggaatg atgtggagga caccaaccat attggcgtcg agtttatcat accagcctta | 480 |
| ctttccatgc tagaaaagga attagatgtt ccatcttttg aatttccatg taggtccatc | 540 |
| ttagagagaa tgcacgggga gaaattaggt catttcgacc tggaacaagt ttacggcaag | 600 |
| ccaagctcat tgttgcactc attggaagca tttctcggta agctagattt tgatcgacta | 660 |
| tcacatcacc tataccacgg cagtatgatg gcatctccat cttcaacggc tgcttatctt | 720 |
| attggggcta caaaatggga tgacgaagcc gaagattacc taagacatgt aatgcgtaat | 780 |
| ggtgcaggac atgggaatgg aggtatttct ggtacatttc caactactca tttcgaatgt | 840 |
| agctggatta tagcaacgtt gttaaaggtt ggctttactt tgaagcaaat tgacggcgat | 900 |
| ggcttaagag gtttatcaac catcttactt gaggcgcttc gtgatgagaa tggtgtcata | 960 |
| ggctttgccc ctagaacagc agatgtagat gacacagcca aagctctatt ggccttgtca | 1020 |
| ttggtaaacc agccagtgtc acctgatatc atgattaagg tctttgaggg caaagaccat | 1080 |
| tttaccactt ttggttcaga aagagatcca tcattgactt ccaacctgca cgtcctttta | 1140 |
| tctttactta aacaatctaa cttgtctcaa taccatcctc aaatcctcaa aacaacatta | 1200 |
| ttcacttgta gatggtggtg gggttccgat cattgtgtca aagacaaatg gaatttgagt | 1260 |
| cacctatatc caactatgtt gttggttgaa gccttcactg aagtgctcca tctcattgac | 1320 |
| ggtggtgaat tgtctagtct gtttgatgaa tcctttaagt gtaagattgg tcttagcatc | 1380 |
| tttcaagcgg tacttagaat aatcctcacc caagacaacg acggctcttg gagaggatac | 1440 |
| agagaacaga cgtgttacgc aatattggct ttagttcaag cgagacatgt atgctttttc | 1500 |
| actcacatgg ttgacagact gcaatcatgt gttgatcgag gtttctcatg gttgaaatct | 1560 |
| tgctcttttc attctcaaga cctgacttgg acctctaaaa cagcttatga agtgggtttc | 1620 |
| gtagctgaag catataaact agctgcttta caatctgctt ccctggaggt tcctgctgcc | 1680 |
| accattggac attctgtcac gtctgccgtt ccatcaagtg atcttgaaaa atacatgaga | 1740 |
| ttggtgagaa aaactgcgtt attctctcca ctggatgagt ggggtctaat ggcttctatc | 1800 |
| atcgaatctt catttttcgt accattactg caggcacaaa gagttgaaat ataccctaga | 1860 |
| gataatatca aggtggacga agataagtac ttgtctatta tcccattcac atgggtcgga | 1920 |
| tgcaataata ggtctagaac tttcgcaagt aacagatggc tatacgatat gatgtacctt | 1980 |
| tcattactcg gctatcaaac cgacgagtac atggaagctg tagctgggcc agtgtttggg | 2040 |
| gatgtttcct tgttacatca aacaattgat aaggtgattg ataatacaat gggtaacctt | 2100 |
| gcgagagcca atggaacagt acacagtggt aatggacatc agcacgaatc tcctaatata | 2160 |
| ggtcaagtcg aggacacctt gactcgtttc acaaattcag tcttgaatca caaagacgtc | 2220 |
| cttaactcta gctcatctga tcaagatact ttgagaagag agtttagaac attcatgcac | 2280 |
| gctcatataa cacaaatcga agataactca cgattcagta agcaagcctc atccgatgcg | 2340 |
| ttttcctctc ctgaacaatc ttactttcaa tgggtgaact caactggtgg ctcacatgtc | 2400 |
| gcttgcgcct attcatttgc cttctctaat tgcctcatgt ctgcaaattt gttgcagggt | 2460 |
| aaagacgcat ttccaagcgg aacgcaaaag tacttaatct cctaLgttat gagacatgcc | 2520 |
| acaaacatgt gtagaatgta taacgacttt ggctctattg ccagagacaa cgctgagaga | 2580 |
| aatgttaata gtattcattt tcctgagttt actctctgta acggaacttc tcaaaaccta | 2640 |
| gatgaaagga aggaaagact tctgaaaatc gcaacttacg aacaagggta tttggataga | 2700 |
| gcactagagg ccttggaaag acagagtaga gatgatgccg gagacagagc tggatctaaa | 2760 |
| gatatgagaa agttgaaaat cgttaagtta ttctgtgatg ttacggactt atacgatcag | 2820 |
| ctctacgtta tcaaagattt gtcatcctct atgaagtaa | 2859 |
| SEQ ID NO: 58 | |
| MPGKIENGTP KDLKTGNDFV SAAKSLLDRA FKSHHSYYGL CSTSCQVYDT AWVAMIPKTR | 60 |
| DNVKQWLFPE CFHYLLKTQA ADGSWGSLPT TQTAGILDTA SAVLALLCHA QEPLQILDVS | 120 |
| PDEMGLRIEH GVTSLKRQLA VWNDVEDTNH IGVEFIIPAL LSMLEKELDV PSFEFPCRSI | 180 |
| LERMHGEKLG HFDLEQVYGK PSSLLHSLEA FLGKLDFDRL SHHLYHGSMM ASPSSTAAYL | 240 |
| IGATKWDDEA EDYLRHVMRN GAGHGNGGIS GTFPTTHFEC SWIIATLLKV GFTLKQIDGD | 300 |
| GLRGLSTILL EALRDENGVI GFAPRTADVD DTAKALLALS LVNQPVSPDI MIKVFEGKDH | 360 |
| FTTEGSERDP SLTSNLHVLL SLLKQSNLSQ YHPQILKTTL FTCRWWWGSD HCVKDKWNLS | 420 |
| HLYPTMLLVE AFTEVLHLID GGELSSLFDE SFKCKIGLSI FQAVLRIILT QDNDGSWRGY | 480 |
| REQTCYAILA LVQARHVCFF THMVDRLQSC VDRGFSWLKS CSFHSQDLTW TSKTAYEVGF | 540 |
| VAEAYKLAAL QSASLEVPAA TIGHSVTSAV PSSDLEKYMR LVRKTALFSP LDEWGLMASI | 600 |
| IESSFFVPLL QAQRVEIYPR DNIKVDEDKY LSIIPFTWVG CNNRSRTFAS NRWLYDMMYL | 660 |
| SLLGYQTDEY MEAVAGPVFG DVSLLHQTID KVIDNTMGNL ARANGTVHSG NGHQHESPNI | 720 |
| GQVEDTLTRF TNSVLNHKDV LNSSSSDQDT LRREFRTFMH AHITQIEDNS RFSKQASSDA | 780 |
| FSSPEQSYFQ WVNSTGGSHV ACAYSFAFSN CLMSANLLQG KDAFPSGTQK YLISSVMRHA | 840 |
| TNMCRMYNDF GSIARDNAER NVNSIHFPEF TLCNGTSQNL DERKERLLKI ATYEQGYLDR | 900 |
| ALEALERQSR DDAGDRAGSK DMRKLKIVKL FCDVTDLYDQ LYVIKDLSSS MK | 952 |
| SEQ ID NO: 59 | |
| atggatgctg tgacgggttt gttaactgtc ccagcaaccg ctataactat tggtggaact | 60 |
| gctgtagcat tggcggtagc gctaatcttt tggtacctga aatcctacac atcagctaga | 120 |
| agatcccaat caaatcatct tccaagagtg cctgaagtcc caggtgttcc attgttagga | 180 |
| aatctgttac aattgaagga gaaaaagcca tacatgactt ttacgagatg ggcagcgaca | 240 |
| tatggaccta tctatagtat caaaactggg gctacaagta tggttgtggt atcatctaat | 300 |
| gagatagcca aggaggcatt ggtgaccaga ttccaatcca tatctacaag gaacttatct | 360 |
| aaagccctga aagtacttac agcagataag acaatggtcg caatgtcaga ttatgatgat | 420 |
| tatcataaaa cagttaagag acacatactg accgccgtct tgggtcctaa tgcacagaaa | 480 |
| aagcatagaa ttcacagaga tatcatgatg gataacatat ctactcaact tcatgaattc | 540 |
| gtgaaaaaca acccagaaca ggaagaggta gaccttagaa aaatctttca atctgagtta | 600 |
| ttcggcttag ctatgagaca agccttagga aaggatgttg aaagtttgta cgttgaagac | 660 |
| ctgaaaatca ctatgaatag agacgaaatc tttcaagtcc ttgttgttga tccaatgatg | 720 |
| ggagcaatcg atgttgattg gagagacttc tttccatacc taaagtgggt cccaaacaaa | 780 |
| aagttcgaaa atactattca acaaatgtac atcagaagag aagctgttat gaaatcttta | 840 |
| atcaaagagc acaaaaagag aatagcgtca ggcgaaaagc taaatagtta tatcgattac | 900 |
| cttttatctg aagctcaaac tttaaccgat cagcaactat tgatgtcctt gtgggaacca | 960 |
| atcattgaat cttcagatac aacaatggtc acaacagaat gggcaatgta cgaattagct | 1020 |
| aaaaacccta aattgcaaga taggttgtac agagacatta agtccgtctg tggatctgaa | 1080 |
| aagataaccg aagagcatct atcacagctg ccttacatta cagctatttt ccacgaaaca | 1140 |
| ctgagaagac actcaccagt tcctatcatt cctctaagac atgtacatga agataccgtt | 1200 |
| ctaggcggct accatgttcc tgctggcaca gaacttgccg ttaacatcta cggttgcaac | 1260 |
| atggacaaaa acgtttggga aaatccagag gaatggaacc cagaaagatt catgaaagag | 1320 |
| aatgagacaa ttgattttca aaagacgatg gccttcggtg gtggtaagag agtttgtgct | 1380 |
| ggttccttgc aagccctttt aactgcatct attgggattg ggagaatggt tcaagagttc | 1440 |
| gaatggaaac tgaaggatat gactcaagag gaagtgaaca cgataggcct aactacacaa | 1500 |
| atgttaagac cattgagagc tattatcaaa cctaggatct aa | 1542 |
| SEQ ID NO: 60 | |
| MDAVTGLLTV PATAITIGGT AVALAVALIF WYLKSYTSAR RSQSNHLPRV PEVPGVPLLG | 60 |
| NLLQLKEKKP YMTFTRWAAT YGPIYSIKTG ATSMVVVSSN EIAKEALVTR FQSISTRNLS | 120 |
| KALKVLTADK TMVAMSDYDD YHKTVKRHIL TAVLGPNAQK KHRIHRDIMM DNISTQLHEF | 180 |
| VKNNPEQEEV DLRKIFQSEL FGLAMRQALG KDVESLYVED LKITMNRDEI FQVLVVDPMM | 240 |
| GAIDVDWRDF FPYLKWVPNK KFENTIQQMY IRREAVMKSL IKEHKKRIAS GEKLNSYIDY | 300 |
| LLSEAQTLTD QQLLMSLWEP IIESSDTTMV TTEWAMYELA KNPKLQDRLY RDIKSVCGSE | 360 |
| KITEEHLSQL PYITAIFHET LRRHSPVPII PLRHVHEDTV LGGYHVPAGT ELAVNIYGCN | 420 |
| MDKNVWENPE EWNPERFMKE NETIDFQKTM AFGGGKRVCA GSLQALLTAS IGIGRMVQEF | 480 |
| EWKLKDMTQE EVNTIGLTTQ MLRPLRAIIK PRI | 513 |
| SEQ ID NO: 61 | |
| aagcttacta gtaaaatgga cggtgtcatc gatatgcaaa ccattccatt gagaaccgct | 60 |
| attgctattg gtggtactgc tgttgctttg gttgttgcat tatacttttg gttcttgaga | 120 |
| tcctacgctt ccccatctca tcattctaat catttgccac cagtacctga agttccaggt | 180 |
| gttccagttt tgggtaattt gttgcaattg aaagaaaaaa agccttacat gaccttcacc | 240 |
| aagtgggctg aaatgtatgg tccaatctac tctattagaa ctggtgctac ttccatggtt | 300 |
| gttgtctctt ctaacgaaat cgccaaagaa gttgttgtta ccagattccc atctatctct | 360 |
| accagaaaat tgtcttacgc cttgaaggtt ttgaccgaag ataagtctat ggttgccatg | 420 |
| tctgattatc acgattacca taagaccgtc aagagacata ttttgactgc tgttttgggt | 480 |
| ccaaacgccc aaaaaaagtt tagagcacat agagacacca tgatggaaaa cgtttccaat | 540 |
| gaattgcatg ccttcttcga aaagaaccca aatcaagaag tcaacttgag aaagatcttc | 600 |
| caatcccaat tattcggttt ggctatgaag caagccttgg gtaaagatgt tgaatccatc | 660 |
| tacgttaagg atttggaaac caccatgaag agagaagaaa tcttcgaagt tttggttgtc | 720 |
| gatccaatga tgggtgctat tgaagttgat tggagagact ttttcccata cttgaaatgg | 780 |
| gttccaaaca agtccttcga aaacatcatc catagaatgt acactagaag agaagctgtt | 840 |
| atgaaggcct tgatccaaga acacaagaaa agaattgcct ccggtgaaaa cttgaactcc | 900 |
| tacattgatt acttgttgtc tgaagcccaa accttgaccg ataagcaatt attgatgtct | 960 |
| ttgtgggaac ctattatcga atcttctgat accactatgg ttactactga atgggctatg | 1020 |
| tacgaattgg ctaagaatcc aaacatgcaa gacagattat acgaagaaat ccaatccgtt | 1080 |
| tgcggttccg aaaagattac tgaagaaaac ttgtcccaat tgccatactt gtacgctgtt | 1140 |
| ttccaagaaa ctttgagaaa gcactgtcca gttcctatta tgccattgag atatgttcac | 1200 |
| gaaaacaccg ttttgggtgg ttatcatgtt ccagctggta ctgaagttgc tattaacatc | 1260 |
| tacggttgca acatggataa gaaggtctgg gaaaatccag aagaatggaa tccagaaaga | 1320 |
| ttcttgtccg aaaaagaatc catggacttg tacaaaacta tggcttttgg tggtggtaaa | 1380 |
| agagtttgcg ctggttcttt acaagccatg gttatttctt gcattggtat cggtagattg | 1440 |
| gtccaagatt ttgaatggaa gttgaaggat gatgccgaag aagatgttaa cactttgggt | 1500 |
| ttgactaccc aaaagttgca tccattattg gccttgatta acccaagaaa gtaactcgag | 1560 |
| ccgcgg | 1566 |
| SEQ ID NO: 62 | |
| MDGVIDMQTI PLRTAIAIGG TAVALVVALY FWFLRSYASP SHHSNHLPPV PEVPGVPVLG | 60 |
| NLLQLKEKKP YMTFTKWAEM YGPIYSIRTG ATSMVVVSSN EIAKEVVVTR FPSISTRKLS | 120 |
| YALKVLTEDK SMVAMSDYHD YHKTVKRHIL TAVLGPNAQK KFRAHRDTMM ENVSNELHAF | 180 |
| FEKNPNQEVN LRKIFQSQLF GLAMKQALGK DVESIYVKDL ETTMKREEIF EVLVVDPMMG | 240 |
| AIEVDWRDFF PYLKWVPNKS FENIIHRMYT RREAVMKALI QEHKKRIASG ENLNSYIDYL | 300 |
| LSEAQTLTDK QLLMSLWEPI IESSDTTMVT TEWAMYELAK NPNMQDRLYE EIQSVCGSEK | 360 |
| ITEENLSQLP YLYAVFQETL RKHCPVPIMP LRYVHENTVL GGYHVPAGTE VAINIYGCNM | 420 |
| DKKVWENPEE WNPERFLSEK ESMDLYKTMA FGGGKRVCAG SLQAMVISCI GIGRLVQDFE | 480 |
| WKLKDDAEED VNTLGLTTQK LHPLLALINP RK | 512 |
| SEQ ID NO: 63 | |
| atggccaccc tccttgagca tttccaagct atgccctttg ccatccctat tgcactggct | 60 |
| gctctgtctt ggctgttcct cttttacatc aaagtttcat tcttttccaa caagagtgct | 120 |
| caggctaagc tccctcctgt gccagtggtt cctgggctgc cggtgattgg gaatttactg | 180 |
| caactcaagg agaagaaacc ctaccagact tttacaaggt gggctgagga gtatggacca | 240 |
| atctattcta tcaggactgg tgcttccacc atggtcgttc tcaataccac ccaagttgca | 300 |
| aaagaggcca tggtgaccag atatttatcc atctcaacca gaaagctatc aaacgcacta | 360 |
| aagattctta ctgctgataa atgtatggtt gcaataagtg actacaacga ttttcacaag | 420 |
| atgataaagc gatacatact ctcaaatgtt cttggaccca gtgctcagaa gcgtcaccgg | 480 |
| agcaacagag ataccttgag agctaatgtc tgcagccgat tgcattctca agtaaagaac | 540 |
| tctcctcgag aagctgtgaa tttcagaaga gtttttgagt gggaactctt tggaattgca | 600 |
| ttgaagcaag cctttggaaa ggacatagaa aagcccattt atgtggagga acttggcact | 660 |
| acactgtcaa gagatgagat ctttaaggtt ctagtgcttg acataatgga gggtgcaatt | 720 |
| gaggttgatt ggagagattt cttcccttac ctgagatgga ttccgaatac gcgcatggaa | 780 |
| acaaaaattc agcgactcta tttccgcagg aaagcagtga tgactgccct gatcaacgag | 840 |
| cagaagaagc gaattgcttc aggagaggaa atcaactgtt atatcgactt cttgcttaag | 900 |
| gaagggaaga cactgacaat ggaccaaata agtatgttgc tttgggagac ggttattgaa | 960 |
| acagcagata ctacaatggt aacgacagaa tgggctatgt atgaagttgc taaagactca | 1020 |
| aagcgtcagg atcgtctcta tcaggaaatc caaaaggttt gtggatcgga gatggttaca | 1080 |
| gaggaatact tgtcccaact gccgtacctg aatgcagttt tccatgaaac gctaaggaag | 1140 |
| cacagtccgg ctgcgttagt tcctttaaga tatgcacatg aagataccca actaggaggt | 1200 |
| tactacattc cagctggaac tgagattgct ataaacatat acgggtgtaa catggacaag | 1260 |
| catcaatggg aaagccctga ggaatggaaa ccggagagat ttttggaccc gaaatttgat | 1320 |
| cctatggatt tgtacaagac catggctttt ggggctggaa agagggtatg tgctggttct | 1380 |
| cttcaggcaa tgttaatagc gtgcccgacg attggtaggc tggtgcagga gtttgagtgg | 1440 |
| aagctgagag atggagaaga agaaaatgta gatactgttg ggctcaccac tcacaaacgc | 1500 |
| tatccaatgc atgcaatcct gaagccaaga agtta | 1535 |
| SEQ ID NO: 64 | |
| atggctacct tgttggaaca ttttcaagct atgccattcg ctattccaat tgctttggct | 60 |
| gctttgtctt ggttgttttt gttctacatc aaggtttctt tcttctccaa caaatccgct | 120 |
| caagctaaat tgccaccagt tccagttgtt ccaggtttgc cagttattgg taatttgttg | 180 |
| caattgaaag aaaagaagcc ataccaaacc ttcactagat gggctgaaga atatggtcca | 240 |
| atctactcta ttagaactgg tgcttctact atggttgtct tgaacactac tcaagttgcc | 300 |
| aaagaagcta tggttaccag atacttgtct atctctacca gaaagttgtc caacgccttg | 360 |
| aaaattttga ccgctgataa gtgcatggtt gccatttctg attacaacga tttccacaag | 420 |
| atgatcaaga gatatatctt gtctaacgtt ttgggtccat ctgcccaaaa aagacataga | 480 |
| tctaacagag ataccttgag agccaacgtt tgttctagat tgcattccca agttaagaac | 540 |
| tctccaagag aagctgtcaa ctttagaaga gttttcgaat gggaattatt cggtatcgct | 600 |
| ttgaaacaag ccttcggtaa ggatattgaa aagccaatct acgtcgaaga attgggtact | 660 |
| actttgtcca gagatgaaat cttcaaggtt ttggtcttgg acattatgga aggtgccatt | 720 |
| gaagttgatt ggagagattt tttcccatac ttgcgttgga ttccaaacac cagaatggaa | 780 |
| actaagatcc aaagattata ctttagaaga aaggccgtta tgaccgcctt gattaacgaa | 840 |
| caaaagaaaa gaattgcctc cggtgaagaa atcaactgct acatcgattt cttgttgaaa | 900 |
| gaaggtaaga ccttgaccat ggaccaaatc tctatgttgt tgtgggaaac cgttattgaa | 960 |
| actgctgata ccacaatggt tactactgaa tgggctatgt acgaagttgc taaggattct | 1020 |
| aaaagacaag acagattata ccaagaaatc caaaaggtct gcggttctga aatggttaca | 1080 |
| gaagaatact tgtcccaatt gccatacttg aatgctgttt tccacgaaac tttgagaaaa | 1140 |
| cattctccag ctgctttggt tccattgaga tatgctcatg aagatactca attgggtggt | 1200 |
| tattacattc cagccggtac tgaaattgcc attaacatct acggttgcaa catggacaaa | 1260 |
| caccaatggg aatctccaga agaatggaag ccagaaagat ttttggatcc taagtttgac | 1320 |
| ccaatggact tgtacaaaac tatggctttt ggtgctggta aaagagtttg cgctggttct | 1380 |
| ttacaagcta tgttgattgc ttgtccaacc atcggtagat tggttcaaga atttgaatgg | 1440 |
| aagttgagag atggtgaaga agaaaacgtt gatactgttg gtttgaccac ccataagaga | 1500 |
| tatccaatgc atgctatttt gaagccaaga tcttaa | 1536 |
| SEQ ID NO: 65 | |
| aagcttacta gtaaaatggc ctccatcacc catttcttac aagattttca agctactcca | 60 |
| ttcgctactg cttttgctgt tggtggtgtt tctttgttga tattcttctt cttcatccgt | 120 |
| ggtttccact ctactaagaa aaacgaatat tacaagttgc caccagttcc agttgttcca | 180 |
| ggtttgccag ttgttggtaa tttgttgcaa ttgaaagaaa agaagccata caagactttc | 240 |
| ttgagatggg ctgaaattca tggtccaatc tactctatta gaactggtgc ttctaccatg | 300 |
| gttgttgtta actctactca tgttgccaaa gaagctatgg ttaccagatt ctcttcaatc | 360 |
| tctaccagaa agttgtccaa ggctttggaa ttattgacct ccaacaaatc tatggttgcc | 420 |
| acctctgatt acaacgaatt tcacaagatg gtcaagaagt acatcttggc cgaattattg | 480 |
| ggtgctaatg ctcaaaagag acacagaatt catagagaca ccttgatcga aaacgtcttg | 540 |
| aacaaattgc atgcccatac caagaattct ccattgcaag ctgttaactt cagaaagatc | 600 |
| ttcgaatctg aattattcgg tttggctatg aagcaagcct tgggttatga tgttgattcc | 660 |
| ttgttcgttg aagaattggg tactaccttg tccagagaag aaatctacaa cgttttggtc | 720 |
| agtgacatgt tgaagggtgc tattgaagtt gattggagag actttttccc atacttgaaa | 780 |
| tggatcccaa acaagtcctt cgaaatgaag attcaaagat tggcctctag aagacaagcc | 840 |
| gttatgaact ctattgtcaa agaacaaaag aagtccattg cctctggtaa gggtgaaaac | 900 |
| tgttacttga attacttgtt gtccgaagct aagactttga ccgaaaagca aatttccatt | 960 |
| ttggcctggg aaaccattat tgaaactgct gatacaactg ttgttaccac tgaatgggct | 1020 |
| atgtacgaat tggctaaaaa cccaaagcaa caagacagat tatacaacga aatccaaaac | 1060 |
| gtctgcggta ctgataagat taccgaagaa catttgtcca agttgcctta cttgtctgct | 1140 |
| gtttttcacg aaaccttgag aaagtattct ccatctccat tggttccatt gagatacgct | 1200 |
| catgaagata ctcaattggg tggttattat gttccagccg gtactgaaat tgctgttaat | 1260 |
| atctacggtt gcaacatgga caagaatcaa tgggaaactc cagaagaatg gaagccagaa | 1320 |
| agatttttgg acgaaaagta cgatccaatg gacatgtaca agactatgtc ttttggttcc | 1380 |
| ggtaaaagag tttgcgctgg ttctttacaa gctagtttga ttgcttgtac ctccatcggt | 1440 |
| agattggttc aagaatttga atggagattg aaagacggtg aagttgaaaa cgttgatacc | 1500 |
| ttgggtttga ctacccataa gttgtatcca atgcaagcta tcttgcaacc tagaaactga | 1560 |
| ctcgagccgc gg | 1572 |
| SEQ ID NO: 66 | |
| MASITHFLQD FQATPFATAF AVGGVSLLIF FFFIRGFHST KKNEYYKLPP VPVVPGLPVV | 60 |
| GNLLQLKEKK PYKTFLRWAE IHGPIYSIRT GASTMVVVNS THVAKEAMVT RFSSISTRKL | 120 |
| SKALELLTSN KSMVATSDYN EFHKMVKKYI LAELLGANAQ KRHRIHRDTL IENVLNKLHA | 180 |
| HTKNSPLQAV NFRKIFESEL FGLAMKQALG YDVDSLFVEE LGTTLSREEI YNVLVSDMLK | 240 |
| GAIEVDWRDF FPYLKWIPNK SFEMKIQRLA SRRQAVMNSI VKEQKKSIAS GKGENCYLNY | 300 |
| LLSEAKTLTE KQISILAWET IIETADTTVV TTEWAMYELA KNPKQQDRLY NEIQNVCGTD | 360 |
| KITEEHLSKL PYLSAVFHET LRKYSPSPLV PLRYAHEDTQ LGGYYVPAGT EIAVNIYGCN | 420 |
| MDKNQWETPE EWKPERFLDE KYDPMDMYKT MSFGSGKRVC AGSLQASLIA CTSIGRLVQE | 480 |
| FEWRLKDGEV ENVDTLGLTT HKLYPMQAIL QPRN | 514 |
| SEQ ID NO: 67 | |
| atgatttcct tgttgttggg ttttgttgtc tcctccttct tgtttatctt cttcttgaaa | 60 |
| aaattgttgt tcttcttcag tcgtcacaaa atgtccgaag tttctagatt gccatctgtt | 120 |
| ccagttccag gttttccatt gattggtaac ttgttgcaat tgaaagaaaa gaagccacac | 180 |
| aagactttca ccaagtggtc tgaattatat ggtccaatct actctatcaa gatgggttcc | 240 |
| tcttctttga tcgtcttgaa ctctattgaa accgccaaag aagctatggt cagtagattc | 300 |
| tcttcaatct ctaccagaaa gttgtctaac gctttgactg ttttgacctg caacaaatct | 360 |
| atggttgcta cctctgatta cgatgacttt cataagttcg tcaagagatg cttgttgaac | 420 |
| ggtttgttgg gtgctaatgc tcaagaaaga aaaagacatt acagagatgc cttgatcgaa | 480 |
| aacgttacct ctaaattgca tgcccatacc agaaatcatc cacaagaacc agttaacttc | 540 |
| agagccattt tcgaacacga attattcggt gttgctttga aacaagcctt cggtaaagat | 600 |
| gtcgaatcca tctatgtaaa agaattgggt gtcaccttgt ccagagatga aattttcaag | 660 |
| gttttggtcc acgacatgat ggaaggtgct attgatgttg attggagaga tttcttccca | 720 |
| tacttgaaat ggatcccaaa caactctttc gaagccagaa ttcaacaaaa gcacaagaga | 780 |
| agattggctg ttatgaacgc cttgatccaa gacagattga atcaaaacga ttccgaatcc | 840 |
| gatgatgact gctacttgaa tttcttgatg tctgaagcta agaccttgac catggaacaa | 900 |
| attgctattt tggtttggga aaccattatc gaaactgctg ataccacttt ggttactact | 960 |
| gaatgggcta tgtacgaatt ggccaaacat caatctgttc aagatagatt attcaaagaa | 1020 |
| atccaatccg tctgcggtgg tgaaaagatc aaagaagaac aattgccaag attgccttac | 1080 |
| gtcaatggtg tttttcacga aaccttgaga aagtattctc cagctccatt ggttccaatt | 1140 |
| agatacgctc atgaagatac ccaaattggt ggttatcata ttccagccgg ttctgaaatt | 1200 |
| gccattaaca tctacggttg caacatggat aagaagagat gggaaagacc tgaagaatgg | 1260 |
| tggccagaaa gatttttgga agatagatac gaatcctccg acttgcataa gactatggct | 1320 |
| tttggtgctg gtaaaagagt ttgtgctggt gctttacaag ctagtttgat ggctggtatt | 1380 |
| gctatcggta gattggttca agaattcgaa tygaagttga gagatggtga agaagaaaac | 1440 |
| gttgatactt acggtttgac ctcccaaaag ttgtatccat tgatggccat tatcaaccca | 1500 |
| agaagatctt aa | 1512 |
| SEQ ID NO: 68 | |
| MASMISLLLG FVVSSFLFIF FLKKLLFFFS RHKMSEVSRL PSVPVPGFPL IGNLLQLKEK | 60 |
| KPHKTFTKWS ELYGPIYSIK MGSSSLIVLN SIETAKEAMV SRFSSISTRK LSNALTVLTC | 120 |
| NKSMVATSDY DDFHKEVKRC LLNGLLGANA QERKRHYRDA LIENVTSKLH AHTRNHPQEP | 180 |
| VNFRAIFEHE LFGVALKQAF GKDVESIYVK ELGVTLSRDE IFKVLVHDMM EGAIDVDWRD | 240 |
| FFPYLKWIPN NSFEARIQQK HKRRLAVMNA LIQDRLNQND SESDDDCYLN FLMSEAKTLT | 300 |
| MEQIAILVWE TIIETADTTL VTTEWAMYEL AKHQSVQDRL FKEIQSVCGG EKIKEEQLPR | 360 |
| LPYVNGVFHE TLRKYSPAPL VPIRYAHEDT QIGGYHIPAG SEIAINIYGC NMDKKRWERP | 420 |
| EEWWPERFLE DRYESSDLHK TMAFGAGKRV CAGALQASLM AGIAIGRLVQ EFEWKLRDGE | 480 |
| EENVDTYGLT SQKLYPLMAI INPRRS | 506 |
| SEQ ID NO: 69 | |
| aagcttacta gtaaaatgga catgatgggt attgaagctg ttccatttgc tactgctgtt | 60 |
| gttttgggtg gtatttcctt ggttgttttg atcttcatca gaagattcgt ttccaacaga | 120 |
| aagagatccg ttgaaggttt gccaccagtt ccagatattc caggtttacc attgattggt | 180 |
| aacttgttgc aattgaaaga aaagaagcca cataagacct ttgctagatg ggctgaaact | 240 |
| tacggtccaa ttttctctat tagaactggt gcttctacca tgatcgtctt gaattcttct | 300 |
| gaagttgcca aagaagctat ggtcactaga ttctcttcaa tctctaccag aaagttgtcc | 360 |
| aacgccttga agattttgac cttcgataag tgtatggttg ccacctctga ttacaacgat | 420 |
| tttcacaaaa tggtcaaggg tttcatcttg agaaacgttt taggtgctcc agcccaaaaa | 480 |
| agacatagat gtcatagaga taccttgatc gaaaacatct ctaagtactt gcatgcccat | 540 |
| gttaagactt ctccattgga accagttgtc ttgaagaaga ttttcgaatc cgaaattttc | 600 |
| ggtttggctt tgaaacaagc cttgggtaag gatatcgaat ccatctatgt tgaagaattg | 660 |
| ggtactacct tgtccagaga agaaattttt gccgttttgg ttgttgatcc aatggctggt | 720 |
| gctattgaag ttgattggag agattttttc ccatacttgt cctggattcc aaacaagtct | 780 |
| atggaaatga agatccaaag aatggatttt agaagaggtg ctttgatgaa ggccttgatt | 840 |
| ggtgaacaaa agaaaagaat cggttccggt gaagaaaaga actcctacat tgatttcttg | 900 |
| ttgtctgaag ctaccacttt gaccgaaaag caaattgcta tgttgatctg ggaaaccatc | 960 |
| atcgaaattt ccgatacaac tttggttacc tctgaatggg ctatgtacga attggctaaa | 1020 |
| gacccaaata gacaagaaat cttgtacaga gaaatccaca aggtttgcgg ttctaacaag | 1080 |
| ttgactgaag aaaacttgtc caagttgcca tacttgaact ctgttttcca cgaaaccttg | 1140 |
| agaaagtatt ctccagctcc aatggttcca gttagatatg ctcatgaaga tactcaattg | 1200 |
| ggtggttacc atattccagc tggttctcaa attgccatta acatctacgg ttgcaacatg | 1260 |
| aacaaaaagc aatgggaaaa tcctgaagaa tggaagccag aaagattctt ggacgaaaag | 1320 |
| tatgacttga tggacttgca taagactatg gcttttggtg gtggtaaaag agtttgtgct | 1380 |
| ggtgctttac aagcaatgtt gattgcttgc acttccatcg gtagattcgt tcaagaattt | 1440 |
| gaatggaagt tgatgggtgg tgaagaagaa aacgttgata ctgttgcttt gacctcccaa | 1500 |
| aaattgcatc caatgcaagc cattattaag gccagagaat gactcgagcc gcgg | 1554 |
| SEQ ID NO: 70 | |
| MDMMGIEAVP FATAVVLGGI SLVVLIFIRR FVSNRKRSVE GLPPVPDIPG LPLIGNLLQL | 60 |
| KEKKPHKTFA RWAETYGPIF SIRTGASTMI VLNSSEVAKE AMVTRFSSIS TRKLSNALKI | 120 |
| LTFDKCMVAT SDYNDFHKMV KGFILRNVLG APAQKRHRCH RDTLIENISK YLHAHVKTSP | 180 |
| LEPVVLKKIF ESEIFGLALK QALGKDIESI YVEELGTTLS REEIFAVLVV DPMAGAIEVD | 240 |
| WRDFFPYLSW IPNKSMEMKI QRMDFRRGAL MKALIGEQKK RIGSGEEKNS YIDFLLSEAT | 300 |
| TLTEKQIAML IWETIIEISD TTLVTSEWAM YELAKDPNRQ EILYREIHKV CGSNKLTEEN | 360 |
| LSKLPYLNSV FHETLRKYSP APMVPVRYAH EDTQLGGYHI PAGSQIAINI YGCNMNKKQW | 420 |
| ENPEEWKPER FLDEKYDLMD LHKTMAFGGG KRVCAGALQA MLIACTSIGR FVQEFEWKLM | 480 |
| GGEEENVDTV ALTSQKLHPM QAIIKARE | 508 |
| SEQ ID NO: 71 | |
| aagcttaaaa tgagtaagtc taatagtatg aattctacat cacacgaaac cctttttcaa | 60 |
| caattggtct tgggtttgga ccgtatgcca ttgatggatg ttcactggtt gatctacgtt | 120 |
| gctttcggcg catggttatg ttcttatgtg atacatgttt tatcatcttc ctctacagta | 180 |
| aaagtgccag ttgttggata caggtctgta ttcgaaccta catggttgct tagacttaga | 240 |
| ttcgtctggg aaggtggctc tatcataggt caagggtaca ataagtttaa agactctatt | 300 |
| ttccaagtta ggaaattggg aactgatatt gtcattatac cacctaacta tattgatgaa | 360 |
| gtgagaaaat tgtcacagga caagactaga tcagttgaac ctttcattaa tgattttgca | 420 |
| ggtcaataca caagaggcat ggttttcttg caatctgact tacaaaaccg tgttatacaa | 480 |
| caaagactaa ctccaaaatt ggtttccttg accaaggtca tgaaggaaga gttggattat | 540 |
| gctttaacaa aagagatgcc tgatatgaaa aatgacgaat gggtagaagt agatatcagt | 600 |
| agtataatgg tgagattgat ttccaggatc tccgccagag tatttctagg gcctgaacac | 660 |
| tgtcgtaacc aggaatggtt gactactaca gcagaatatt cagaatcact tttcattaca | 720 |
| gggtttatct taagagttgt acctcatatc ttaagaccat tcatcgcccc tctattacct | 780 |
| tcatacagga ctctacttag aaacgtttca agtggtagaa gagtcatcgg tgacatcata | 840 |
| agatctcagc aaggggatgg taacgaagat atactttcct ggatgagaga tgctgccaca | 900 |
| ggagaggaaa agcaaatcga taacattgct cagagaatgt taattctttc tttagcatca | 960 |
| atccacacta ctgcgatgac catgacacat gccatgtacg atctatgtgc ttgccctgag | 1020 |
| tacattgaac cattaagaga tgaagttaaa tctgttgttg gggcttctgg ctgggacaag | 1080 |
| acagcgttaa acagatttca taagttggac tccttcctaa aagagtcaca aagattcaac | 1140 |
| ccagtattct tattgacatt caatagaatc taccatcaat ctatgacctt atcagatggc | 1200 |
| actaacattc catctggaac acgtattgct gttccatcac acgcaatgtt gcaagattct | 1260 |
| gcacatgtcc caggtccaac cccacctact gaatttgatg gattcagata tagtaagata | 1320 |
| cgttctgata gtaactacgc acaaaagtac ctattctcca tgaccgattc ttcaaacatg | 1380 |
| gctttcggat acggcaagta tgcttgtcca ggtagatttt acgcgtctaa tgagatgaaa | 1440 |
| ctaacattag ccattttgtt gctacaattt gagttcaaac taccagatgg taaaggtcgt | 1500 |
| cctagaaata tcactatcga ttctgatatg attccagacc caagagctag actttgcgtc | 1560 |
| agaaaaagat cacttagaga tgaatgaccg cgg | 1593 |
| SEQ ID NO: 72 | |
| MSKSNSMNST SHETLFQQLV LGLDRMPLMD VHWLIYVAFG AWLCSYVIHV LSSSSTVKVP | 60 |
| VVGYRSVFEP TWLLRLRFVW EGGSIIGQGY NKFKDSIFQV RKLGTDIVII PPNYIDEVRK | 120 |
| LSQDKTRSVE PFINDFAGQY TRGMVFLQSD LQNRV1QQRL TPKLVSLTKV MKEELDYALT | 180 |
| KEMPDMKNDE WVEVDISSIM VRLISRISAR VFLGPEHCRN QEWLTTTAEY SESLFITGFI | 240 |
| LRVVPHILRP FIAPLLPSYR TLLRNVSSGR RVIGDIIRSQ QGDGNEDILS WMRDAATGEE | 300 |
| KQIDNIAQRM LILSLASIHT TAMTMTHAMY DLCACPEYIE PLRDEVKSVV GASGWDKTAL | 360 |
| NRFHKLDSFL KESQRFNPVF LLTFNRIYHQ SMTLSDGTNI PSGTRIAVPS HAMLQDSAHV | 420 |
| PGPIPPTEFD GFRYSKIRSD SNYAQKYLFS MTDSSNMAFG YGKYACPGRF YASNEMKLTL | 480 |
| AILLLQFEFK LPDGKGRPRN ITIDSDMIPD PRARLCVRKR SLRDE | 525 |
| SEQ ID NO: 73 | |
| aagcttaaaa tggaagatcc tactgtctta tatgcttgtc ttgccattgc agttgcaact | 60 |
| ttcgttgtta gatggtacag agatccattg agatccatcc caacagttgg tggttccgat | 120 |
| ttgcctattc tatcttacat cggcgcacta agatggacaa gacgtggcag agagatactt | 180 |
| caagagggat atgatggcta cagaggatct acattcaaaa tcgcgatgtt agaccgttgg | 240 |
| atcgtgatcg caaatggtcc taaactagct gatgaagtca gacgtagacc agatgaagag | 300 |
| ttaaacttta tggacggatt aggagcattc gtccaaacta agtacacctt aggtgaagct | 360 |
| attcataacg atccatacca tgtcgatatc ataagagaaa aactaacaag aggccttcca | 420 |
| gccgtgcttc ctgatgtcat tgaagagttg acacttgcgg ttagacagta cattccaaca | 480 |
| gaaggtgatg aatgggtgtc cgtaaactgt tcaaaggccg caagagatat tgttgctaga | 540 |
| gcttctaata gagtctttgt aggtttgcct gcttgcagaa accaaggtta cttagatttg | 600 |
| gcaatagact ttacattgtc tgttgtcaag gatagagcca tcatcaatat gtttccagaa | 660 |
| ttgttgaagc caatagttgg cagagttgta ggtaacgcca ccagaaatgt tcgtagagct | 720 |
| gttccttttg ttgctccatt ggtggaggaa agacgtagac ttatggaaga gtacggtgaa | 780 |
| gactggtctg aaaaacctaa tgatatgtta cagtggataa tggatgaagc tgcatccaga | 840 |
| gatagttcag tgaaggcaat cgcagagaga ttgttaatgg tgaacttcgc ggctattcat | 900 |
| acctcatcaa acactatcac tcatgctttg taccaccttg ccgaaatgcc tgaaactttg | 960 |
| caaccactta gagaagagat cgaaccatta gtcaaagagg agggctggac caaggctgct | 1020 |
| atgggaaaaa tgtggtggtt agattcattt ctaagagaat ctcaaagata caatggcatt | 1060 |
| aacatcgtat ctttaactag aatggctgac aaagatatta cattgagtga tggcacattt | 1140 |
| ttgccaaaag gtactctagt ggccgttcca gcgtattcta ctcatagaga tgatgctgtc | 1200 |
| tacgctgatg ccttagtatt cgatcctttc agattctcac gtatgagagc gagagaaggt | 1260 |
| gaaggtacaa agcaccagtt cgttaatact tcagtcgagt acgttccatt tggtcacgga | 1320 |
| aagcatgctt gtccaggaag attcttcgcc gcaaacgaat tgaaagcaat gttggcttac | 1380 |
| attgttctaa actatgatgt aaagttgcct ggtgacggta aacgtccatt gaacatgtat | 1440 |
| tggggtccaa cagttttgcc tgcaccagca ggccaagtat tgttcagaaa gagacaagtt | 1500 |
| agtctataac cgcgg | 1515 |
| SEQ ID NO: 74 | |
| MEDPTVLYAC LAIAVATFVV RWYRDPLRSI PTVGGSDLPI LSYIGALRWT RRGREILQEG | 60 |
| YDGYRGSTFK IAMLDRWIVI ANGPKLADEV RRRPDEELNF MDGLGAFVQT KYTLGEAIHN | 120 |
| DPYHVDIIRE KLTRGLPAVL PDVIEELTLA VRQYIPTEGD EWVSVNCSKA ARDIVARASN | 180 |
| RVFVGLPACR NQGYLDLAID FTLSVVKDRA IINMFPELLK PIVGRVVGNA TRNVRRAVPF | 240 |
| VAPLVEERRR LMEEYGEDWS EKPNDMLQWI MDEAASRDSS VKAIAERLLM VNFAAIHTSS | 300 |
| NTITHALYHL AEMPETLQPL REEIEPLVKE EGWTKAAMGK MWWLDSFLRE SQRYNGINIV | 360 |
| SLTRMADKDI TLSDGTFLPK GTLVAVPAYS THRDDAVYAD ALVFDPERFS RMRAREGEGT | 420 |
| KHQFVNTSVE YVPFGHGKHA CPGRFFAANE LKAMLAYIVL NYDVKLPGDG KRPLNMYWGP | 480 |
| TVLPAPAGQV LFRKRQVSL | 499 |
| SEQ ID NO: 75 | |
| atggcatttt tctctatgat ttcaattttg ttgggatttg ttatttcttc tttcatcttc | 60 |
| atctttttct tcaaaaagtt acttagtttt agtaggaaaa acatgtcaga agtttctact | 120 |
| ttgccaagtg ttccagtagt gcctggtttt ccagttattg ggaatttgtt gcaactaaag | 180 |
| gagaaaaagc ctcataaaac tttcactaga tggtcagaga tatatggacc tatctactct | 240 |
| ataaagatgg gttcttcatc tcttattgta ttgaacagta cagaaactgc taaggaagca | 300 |
| atggtcacta gattttcatc aatatctacc agaaaattgt caaacgccct aacagttcta | 360 |
| acctgcgata agtctatggt cgccacttct gattatgatg acttccacaa attagttaag | 420 |
| agatgtttgc taaatggact tcttggtgct aatgctcaaa agagaaaaag acactacaga | 480 |
| gatgctttga ttgaaaatgt gagttccaag ctacatgcac acgctagaga tcatccacaa | 540 |
| gagccagtta actttagagc aattttcgaa cacgaattgt ttggtgtagc attaaagcaa | 600 |
| gccttcggta aagacgtaga atccatatac gtcaaggagt taggcgtaac attatcaaaa | 660 |
| gatgaaatct ttaaggtgct tgtacatgat atgatggagg gtgcaattga tgtagattgg | 720 |
| agagatttct tcccatattt gaaatggatc cctaataagt cttttgaagc taggatacaa | 780 |
| caaaagcaca agagaagact agctgttatg aacgcactta tacaggacag attgaagcaa | 840 |
| aatgggtctg aatcagatga tgattgttac cttaacttct taatgtctga ggctaaaaca | 900 |
| ttgactaagg aacagatcgc aatccttgtc tgggaaacaa tcattgaaac agcagatact | 960 |
| accttagtca caactgaatg ggccatatac gagctagcca aacatccatc tgtgcaagat | 1020 |
| aggttgtgta aggagatcca gaacgtgtgt ggtggagaga aattcaagga agagcagttg | 1080 |
| tcacaagttc cttaccttaa cggcgttttc catgaaacct tgagaaaata ctcacctgca | 1140 |
| ccattagttc ctattagata cgcccacgaa gatacacaaa tcggtggcta ccatgttcca | 1200 |
| gctgggtccg aaattgctat aaacatctac gggtgcaaca tggacaaaaa gagatgggaa | 1260 |
| agaccagaag attggtggcc agaaagattc ttagatgatg gcaaatatga aacatctgat | 1320 |
| ttgcataaaa caatggcttt cggagctggc aaaagagtgt gtgccggtgc tctacaagcc | 1380 |
| tccctaatgg ctggtatcgc tattggtaga ttggtccaag agttcgaatg gaaacttaga | 1440 |
| gatggtgaag aggaaaatgt cgatacttat gggttaacat ctcaaaagtt atacccacta | 1500 |
| atggcaatca tcaatcctag aagatcctaa | 1530 |
| SEQ ID NO: 76 | |
| MAFFSMISIL LGFVISSFIF IFFEKKLLSE SRKNMSEVST LPSVPVVPGF PVIGNLLQLK | 60 |
| EKKPMKTFTR WSEIYGPIYS IKMGSSSLIV LNSTETAKEA MVTRFSSIST RKLSNALTVL | 120 |
| TCDKSMVATS DYDDFHKLVK RCLLNGLLGA NAQKRKRHYR DALIENVSSK LHAHARDHPQ | 180 |
| EPVNFRAIFE HELFGVALKQ AFGKDVESIY VKELGVTLSK DEIFKVLVHD MMEGAIDVDW | 240 |
| RDFFPYLKWI PNKSFEARIQ QKHKRRLAVM NALIQDRLKQ NGSESDDDCY LNFLMSEAKT | 300 |
| LTKEQIAILV WETIIETADT TLVTTEWAIY ELAKHPSVQD RLCKEIQNVC GGEKEKEEQL | 360 |
| SQVPYLNGVF HETLRKYSPA PLVPIRYAHE DTQIGGYHVP AGSEIAINIY GCNMDKKRWE | 420 |
| RPEDWWPERF LDDGKYETSD LHKTMAFGAG KRVCAGALQA SLMAGIAIGR LVQEFEWKLR | 480 |
| DGEEENVDTY GLTSQKLYPL MAIINPRRS | 509 |
| SEQ ID NO: 77 | |
| atgcaatcag attcagtcaa agtctctcca tttgatttgg tttccgctgc tatgaatggc | 60 |
| aaggcaatgg aaaagttgaa cgctagtgaa tctgaagatc caacaacatt gcctgcacta | 120 |
| aagatgctag ttgaaaatag agaattgttg acactgttca caacttcctt cgcagttctt | 180 |
| attgggtgtc ttgtatttct aatgtggaga cgttcatcct ctaaaaagct ggtacaagat | 240 |
| ccagttccac aagttatcgt tgtaaagaag aaagagaagg agtcagaggt tgatgacggg | 300 |
| aaaaagaaag tttctatttt ctacggcaca caaacaggaa ctgccgaagg ttttgctaaa | 360 |
| gcattagtcg aggaagcaaa agtgagatat gaaaagacct ctttcaaggt tatcgatcta | 420 |
| gatgactacg ctgcagatga tgatgaatat gaggaaaaac tgaaaaagga atccttagcc | 480 |
| ttcttcttct tggccacata cggtgatggt gaacctactg ataatgctgc taacttctac | 540 |
| aagtggttca cagaaggcga cgataaaggt gaatggctga aaaagttaca atacggagta | 600 |
| tttggtttag gtaacagaca atatgaacat ttcaacaaga tcgctattgt agttgatgat | 660 |
| aaacttactg aaatgggagc caaaagatta gtaccagtag gattagggga tgatgatcag | 720 |
| tgtatagaag atgacttcac cgcctggaag gaattggtat ggccagaatt ggatcaactt | 780 |
| ttaagggacg aagatgatac ttctgtgact accccataca ctgcagccgt attggagtac | 840 |
| agagtggttt accatgataa accagcagac tcatatgctg aagatcaaac ccatacaaac | 900 |
| ggtcatgttg ttcatgatgc acagcatcct tcaagatcta atgtggcttt caaaaaggaa | 960 |
| ctacacacct ctcaatcaga taggtcttgt actcacttag aattcgatat ttctcacaca | 1020 |
| ggactgtctt acgaaactgg cgatcacgtt ggcgtttatt ccgagaactt gtccgaagtt | 1080 |
| gtcgatgaag cactaaaact gttagggtta tcaccagaca catacttctc agtccatgct | 1140 |
| gataaggagg atgggacacc tatcggtggt gcttcactac caccaccttt tcctccttgc | 1200 |
| acattgagag acgctctaac cagatacgca gatgtcttat cctcacctaa aaaggtagct | 1260 |
| ttgctggcat tggctgctca tgctagtgat cctagtgaag ccgataggtt aaagttcctg | 1320 |
| gcttcaccag ccggaaaaga tgaatatgca caatggatcg tcgccaacca acgttctttg | 1380 |
| ctagaagtga tgcaaagttt tccatctgcc aagcctccat taggtgtgtt cttcgcagca | 1440 |
| gtagctccac gtttacaacc aagatactac tctatcagtt catctcctaa gatgtctcct | 1500 |
| aacagaatac atgttacatg tgctttggtg tacgagacta ctccagcagg cagaattcac | 1560 |
| agaggattgt gttcaacctg gatgaaaaat gctgtccctt taacagagtc acctgattgc | 1620 |
| tctcaagcat ccattttcgt tagaacatca aatttcagac ttccagtgga tccaaaagtt | 1680 |
| ccagtcatta tgataggacc aggcactggt cttgccccat tcaggggctt tcttcaagag | 1740 |
| agattggcct tgaaggaatc tggtacagaa ttgggttctt ctatcttttt ctttggttgc | 1800 |
| cgtaatagaa aagttgactt tatctacgag gacgagctta acaattttgt tgagacagga | 1860 |
| gcattgtcag aattgatcgt cgcattttca agagaaggga ctgccaaaga gtacgttcag | 1920 |
| cacaagatga gtcaaaaagc ctccgatata tggaaacttc taagtgaagg tgcctatctt | 1980 |
| tatgtctgtg gcgatgcaaa gggcatggcc aaggatgtcc atagaactct gcatacaatt | 2040 |
| gttcaggaac aagggagtct ggattcttcc aaggctgaat tgtacgtcaa aaacttacag | 2100 |
| atgtctggaa gatacttaag agatgtttgg taa | 2133 |
| SEQ ID NO: 78 | |
| MQSDSVKVSP FDLVSAAMNG KAMEKLNASE SEDPTTLPAL KMLVENRELL TLFTTSFAVL | 60 |
| IGCLVFLMWR RSSSKKLVQD PVPQVIVVKK KEKESEVDDG KKKVSIFYGT QTGTAEGFAK | 120 |
| ALVEEAKVRY EKTSFKVIDL DDYAADDDEY EEKLKKESLA FFFLATYGDG EPTDNAANFY | 180 |
| KWFTEGDDKG EWLKKLQYGV FGLGNRQYEH FNKIAIVVDD KLTEMGAKRL VPVGLGDDDQ | 240 |
| CIEDDFTAWK ELVWPELDQL LRDEDDTSVT TPYTAAVLEY RVVYHDKPAD SYAEDQTHTN | 300 |
| GHVVHDAQHP SRSNVAFKKE LHTSQSDRSC THLEFDISHT GLSYETGDHV GVYSENLSEV | 360 |
| VDEALKLLGL SPDTYFSVHA DKEDGTPIGG ASLPPPFPPC TLRDALTRYA DVLSSPKKVA | 420 |
| LLALAAHASD PSEADRLKFL ASPAGKDEYA QWIVANQRSL LEVMQSFPSA KPPLGVFFAA | 480 |
| VAPRLQPRYY SISSSPKMSP NRIHVTCALV YETTPAGRIH RGLCSTWMKN AVPLTESPDC | 540 |
| SQASIFVRTS NFRLPVDPKV PVIMIGPGTG LAPFRGFLQE RLALKESGTE LGSSIFFFGC | 600 |
| RNRKVDFIYE DELNNFVETG ALSELIVAFS REGTAKEYVQ HKMSQKASDI WKLLSEGAYL | 660 |
| YVCGDAKGMA KDVHRTLHTI VQEQGSLDSS KAELYVKNLQ MSGRYLRDVW | 710 |
| SEQ ID NO: 79 | |
| atgaaggtca gtccattcga attcatgtcc gctattatca agggtagaat ggacccatct | 60 |
| aactcctcat ttgaatctac tggtgaagtt gcctccgtta tctttgaaaa cagagaattg | 120 |
| gttgccatct tgaccacttc tattgctgtt atgattggtt gcttcgttgt cttgatgtgg | 180 |
| agaagagctg gttctagaaa ggttaagaat gtcgaattgc caaagccatt gattgtccat | 240 |
| gaaccagaac ctgaagttga agatggtaag aagaaggttt ccatcttctt cggtactcaa | 300 |
| actggtactg ctgaaggttt tgctaaggct ttggctgatg aagctaaagc tagatacgaa | 360 |
| aaggctacct tcagagttgt tgatttggat gattatgctg ccgatgatga ccaatacgaa | 420 |
| gaaaaattga agaacgaatc cttcgccgtt ttcttgttgg ctacttatgg tgatggtgaa | 480 |
| cctactgata atgctgctag attttacaag tggttcgccg aaggtaaaga aagaggtgaa | 540 |
| tggttgcaaa acttgcacta tgctgttttt ggtttgggta acagacaata cgaacacttc | 600 |
| aacaagattg ctaaggttgc cgacgaatta ttggaagctc aaggtggtaa tagattggtt | 660 |
| aaggttggtt taggtgatga cgatcaatgc atcgaagatg atttttctgc ttggagagaa | 720 |
| tctttgtggc cagaattgga tatgttgttg agagatgaag atgatgctac tactgttact | 780 |
| actccatata ctgctgctgt cttggaatac agagttgtct ttcatgattc tgctgatgtt | 840 |
| gctgctgaag ataagtcttg gattaacgct aatggtcatg ctgttcatga tgctcaacat | 900 |
| ccattcagat ctaacgttgt cgtcagaaaa gaattgcata cttctgcctc tgatagatcc | 960 |
| tgttctcatt tggaattcaa catttccggt tccgctttga attacgaaac tggtgatcat | 1020 |
| gttggtgtct actgtgaaaa cttgactgaa actgttgatg aagccttgaa cttgttgggt | 1080 |
| ttgtctccag aaacttactt ctctatctac accgataacg aagatggtac tccattgggt | 1140 |
| ggttcttcat tgccaccacc atttccatca tgtactttga gaactgcttt gaccagatac | 1200 |
| gctgatttgt tgaactctcc aaaaaagtct gctttgttgg ctttagctgc tcatgcttct | 1260 |
| aatccagttg aagctgatag attgagatac ttggcttctc cagctggtaa agatgaatat | 1320 |
| gcccaatctg ttatcggttc ccaaaagtct ttgttggaag ttatggctga attcccatct | 1380 |
| gctaaaccac cattaggtgt tttttttgct gctgttgctc caagattgca acctagattc | 1440 |
| tactccattt catcctctcc aagaatggct ccatctagaa tccatgttac ttgtgctttg | 1500 |
| gtttacgata agatgccaac tggtagaatt cataagggtg tttgttctac ctggatgaag | 1560 |
| aattctgttc caatggaaaa gtcccatgaa tgttcttggg ctccaatttt cgttagacaa | 1620 |
| tccaatttta agttgccagc cgaatccaag gttccaatta tcatggttgg tccaggtact | 1680 |
| ggtttggctc cttttagagg ttttttacaa gaaagattgg ccttgaaaga atccggtgtt | 1740 |
| gaattgggtc catccatttt gtttttcggt tgcagaaaca gaagaatgga ttacatctac | 1800 |
| gaagatgaat tgaacaactt cgttgaaacc ggtgctttgt ccgaattggt tattgctttt | 1860 |
| tctagagaag gtcctaccaa agaatacgtc caacataaga tggctgaaaa ggcttctgat | 1920 |
| atctggaact tgatttctga aggtgcttac ttgtacgttt gtggtgatgc taaaggtatg | 1980 |
| gctaaggatg ttcatagaac cttgcatacc atcatgcaag aacaaggttc tttggattct | 2040 |
| tccaaagctg aatccatggt caagaacttg caaatgaatg gtagatactt aagagatgtt | 2100 |
| tggtaa | 2106 |
| SEQ ID NO: 80 | |
| MKVSPFEFMS AIIKGRMDPS NSSFESTGEV ASVIFENREL VAILTTSIAV MIGCFVVLMW | 60 |
| RRAGSRKVKN VELPKPLIVH EPEPEVEDGK KKVSIFFGTQ TGTAEGFAKA LADEAKARYE | 120 |
| KATFRVVDLD DYAADDDQYE EKLKNESFAV FLLATYGDGE PTDNAARFYK WFAEGKERGE | 180 |
| WLQNLHYAVF GLGNRQYEHF NKIAKVADEL LEAQGGNRLV KVGLGDDDQC IEDDFSAWRE | 240 |
| SLWPELDMLL RDEDDATTVT TPYTAAVLEY RVVFHDSADV AAEDKSWINA NGHAVHDAQH | 300 |
| PFRSNVVVRK ELHTSASDRS CSHLEFNISG SALNYETGDH VGVYCENLTE TVDEALNLLG | 360 |
| LSPETYFSIY TDNEDGTPLG GSSLPPPFPS CTLRTALTRY ADLLNSPKKS ALLALAAHAS | 420 |
| NPVEADRLRY LASPAGKDEY AQSVIGSQKS LLEVMAEFPS AKPPLGVFFA AVAPRLQPRF | 480 |
| YSISSSPRMA PSRIHVTCAL VYDKMPTGRI HKGVCSTWMK NSVPMEKSHE CSWAPIFVRQ | 540 |
| SNFKLPAESK VPIIMVGPGT GLAPFRGFLQ ERLALKESGV ELGPSILFFG CRNRRMDYIY | 600 |
| EDELNNFVET GALSELVIAF SREGPTKEYV QHKMAEKASD IWNLISEGAY LYVCGDAKGM | 660 |
| AKDVHRTLHT IMQEQGSLDS SKAESMVKNL QMNGRYLRDV W | 701 |
| SEQ ID NO: 81 | |
| atggcagaat tagatacact tgatatagta gtattaggtg ttatcttttt gggtactgtg | 60 |
| gcatacttta ctaagggtaa attgtggggt gttaccaagg atccatacgc taacggattc | 120 |
| gctgcaggtg gtgcttccaa gcctggcaga actagaaaca tcgtcgaagc tatggaggaa | 180 |
| tcaggtaaaa actgtgtLgt tttctacggc agtcaaacag gtacagcgga ggattacgca | 240 |
| tcaagacttg caaaggaagg aaagtccaga ttcggtttga acactatgat cgccgatcta | 300 |
| gaagattatg acttcgataa cttagacact gttccatctg ataacatcgt tatgtttgta | 360 |
| ttggctactt acggtgaagg cgaaccaaca gataacgccg tggatttcta tgagttcatt | 420 |
| actggcgaag atgcctcttt caatgagggc aacgatcctc cactaggtaa cttgaattac | 480 |
| gttgcgttcg gtctgggcaa caatacctac gaacactaca actcaatggt caggaacgtt | 540 |
| aacaaggctc tagaaaagtt aggagctcat agaattggag aagcaggtga gggtgacgac | 600 |
| ggagctggaa ctatggaaga ggacttttta gcttggaaag atccaatgtg ggaagccttg | 660 |
| gctaaaaaga tgggcttgga ggaaagagaa gctgtatatg aacctatttt cgctatcaat | 720 |
| gagagagatg atttgacccc tgaagcgaat gaggtatact tgggagaacc taataagcta | 780 |
| cacttggaag gtacagcgaa aggtccattc aactcccaca acccatatat cgcaccaatt | 840 |
| gcagaatcat acgaactttt ctcagctaag gatagaaatt gtctgcatat ggaaattgat | 900 |
| atttctggta gtaatctaaa gtatgaaaca ggcgaccata tcgcgatctg gcctaccaac | 960 |
| ccaggtgaag aggtcaacaa atttcttgac attctagatc tgtctggtaa gcaacattcc | 1020 |
| gtcgtaacag tgaaagcctt agaacctaca gccaaagttc cttttccaaa tccaactacc | 1080 |
| tacgatgcta tattgagata ccatctggaa atatgcgctc cagtttctag acagtttgtc | 1140 |
| tcaactttag cagcattcgc ccctaatgat gatatcaaag ctgagatgaa ccgtttggga | 1200 |
| tcagacaaag attacttcca cgaaaagaca ggaccacatt actacaatat cgctagattt | 1260 |
| ttggcctcag tctctaaagg tgaaaaatgg acaaagatac cattttctgc tttcatagaa | 1320 |
| ggccttacaa aactacaacc aagatactat tctatctctt cctctagttt agttcagcct | 1380 |
| aaaaagatta gtattactgc tgttgtcgaa tctcagcaaa ttccaggtag agatgaccca | 1440 |
| ttcagaggtg tagcgactaa ctacttgttc gctttgaagc agaaacaaaa cggtgatcca | 1500 |
| aatccagctc cttttggcca atcatacgag ttgacaggac caaggaataa gtatgatggt | 1560 |
| atacatgttc cagtccatgt aagacattct aactttaagc taccatctga tccaggcaaa | 1620 |
| cctattatca tgatcggtcc aggtaccggt gttgcccctt ttagaggctt cgtccaagag | 1680 |
| agggcaaaac aagccagaga tggtgtagaa gttggtaaaa cactgctgtt ctttggatgt | 1740 |
| agaaagagta cagaagattt catgtatcaa aaagagtggc aagagtacaa ggaagctctt | 1800 |
| ggcgacaaat tcgaaatgat tacagctttt tcaagagaag gatctaaaaa ggtttatgtt | 1860 |
| caacacagac tgaaggaaag atcaaaggaa gtttctgatc ttctatccca aaaagcatac | 1920 |
| ttctacgttt gcggagacgc cgcacatatg gcacgtgaag tgaacactgt gttagcacag | 1980 |
| atcatagcag aaggccgtgg tgtatcagaa gccaagggtg aggaaattgt caaaaacatg | 2040 |
| agatcagcaa atcaatacca agtgtgttct gatttcgtaa ctttacactg taaagagaca | 2100 |
| acatacgcga attcagaatt gcaagaggat gtctggagtt aa | 2142 |
| SEQ ID NO: 82 | |
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQVCS DFVTLHCKET TYANSELQED VWS | 713 |
| SEQ ID NO: 83 | |
| atgcaatcgg aatccgttga agcatcgacg attgatttga tgactgctgt tttgaaggac | 60 |
| acagtgatcg atacagcgaa cgcatctgat aacggagact caaagatgcc gccggcgttg | 120 |
| gcgatgatgt tcgaaattcg tgatctgttg ctgattttga ctacgtcagt tgctgttttg | 180 |
| gtcggatgtt tcgttgtttt ggtgtggaag agatcgtccg ggaagaagtc cggcaaggaa | 240 |
| ttggagccgc cgaagatcgt tgtgccgaag aggcggctgg agcaggaggt tgatgatggt | 300 |
| aagaagaagg ttacgatttt cttcggaaca caaactggaa cggctgaagg tttcgctaag | 360 |
| gcacttttcg aagaagcgaa agcgcgatat gaaaaggcag cgtttaaagt gattgatttg | 420 |
| gatgattatg ctguLgattt ggatgagtat gcagagaagc tgaagaagga aacatatgct | 480 |
| ttcttcttct tggctacata tggagatggt gagccaactg ataatgctgc caaattttat | 540 |
| aaatggttta ctgagggaga cgagaaaggc gtttggcttc aaaaacttca atatggagta | 600 |
| tttggtcttg gcaacagaca atatgaacat ttcaacaaga ttggaatagt ggttgatgat | 660 |
| ggtctcaccg agcagggtgc aaaacgcatt gttcccgttg gtcttggaga cgacgatcaa | 720 |
| tcaattgaag acgatttttc ggcatggaaa gagttagtgt ggcccgaatt ggatctattg | 780 |
| cttcgcgatg aagatgacaa agctgctgca actccttaca cagctgcaat ccctgaatac | 840 |
| cgcgtcgtat ttcatgacaa acccgatgcg ttttctgatg atcatactca aaccaatggt | 900 |
| catgctgttc atgatgctca acatccatgc agatccaatg tggctgttaa aaaagagctt | 960 |
| catactcctg aatccgatcg ttcatgcaca catcttgaat ttgacatttc tcacactgga | 1020 |
| ttatcttatg aaactgggga tcatgttggt gtatactgtg aaaacctaat tgaagtagtg | 1080 |
| gaagaagctg ggaaattgtt aggattatca acagatactt atttctcgtt acatattgat | 1140 |
| aacgaagatg gttcaccact tggtggacct tcattacaac ctccttttcc tccttgtact | 1200 |
| ttaagaaaag cattgactaa ttatgcagat ctgttaagct ctcccaaaaa gtcaactttg | 1260 |
| cttgctctag ctgctcatgc ttccgatccc actgaagctg atcgtttaag atttcttgca | 1320 |
| tctcgcgagg gcaaggatga atatgctgaa tgggttgttg caaaccaaag aagtcttctt | 1380 |
| gaagtcatgg aagctttccc gtcagctaga ccgccacttg gtgttttctt tgcagcggtt | 1440 |
| gcaccgcgtt tacagcctcg ttactactct atttcttcct ccccaaagat ggaaccaaac | 1500 |
| aggattcatg ttacttgcgc gttggtttat gaaaaaactc ccgcaggtcg tatccacaaa | 1560 |
| ggaatctgct caacctggat gaagaacgct gtacctttga ccgaaagtca agattgcagt | 1620 |
| tgggcaccga tttttgttag aacatcaaac ttcagacttc caattgaccc gaaagtcccg | 1680 |
| gttatcatga ttggtcctgg aaccgggttg gctccattta ggggttttct tcaagaaaga | 1740 |
| ttggctctta aagaatccgg aaccgaactc gggtcatcta ttttattctt cggttgtaga | 1800 |
| aaccgcaaag tggattacat atatgagaat gaactcaaca actttgttga aaatggtgcg | 1860 |
| ctttctgagc ttgatgttgc tttctcccgc gatggcccga cgaaagaata cgtgcaacat | 1920 |
| aaaatgaccc aaaaggcttc tgaaatatgg aatatgcttt ctgagggagc atatttatat | 1980 |
| gtatgtggtg atgctaaagg catggctaaa gatgtacacc gtacacttca caccattgtg | 2040 |
| caagaacagg gaagtttgga ctcgtctaaa gcggagttgt atgtgaagaa tctacaaatg | 2100 |
| tcaggaagat acctccgtga tgtttggtaa | 2130 |
| SEQ ID NO: 84 | |
| MQSESVEAST IDLMTAVLKD TVIDTANASD NGDSKMPPAL AMMFEIRDLL LILTTSVAVL | 60 |
| VGCFVVLVWK RSSGKKSGKE LEPPKIVVPK RRLEQEVDDG KKKVTIFFGT QTGTAEGFAK | 120 |
| ALFEEAKARY EKAAFKVIDL DDYAADLDEY AEKLKKETYA FFFLATYGDG EPTDNAAKFY | 180 |
| KWFTEGDEKG VWLQKLQYGV FGLGNRQYEH FNKIGIVVDD GLTEQGAKRI VPVGLGDDDQ | 240 |
| SIEDDFSAWK ELVWPELDLL LRDEDDKAAA TPYTAAIPEY RVVFHDKPDA FSDDHTQTNG | 300 |
| HAVHDAQHPC RSNVAVKKEL HTPESDRSCT HLEFDISHTG LSYETGDHVG VYCENLIEVV | 360 |
| EEAGKLLGLS TDTYFSLHID NEDGSPLGGP SLQPPFPPCT LRKALTNYAD LLSSPKKSTL | 420 |
| LALAAHASDP TEADRLRFLA SREGKDEYAE WVVANQRSLL EVMEAFPSAR PPLGVFFAAV | 480 |
| APRLQPRYYS ISSSPKMEPN RIHVTCALVY EKTPAGRIHK GICSTWMKNA VPLTESQDCS | 540 |
| WAPIFVRTSN FRLPIDPKVP VIMIGPGTGL APFRGFLQER LALKESGTEL GSSILFFGCR | 600 |
| NRKVDYIYEN ELNNFVENGA LSELDVAFSR DGPTKEYVQH KMTQKASEIW NMLSEGAYLY | 660 |
| VCGDAKGMAK DVHRTLHTIV QEQGSLDSSK AELYVKNLQM SGRYLRDVW | 709 |
| SEQ ID NO: 85 | |
| atgcaatcta actccgtgaa gatttcgccg cttgatctgg taactgcgct gtttagcggc | 60 |
| aaggttttgg acacatcgaa cgcatcggaa tcgggagaat ctgctatgct gccgactata | 120 |
| gcgatgatta tggagaatcg tgagctgttg atgatactca caacgtcggt tgctgtattg | 180 |
| atcggatgcg ttgtcgtttt ggtgtggcgg agatcgtcta cgaagaagtc ggcgttggag | 240 |
| ccaccggtga ttgtggttcc gaagagagtg caagaggagg aagttgatga tggtaagaag | 300 |
| aaagttacgg ttttcttcgg cacccaaact ggaacagctg aaggcttcgc taaggcactt | 360 |
| gttgaggaag ctaaagctcg atatgaaaag gctgtcttta aagtaattga tttggatgat | 420 |
| tatgctgctg atgacgatga gtatgaggag aaactaaaga aagaatcttt ggcctttttc | 480 |
| tttttggcta cgtatggaga tggtgagcca acagataatg ctgccagatt ttataaatgg | 540 |
| tttactgagg gagatgcgaa aggagaatgg cttaataagc ttcaatatgg agtatttggt | 600 |
| ttgggtaaca gacaatatga acattttaac aagatcgcaa aagtggttga tgatggtctt | 660 |
| gtagaacagg gLgcaaagcg tcttgttcct gttggacttg gagatgatga tcaatgtatt | 720 |
| gaagatgact tcaccgcatg gaaagagtta gtatggccgg agttggatca attacttcgt | 780 |
| gatgaggatg acacaactgt tgctactcca tacacagctg ctgttgcaga atatcgcgtt | 840 |
| gtttttcatg aaaaaccaga cgcgctttct gaagattata gttatacaaa tggccatgct | 900 |
| gttcatgatg ctcaacatcc atgcagatcc aacgtggctg tcaaaaagga acttcatagt | 960 |
| cctgaatctg accggtcttg cactcatctt gaatttgaca tctcgaacac cggactatca | 1020 |
| tatgaaactg gggaccatgt tggagtttac tgtgaaaact tgagtgaagt tgtgaatgat | 1080 |
| gctgaaagat tagtaggatt accaccagac acttactcct ccatccacac tgatagtgaa | 1140 |
| gacgggtcgc cacttggcgg agcctcattg ccgcctcctt tcccgccatg cactttaagg | 1200 |
| aaagcattga cgtgttatgc tgatgttttg agttctccca agaagtcggc tttgcttgca | 1260 |
| ctagctgctc atgccaccga tcccagtgaa gctgatagat tgaaatttct tgcatccccc | 1320 |
| gccggaaagg atgaatattc tcaatggata gttgcaagcc aaagaagtct ccttgaagtc | 1380 |
| atggaagcat tcccgtcagc taagccttca cttggtgttt tctttgcatc tgttgccccg | 1440 |
| cgcttacaac caagatacta ctctatttct tcctcaccca agatggcacc ggataggatt | 1500 |
| catgttacat gtgcattagt ctatgagaaa acacctgcag gccgcatcca caaaggagtt | 1560 |
| tgttcaactt ggatgaagaa cgcagtgcct atgaccgaga gtcaagattg cagttgggcc | 1620 |
| ccaatatacg tccgaacatc caatttcaga ctaccatctg accctaaggt cccggttatc | 1680 |
| atgattggac ctggcactgg tttggctcct tttagaggtt tccttcaaga gcggttagct | 1740 |
| ttaaaggaag ccggaactga cctcggttta tccattttat tcttcggatg taggaatcgc | 1800 |
| aaagtggatt tcatatatga aaacgagctt aacaactttg tggagactgg tgctctttct | 1860 |
| gagcttattg ttgctttctc ccgtgaaggc ccgactaagg aatatgtgca acacaagatg | 1920 |
| agtgagaagg cttcggatat ctggaacttg ctttctgaag gagcatattt atacgtatgt | 1980 |
| ggtgatgcca aaggcatggc caaagatgta catcgaaccc tccacacaat tgtgcaagaa | 2040 |
| cagggatctc ttgactcgtc aaaggcagaa ctctacgtga agaatctaca aatgtcagga | 2100 |
| agatacctcc gtgacgtttg gtaa | 2124 |
| SEQ ID NO: 86 | |
| MQSNSVKISP LDLVTALFSG KVLDTSNASE SGESAMLPTI AMIMENRELL MILTTSVAVL | 60 |
| IGCVVVLVWR RSSTKKSALE PPVIVVPKRV QEEEVDDGKK KVTVFFGTQT GTAEGFAKAL | 120 |
| VEEAKARYEK AVFKVIDLDD YAADDDEYEE KLKKESLAFF FLATYGDGEP TDNAARFYKW | 180 |
| FTEGDAKGEW LNKLQYGVEG LGNRQYEHFN KIAKVVDDGL VEQGAKRLVP VGLGDDDQCI | 240 |
| EDDFTAWKEL VWPELDQLLR DEDDTTVATP YTAAVAEYRV VFHEKPDALS EDYSYTNGHA | 300 |
| VHDAQHPCRS NVAVKKELHS PESDRSCTHL EFDISNTGLS YETGDHVGVY CENLSEVVND | 360 |
| AERLVGLPPD TYSSIHTDSE DGSPLGGASL PPPFPPCTLR KALTCYADVL SSPKKSALLA | 420 |
| LAAHATDPSE ADRLKFLASP AGKDEYSQWI VASQRSLLEV MEAFPSAKPS LGVFFASVAP | 480 |
| RLQPRYYSIS SSPKMAPDRI HVTCALVYEK TPAGRIHKGV CSTWMKNAVP MTESQDCSWA | 540 |
| PIYVRTSNFR LPSDPKVPVI MIGPGTGLAP FRGFLQERLA LKEAGTDLGL SILFFGCRNR | 600 |
| KVDFIYENEL NNFVETGALS ELIVAFSREG PTKEYVQHKM SEKASDIWNL LSEGAYLYVC | 660 |
| GDAKGMAKDV HRTLHTIVQE QGSLDSSKAE LYVKNLQMSG RYLRDVW | 707 |
| SEQ ID NO: 87 | |
| atgtcctcca actccgattt ggtcagaaga ttggaatctg ttttgggtgt ttctttcggt | 60 |
| ggttctgtta ctgattccgt tgttgttatt gctaccacct ctattgcttt ggttatcggt | 120 |
| gttttggttt tgttgtggag aagatcctct gacagatcta gagaagttaa gcaattggct | 180 |
| gttccaaagc cagttactat cgttgaagaa gaagatgaat tcgaagttgc ttctggtaag | 240 |
| accagagttt ctattttcta cggtactcaa actggtactg ctgaaggttt tgctaaggct | 300 |
| ttggctgaag aaatcaaagc cagatacgaa aaagctgccg ttaaggttat tgatttggat | 360 |
| gattacacag ccgaagatga caaatacggt gaaaagttga agaaagaaac tatggccttc | 420 |
| ttcatgttgg ctacttatgg tgatggtgaa cctactgata atgctgctag attttacaag | 480 |
| tggttcaccg aaggtactga tagaggtgtt tggttggaac atttgagata cggtgtattc | 540 |
| ggtttgggta acagacaata cgaacacttc aacaagattg ccaaggttgt tgatgatttg | 600 |
| ttggttgaac aaggtgccaa gagattggtt actgttggtt tgggtgatga tgatcaatgc | 660 |
| atcgaagatg atttctccgc ttggaaagaa gccttgtggc cagaattgga tcaattattg | 720 |
| caagatgata ccaacaccgt ttctactcca tacactgctg ttattccaga atacagagtt | 780 |
| gttatccacg atccatctgt tacctcttat gaagatccat actctaacat ggctaacggt | 840 |
| aatgcctctt acgatattca tcatccatgt agagctaacg ttgccgtcca aaaagaattg | 900 |
| cataagccag aatctgacag aagttgcatc catttggaat tcgatatttt cgctactggt | 960 |
| ttgacttacg aaaccggtga tcatgttggt gtttacgctg ataattgtga tgatactgta | 1020 |
| gaagaagccg ctaagttgtt gggtcaacca ttggatttgt tgttctccat tcataccgat | 1080 |
| aacaacgacg gtacttcttt gggttcttct ttgccaccac catttccagg tccatgtact | 1140 |
| ttgagaactg ctttggctag atatgccgat ttgttgaatc caccaaaaaa ggctgctttg | 1200 |
| attgctttag ctgctcatgc tgatgaacca tctgaagctg aaagattgaa gttcttgtca | 1260 |
| tctccacaag gtaaggacga atattctaaa tgggttgtcg gttcccaaag atccttggtt | 1320 |
| gaagttatgg ctgaatttcc atctgctaaa ccaccattgg gtgtattttt tgctgctgtt | 1380 |
| gttcctagat tgcaacctag atattactcc atctcttcca gtccaagatt tgctccacat | 1440 |
| agagttcatg ttacttgcgc tttggtttat ggtccaactc caactggtag aattcacaga | 1500 |
| ggtgtatgtt cattctggat gaagaatgtt gtcccattgg aaaagtctca aaactgttct | 1560 |
| tgggccccaa ttttcatcag acaatctaat ttcaagttgc cagccgatca ttctgttcca | 1620 |
| atagttatgg ttggtccagg tactggttta gctcctttta gaggtttctt acaagaaaga | 1680 |
| ttggccttga aagaagaagg tgctcaagtt ggtcctgctt tgttgttttt tggttgcaga | 1740 |
| aacagacaaa tggacttcat ctacgaagtc gaattgaaca actttgtcga acaaggtgct | 1800 |
| ttgtccgaat tgatcgttgc tttttcaaga gaaggtccat ccaaagaata cgtccaacat | 1860 |
| aagatggttg aaaaggcagc ttacatgtgg aacttgattt ctcaaggtgg ttacttctac | 1920 |
| gtttgtggtg atgctaaagg tatggctaga gatgttcata gaacattgca taccatcgtc | 1980 |
| caacaagaag aaaaggttga ttctaccaag gccgaatcca tcgttaagaa attgcaaatg | 2040 |
| gacggtagat acttgagaga tgtttggtga | 2070 |
| SEQ ID NO: 88 | |
| MSSNSDLVRR LESVLGVSFG GSVTDSVVVI ATTSIALVIG VLVLLWRRSS DRSREVKQLA | 60 |
| VPKPVTIVEE EDEFEVASGK TRVSIFYGTQ TGTAEGFAKA LAEEIKARYE KAAVKVIDLD | 120 |
| DYTAEDDKYG EKLKKETMAF FMLATYGDGE PTDNAARFYK WFTEGTDRGV WLEHLRYGVF | 180 |
| GLGNRQYEHF NKIAKVVDDL LVEQGAKRLV TVGLGDDDQC IEDDFSAWKE ALWPELDQLL | 240 |
| QDDTNTVSTP YTAVIPEYRV VIHDPSVTSY EDPYSNMANG NASYDIHHPC RANVAVQKEL | 300 |
| HKPESDRSCI HLEFDIFATG LTYETGDHVG VYADNCDDTV EEAAKLLGQP LDLLFSIHTD | 360 |
| NNDGTSLGSS LPPPFPGPCT LRTALARYAD LLNPPKKAAL IALAAHADEP SEAERLKFLS | 420 |
| SPQGKDEYSK WVVGSQRSLV EVMAEFPSAK PPLGVFFAAV VPRLQPRYYS ISSSPRFAPH | 480 |
| RVHVTCALVY GPTPTGRIHR GVCSFWMKNV VPLEKSQNCS WAPIFIRQSN FKLPADHSVP | 540 |
| IVMVGPGTGL APFRGFLQER LALKEEGAQV GPALLFFGCR NRQMDFIYEV ELNNEVEQGA | 600 |
| LSELIVAFSR EGPSKEYVQH KMVEKAAYMW NLISQGGYFY VCGDAKGMAR DVHRTLHTIV | 660 |
| QQEEKVDSTK AESIVKKLQM DGRYLRDVW | 689 |
| SEQ ID NO: 89 | |
| atgacttctg cactttatgc ctccgatctt ttcaaacaat tgaaaagtat catgggaacg | 60 |
| gattctttgt ccgatgatgt tgtattagtt attgctacaa cttctctggc actggttgct | 120 |
| ggtttcgttg tcttattgtg gaaaaagacc acggcagatc gttccggcga gctaaagcca | 180 |
| ctaatgatcc ctaagtctct gatggcgaaa gatgaggatg atgacttaga tctaggttct | 240 |
| ggaaaaacga gagtctctat cttcttcggc acacaaaccg gaacagccga aggattcgct | 300 |
| aaagcacttt cagaagagat caaagcaaga tacgaaaagg cggctgtaaa agtaatcgat | 360 |
| ttggatgatt acgctgccga tgatgaccaa tatgaggaaa agttgaaaaa ggaaacattg | 420 |
| gctttctttt gtgtagccac gtatggtgat ggtgaaccaa ccgataacgc cgcaagattc | 480 |
| tacaagtggt ttactgaaga gaacgaaaga gatatcaagt tgcagcaact tgcttacggc | 540 |
| gtttttgcct taggtaacag acaatacgag cactttaaca agataggtat tgtcttagat | 600 |
| gaagagttat gcaaaaaggg tgcgaagaga ttgattgaag tcggtttagg agatgatgat | 660 |
| caatctatcg aggatgactt taatgcatgg aaggaatctt tgtggtctga attagataag | 720 |
| ttacttaagg acgaagatga taaatccgtt gccactccat acacagccgt cattccagaa | 780 |
| tatagagtag ttactcatga tccaagattc acaacacaga aatcaatgga aagtaatgtg | 840 |
| gctaatggta atactaccat cgatattcat catccatgta gagtagacgt tgcagttcaa | 900 |
| aaggaattgc acactcatga atcagacaga tcttgcatac atcttgaatt tgatatatca | 960 |
| cgtactggta tcacttacga aacaggtgat cacgtgggtg tctacgctga aaaccatgtt | 1020 |
| gaaattgtag aggaagctgg aaagttgttg ggccatagtt tagatcttgt tttctcaatt | 1080 |
| catgccgata aagaggatgg ctcaccacta gaaagtgcag tgcctccacc atttccagga | 1140 |
| ccatgcaccc taggtaccgg tttagctcgt tacgcggatc tgttaaatcc tccacgtaaa | 1200 |
| tcagcLctag tggccttggc tgcgtacgcc acagaacctt ctgaggcaga aaaactgaaa | 1260 |
| catctaactt caccagatgg taaggatgaa tactcacaat ggatagtagc tagtcaacgt | 1320 |
| tctttactag aagttatggc tgctttccca tccgctaaac ctcctttggg tgttttcttc | 1380 |
| gccgcaatag cgcctagact gcaaccaaga tactattcaa tttcatcctc acctagactg | 1440 |
| gcaccatcaa gagttcatgt cacatccgct ttagtgtacg gtccaactcc tactggtaga | 1500 |
| atccataagg gcgtttgttc aacatggatg aaaaacgcgg ttccagcaga gaagtctcac | 1560 |
| gaatgttctg gtgctccaat ctttatcaga gcctccaact tcaaactgcc ttccaatcct | 1620 |
| tctactccta ttgtcatggt cggtcctggt acaggtcttg ctccattcag aggtttctta | 1680 |
| caagagagaa tggccttaaa ggaggatggt gaagagttgg gatcttcttt gttgtttttc | 1740 |
| ggctgtagaa acagacaaat ggatttcatc tacgaagatg aactgaataa ctttgtagat | 1800 |
| caaggagtta tttcagagtt gataatggct ttttctagag aaggtgctca gaaggagtac | 1860 |
| gtccaacaca aaatgatgga aaaggccgca caagtttggg acttaatcaa agaggaaggc | 1920 |
| tatctatatg tctgtggtga tgcaaagggt atggcaagag atgttcacag aacacttcat | 1980 |
| actatagtcc aggaacagga aggcgttagt tcttctgaag cggaagcaat tgtgaaaaag | 2040 |
| ttacaaacag agggaagata cttgagagat gtgtggtaa | 2079 |
| SEQ ID NO: 90 | |
| MTSALYASDL FKQLKSIMGT DSLSDDVVLV IATTSLALVA GFVVLLWKKT TADRSGELKP | 60 |
| LMIPKSLMAK DEDDDLDLGS GKTRVSIFFG TQTGTAEGFA KALSEEIKAR YEKAAVKVID | 120 |
| LDDYAADDDQ YEEKLKKETL AFFCVATYGD GEPTDNAARF YKWFTEENER DIKLQQLAYG | 180 |
| VFALGNRQYE HFNKIGIVLD EELCKKGAKR LIEVGLGDDD QSIEDDFNAW KESLWSELDK | 240 |
| LLKDEDDKSV ATPYTAVIPE YRVVTHDPRF TTQKSMESNV ANGNTTIDIH HPCRVDVAVQ | 300 |
| KELHTHESDR SCIHLEFDIS RTGITYETGD HVGVYAENHV EIVEEAGKLL GHSLDLVFSI | 360 |
| HADKEDGSPL ESAVPPPFPG PCTLGTGLAR YADLLNPPRK SALVALAAYA TEPSEAEKLK | 420 |
| HLTSPDGKDE YSQWIVASQR SLLEVMAAFP SAKPPLGVFF AAIAPRLQPR YYSISSSPRL | 480 |
| APSRVHVTSA LVYGPTPTGR IHKGVCSTWM KNAVPAEKSH ECSGAPIFIR ASNFKLPSNP | 540 |
| STPIVMVGPG TGLAPERGEL QERMALKEDG EELGSSLLFF GCRNRQMDFI YEDELNNFVD | 600 |
| QGVISELIMA FSREGAQKEY VQHKMMEKAA QVWDLIKEEG YLYVCGDAKG MARDVHRTLH | 660 |
| TIVQEQEGVS SSEAEAIVKK LQTEGRYLRD VW | 692 |
| SEQ ID NO: 91 | |
| atgtcttcct cttcctcttc cagtacctct atgattgatt tgatggctgc tattattaaa | 60 |
| ggtgaaccag ttatcgtctc cgacccagca aatgcctctg cttatgaatc agttgctgca | 120 |
| gaattgtctt caatgttgat cgaaaacaga caattcgcca tgatcgtaac tacatcaatc | 180 |
| gctgttttga tcggttgtat tgtcatgttg gtatggagaa gatccggtag tggtaattct | 240 |
| aaaagagtcg aacctttgaa accattagta attaagccaa gagaagaaga aatagatgac | 300 |
| ggtagaaaga aagttacaat atttttcggt acccaaactg gtacagctga aggttttgca | 360 |
| aaagccttag gtgaagaagc taaggcaaga tacgaaaaga ctagattcaa gatagtcgat | 420 |
| ttggatgact atgccgctga tgacgatgaa tacgaagaaa agttgaagaa agaagatgtt | 480 |
| gcatttttct ttttggcaac ctatggtgac ggtgaaccaa ctgacaatgc agccagattc | 540 |
| tacaaatggt ttacagaggg taatgatcgt ggtyaatggt tgaaaaactt aaagtacggt | 600 |
| gttttcggtt tgggtaacag acaatacgaa catttcaaca aagttgcaaa ggttgtcgac | 660 |
| gatattttgg tcgaacaagg tgctcaaaga ttagtccaag taggtttggg tgacgatgac | 720 |
| caatgtatag aagatgactt tactgcctgg agagaagctt tgtggcctga attagacaca | 780 |
| atcttgagag aagaaggtga caccgccgtt gctaccccat atactgctgc agtattagaa | 840 |
| tacagagttt ccatccatga tagtgaagac gcaaagttta atgatatcac tttggccaat | 900 |
| ggtaacggtt atacagtttt cgatgcacaa cacccttaca aagctaacgt tgcagtcaag | 960 |
| agagaattac atacaccaga atccgacaga agttgtatac acttggaatt tgatatcgct | 1020 |
| ggttccggtt taaccatgaa gttgggtgac catgtaggtg ttttatgcga caatttgtct | 1080 |
| gaaactgttg atgaagcatt gagattgttg gatatgtccc ctgacactta ttttagtttg | 1140 |
| cacgctgaaa aagaagatgg tacaccaatt tccagttctt taccacctcc attccctcca | 1200 |
| tgtaacttaa gaacagcctt gaccagatac gcttgcttgt tatcatcccc taaaaagtcc | 1260 |
| gccttggttg ctttagccgc tcatgctagt gatcctactg aagcagaaag attgaaacac | 1320 |
| ttagcatctc cagccggtaa agatgaatat tcaaagtggg tagttgaatc tcaaagatca | 1380 |
| ttgttagaag ttatggcaga atttccatct gccaagcctc cattaggtgt cttctttgct | 1440 |
| ggtgtagcac ctagattgca accaagattc tactcaatca gttcttcacc taagatcgct | 1500 |
| gadactagaa ttcatgttac atgtgcatta gtctacgaaa agatgccaac cggtagaatt | 1560 |
| cacaagggtg tatgctctac ttggatgaaa aatgctgttc cttacgaaaa atcagaaaag | 1620 |
| ttgttcttag gtagaccaat cttcgtaaga caatcaaact tcaagttgcc ttctgattca | 1680 |
| aaggttccaa taatcatgat aggtcctggt acaggtttag ccccattcag aggtttcttg | 1740 |
| caagaaagat tggctttagt tgaatctggt gtcgaattag gtccttcagt tttgttcttt | 1800 |
| ggttgtagaa acagaagaat ggatttcatc tatgaagaag aattgcaaag attcgtcgaa | 1860 |
| tctggtgcat tggccgaatt atctgtagct ttttcaagag aaggtccaac taaggaatac | 1920 |
| gttcaacata agatgatgga taaggcatcc gacatatgga acatgatcag tcaaggtgct | 1980 |
| tatttgtacg tttgcggtga cgcaaagggt atggccagag atgtccatag atctttgcac | 2040 |
| acaattgctc aagaacaagg ttccatggat agtaccaaag ctgaaggttt cgtaaagaac | 2100 |
| ttacaaactt ccggtagata cttgagagat gtctggtga | 2139 |
| SEQ ID NO: 92 | |
| MSSSSSSSTS MIDLMAAIIK GEPVIVSDPA NASAYESVAA ELSSMLIENR QFAMIVTTSI | 60 |
| AVLIGCIVML VWRRSGSGNS KRVEPLKPLV IKPREEEIDD GRKKVTIFFG TQTGTAEGFA | 120 |
| KALGEEAKAR YEKTRFKIVD LDDYAADDDE YEEKLKKEDV AFFFLATYGD GEPTDNAARF | 180 |
| YKWFTEGNDR GEWLKNLKYG VFGLGNRQYE HFNKVAKVVD DILVEQGAQR LVQVGLGDDD | 240 |
| QCIEDDFTAW REALWPELDT ILREEGDTAV ATPYTAAVLE YRVSIHDSED AKFNDITLAN | 300 |
| GNGYTVFDAQ HPYKANVAVK RELHTPESDR SCIHLEFDIA GSGLTMKLGD HVGVLCDNLS | 360 |
| ETVDEALRLL DMSPDTYFSL HAEKEDGTPI SSSLPPPFPP CNLRTALTRY ACLLSSPKKS | 420 |
| ALVALAAHAS DPTEAERLKH LASPAGKDEY SKWVVESQRS LLEVMAEFPS AKPPLGVFFA | 480 |
| GVAPRLQPRF YSISSSPKIA ETRIHVICAL VYEKMPTGRI HKGVCSTWMK NAVPYEKSEK | 540 |
| LFLGRPIFVR QSNFKLPSDS KVPIIMIGPG TGLAPERGEL QERLALVESG VELGPSVLFF | 600 |
| GCRNRRMDFI YEEELQRFVE SGALAELSVA FSREGPTKEY VQHKMMDKAS DIWNMISQGA | 660 |
| YLYVCGDAKG MARDVHRSLH TIAQEQGSMD STKAEGFVKN LQTSGRYLRD VW | 712 |
| SEQ ID NO: 93 | |
| atggaagcct cttacctata catttctatt ttgcttttac tggcatcata cctgttcacc | 60 |
| actcaactta gaaggaagag cgctaatcta ccaccaaccg tgtttccatc aataccaatc | 120 |
| attggacact tatacttact caaaaagcct ctttatagaa ctttagcaaa aattgccgct | 180 |
| aagtacggac caatactgca attacaactc ggctacagac gtgttctggt gatttcctca | 240 |
| ccatcagcag cagaagagtg ctttaccaat aacgatgtaa tcttcgcaaa tagacctaag | 300 |
| acattgtttg gcaaaatagt gggtggaaca tcccttggca gtttatccta cggcgatcaa | 360 |
| tggcgtaatc taaggagagt agcttctatc gaaatcctat cagttcatag gttgaacgaa | 420 |
| tttcatgata tcagagtgga tgagaacaga ttgttaatta gaaaacttag aagttcatct | 480 |
| tctcctgtta ctcttataac agtcttttat gctctaacat tgaacgtcat tatgagaatg | 540 |
| atctctggca aaagatattt cgacagtggg gatagagaat tggaggagga aggtaagaga | 600 |
| tttcgagaaa tcttagacga aacgttgctt ctagccggtg cttctaatgt tggcgactac | 660 |
| ttaccaatat tgaactggtt gggagttaag tctcttgaaa agaaattgat cgctttgcag | 720 |
| aaaaagagag atgacttttt ccagggtttg attgaacagg ttagaaaatc tcgtggtgct | 780 |
| aaagtaggca aaggtagaaa aacgatgatc gaactcttat tatctttgca agagtcagaa | 840 |
| cctgagtact atacagatgc tatgataaga tcttttgtcc taggtctgct ggctgcaggt | 900 |
| agtgatactt cagcgggcac tatggaatgg gccatgagct tactggtcaa tcacccacat | 960 |
| gtattgaaga aagctcaagc tgaaatcgat agagttatcg gtaataacag attgattgac | 1020 |
| gagtcagaca ttggaaatat cccttacatc gggtgtatta tcaatgaaac tctaagactc | 1080 |
| tatccagcag ggccattgtt gttcccacat gaaagttctg ccgactgcgt tatttccggt | 1140 |
| tacaatatac ctagaggtac aatgttaatc gtaaaccaat gggcgattca tcacgatcct | 1200 |
| aaagtctggg atgatcctga aacctttaaa cctgaaagat ttcaaggatt agaaggaact | 1260 |
| agagatggtt tcaaacttat gccattcggt tctgggagaa gaggatgtcc aggtgaaggt | 1320 |
| ttggcaataa ggctgttagg gatgacacta ggctcagtga tccaatgttt tgattgggag | 1380 |
| agagtaggag atgagatggt tgacatgaca gaaggtttgg gtgtcacact tcctaaggcc | 1440 |
| gttccattag ttgccaaatg taagccacgt tccgaaatga ctaatctcct atccgaactt | 1500 |
| taa | 1503 |
| SEQ ID NO: 94 | |
| MEASYLYISI LLLLASYLFT TQLRRKSANL PPTVFPSIPI IGHLYLLKKP LYRTLAKIAA | 60 |
| KYGPILQLQL GYRRVLVISS PSAAEECFTN NDVIFANRPK ILFGKIVGGT SLGSLSYGDQ | 120 |
| WRNLRRVASI EILSVHRLNE FHDIRVDENR LLIRKLRSSS SPVTLITVFY ALTLNVIMRM | 180 |
| ISGKRYFDSG DRELEEEGKR FREILDETLL LAGASNVGDY LPILNWLGVK SLEKKLIALQ | 240 |
| KKRDDFFQGL IEQVRKSRGA KVGKGRKTMI ELLLSLQESE PEYYTDAMIR SFVLGLLAAG | 300 |
| SDTSAGTMEW AMSLLVNHPH VLKKAQAEID RVIGNNRLID ESDIGNIPYI GCIINETLRL | 360 |
| YPAGPLLFPH ESSADCVISG YNIPRGTMLI VNQWAIHHDP KVWDDPETEK PERFQGLEGT | 420 |
| RDGFKLMPFG SGRRGCPGEG LAIRLLGMTL GSVIQCFDWE RVGDEMVDMT EGLGVTLPKA | 480 |
| VPLVAKCKPR SEMTNLLSEL | 500 |
| SEQ ID NO: 95 | |
| atggaagtaa cagtagctag tagtgtagcc ctgagcctgg tctttattag catagtagta | 60 |
| agatgggcat ggagtgtggt gaattgggtg tggtttaagc cgaagaagct ggaaagattt | 120 |
| ttgagggagc aaggccttaa aggcaattcc tacaggtttt tatatggaga catgaaggag | 180 |
| aactctatcc tgctcaaaca agcaagatcc aaacccatga acctctccac ctcccatgac | 240 |
| atagcacctc aagtcacccc ttttgtcgac caaaccgtga aagcttacgg taagaactct | 300 |
| tttaattggg ttggccccat accaagggtg aacataatga atccagaaga tttgaaggac | 360 |
| gtcttaacaa aaaatgttga ctttgttaag ccaatatcaa acccacttat caagttgcta | 420 |
| gctacaggta ttgcaatcta tgaaggtgag aaatggacta aacacagaag gattatcaac | 480 |
| ccaacattcc attcggagag gctaaagcgt atgttacctt catttcacca aagttgtaat | 540 |
| gagatggtca aggaatggga gagcttggtg tcaaaagagg gttcatcatg tgagttggat | 600 |
| gtctggcctt ttcttgaaaa tatgtcggca gatgtgatct cgagaacagc atttggaact | 660 |
| agctacaaaa aaggacagaa aatctttgaa ctcttgagag agcaagtaat atatgtaacg | 720 |
| aaaggctttc aaagttttta cattccagga tggaggtttc tcccaactaa gatgaacaag | 780 |
| aggatgaatg agattaacga agaaataaaa ggattaatca ggggtattat aattgacaga | 840 |
| gagcaaatca ttaaggcagg tgaagaaacc aacgatgact tattaggtgc acttatggag | 900 |
| tcaaacttga aggacattcg ggaacatggg aaaaacaaca aaaatgttgg gatgagtatt | 960 |
| gaagatgtaa ttcaggagtg taagctgttt tactttgctg ggcaagaaac cacttcagtg | 1020 |
| ttgctggctt ggacaatggt tttacttggt caaaatcaga actggcaaga tcgagcaaga | 1080 |
| caagaggttt tgcaagtctt tggaagcagc aagccagatt ttgatggtct agctcacctt | 1140 |
| aaagtcgtaa ccatgatttt gcttgaagtt cttcgattat acccaccagt cattgaactt | 1200 |
| attcgaacca ttcacaagaa aacacaactt gggaagctct cactaccaga aggagttgaa | 1260 |
| gtccgcttac caacactgct cattcaccat gacaaggaac tgtggggtga tgatgcaaac | 1320 |
| cagttcaatc cagagaggtt ttcggaagga gtttccaaag caacaaagaa ccgactctca | 1380 |
| ttcttcccct tcggagccgg tccacgcatt tgcattggac agaacttttc tatgatggaa | 1440 |
| gcaaagttgg ccttagcatt gatcttgcaa cacttcacct ttgagctttc tccatctcat | 1500 |
| gcacatgctc cttcccatcg tataaccctt caaccacagt atggtgttcg tatcatttta | 1560 |
| catcgaggtt ag | 1572 |
| SEQ ID NO: 96 | |
| atggaagtca ctgtcgcctc ttctgtcgct ttatccttag tcttcatttc cattgtcgtc | 60 |
| agatgggctt ggtccgttgt caactgggtt tggttcaaac caaagaagtt ggaaagattc | 120 |
| ttgagagagc aaggtttgaa gggtaattct tatagattct tgtacggtga catgaaggaa | 180 |
| aattctattt tgttgaagca agccagatcc aaaccaatga acttgtctac ctctcatgat | 240 |
| attgctccac aagttactcc attcgtcgat caaactgtta aagcctacgg taagaactct | 300 |
| ttcaattggg ttggtccaat tcctagagtt aacatcatga acccagaaga tttgaaggat | 360 |
| gtcttgacca agaacgttga cttcgttaag ccaatttcca acccattgat taaattgttg | 420 |
| gctactggta ttgccattta cgaaggtgaa aagtggacta agcatagaag aatcatcaac | 480 |
| cctaccttcc actctgaaag attgaagaga atgttaccat ctttccatca atcctgtaat | 540 |
| gaaatggtta aggaatggga atccttggtt tctaaagaag gttcttcttg cgaattggat | 600 |
| gtttggccat tcttggaaaa tatgtctgct gatgtcattt ccagaaccgc tttcggtacc | 660 |
| tcctacaaga agggtcaaaa gattttcgaa ttgttgagag agcaagttat ttacgttacc | 720 |
| aagggtttcc aatccttcta catcccaggt tggagattct tgccaactaa aatgaacaag | 780 |
| cgtatgaacg agatcaacga agaaattaaa ggtttgatca gaggtattat tatcgacaga | 840 |
| gaacaaatta ttaaagctgg tgaagaaacc aacgatgatt tgttgggtgc tttgatggag | 900 |
| tccaacttga aggatattag agaacatggt aagaacaaca agaatgttgg tatgtctatt | 960 |
| gaagatgtta ttcaagaatg taagttattc tacttcgctg gtcaagagac cacttctgtt | 1020 |
| ttgttagcct ggactatggt cttgttaggt caaaaccaaa attggcaaga tagagctaga | 1080 |
| caagaagttt tgcaagtctt cggttcttcc aagccagact ttgatggttt ggcccacttg | 1140 |
| aaggttgtta ctatgatttt gttagaagtt ttgagattgt acccaccagt cattgagtta | 1200 |
| atcagaacca ttcataaaaa gactcaattg ggtaaattat ctttgccaga aggtgttgaa | 1260 |
| gtcagattac caaccttgtt gattcaccac gataaggaat tatggggtga cgacgctaat | 1320 |
| caatttaatc cagaaagatt ttccgaaggt gtttccaagg ctaccaaaaa ccgtttgtcc | 1380 |
| ttcttcccat ttggtgctgg tccacgtatt tgtatcggtc aaaacttttc catgatggaa | 1440 |
| gccaagttgg ctttggcttt aatcttgcaa cacttcactt tcgaattgtc tccatcccat | 1500 |
| gcccacgctc cttctcatag aatcacttta caaccacaat acggtgtcag aatcatctta | 1560 |
| cacagaagat aa | 1572 |
| SEQ ID NO: 97 | |
| MEVTVASSVA LSLVFISIVV RWAWSVVNWV WEKPKKLERF LREQGLKGNS YRFLYGDMKE | 60 |
| NSILLKQARS KPMNLSTSHD IAPQVTPFVD QTVKAYGKNS FNWVGPIPRV NIMNPEDLKD | 120 |
| VLTKNVDFVK PISNPLIKLL ATGIAIYEGE KWTKHRRIIN PTFHSERLKR MLPSFHQSCN | 180 |
| EMVKEWESLV SKEGSSCELD VWPFLENMSA DVISRTAFGT SYKKGQKIFE LLREQVIYVT | 240 |
| KGFQSFYIPG WRFLPTKMNK RMNEINEEIK GLIRGIIIDR EQIIKAGEET NDDLLGALME | 300 |
| SNLKDIREHG KNNKNVGMSI EDVIQECKLF YFAGQETTSV LLAWTMVLLG QNQNWQDRAR | 360 |
| QEVLQVFGSS KPDFDGLAHL KVVTMILLEV LRLYPPVIEL IRTIHKKTQL GKLSLPEGVE | 420 |
| VRLPTLLIHH DKELWGDDAN QFNPERFSEG VSKATKNRLS FFPFGAGPRI CIGQNFSMME | 480 |
| AKLALALILQ HFTFELSPSH AHAPSHRITL QPQYGVRIIL HRR | 523 |
| SEQ ID NO: 98 | |
| atggaagcat caagggctag ttgtgttgcg ctatgtgttg tttgggtgag catagtaatt | 60 |
| acattggcat ggagggtgct gaattgggtg tggttgaggc caaagaaact agaaagatgc | 120 |
| ttgagggagc aaggccttac aggcaattct tacaggcttt tgtttggaga caccaaggat | 180 |
| ctctcgaaga tgctggaaca aacacaatcc aaacccatca aactctccac ctcccatgat | 240 |
| atagcgccac gagtcacccc atttttccat cgaactgtga actctaatgg caagaattct | 300 |
| tttgtttgga tgggccctat accaagagtg cacatcatga atccagaaga tttgaaagat | 360 |
| gccttcaaca gacatgatga ttttcataag acagtaaaaa atcctatcat gaagtctcca | 420 |
| ccaccgggca ttgtaggcat tgaaggtgag caatgggcta aacacagaaa gattatcaac | 480 |
| ccagcattcc atttagagaa gctaaagggt atggtaccaa tattttacca aagttgtagc | 540 |
| gagatgatta acaaatggga gagcttggtg tccaaagaga gttcatgtga gttggatgtg | 600 |
| tggccttatc ttgaaaattt taccagcgat gtgatttccc gagctgcatt tggaagtagc | 660 |
| tatgaagagg gaaggaaaat atttcaacta ctaagagagg aagcaaaagt ttattcggta | 720 |
| gctctacgaa gtgtttacat tccaggatgg aggtttctac caaccaagca gaacaagaag | 780 |
| acgaaggaaa ttcacaatga aattaaaggc ttacttaagg gcattataaa taaaagggaa | 840 |
| gaggcgatga aggcagggga agccactaaa gatgacttac taggaatact tatggagtcc | 900 |
| aacttcaggg aaattcagga acatgggaac aacaaaaatg ctggaatgag tattgaagat | 960 |
| gtaattggag agtgtaagtt gttttacttt gctgggcaag agaccacttc ggtgttgctt | 1020 |
| gtttggacaa tgattttact aagccaaaat caggattggc aagctcgtgc aagagaagag | 1080 |
| gtcttgaaag tctttggaag caacatccca acctatgaag agctaagtca cctaaaagtt | 1140 |
| gtgaccatga ttttacttga agttcttcga ttatacccat cagtcgttgc gcttcctcga | 1200 |
| accactcaca agaaaacaca gcttggaaaa ttatcattac cagctggagt ggaagtctcc | 1260 |
| ttgcccatac tgcttgttca ccatgacaaa gagttgtggg gtgaggatgc aaatgagttc | 1320 |
| aagccagaga ggttttcaga gggagtttca aaggcaacaa agaacaaatt tacatactta | 1380 |
| cctttcggag ggggtccaag gatttgcatt ggacaaaact ttgccatggt ggaagctaaa | 1440 |
| ttggccttgg ccctgatttt acaacacttt gcctttgagc tttctccatc ctatgctcat | 1500 |
| gctccttctg cagttataac ccttcaacct caatttggtg ctcatatcat tttgcataaa | 1560 |
| cgttga | 1566 |
| SEQ ID NO: 99 | |
| atggaagctt ctagagcatc ttgtgttgct ttgtgtgttg tttgggtttc catcgttatt | 60 |
| actttggctt ggagagtttt gaattgggtc tggttaagac caaaaaagtt ggaaagatgc | 120 |
| ttgagagaac aaggtttgac tggtaactct tacagattgt tgttcggtga taccaaggac | 180 |
| ttgtctaaga tgttggaaca aactcaatcc aagcctatca agttgtctac ctctcatgat | 240 |
| attgctccaa gagttactcc attcttccat agaactgtta actccaacgg taagaactct | 300 |
| tttgtttgga tgggtccaat tccaagagtc catattatga accctgaaga tttgaaggac | 360 |
| gctttcaaca gacatgatga tttccataag accgtcaaga acccaattat gaagtctcca | 420 |
| ccaccaggta tagttggtat tgaaggtgaa caatgggcca aacatagaaa gattattaac | 480 |
| ccagccttcc acttggaaaa gttgaaaggt atggttccaa tcttctacca atcctgctct | 540 |
| gaaatgatta acaagtggga atccttggtt tccaaagaat cttcctgtga attggatgtc | 600 |
| tggccatatt tggaaaactt cacctccgat gttatttcca gagctgcttt tggttcttct | 660 |
| tacgaagaag gtagaaagat cttccaatta ttgagagaag aagccaaggt ttactccgtt | 720 |
| gctttgagat ctgtttacat tccaggttgg agattcttgc caactaagca aaacaaaaag | 780 |
| accaaagaaa tccacaacga aatcaagggt ttgttgaagg gtatcatcaa caagagagaa | 840 |
| gaagctatga aggctggtga agctacaaaa gatgatttgt tgggtatctt gatggaatcc | 900 |
| aacttcagag aaatccaaga acacggtaac aacaagaatg ccggtatgtc tattgaagat | 960 |
| gttatcggtg aatgcaagtt gttctacttt gctggtcaag aaactacctc cgttttgttg | 1020 |
| gtttggacca tgattttgtt gtcccaaaat caagattggc aagctagagc tagagaagaa | 1080 |
| gtcttgaaag ttttcggttc taacatccca acctacgaag aattgtctca cttgaaggtt | 1140 |
| gtcactatga tcttgttgga agtattgaga ttatacccat ccgttgttgc attgccaaga | 1200 |
| actactcata agaaaactca attgggtaaa ttgtccttgc cagctggtgt tgaagtttct | 1260 |
| ttgccaattt tgttagtcca ccacgacaaa gaattgtggg gtgaagatgc taatgaattc | 1320 |
| aagccagaaa gattctccga aggtgtttct aaagctacca agaacaagtt cacttacttg | 1380 |
| ccatttggtg gtggtccaag aatatgtatt ggtcaaaatt tcgctatggt cgaagctaaa | 1440 |
| ttggctttgg ctttgatctt gcaacatttc gctttcgaat tgtcaccatc ttatgctcat | 1500 |
| gctccatctg ctgttattac attgcaacca caatttggtg cccatatcat cttgcataag | 1560 |
| agataac | 1567 |
| SEQ ID NO: 100 | |
| MEASRASCVA LCVVWVSIVI TLAWRVLNWV WLRPKKLERC LREQGLTGNS YRLLFGDTKD | 60 |
| LSKMLEQTQS KPIKLSTSHD IAPRVTPFFH RTVNSNGKNS FVWMGPIPRV HIMNPEDLKD | 120 |
| AFNRHDDFHK TVKNPIMKSP PPGIVGIEGE QWAKHRKIIN PAFHLEKLKG MVPIFYQSCS | 180 |
| EMINKWESLV SKESSCELDV WPYLENFTSD VISRAAFGSS YEEGRKIFQL LREEAKVYSV | 240 |
| ALRSVYIPGW RFLPTKQNKK TKEIHNEIKG LLKGIINKRE EAMKAGEATK DDLLGILMES | 300 |
| NFREIQEHGN NKNAGMSIED VIGECKLFYF AGQETTSVLL VWTMILLSQN QDWQARAREE | 360 |
| VLKVFGSNIP TYEELSHLKV VTMILLEVLR LYPSVVALPR TTHKKTQLGK LSLPAGVEVS | 420 |
| LPILLVHHDK ELWGEDANEF KPERFSEGVS KATKNKFTYL PFGGGPRICI GQNFAMVEAK | 480 |
| LALALILQHF AFELSPSYAH APSAVITLQP QFGAHIILHK R | 521 |
| SEQ ID NO: 101 | |
| ASWVAVLSVV WVSMVIAWAW RVLNWVWLRP KKLEKCLREQ GLAGNSYRLL FGDTKDLSKM | 60 |
| LEQTQSKPIK LSTSHDIAPH VTPFFHQTVN SYGKNSFVWM GPIPRVHIMN PEDLKDTFNR | 120 |
| HDDFHKVVKN PIMKSLPQGI VGIEGEQWAK HRKIINPAFH LEKLKGMVPI FYRSCSEMIN | 180 |
| KWESLVSKES SCELDVWPYL ENFTSDVISR AAFGSSYEEG RKIFQLLREE AKIYTVAMRS | 240 |
| VYIPGWRFLP TKQNKKAKEI HNEIKGLLKG IINKREEAMK AGEATKDDLL GILMESNFRE | 300 |
| IQEHGNNKNA GMSIEDVIGE CKLFYFAGQE TTSVLLVWTM VLLSQNQDWQ ARAREEVLQV | 360 |
| FGSNIPTYEE LSQLKVVTMI LLEVLRLYPS VVALPRTTHK KTQLGKLSLP AGVEVSLPIL | 420 |
| LVHHDKELWG EDANEFKPER FSEGVSKATK NQFTYFPFGG GPRICIGQNF AMMEAKLALS | 480 |
| LILRHFALEL SPLYAHAPSV TITLQPQYGA HIILHKR | 517 |
| SEQ ID NO: 102 | |
| MEASRPSCVA LSVVLVSIVI AWAWRVLNWV WLRPNKLERC LREQGLIGNS YRLLFGDTKE | 60 |
| ISMMVEQAQS KPIKLSTTHD IAPRVIPFSH QIVYTYGRNS FVWMGPTPRV TIMNPEDLKD | 120 |
| AFNKSDEFQR AISNPIVKSI SQGLSSLEGE KWAKHRKIIN PAFHLEKLKG MLPTFYQSCS | 180 |
| EMINKWESLV FKEGSREMDV WPYLENLISD VISRAAFGSS YEEGRKIFQL LREEAKFYTI | 240 |
| AARSVYIPGW RFLPTKQNKR MKEIHKEVRG LLKGIINKRE DAIKAGEAAK GNLLGILMES | 300 |
| NFREIQEHGN NKNAGMSIED VIGECKLFYF AGQETTSVLL VWTLVLLSQN QDWQARAREE | 360 |
| VLQVFGTNIP TYDQLSHLKV VTMILLEVLR LYPAVVELPR TTYKKTQLGK FLLPAGVEVS | 420 |
| LHIMLAHHDK ELWGEDAKEF KPERFSEGVS KATKNQFTYF PFGAGPRICI GQNFAMLEAK | 480 |
| LALSLILQHF TFELSPSYAH APSVTITLHP QFGAHFILHK R | 521 |
| SEQ ID NO: 103 | |
| CVALSVVLVS IVIAWAWRVL NWVWLRPNKL ERCLREQGLT GNSYRLLFGD TKEISMMVEQ | 60 |
| AQSKPIKLST THDIAPRVIP FSHQIVYTYG RNSFVWMGPT PRVTIMNPED LKDAFNKSDE | 120 |
| FQRAISNPIV KSISQGLSSL EGEKWAKHRK IINPAFHLEK LKGMLPTFYQ SCSEMINKWE | 180 |
| SLVFKEGSRE MDVWPYLENL TSDVISRAAF GSSYEEGRKI FQLLREEAKF YTIAARSVYI | 240 |
| PGWRFLPTKQ NKRMKEIHKE VRGLLKGIIN KREDAIKAGE AAKGNLLGIL MESNFREIQE | 300 |
| HGNNKNAGMS IEDVIGECKL FYFAGQETTS VLLVWTLVLL SQNQDWQARA REEVLQVFGT | 360 |
| NIPTYDQLSH LKVVTMILLE VLRLYPAVVE LPRTTYKKTQ LGKFLLPAGV EVSLHIMLAH | 420 |
| HDKELWGEDA KEFKPERFSE GVSKATKNQF TYFPFGAGPR ICIGQNFAML EAKLALSLIL | 480 |
| QHFTFELSPS YAHAPSVTIT LHPQFGAHFI LHKR | 514 |
| SEQ ID NO: 104 | |
| MGPIPRVHIM NPEDLKDTFN RHDDFHKVVK NPIMKSLPQG IVGIEGDQWA KHRKIINPAF | 60 |
| HLEKLKGMVP IFYQSCSEMI NIWKSLVSKE SSCELDVWPY LENFTSDVIS RAAFGSSYEE | 120 |
| GRKIFQLLRE EAKVYTVAVR SVYIPGWRFL PTKQNKKTKE IHNEIKGLLK GIINKREEAM | 180 |
| KAGEATKDDL LGILMESNFR EIQEHGNNKN AGMSIEDVIG ECKLFYFAGQ ETTSVLLVWT | 240 |
| MVLLSQNQDW QARAREEVLQ VFGSNIPTYE ELSHLKVVTM ILLEVLRLYP SVVALPRTTH | 300 |
| KKTQLGKLSL PAGVEVSLPI LLVHHDKELW GEDANEFKPE RFSEGVSKAT KNQFTYFPFG | 360 |
| GGPRICIGQN FAMMEAKLAL SLILQHFTFE LSPQYSHAPS VTITLQPQYG AHLILHKR | 418 |
| SEQ ID NO: 105 | |
| atgggtttgt tcccattaga ggattcctac gcgctggtct ttgaaggact agcaataaca | 60 |
| ctggctttgt actatctact gtctttcatc tacaaaacat ctaaaaagac atgtacacct | 120 |
| cctaaagcat ctggtgaaat cattccaatt acaggaatca tattgaatct gctatctggc | 180 |
| tcaagtggtc tacctattat cttagcactt gcctctttag cagacagatg tggtcctatt | 240 |
| ttcaccatta ggctgggtat taggagagtg ctagtagtat caaattggga aatcgctaag | 300 |
| gagattttca ctacccacga tttgatagtt tctaatagac caaaatactt agccgctaag | 360 |
| attcttggtt tcaattatgt ttcattctct ttcgctccat acggcccata ttgggtcgga | 420 |
| atcagaaaga ttattgctac aaaactaatg tcttcttcca gacttcagaa gttgcaattt | 480 |
| gtaagagttt ttgaactaga aaactctatg aaatctatca gagaatcatg gaaggagaaa | 540 |
| aaggatgaag agggaaaggt attagttgag atgaaaaagt ggttctggga actgaatatg | 600 |
| aacatagtgt taaggacagt tgctggtaaa caatacactg gtacagttga tgatgccgat | 660 |
| gcaaagcgta tctccgagtt attcagagaa tggtttcact acactggcag atttgtcgtt | 720 |
| ggagacgctt ttccttttct aggttggttg gacctgggcg gatacaaaaa gacaatggaa | 780 |
| ttagttgcta gtagattgga ctcaatggtc agtaaatggt tagatgagca tcgtaaaaag | 840 |
| caagctaacg atgacaaaaa ggaggatatg gatttcatgg atatcatgat ctccatgaca | 900 |
| gaagcaaatt caccacttga aggatacggc actgatacta ttatcaagac cacatgtatg | 960 |
| actttgattg tttcaggagt tgatacaacc tcaatcgtac ttacttgggc cttatcactt | 1020 |
| ttgttaaaca acagagatac tttgaaaaag gcacaagagg aattagatat gtgcgtaggt | 1080 |
| aaaggaagac aagtcaacga gtctgatctt gttaacttga tatacttgga agcagtgctt | 1140 |
| aaagaggctt taagacttta cccagcagcg ttcttaggcg gaccaagagc attcttggaa | 1200 |
| gattgtactg ttgctggtta tagaattcca aagggcacct gcttgttgat taacatgtgg | 1260 |
| aaactgcata gagatccaaa catttggagt gatccttgcg aattcaagcc agaaagattt | 1320 |
| ttgacaccta atcaaaagga tgttgatgtg atcggtatgg atttcgaatt gataccattt | 1380 |
| ggtgccggca gaagatattg tccaggtact agattggctt tacagatgtt gcatatcgta | 1440 |
| ttagcgacat tgctgcaaaa cttcgaaatg tcaacaccaa acgatgcgcc agtcgatatg | 1500 |
| actgcttctg ttggcatgac aaatgccaaa gcatcacctt tagaagtctt gctatcacct | 1560 |
| cgtgttaaat ggtcctaa | 1578 |
| SEQ ID NO: 106 | |
| MGLFPLEDSY ALVFEGLAIT LALYYLLSFI YKTSKKTCTP PKASGEHPIT GHLNLLSGSS | 60 |
| GLPHLALASL ADRCGPIFTI RLGIRRVLVV SNWEIAKEIF TTHDLIVSNR PKYLAAKILG | 120 |
| FNYVSFSFAP YGPYWVGIRK IIATKLMSSS RLQKLQFVRV FELENSMKSI RESWKEKKDE | 180 |
| EGKVLVEMKK WFWELNMNIV LRTVAGKQYT GTVDDADAKR ISELFREWFH YTGRFVVGDA | 240 |
| FPFLGWLDLG GYKKTMELVA SRLDSMVSKW LDEHRKKQAN DDKKEDMDFM DIMISMTEAN | 300 |
| SPLEGYGTDT IIKTTCMTLI VSGVDTTSIV LTWALSLLLN NRDTLKKAQE ELDMCVGKGR | 360 |
| QVNESDLVNL IYLEAVLKEA LRLYPAAFLG GPRAFLEDCT VAGYRIPKGT CLLINMWKLH | 420 |
| RDPNIWSDPC EFKPERFLTP NQKDVDVIGM DFELIPFGAG RRYCPGTRLA LQMLHIVLAT | 480 |
| LLQNFEMSTP NDAPVDMTAS VGMTNAKASP LEVLLSPRVK WS | 522 |
| SEQ ID NO: 107 | |
| atgatacaag ttttaactcc aattctactc ttcctcatct tcttcgtttt ctggaaagtc | 60 |
| tacaaacatc aaaagactaa aatcaatcta ccaccaggtt ccttcggctg gccatttttg | 120 |
| ggtgaaacct tagccttact tagagcaggc tgggattctg agccagaaag attcgtaaga | 180 |
| gagcgtatca aaaagcatgg atctccactt gttttcaaga catcactatt tggagacaga | 240 |
| ttcgctgttc tttgcggtcc agctggtaat aagtttttgt tctgcaacga aaacaaatta | 300 |
| gtggcatctt ggtggccagt ccctgtaagg aagttgttcg gtaaaagttt actcacaata | 360 |
| agaggagatg aagcaaaatg gatgagaaaa atgctattgt cttacttggg tccagatgca | 420 |
| tttgccacac attatgccgt tactatggat gttgtaacac gtagacatat tgatgtccat | 480 |
| tggaggggca aggaggaagt taatgtattt caaacagtta agttgtacgc attcgaatta | 540 |
| gcttgtagat tattcatgaa cctagatgac ccaaaccaca tcgcgaaact cggtagtctt | 600 |
| ttcaacattt tcctcaaagg gatcatcgag cttcctatag acgttcctgg aactagattt | 660 |
| tactccagta aaaaggccgc agctgccatt agaattgaat tgaaaaagct cattaaagct | 720 |
| agaaaactcg aattgaagga gggtaaggcg tcttcttcac aggacttgct ttctcatcta | 780 |
| ttaacatcac ctgatgagaa tgggatgttc ttgacagaag aggaaatagt cgataacatt | 840 |
| ctacttttgt tattcgctgg tcacgatacc tctgcactat caataacact tttgatgaaa | 900 |
| accttaggtg aacacagtga tgtgtacgac aaggttttga aggaacaatt agaaatttcc | 960 |
| aaaacaaagg aggcttggga atcactaaag tgggaagata tccagaagat gaagtactca | 1020 |
| tggtcagtaa tctgtgaagt catgagattg aatcctcctg tcatagggac atacagagag | 1080 |
| gcgttggttg atatcgacta tgctggttac actatcccaa aaggatggaa gttgcattgg | 1140 |
| tcagctgttt ctactcaaag agacgaagcc aatttcgaag atgtaactag attcgatcca | 1200 |
| tccagatttg aaggggcagg ccctactcca ttcacatttg tgcctttcgg tggaggtcct | 1260 |
| agaatgtgtt taggcaaaga gtttgccagg ttagaagtgt tagcatttct ccacaacatt | 1320 |
| gttaccaact ttaagtggga tcttctaatc cctgatgaga agatcgaata tgatccaatg | 1380 |
| gctactccag ctaagggctt gccaattaga cttcatccac accaagtcta a | 1431 |
| SEQ ID NO: 108 | |
| MIQVLTPILL FLIFFVFWKV YKHQKTKINL PPGSFGWPFL GETLALLRAG WDSEPERFVR | 60 |
| ERIKKHGSPL VEKTSLFGDR FAVLCGPAGN KFLFCNENKL VASWWPVPVR KLFGKSLLTI | 120 |
| RGDEAKWMRK MLLSYLGPDA FATHYAVTMD VVTRRHIDVH WRGKEEVNVF QTVKLYAFEL | 180 |
| ACRLFMNLDD PNHIAKLGSL FNIFLKGIIE LPIDVPGTRF YSSKKAAAAI RIELKKLIKA | 240 |
| RKLELKEGKA SSSQDLLSHL LTSPDENGMF LTEEEIVDNI LLLLFAGHDT SALSITLLMK | 300 |
| TLGEHSDVYD KVLKEQLEIS KTKEAWESLK WEDIQKMKYS WSVICEVMRL NPPVIGTYRE | 360 |
| ALVDIDYAGY TIPKGWKLHW SAVSTQRDEA NFEDVTRFDP SRFEGAGPTP FTFVPFGGGP | 420 |
| RMCLGKEFAR LEVLAFLHNI VTNFKWDLLI PDEKIEYDPM ATPAKGLPIR LHPHQV | 476 |
| SEQ ID NO: 109 | |
| atggagtctt tagtggttca tacagtaaat gctatctggt gtattgtaat cgtcgggatt | 60 |
| ttctcagttg gttatcacgt ttacggtaga gctgtggtcg aacaatggag aatgagaaga | 120 |
| tcactgaagc tacaaggtgt taaaggccca ccaccatcca tcttcaatgg taacgtctca | 180 |
| gaaatgcaac gtatccaatc cgaagctaaa cactgctctg gcgataacat tatctcacat | 240 |
| gattattctt cttcattatt cccacacttc gatcactgga gaaaacagta cggcagaatc | 300 |
| tacacatact ctactggatt aaagcaacac ttgtacatca atcatccaga aatggtgaag | 360 |
| gagctatctc agactaacac attgaacttg ggtagaatca cccatataac caaaagattg | 420 |
| aatcctatct taggtaacgg aatcataacc tctaatggtc ctcattgggc ccatcagcgt | 480 |
| agaattatcg cctacgagtt tactcatgat aagatcaagg gtatggttgg tttgatggtt | 540 |
| gagtctgcta tgcctatgtt gaataagtgg gaggagatgg taaagagagg cggagaaatg | 600 |
| ggatgcgaca taagagttga tgaggacttg aaagatgttt cagcagatgt gattgcaaaa | 660 |
| gcctgtttcg gatcctcatt ttctaaaggt aaggctattt tctctatgat aagagatttg | 720 |
| cttacagcta tcacaaagag aagtgttcta ttcagattca acggattcac tgatatggtc | 780 |
| tttgggagta aaaagcatgg tgacgttgat atagacgctt tagaaatgga attggaatca | 840 |
| tccatttggg aaactgtcaa ggaacgLgaa atagaatgta aagatactca caaaaaggat | 906 |
| ctgatgcaat tgattttgga aggggcaatg cgttcatgtg acggtaacct ttgggataaa | 960 |
| tcagcatata gaagatttgt tgtagataat tgtaaatcta tctacttcgc agggcatgat | 1020 |
| agtacagctg tctcagtgtc atggtgtttg atgttactgg ccctaaaccc atcatggcaa | 1080 |
| gttaagatcc gtgatgaaat tctgtcttct tgcaaaaatg gtattccaga tgccgaaagt | 1140 |
| atcccaaacc ttaaaacagt gactatggtt attcaagaga caatgagatt ataccctcca | 1200 |
| gcaccaatcg tcgggagaga agcctctaaa gatatcagat tgggcgatct agttgttcct | 1260 |
| aaaggcgtct gtatatggac actaatacca gctttacaca gagatcctga gatttgggga | 1320 |
| ccagatgcaa acgatttcaa accagaaaga ttttctgaag gaatttcaaa ggcttgtaag | 1380 |
| tatcctcaaa gttacattcc atttggtctg ggtcctagaa catgcgttgg taaaaacttt | 1440 |
| ggcatgatgg aagtaaaggt tcttgtttcc ctgattgtct ccaagttctc tttcactcta | 1500 |
| tctcctacct accaacatag tcctagtcac aaacttttag tagaaccaca acatggggtg | 1560 |
| gtaattagag tggtttaa | 1578 |
| SEQ ID NO: 110 | |
| MESLVVHTVN AIWCIVIVGI FSVGYHVYGR AVVEQWRMRR SLKLQGVKGP PPSIFNGNVS | 60 |
| EMQRIQSEAK HCSGDNIISH DYSSSLEPHF DHWRKQYGRI YTYSTGLKQH LYINHPEMVK | 120 |
| ELSQTNTLNL GRITHITKRL NPILGNGIIT SNGPHWAHQR RIIAYEFTHD KIKGMVGLMV | 180 |
| ESAMPMLNKW EEMVKRGGEM GCDIRVDEDL KDVSADVIAK ACFGSSFSKG KAIFSMIRDL | 240 |
| LTAITKRSVL FRENGFTDMV FGSKKHGDVD IDALEMELES SIWETVKERE IECKDTHKKD | 300 |
| LMQLILEGAM RSCDGNLWDK SAYRRFVVDN GKSIYFAGED STAVSVSWCL MLLALNPSWQ | 360 |
| VKIRDEILSS CKNGIPDAES IPNLKTVTMV IQETMRLYPP APIVGREASK DIRLGDLVVP | 420 |
| KGVCIWTLIP ALHRDPEIWG PDANDFKPER FSEGISKACK YPQSYIPFGL GPRICVGKNE | 480 |
| GMMEVKVLVS LIVSKFSFTL SPTYQHSPSH KLLVEPQHGV VIRVV | 525 |
| SEQ ID NO: 111 | |
| atgtacttcc tactacaata cctcaacatc acaaccgttg gtgtctttgc cacattgttt | 60 |
| ctctcttatt gtttacttct ctggagaagt agagcgggta acaaaaagat tgccccagaa | 120 |
| gctgccgctg catggcctat tatcggccac ctccacttac ttgcaggtgg atcccatcaa | 180 |
| ctaccacata ttacattggg taacatggca gataagtacg gtcctgtatt cacaatcaga | 240 |
| ataggcttgc atagagctgt agttgtctca tcttgggaaa tggcaaagga atgttcaaca | 300 |
| gctaatgatc aagtgtcttc ttcaagacct gaactattag cttctaagtt gttgggttat | 360 |
| aactacgcca tgtttggttt ttcaccatac ggttcatact ggagagaaat gagaaagatc | 420 |
| atctctctcg aattactatc taattccaga ttggaactat tgaaagatgt tagagcctca | 480 |
| gaagttgtca catctattaa ggaactatac aaattgtggg cggaaaagaa gaatgagtca | 540 |
| ggattggttt ctgtcgagat gaaacaatgg ttcggagatt tgactttaaa cgtgatcttg | 600 |
| agaatggtgg ctggtaaaag atacttctcc gcgagtgacg cttcagaaaa caaacaggcc | 660 |
| cagcgttgta gaagagtctt cagagaattc ttccatctct ccggcttgtt tgtggttgct | 720 |
| gatgctatac cttttcttgg atggctcgat tggggaagac acgagaagac cttgaaaaag | 780 |
| accgccatag aaatggattc catcgcccag gagtggcttg aggaacatag acgtagaaaa | 840 |
| gattctggag atgataattc tacccaagat ttcatggacg ttatgcaatc tgtgctagat | 900 |
| ggcaaaaatc taggcggata cgatgctgat acgattaaca aggctacatg cttaactctt | 960 |
| atatcaggtg gcagtgatac tactgtagtt tctttgacat gggctcttag tcttgtgtta | 1020 |
| aacaatagag atactttgaa aaaggcacag gaagagttag acatccaagt cggtaaggaa | 1080 |
| agattggtta acgagcaaga catcagtaag ttagtttact tgcaagcaat agtaaaagag | 1140 |
| acactcagac tttatccacc aggtcctttg ggtggtttga gacaattcac tgaagattgt | 1200 |
| acactaggtg gctatcacgt ttcaaaagga actagattaa tcatgaactt atccaagatt | 1260 |
| caaaaagatc cacgtatttg gtctgatcct actgaattcc aaccagagag attccttacg | 1320 |
| actcataaag atgtcgatcc acgtggtaaa cactttgaat tcattccatt cggtgcagga | 1380 |
| agacgtgcat gtcctggtat cacattcgga ttacaagtac tacatctaac attggcatct | 1440 |
| ttcttgcatg cgtttgaatt ttcaacacca tcaaatgagc aggttaacat gagagaatca | 1500 |
| ttaggtctta cgaatatgaa atctacccca ttagaagttt tgatttctcc aagactatcc | 1560 |
| cttaattgct tcaaccttat gaaaatttga | 1590 |
| SEQ ID NO: 112 | |
| MYFLLQYLNI TTVGVFATLF LSYCLLLWRS RAGNKKIAPE AAAAWPIIGH LHLLAGGSHQ | 60 |
| LPHITLGNMA DKYGPVFTIR IGLHRAVVVS SWEMAKECST ANDQVSSSRP ELLASKLLGY | 120 |
| NYAMFGFSPY GSYWREMRKI ISLELLSNSR LELLKDVRAS EVVTSIKELY KLWAEKKNES | 180 |
| GLVSVEMKQW FGDLTLNVIL RMVAGKRYFS ASDASENKQA QRCRRVFREF FHLSGLFVVA | 240 |
| DAIPFLGWLD WGRHEKTLKK TAIEMDSIAQ EWLEEHRRRK DSGDDNSTQD FMDVMQSVLD | 300 |
| GKNLGGYDAD TINKATCLTL ISGGSDTTVV SLTWALSLVL NNRDTLKKAQ EELDIQVGKE | 360 |
| RLVNEQDISK LVYLQAIVKE TLRLYPPGPL GGLRQFTEDC TLGGYHVSKG TRLIMNLSKI | 420 |
| QKDPRIWSDP TEFQPERFLT THKDVDPRGK HFEFIPFGAG RRACPGITFG LQVLHLTLAS | 480 |
| FLHAFEFSTP SNEQVNMRES LGLTNMKSTP LEVLISPRLS SCSLYN | 526 |
| SEQ ID NO: 113 | |
| atggaaccta acttttactt gtcattacta ttgttgttcg tgaccttcat ttctttaagt | 60 |
| ctgtttttca tcttttacaa acaaaagtcc ccattgaatt tgccaccagg gaaaatgggt | 120 |
| taccctatca taggtgaaag tttagaattc ctatccacag gctggaaggg acatcctgaa | 180 |
| aagttcatat ttgatagaat gcgtaagtac agtagtgagt tattcaagac ttctattgta | 240 |
| ggcgaatcca cagttgtttg ctgtggggca gctagtaaca aattcctatt ctctaacgaa | 300 |
| aacaaactgg taactgcctg gtggccagat tctgttaaca aaatcttccc aacaacttca | 360 |
| ctggattcta atttgaagga ggaatctata aagatgagaa agttgctgcc acagttcttc | 420 |
| aaaccagaag cacttcaaag atacgtcggc gttatggatg taatcgcaca aagacatttt | 480 |
| gtcactcact gggacaacaa aaatgagatc acagtttatc cacttgctaa aagatacact | 540 |
| ttcttgcttg cgtgtagact gttcatgtct gttgaggatg aaaatcatgt ggcgaaattc | 600 |
| tcagacccat tccaactaat cgctgcaggc atcatttcac ttcctatcga tcttcctggt | 660 |
| actccattca acaaggccat aaaggcttca aatttcatta gaaaagagct gataaagatt | 720 |
| atcaaacaaa gacgtgttga tctggcagag ggtacagcat ctccaaccca ggatatcttg | 780 |
| tcacatatgc tattaacatc tgatgaaaac ggtaaatcta tgaacgagtt gaacattgcc | 840 |
| gacaagattc ttggactatt gataggaggc cacgatacag cttcagtagc ttgcacattt | 900 |
| ctagtgaagt acttaggaga attaccacat atctacgata aagtctacca agagcaaatg | 960 |
| gaaattgcca agtccaaacc tgctggggaa ttgttgaatt gggatgactt gaaaaagatg | 1020 |
| aagtattcat ggaatgtggc atgtgaggta atgagattgt caccaccttt acaaggtggt | 1080 |
| tttagagagg ctataactga ctttatgttt aacggtttct ctattccaaa agggtggaag | 1140 |
| ttatactggt ccgccaactc tacacacaaa aatgcagaat gtttcccaat gcctgagaaa | 1200 |
| ttcgatccta ccagatttga aggtaatggt ccagcgcctt atacatttgt accattcggt | 1260 |
| ggaggcccta gaatgtgtcc tggaaaggaa tacgctagat tagaaatctt ggttttcatg | 1320 |
| cataatctgg tcaaacgttt taagtgggaa aaggttattc cagacgaaaa gattattgtc | 1380 |
| gatccattcc caatcccagc taaagatctt ccaatccgtt tgtatcctca caaagcttaa | 1440 |
| SEQ ID NO: 114 | |
| MEPNFYLSLL LLFVTFISLS LFFIFYKQKS PLNLPPGKMG YPIIGESLEF LSTGWKGHPE | 60 |
| KFIFDRMRKY SSELEKTSIV GESTVVCCGA ASNKFLFSNE NKLVTAWWPD SVNKIFPTTS | 120 |
| LDSNLKEESI KMRKLLPQFF KPEALQRYVG VMDVIAQRHF VTHWDNKNEI TVYPLAKRYT | 180 |
| FLLACRLFMS VEDENHVAKF SDPFQLIAAG IISLPIDLPG TPFNKAIKAS NFIRKELIKI | 240 |
| IKQRRVDLAE GTASPTQDIL SHMLLTSDEN GKSMNELNIA DKILGLLIGG HDTASVACTF | 300 |
| LVKYLGELPH IYDKVYQEQM EIAKSKPAGE LLNWDDLKKM KYSWNVACEV MRLSPPLQGG | 360 |
| FREAITDFMF NGFSIPKGWK LYWSANSTHK NAECFPMPEK FDPTRFEGNG PAPYTFVPFG | 420 |
| GGPRMCPGKE YARLEILVFM HNLVKRFKWE KVIPDEKIIV DPFPIPAKDL PIRLYPHKA | 479 |
| SEQ ID NO: 115 | |
| atggcctctg ttactttggg ttcctggatc gtcgtccacc accataacca tcaccatcca | 60 |
| tcatctatcc taactaaatc tcgttcaaga tcctgtccta ttacactaac caaaccaatc | 120 |
| tcttttcgtt caaagagaac agtttcctct agtagttcta tcgtgtcctc tagtgtcgtc | 180 |
| actaaggaag acaatctgag acagtctgaa ccttcttcct ttgatttcat gtcatatatc | 240 |
| attactaagg cagaactagt gaataaggct cttgattcag cagttccatt aagagagcca | 300 |
| ttgaaaatcc atgaagcaat gagatactct cttctagctg gcgggaagag agtcagacct | 360 |
| gtactctgca tagcagcgtg cgaattagtt ggtggcgagg aatcaaccgc tatgcctgcc | 420 |
| gcttgtgctg tagaaatgat tcatacaatg tcactgatac acgatgattt gccatgtatg | 480 |
| gataacgatg atctgagaag gggtaagcca actaaccata aggttttcgg cgaagatgtt | 540 |
| gccgtcttag ctggtgatgc tttgttatct ttcgcgttcg aacatttggc atccgcaaca | 600 |
| tcaagtgatg ttgtgtcacc agtaagagta gttagagcag ttggagaact ggctaaagct | 660 |
| attggaactg agggtttagt tgcaggtcaa gtcgtcgata tctcttccga aggtcttgat | 720 |
| ttgaatgatg taggtcttga acatctcgaa ttcatccatc ttcacaagac agctgcactt | 780 |
| ttagaagcca gtgcggttct cggcgcaatt gttggcggag ggagtgatga cgaaattgag | 840 |
| agattgagga agtttgctag atgtatagga ttactgttcc aagtagtaga cgatatacta | 900 |
| gatgtgacaa agtcttccaa agagttggga aaaacagctg gtaaagattt gattgccgac | 960 |
| aaattgacct accctaagat tatggggcta gaaaaatcaa gagaatttgc cgagaaactc | 1020 |
| aatagagagg cgcgtgatca actgttgggt ttcgattctg ataaagttgc accactctta | 1080 |
| gccttagcca actacatcgc ttacagacaa aactaa | 1116 |
| SEQ ID NO: 116 | |
| MASVTLGSWI VVHHHNHHHP SSILTKSRSR SCPITLTKPI SFRSKRTVSS SSSIVSSSVV | 60 |
| TKEDNLRQSE PSSFDEMSYI ITKAELVNKA LDSAVPLREP LKIHEAMRYS LLAGGKRVRP | 120 |
| VLCIAACELV GGEESTAMPA ACAVEMIHTM SLIHDDLPCM DNDDLRRGKP TNHKVFGEDV | 180 |
| AVLAGDALLS FAFEHLASAT SSDVVSPVRV VRAVGELAKA IGTEGLVAGQ VVDISSEGLD | 240 |
| LNDVGLEHLE FIHLHKTAAL LEASAVLGAI VGGGSDDEIE RLRKFARCIG LLFQVVDDIL | 300 |
| DVIKSSKELG KTAGKDLIAD KLTYPKIMGL EKSREFAEKL NREARDQLLG FDSDKVAPLL | 360 |
| ALANYIAYRQ N | 371 |
| SEQ ID NO: 117 | |
| MATLLEHFQA MPFAIPIALA ALSWLFLFYI KVSFFSNKSA QAKLPPVPVV PGLPVIGNLL | 60 |
| QLKEKKPYQT FTRWAEEYGP IYSIRTGAST MVVLNTTQVA KEAMVTRYLS ISTRKLSNAL | 120 |
| KILTADKCMV AISDYNDFHK MIKRYILSNV LGPSAQKRHR SNRDTLRANV CSRLHSQVKN | 180 |
| SPREAVNFRR VFEWELFGIA LKQAFGKDIE KPIYVEELGT TLSRDEIFKV LVLDIMEGAI | 240 |
| EVDWRDFFPY LRWIPNTRME TKIQRLYFRR KAVMTALINE QKKRIASGEE INCYIDFLLK | 300 |
| EGKTLTMDQI SMLLWETVIE TADTTMVITE WAMYEVAKDS KRQDRLYQEI QKVCGSEMVT | 360 |
| EEYLSQLPYL NAVFHETLRK HSPAALVPLR YAHEDTQLGG YYIPAGTEIA INIYGCNMDK | 420 |
| HQWESPEEWK PERFLDPKFD PMDLYKTMAF GAGKRVCAGS LQAMLIACPT IGRLVQEFEW | 480 |
| KLRDGEEENV DTVGLITHKR YPMHAILKPR S | 511 |
| SEQ ID NO: 118 | |
| atggattcca aggccttgaa gaagccacat gcagtttgtg taccattccc tactcaaggt | 60 |
| cacataaacc cgttgctaaa attggcaaaa ctccttcact acagaggctt ccatataacc | 120 |
| tttgtcaaca cagagtacaa ccataagcgc ctgcttcaat cccgaggtac caactctctt | 180 |
| gatggcctgc cgtcctttag gttcaaatcg attcccgatg gcctccctcc tactgatgcc | 240 |
| aacgccaccc aagaagtaac atccctagtc gattccacta gaaaaaactg cttagcaccc | 300 |
| ttcaggtgcc ttctttcaga actcaacaca tccccaaatt cccctcctgt tactctcata | 360 |
| gtttctgatg gtggcatgtt cttcactctt gatgcagccc aagaacttgg tcttccagaa | 420 |
| gttatattca agccaacttg tgcttcttca ttcatgtgct accatcattg tgcccatgtc | 480 |
| attgaaaagg gtctcgttcc tcttaaagat gccagttatg tgacaaatgg ctatttggac | 540 |
| actgtcatag attggatacc aggcataagc agtatccgtt tgagggatat gccgagcttc | 600 |
| attagaacca cagacccaaa tgacatcttg ctgaaattta tgatggcaga gacggaaaga | 660 |
| acccaaagag cgtctgcaat cattattaac acatttgatg cgttggagca tgaagtttta | 720 |
| gatgcacttt caacttttct accacctatt tattccattg gacctctaca tctacaactc | 780 |
| agtcagatcc cagaggatga ggactcaagg tcgattgggt caaatctgtg gagagaagaa | 840 |
| ccagagtgtc ttgaatggct cgactctaaa gcaccaaatt ctgtggttta tgtaaacttc | 900 |
| ggaagtatca cagtcatgac aaacgagcag ctgattgagt ttgcttgggg tcttgcaaac | 960 |
| agtaacatga cctttttatg ggtgattagg cccgaccttg ttgctgggaa atcagctgtt | 1020 |
| gttcctccag agtttgcgga agtgaccaag ggaaggagtt tattggcgag ttggtgccct | 1080 |
| caagaacaag tgttgaacca cccggctgta ggaggattct tgacacacag tggatggaac | 1140 |
| tctacaattg aaagtatatg cggtggagtt cccatgatct gttggccctt ctttgctgat | 1200 |
| caacagacaa attgtaggtt ctgttgcaaa gaatggggta taggcctgga gatagagggt | 1260 |
| gatgttaaaa ggaataacat agaagacctt gtaagaaagt taatggaagg aaaggaaggc | 1320 |
| caagaaatga ggacgaaagc cttggaatgg aagaagttgg cagaggaggc tactactgct | 1380 |
| cccaatggtt catctttctt gaatttggac aagatggtta accaggtgct tcttcgtcct | 1440 |
| aagaattag | 1449 |
| SEQ ID NO: 119 | |
| MDSKALKKPH AVCVPFPTQG HINPLLKLAK LLHYRGFHIT FVNTEYNHKR LLQSRGTNSL | 60 |
| DGLPSFRFKS IPDGLPPTDA NATUVISLV DSTRKNCLAP FRCLLSELNT SPNSPPVTLI | 120 |
| VSDGGMFFTL DAAQELGLPE VIFKPTCASS FMCYHHCAHV IEKGLVPLKD ASYVTNGYLD | 180 |
| TVIDWIPGIS SIRLRDMPSF IRTTDPNDIL LKFMMAETER TQRASAIIIN TFDALEHEVL | 240 |
| DALSTFLPPI YSIGPLHLQL SQIPEDEDSR SIGSNLWREE PECLEWLDSK APNSVVYVNF | 300 |
| GSITVMTNEQ LIEFAWGLAN SNMTFLWVIR PDLVAGKSAV VPPEFAEVTK GRSLLASWCP | 360 |
| QEQVLNHPAV GGFLTHSGWN STIESICGGV PMICWPFFAD QQTNCRFCCK EWGIGLEIEG | 420 |
| DVKRNNIEDL VRKLMEGKEG QEMRTKALEW KKLAEEATTA PNGSSFLNLD KMVNQVLLRP | 480 |
| KN | 482 |

1. A recombinant host cell, comprising a recombinant gene encoding a polypeptide capable of glycosylating a steviol or a steviol glycoside at its C-19 carboxyl group and having at least 50% sequence identity to the amino acid sequence set forth in SEQ ID NOs:2 or 119, wherein the recombinant host cell is capable of producing the steviol glycoside or a steviol glycoside composition.

2. The recombinant host cell of claim 1, further comprising:
(a) a gene encoding a polypeptide capable of synthesizing geranylgeranyl pyrophosphate (GGPP) from farnesyl diphosphate (FPP) and isopentenyl diphosphate (IPP);
(b) a gene encoding a polypeptide capable of synthesizing ent-copalyl diphosphate from GGPP;
(c) a gene encoding an a polypeptide capable of synthesizing ent-kaurene from ent-copalyl pyrophosphate;
(d) a gene encoding a polypeptide capable of synthesizing ent-kaurenoic acid from ent-kaurene;
(e) a gene encoding a polypeptide capable of reducing cytochrome P450 complex;
(f) a gene encoding a polypeptide capable of synthesizing steviol from ent-kaurenoic acid;
(g) a gene encoding a polypeptide capable of glycosylating the steviol or the steviol glycoside at its C-13 hydroxyl group;
(h) a gene encoding a polypeptide capable of beta 1,3 glycosylation of the C3′ of the 13-O-glucose, 19-O-glucose, or both 13-O-glucose and 19-O-glucose of a steviol glycoside;
(i) a gene encoding a second polypeptide capable of glycosylating the steviol or the steviol glycoside at its C-19 carboxyl group;
(j) a gene encoding a polypeptide capable of beta 1,2 glycosylation of the C2′ of the 13-O-glucose, 19-O-glucose, or both 13-O-glucose and 19-O-glucose of a steviol glycoside; and/or
(k) a gene encoding a bifunctional polypeptide capable of synthesizing ent-copalyl diphosphate from GGPP and synthesizing ent-kaurene from ent-copalyl pyrophosphate.
wherein at least one of the genes is a recombinant gene.

3. The recombinant host cell of claim 2, wherein:
(a) the polypeptide capable of synthesizing GGPP comprises a polypeptide having at least 70% sequence identity to the amino acid sequence set forth in any one of SEQ ID NOs:20, 22, 24, 26, 28, 30, 32, or 116;
(b) the polypeptide capable of synthesizing ent-copalyl diphosphate comprises a polypeptide having at least 70% sequence identity to the amino acid sequence set forth in any one of SEQ ID NOs:34, 36, 38, 40, or 42;
(c) the polypeptide capable of synthesizing ent-kaurene comprises a polypeptide having at least 70% sequence identity to the amino acid sequence set forth in any one of SEQ ID NOs:44, 46, 48, 50, or 52;
(d) the polypeptide capable of synthesizing ent-kaurenoic acid comprises a polypeptide having at least 70% sequence identity to the amino acid sequence set forth in any one of SEQ ID NOs:60, 62, 66, 68, 70, 72, 74, 76, or 117;
(e) the polypeptide capable of reducing cytochrome P450 complex comprises a polypeptide having at least 70% sequence identity to the amino acid sequence set forth in any one of SEQ ID NOs:78, 80, 82, 84, 86, 88, 90, or 92;
(f) the polypeptide capable of synthesizing steviol comprises a polypeptide having at least 70% sequence identity to the amino acid sequence set forth in any one of SEQ ID NOs:94, 97, 100, 101, 102, 103, 104, 106, 108, 110, 112, or 114;
(g) the polypeptide capable of glycosylating the steviol or the steviol glycoside at its C-13 hydroxyl group thereof comprises a polypeptide having at least 55% sequence identity to the amino acid sequence set forth in SEQ ID NO:7;
(h) the polypeptide capable of beta 1,3 glycosylation of the C3′ of the 13-O-glucose, 19-O-glucose, or both 13-O-glucose and 19-O-glucose of a steviol glycoside comprises a polypeptide having at least 50% sequence identity to the amino acid sequence set forth in SEQ ID NO:9;
(i) the second polypeptide capable of glycosylating the steviol or the steviol glycoside at its C-19 carboxyl group thereof comprises a polypeptide having at least 55% sequence identity to the amino acid sequence set forth in SEQ ID NO:4;
(j) the polypeptide capable of beta 1,2 glycosylation of the C2′ of the 13-O-glucose, 19-O-glucose, or both 13-O-glucose and 19-O-glucose of a steviol glycoside comprises a polypeptide having at least 80% sequence identity to the amino acid sequence set forth in SEQ ID NOs:11 or 13; or a polypeptide having at least 65% sequence identity to the amino acid sequence set forth in SEQ ID NO:16; and
(k) the bifunctional polypeptide capable of synthesizing ent-copalyl diphosphate from GGPP and synthesizing ent-kaurene from ent-copalyl pyrophosphate comprises a polypeptide having at least 50% sequence identity to the amino acid sequence set forth in any one of SEQ ID NOs:54, 56, or 58.

4. The recombinant host cell of claim 1, wherein the recombinant host cell is a plant cell, a mammalian cell, an insect cell, a fungal cell, an algal cell, an archeal cell, or a bacterial cell.

5. The recombinant host cell of claim 4, wherein the bacterial cell is an Escherichia cell, a Lactobacillus cell, a Lactococcus cell, a Cornebacterium cell, an Acetobacter cell, an Acinetobacter cell, or a Pseudomonas cell.

6. The recombinant host cell of claim 4, wherein the fungal cell comprises a yeast cell.

7. The recombinant host cell of claim 6, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

8. The recombinant host cell of claim 6, wherein
the yeast cell is a Saccharomycete.

9. The recombinant host cell of claim 6, wherein the yeast cell is a cell from the Saccharomyces cerevisiae species.

10. A method of producing a steviol glycoside or a steviol glycoside composition, comprising growing the recombinant host cell of claim 1, in a cell culture medium, under conditions in which the genes are expressed, wherein the steviol glycoside or the steviol glycoside composition is produced by the recombinant host cell.

11. A method for producing a steviol glycoside or a steviol glycoside composition, comprising whole-cell bioconversion of a plant-derived or synthetic steviol and/or steviol glycosides in a cell culture medium using a recombinant polypeptide capable of glycosylating the steviol or the steviol glycoside at its C-19 carboxyl group and having at least 50% sequence identity to the amino acid sequence set forth in SEQ ID NOs:2 or 119 produced by the recombinant host cell of claim 1, and synthesizing the steviol glycoside thereby.

12. The method of claim 10, further comprising isolating the produced steviol glycoside or the steviol glycoside composition.

13. The method of claim 12, wherein the isolating step comprises separating a liquid phase of the cell culture from a solid phase of the cell culture to obtain a supernatant comprising the produced steviol glycoside or the steviol glycoside composition, and:
(a) contacting the supernatant with one or more adsorbent resins in order to obtain at least a portion of the produced steviol glycoside or the steviol glycoside composition; or
(b) contacting the supernatant with one or more ion exchange or reversed-phase chromatography columns in order to obtain at least a portion of the produced steviol glycoside or the steviol glycoside composition; or
(c) crystallizing or extracting the produced steviol glycoside or the steviol glycoside composition;
thereby isolating the produced steviol glycoside or the steviol glycoside composition.

14-15. (canceled)

16. The method of claim 10, further comprising recovering the steviol glycoside alone or as a composition comprising the steviol glycoside.

17. The method of claim 16, wherein the recovered composition is enriched for the one or more steviol glycosides or glycosides of the steviol precursor relative to a steviol glycoside composition of Stevia plant and has a reduced level of Stevia plant-derived components relative to a steviol glycoside composition obtained from a plant-derived Stevia extract.

18-19. (canceled)

20. The method of claim 10, wherein the steviol glycoside is produced in a permeabilized recombinant host cell which has been transformed with the gene encoding the polypeptide capable of glycosylating the steviol or the steviol glycoside at its C-19 carboxyl group and having at least 50% sequence identity to the amino acid sequence set forth in SEQ ID NOs:2 or 119.

21-26. (canceled)

27. An in vitro method for producing steviol-19-O-glucoside (19-SMG), comprising adding a recombinant polypeptide capable of glycosylating a steviol or a steviol glycoside at its C-19 carboxyl group and having at least 50% sequence identity to the amino acid sequence set forth in SEQ ID NOs:2 or 119 and a plant-derived or synthetic steviol to a reaction mixture; and synthesizing 19-SMG thereby.

28. The method of claim 27, wherein the reaction mixture comprises:
(a) 19-SMG produced in the reaction mixture;
(b) the recombinant polypeptide;
(c) glucose, fructose, and/or sucrose, uridine diphosphate (UDP)-glucose, UDP-rhamnose, UDP-xylose, and/or N-acetyl-glucosamine; and
(d) reaction buffer and/or salts.

29. The method of claim 10, wherein the steviol glycoside comprises steviol-13-O-glucoside (13-SMG), steviol-1,2-bioside, steviol-1,3-bioside, steviol-19-O-glucoside (19-SMG), stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, or isomers thereof.

30. A cell culture, comprising the recombinant host cell of claim 1, the cell culture further comprising:
(a) the one or more steviol glycosides produced by the recombinant host cell;
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, YNB, and/or amino acids;
wherein the one or more steviol glycosides are present at a concentration of at least 1 mg/L of the cell culture;
wherein the cell culture is enriched for the one or more steviol glycosides relative to a steviol glycoside composition from a Stevia plant and has a reduced level of Stevia plant-derived components relative to a plant-derived Stevia extract.

31-33. (canceled)

34. A cell lysate from the recombinant host cell of claim 1 grown in the cell culture, comprising:
(a) the one or more steviol glycosides produced by the recombinant host cell;
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, yeast nitrogen base, YNB, and/or amino acids;
wherein the one or more steviol glycosides or glycosides of the steviol precursor produced by the recombinant host cell is present at a concentration of at least 1 mg/L of the cell culture.

35. A steviol glycoside produced by the recombinant host cell of claim 1.

36. (canceled)

37. A sweetener composition, comprising the steviol glycoside of claim 35.

38. A food product comprising the sweetener composition of claim 37.

39. A beverage or a beverage concentrate, comprising the sweetener composition of claim 38.

40. A composition of steviol glycosides produced by the recombinant host of claim 1, wherein the relative levels of steviol glycosides in the composition correspond to the relative levels of steviol glycosides in the recombinant host.

41. The composition of claim 40, further comprising an increased level of 19-SMG relative to a composition of steviol glycosides produced by a corresponding recombinant host cell lacking the gene encoding a polypeptide capable of glycosylating the steviol or the steviol glycoside at its C-19 carboxyl group and having at least 50% sequence identity to the amino acid sequence set forth in SEQ ID NOs:2 or 119.

42. A method for transferring a sugar moiety to a C-19 carboxyl group of a steviol or a steviol glycoside, comprising contacting the steviol or the steviol glycoside with a recombinant polypeptide capable of glycosylating the steviol or the steviol glycoside at its C-19 carboxyl group and having at least 50% sequence identity to the amino acid sequence set forth in SEQ ID NOs:2 or 119 and a UDP-sugar under suitable conditions for the transfer of the sugar moiety to the steviol glycoside;
wherein the steviol glycoside comprises a steviol-13-O-glucoside (13-SMG), a steviol-1,2-bioside, a steviol-1,3-bioside, and/or a Rebaudioside B (RebB); and
wherein a steviol-19-O-glucoside (19-SMG), a rubusoside, a stevioside, a 1,3-stevioside (RebG), a Rebaudioside A (RebA) and/or a steviol glycoside composition thereof is produced upon transfer of the sugar moiety.
